(19) United States
(12) Patent Application Publication
(10) Pub. No.: US 2021/0040558 A1
(43) Pub. Date: Feb. 11, 2021

(54) METHOD TO ISOLATE TCR GENES

(71) Applicant: NEOGENE THERAPEUTICS B.V., Amsterdam (NL)

(72) Inventors: Antonius Nicolaas Maria Schumacher, Amsterdam (NL); Carsten Linnemann, Amsterdam (NL); Thomas Kuilman, Amstelveen (NL); Gavin M. Bendle, Amsterdam (NL); Jules F.C. Gadiot, Heiloo (NL); Jeroen W.J. van Heijst, Amstelveen (NL); Raquel Gomez-Eerland, Zaandam (NL); Deborah Sophie Schrikkema, Amsterdam (NL)

(21) Appl. No.: 16/927,661

(22) Filed: Jul. 13, 2020

Related U.S. Application Data
(60) Provisional application No. 62/874,125, filed on Jul. 15, 2019.
(60) Provisional application No. 62/975,924, filed on Feb. 13, 2020.
(60) Provisional application No. 63/024,341, filed on May 13, 2020.
(60) Provisional application No. 63/034,157, filed on Jun. 3, 2020.
(60) Provisional application No. 63/039,346, filed on Jun. 15, 2020.

(51) Int. Cl. C12Q 1/6881; C12N 15/10; A61K 39/00; A61P 35/00; A61P 37/02; C12N 5/0783

(52) U.S. Cl. CPC C12Q 1/6881; C12N 15/1058; C12N 5/0636; A61P 35/00; A61P 37/02; A61K 39/0011

(57) ABSTRACT

The present disclosure provides methods to recover repertoires of T cell receptors (TCRs). In some embodiments, TCR repertoires are recovered from non-viable samples. In some embodiments, libraries of TCRαβ pairs are created. In some embodiments, the methods disclosed are used for cancer immunotherapy or diagnostic purposes.

### CROSS REFERENCE TO RELATED APPLICATIONS

This application claims priority to U.S. Provisional Patent Application No. 62/874125, filed Jul. 15, 2019, U.S. Provisional Patent Application No. 62/975924, filed Feb. 13, 2020, U.S. Provisional Patent Application No. 63/024341, filed May 13, 2020, U.S. Provisional Patent Application No. 63/034157, filed Jun. 3, 2020, and U.S. Provisional Patent Application No. 63/039346, filed Jun. 15, 2020, which are incorporated herein by reference in their entireties.

### REFERENCE TO SEQUENCE LISTING

The present application is being filed along with a Sequence Listing in electronic format. The Sequence Listing is provided as a file entitled “NTBV001ASEQLIST.txt”, which was created on Jul. 13, 2020, and is 21,764 bytes in size. The information in the electronic Sequence Listing is hereby incorporated by reference in its entirety.

### Field

The present technology generally relates to the isolation of T cell receptor (TCR) gene sequences to recover a repertoire of TCRs. Compositions and methods of treatment are also provided.

### Description of the Related Art

PCR-based techniques and TCR bulk chain sequencing have been employed to detect TCRαβ pairs (Kobayashi et al. Nat Med 2013, Linnemann et al. Nat Med. 2013, Tran et al. Science 2015; Fran et al. N Engl J Med 2016; Tsuji et al Cancer Immunol Res 2018).

### SUMMARY

Described herein, in some embodiments, are methods of identifying nucleotide sequences encoding T cell receptor α (TCRα)- and TCRβ-chains from a combinatorial library of nucleic acids. The method comprises (I) providing a library comprising a plurality of variant nucleic acids encoding TCRα- and TCRβ-chains, (II) introducing the library into a population of cells able to express TCRα- and TCRβ-chains encoded by a member of the plurality of variant nucleic acids, (III) selecting a subpopulation of the population of cells based on an expression of a marker above a threshold level in response to antigen, wherein the subpopulation comprises a plurality of cells, (IV) isolating a subset of the plurality of variant nucleic acids from the subpopulation, (V) determining nucleotide sequences of the variant nucleic acids, and (VI) identifying at least one variant nucleotide sequence based on an enrichment of the nucleotide sequences within the subset relative to a control.

Other some embodiments relate to methods to recover a repertoire of T cell receptors (TCRs) from diverse T cell populations, the methods comprising: I) determining TCR-α andβ nucleotide or amino acid sequences within a subject's sample; II) selecting one or more subsets of TCRα- and β-chain sequences from the total repertoire; III) creating a TCR repertoire by combinatorial pairing of selected TCRα- and β-chain sequences creating a library of TCRαβ pairs; and IV) identifying at least one TCRαβ pair with desired features from the created TCR repertoire.

In some embodiments, the one or more subsets of TCRα- and β-chain sequences from the total repertoire is selected based on at least one criterion: a) on frequency within the T cell population; b) on relative enrichment compared to a second T cell population; c) on relative difference of DNA and RNA copy numbers of a given TCR chain, d) on biological properties of the TCR chain, wherein the properties are selected from at least one of: (predicted) antigen-specificity, sequence motif(s), (predicted) HILA-restriction, affinity, co-receptor dependency or parental T cell lineage (e.g. CD4 or CD8 T cell); e) on spatial patterns of gene expression, wherein spatial gene expression patterns are derived from at least one of: originating region in the tissue or co-expression patterns of other genes; on co-occurrence or occurrence at a similar frequency in multiple samples, for example occurrence in multiple tumor lesions; g) selection into multiple groups to separately recover specific parts of the TCR repertoire; h) on a combination of multiple criteria as defined in the different embodiments. In some embodiments, selection based on frequency within the T cell population is based upon data of the frequency of TCR sequences, which is used to create a separate rank order for TCRα- and β-chains or a combined rank order for TCRα- and β-chains. In some embodiments, the methods further comprise determining a frequency threshold that is defined based on the desired depth for TCR repertoire recovery and used to select collections of TCRα- and β-chains based on frequency. In some embodiments, determining TCR-α and β sequences is achieved by at least one of: a) multiplex PCR; b) TCR-sequence recovery by target enrichment; c) TCR-sequence recovery by 5′RACE and PER; d) TCR-sequence recovery by spatial sequencing; or e) TCR-sequence recovery from RNA-seq data. In sonic embodiments, a recovered TCR-chain sequence is defined as the CDR3 nucleotide sequence together with sufficient 5′- and 3′-nucleotide sequence information to select at least one TCR V- and at least one TCR J-segment family based on nucleotide sequence alignment to assemble a complete TCR chain sequence. In some embodiments, nucleotide sequence alignment is based on 65% sequence identity, 70% sequence identity, 75% sequence identity, 80% sequence identity, 85% sequence identity, 90% sequence identity, 95% sequence identity, 96% sequence identity, 97% sequence identity, 98% sequence identity, 99% sequence identity, 100% sequence identity, and any number or range in between. In some embodiments, optimal sequence alignment is based on minimizing a distance measure according to read mapping algorithms known to the skilled artisan. In some embodiments, the best alignment is sought.

In some embodiments, step III is achieved by at least one of the following: i) TCR chain sequences are used to synthesize separate libraries of TCRα- and β-chain DNA fragments which are subsequently linked into one DNA or RNA fragment (optionally, in which exactly one TCRα- and one β-chain are linked), ii) combinations of TCRα- and β-chains are generated by directly synthesizing DNA or RNA fragments in which exactly one TCRα- and one β-chain are linked, or iii) combinations of TCRα- and β-chains are created intracellularly by modification of a pool of cells with separate collections of TCRα- and β-genes encoded in form of DNA- or RNA vectors in such a way that cells will express one TCRα- and one β-chain; (iv) combinations of TCRα- and β-chains are linked in a single-chain TCR construct containing both TCR chain fragments as well as CD3ζ or CD3ϵ signaling domains alone or in combination with CD28 signaling domains. In some embodiments, directly synthesizing DNA or RNA fragments in which exactly one TCRα- and one β-chain are linked eliminates the need to perform in silico random pairing and synthesizing all resulting combinations as one DNA fragment. In some embodiments, step IV is achieved by at least one of the following: i) a pool of reporter cells or T cells modified with the library of generated TCRαβ pairs is stimulated by antigen presenting cells presenting at least one antigen of interest and antigen-reactive reporter cells or cells are isolated based on at least one activation marker for TCR isolation; ii) a pool of reporter cells or cells modified with the library of generated TCRαβ pairs is labelled with a fluorescent dye suitable to trace cell proliferation, stimulated by antigen presenting cells expressing at least one antigen of interest, and antigen-reactive reporter cells or cells are isolated based on proliferation for TCR isolation; iii) a pool of reporter cells or T cells modified with the library of generated TCRαβ pairs is divided into at least two samples; samples are stimulated by antigen presenting cells expressing at least one antigen of interest or not, respectively; after stimulation, both reporter cell or cell populations are incubated for a period of time and subsequently both reporter cell or T cell populations are analyzed by TCR isolation; comparison of TCRαβ pairs obtained from both samples will identify TCR genes with higher abundance in the sample exposed to at least one antigen; iv) a pool of reporter cells or T cells modified with the library of generated TCRαβ pairs is stimulated by antigen presenting cells presenting at least one antigen of interest and antigen-reactive reporter cells or T cells are isolated based on at least one reporter gene, such as NFAT-GFP or NFAT-YFP that reports on TCR triggering; v) a pool of reporter cells or T cells modified with the library of generated TCRαβ pairs is stimulated by antigen presenting cells presenting at least one antigen of interest, and antigen-reactive reporter cells or T cells are isolated for TCR isolation based on selection of antigen-specific reporter cells or I cells based on acquired antibiotic resistance upon TCR signaling, for example by use of a NFAT-puromycin transgene; vi) a pool of reporter cells or cells modified with the library of generated TCRαβ pairs is exposed to one or multiple MHC complexes that carry an antigen of interest; reporter cells or cells bind to an MHC complex are isolated for TCR isolation; vii) a pool of reporter cells or T cells modified with the library of generated TCRαβ pairs is stimulated by antigen presenting cells expressing at least one antigen of interest; subsequently, TCRαβ pairs of interest are identified using single-cell based droplet PCR or microfluidic approaches to combine TCR isolation with the detection of transcript levels for at least one activation marker; thereby, single reporter cells or T cells within the pool of reporter cells or T cells in which TCRαβ transcripts are co-expressed with increased levels of activation marker are detected. In some embodiments, TCR isolation in steps i)-vi) be achieved by (i) DNA or RNA isolation from bulk antigen-reactive reporter cells or T cells to generate TCRαβ specific PCR product which is analyzed by DNA-sequencing to determine TCRαβ gene sequences of antigen-reactive reporter cells or T cells or (ii) single-cell based droplet PCR or microfluidic approaches to analyze the TCRαβ gene sequences expressed in analyzed single T cells. In some embodiments, the reporter cells are T cells. In some embodiments, reporter cells monitor TCR engagement. In some embodiments, the pool of reporter cells can be TCR a/b-null.

In some embodiments, the subject's sample comprises non-viable starting material. In some embodiments, a defined part of the identified TCR repertoire is recovered. In some embodiments, defined or selective recovery is performed by selecting only part or all detected TCR chains based on criteria including, but not limited to a) on frequency within the cell population, h) on relative enrichment compared to a second T cell population, c) on relative difference of DNA and RNA copy numbers of a given TCR chain, d) on biological properties of the TCR chain, wherein the properties are selected from at least one of: (predicted) antigen-specificity, (predicted) HLA-restriction, affinity, co-receptor dependency, parental T cell lineage (e.g. CD4 or CD8 T cell) or TCR sequence motifs, e) on spatial patterns of gene expression, wherein spatial gene expression patterns are derived from at least one of: originating region in the tissue or co-expression patterns of other genes, f) on co-occurrence or occurrence at a similar frequency in multiple samples, for example occurrence in multiple tumor lesions, g) selection into multiple groups to separately recover specific parts of the TCR repertoire, on a combination of multiple criteria as defined in the different embodiments. In some embodiments, selective recovery of TCR sequences refers to recovery of TCR sequences that contain certain V gene segments.

In some embodiments, antigen-specific TCR sequences are recovered. In some embodiments, therapeutic TCR sequences are recovered. In some embodiments, tumor-reactive TCR sequences are recovered. In some embodiments, neo-antigen specific TCR sequences are recovered. In some embodiments, the methods described herein further comprise the step of administering T cells expressing the neo-antigen specific TCR sequences as a cancer therapy. In some embodiments, the methods described herein are for a diagnostic. In some embodiments, the diagnostic is to recover TCR repertoires from pathological sites of infection or autoimmunity. In some embodiments, the methods described herein are for the recovery of BCR/antibody repertoires. In sonic embodiments, the methods described herein further comprise isolating nucleic acids from a subject that comprise the TCR-α and β nucleotide sequences. In some embodiments, the activation marker is a CD4 or CD8 T cell activation marker. Any CD4 or CD8 T cell activation marker can be used. In some embodiments of the methods described herein, the activation marker is selected from the group consisting of: CD69, CD137, IFN-γ, IL-2, TNF-α, GM-CSF. In some embodiments, in the methods described herein, DNA and RNA is isolated from a T cell population that is a mixture of different cell types or part of a tissue sample (such as blood or tumor tissue). In some embodiments, the subject's sample comprises cells isolated from a body fluid. In some embodiments, the cells are tumor-specific T cells. In some embodiments, the body fluid is selected from the group consisting of blood, urine, serum, serosal fluid, plasma, lymph, cerebrospinal fluid, saliva, sputum, mucosal secretion, vaginal fluid, ascites fluid, pleural fluid, pericardial fluid, peritoneal fluid, and abdominal fluid. In some embodiments, the methods described herein further comprise using the TCRαβ chain sequences to treat a subject suffering from cancer, an immunological disorder, an autoimmune disease, or an infectious disease. In some embodiments, step IV of the methods described herein is achieved by at least one of the following: (i) identification or selection based on at least one activation marker; (ii) identification or selection based on proliferation in response to antigen; (iii) identification or selection based on identification of TCR genes of higher abundance in antigen-stimulated cells as compared to unstimulated cells; (iv) identification or selection based on reporter gene activation by TCR triggering; (v) identification or selection based on selective survival, including but not limited to acquired antibiotic-resistance, upon TCR signaling; (vi) identification or selection based on binding to one or more MHC complexes; (vii) identification or selection using single-cell based droplet PCR or microfluidics; or any combination thereof In some embodiments, step (vii) further comprises determination of co-expression of activation-associated genes.

Disclosed herein, in some embodiments, are methods of creating multiple T cell libraries, the methods comprising: (a) recovering a repertoire of T cell receptors (TCRs) according to the methods described herein; (b) selection of TCRα- and β-chain sequences from the total repertoire into multiple groups to separately recover specific parts of the TCR repertoire, wherein multiple T cell libraries are created that are of smaller complexity or that recover specific parts of the TCR repertoire. In some embodiments, selection of TCRα- and β-chain sequences is based on frequency range.

Described herein, in some embodiments, are methods of creating multiple T cell libraries, the methods comprising: (a) recovering a repertoire of T cell receptors (TCRs) according to the methods described herein; (b) selection of TCRα- and β-chain sequences from the total repertoire into multiple groups to separately recover specific parts of the TCR repertoire, wherein multiple T cell libraries are created that are of smaller complexity or that recover specific parts of the TCR repertoire.

In some embodiments, selection of TCRα- and β-chain sequences is based on frequency range.

Described herein, in some embodiments are methods to recover a repertoire of T cell receptors (TCRs) from diverse T cell populations, the methods comprising: I) determining TCR-α and β nucleotide or amino acid sequences within a subject's sample; II) selecting one or more of a subset of TCRα- and β-chain sequences from the total repertoire; III) creating a TCR repertoire by combinatorial pairing of selected TCRα- and β-chain sequences creating a library of TCRαβ pairs; and IV) identifying at least one TCRαβ pair with desired features from the created TCR repertoire.

In some embodiments, a method of identifying a nucleotide sequence from a combinatorial library of nucleic acids is provided. The method comprises providing a combinatorial library comprising a plurality of variant nucleic acids, each of the plurality of variant nucleic acids comprises a contiguous portion of at least 600 bp. The method further comprises introducing the library into a population of cells configured to express one or more polypeptides encoded by a member of the plurality of variant nucleic acids. The method further comprises selecting a subpopulation of the population of cells based on at least one functional property dependent on the contiguous portion of at least 600 bp, wherein the subpopulation comprises a plurality of cells. The method further comprises isolating a subset of the plurality of variant nucleic acids from the subpopulation. The method further comprises determining nucleotide sequences of the contiguous portion of individual members of the subset. The method further comprises identifying the contiguous portion of at least 600 by based on the nucleotide sequences. In some embodiments, the method can also be one in which the contiguous portion of at least 600 bp is distributed throughout 600 basepairs.

In some embodiments, the method can include one or more of steps (1)-(7) described below. Step (1) Obtaining a sample. The sample can be tissues, blood, or body fluids from a patient suffering infectious diseases, autoimmune diseases, or cancers. The sample can be viable or non-viable. Step (2) Sequencing TCR-α and β chains in the sample. Step (3) Selecting and combinatorial pairing TCRα- and β-chain sequences to create a library of TCRαβ pairs. Step (4) introducing the library of TCRαβ pairs into a pool of reporter cells, for example, Jurkat reporter. T cells. Step (5) Stimulating the reporter cells that are modified with the library of TCRαβ pairs with antigen presenting cells presenting at least one antigen of interest. The at least one antigen of interest can be autologous or allogeneic. Step (6) Determining TCRαβ pairs specific to the at least one antigen of interest. Step (7) Introducing the TCRαβ pairs into cells and selecting cells containing the TCRαβ pairs. In some embodiments, the method can involve one or more of the steps (1)-(7) described above. Any of the steps can be omitted, repeated, or substituted by other embodiments provided herein, as appropriate. Additional intervening steps can also be added.

Some embodiments relate to a nucleotide library comprises the repertoire of T cell receptors recovered according to any one of the above embodiments. In some embodiments, a nucleotide construct comprising the nucleotide sequence identified according to any one of the above embodiments. In some embodiments, a cell comprises the nucleotide construct described herein.

In some embodiments, a method to recover a repertoire of T cell receptors (TCRs) from diverse T cell populations is provided. The method comprises determining TCR-α and β nucleotide or amino acid sequences within a subject's sample; selecting one or more subsets of TCRα- and β-chain sequences from the total repertoire; creating a TCR repertoire by combinatorial pairing of selected TCRα- and β-chain sequences creating a library of TCRαβ pairs; and identifying at least one TCRαβ pair with desired features from the created TCR repertoire. In some embodiments, a method of creating multiple T cell libraries is provided. The method comprises recovering a repertoire of T cell receptors (TCRs) according to the method of above, selection of TCRα- and β-chain sequences from the total repertoire into multiple groups to separately recover specific parts of the TCR repertoire, wherein multiple T cell libraries are created that are of smaller complexity or that recover specific parts of the TCR repertoire.

In some embodiments, a method of identifying a nucleotide sequence from a combinatorial library of nucleic acids is provided. The method comprises providing a combinatorial library comprising a plurality of variant nucleic acids, each of the plurality of variant nucleic acids comprising a contiguous portion of at least 600 bp, wherein the contiguous portion comprises a combination of two or more variant nucleotide subsequences, wherein a first variant nucleotide subsequence of the two or more variant nucleotide subsequences defines a first end of the contiguous portion and a second variant nucleotide subsequence of the two or more variant nucleotide subsequences defines a second end of the contiguous portion opposite the first end; introducing the library into a population of cells configured to express one or more polypeptides encoded by a member of the plurality of variant nucleic acids; selecting a subpopulation of the population of cells based on at least one functional property dependent on the combination of the two or more variant nucleotide subsequences, wherein the subpopulation comprises a plurality of cells; isolating a subset of the plurality of variant nucleic acids from the subpopulation.; determining nucleotide sequences of the contiguous portion of individual members of the subset; and identifying at least one combination of the two or more variant nucleotide subsequences based on the nucleotide sequences.

In some embodiments, a method of identifying nucleotide sequences encoding T cell receptor α (TCRα)- and TCRβ-chains from a combinatorial library of nucleic acids is provided. The method comprises: providing a library comprising a plurality of variant nucleic acids, each of the plurality of variant nucleic acids comprising a contiguous portion of at least 600 bp, wherein the contiguous portion comprises: a combination of: a first variant nucleotide subsequence encoding a TCRα variant amino acid sequence and defining a first end of the contiguous portion, and a second variant nucleotide subsequence encoding a TCRβ variant amino acid sequence and defining a second end of the contiguous portion opposite the first end. The method can further comprise introducing the library into a population of immortalized T cells configured to express TCRα- and TCRβ-chains encoded by a member of the plurality of variant nucleic acids. The method can further comprise selecting a subpopulation of the population of immortalized T cells based on an expression of a T cell activation marker above a threshold level in response to contacting the immortalized T cells with immortalized B cells expressing an antigen, wherein the subpopulation comprises a plurality of T cells. The method can further comprise isolating a subset of the plurality of variant nucleic acids from the subpopulation. The method can further comprise determining nucleotide sequences of the contiguous portion of individual members of the subset; and identifying at least one combination of the first and second variant nucleotide subsequences based on an enrichment of the at least one combination in the nucleotide sequences of the subset relative to a control.

In some embodiments, a method of identifying a nucleotide sequence encoding a chimeric antigen receptor (CAR) hinge domain, transmembrane domain, and/or an intracellular signaling domain from a combinatorial library of nucleic acids is provided. The method can comprise: providing a library comprising a plurality of variant nucleic acids, each of the plurality of variant nucleic acids comprising a contiguous portion of at least 600 bp, wherein the contiguous portion comprises a combination of two or more of: a first variant nucleotide subsequence encoding a CAR hinge domain; a second variant nucleotide subsequence encoding a CAR transmembrane domain; and a third variant nucleotide subsequence encoding a CAR intracellular signaling domain. One of the first, second or third variant nucleotide subsequences define a first end of the contiguous portion, and wherein another one of the first, second, or third variant nucleotide subsequences defines a second end of the contiguous portion opposite the first end. The method can further comprise introducing the library into a population of cells configured to express a CAR encoded by a member of the plurality of variant nucleic acids. The population of cells comprises a population of immortalized T cells or primary human T cells. The method can further comprise selecting a subpopulation of the population of cells based on cell proliferation above a threshold level in response to contacting the cells with antigen-presenting cells expressing an antigen specific to an antigen-binding domain of the CAR, wherein the subpopulation comprises a plurality of cells. The method can further comprise isolating a subset of the plurality of variant nucleic acids from the subpopulation. The method can further comprise determining nucleotide sequences of the contiguous portion of individual members of the subset; and identifying at least one combination of the first, second, and third variant nucleotide subsequences based on an enrichment of the at least one combination in the nucleotide sequences of the subset relative to a control.

In some embodiments, a method of identifying a nucleotide sequence from a combinatorial library of nucleic acids is provided. The method can comprise: providing a combinatorial library comprising a plurality of variant nucleic acids, each of the plurality of variant nucleic acids comprises a contiguous portion of at least 600 bp; introducing the library into a population of cells configured to express one or more polypeptides encoded by a member of the plurality of variant nucleic acids; selecting a subpopulation of the population of cells based on at least one functional property dependent on the contiguous portion of at least 600 bp, wherein the subpopulation comprises a plurality of cells; isolating a subset of the plurality of variant nucleic acids from the subpopulation.; determining nucleotide sequences of the contiguous portion of individual members of the subset; and identifying the contiguous portion of at least 600 bp based on the nucleotide sequences.

In some embodiments, a method of identifying nucleotide sequences encoding antigen-specific T cell receptor α (TCRα)- and TCRβ-chain pairs from a library of nucleic acids is provided. The method comprises introducing a library into a population of cells able to express TCRα- and TCRβ-chains encoded by a member of a plurality of variant nucleic acids, selecting a subpopulation of the population of cells based on an expression of a marker above a threshold level in response to an antigen, wherein the subpopulation comprises a plurality of cells. The method can further comprise isolating a subset of the plurality of variant nucleic acids from the subpopulation. The method can further comprise determining nucleotide sequences of the variant nucleic acids, and identifying at least one variant nucleotide sequence based on an enrichment of the nucleotide sequences within the subset relative to a control.

In some embodiments, a method of identifying nucleotide sequences encoding T cell receptor α (TCRα)- and TCRβ-chains from a sample is provided. The method can comprise sequencing TCR-α and β chains in a sample, selecting and combinatorial pairing TCRα- and β-chain sequences to create a library of TCRαβ pairs, introducing the library of TCRαβ pairs into a pool of reporter cells, stimulating the reporter cells that are modified with the library of TCRαβ pairs with antigen presenting cells presenting at least one antigen of interest (wherein the antigen can be from a same host that the TCRalpha and TCR beta chains are from), determining TCRαβ pairs specific to the at least one antigen of interest, and introducing the TCRαβ pairs into cells and selecting cells containing the TCRαβ pairs.

In some embodiments, a nucleotide library comprising the repertoire of T cell receptors recovered according to any one of methods above are provided.

In some embodiments, a nucleotide construct comprising the nucleotide sequence identified according to any one of methods herein is provided.

In some embodiments, a cell comprising the nucleotide construct according to any of the nucleotides provided herein is provided.

In some embodiments, a method of identifying a nucleotide sequence encoding an antigen-specific T cell receptor α (TCRα)- and TCRβ-chain pair from a library of nucleic acids is provided. The method can comprise: introducing the nucleic acid library into a population of cells able to express TCRα- and TCRβ-chains to make a library of cells; selecting a first population of the library of cells based on an expression of a marker above a first threshold level in response to an antigen; and isolating a first population of variant nucleic acids from the first population of the library. In some embodiments, the antigen can be one or more and both the antigen(s) and the TCRalpha and TCR betas sequences can be found in a single subject.

In some embodiments, a method of identifying a nucleotide sequence encoding a T cell receptor α (TCRα)- and TCRβ-chain from a library of nucleic acids is provide. The method can comprise: introducing the nucleic acid library into a population of cells able to express TCRα- and TCRβ-chains to make a library of cells; and determining at least one nucleotide sequence or nucleic acid identity of the first population of variant nucleic acids based on an enrichment of the nucleotide sequence within the subset relative to a control.

In some embodiments, a method of identifying a nucleotide sequence from a library of nucleic acids is provided. The method can comprise introducing the library of nucleic acids into a population of cells to form a library of cells; contacting the library of cells with a first population of cells; selecting a sub-population of the library of cells based on expression of at least one marker by magnetic bead enrichment; and identifying at least one nucleotide sequence based on a statistically significant enrichment or depletion of the nucleotide sequences within the sub-population relative to a control.

In some embodiments, a collection of cells is provided. The collection comprises a set of at least two T cells, wherein each is configured to express at least one TCR alpha and TCR beta pair, wherein the TCR alpha and the TCR beta are each from a subject, wherein the T cells do not express an endogenous TCR, and wherein the set are configured for activation of one or more T cell activation markers; and a set of at least two B cells, wherein each of the at least two B cells is configured to express at least one exogenous neo-antigen (or antigen), such that there are at least two exogenous neo-antigens (or antigens) capable of being produced, and wherein the at least two exogenous neo-antigens (or antigens) are the same as those in the subject.

In some embodiments, a library of TCR expressing cells is provided. The library of TCR expressing cells comprises: a set of at least three T cells, wherein at least two of the T cells are configured to express at least two TCR alpha and TCR beta pairs (at least two TCR pairs), wherein the at least two TCR pairs are from a subject, wherein the at least three T cells do not express an endogenous TCR, wherein the at least three T cells are configured for activation of one or more T cell activation markers, upon binding to an antigen (or neo-antigen), presented by a B cell, wherein an amount of genomic copies of each TCR pair as reflected in a number of TCR cells is such that one gets a read on every TCR in the sample, and wherein at least one of the TCRs is not distributed equally throughout a composition comprising the library.

In some embodiments, a method of treating a subject is provided. The method comprises identifying a subject having a tumor; providing a set of at least two T cells, each of which is configured to express at least one different TCR alpha and TCR beta pair, wherein each of the TCR alpha and the TCR beta are from the subject, providing a set of at least two B cells, wherein the set of B cells is configured to express at least two exogenous neo-antigens, and wherein the at least two exogenous neoantigens are the same as those neo-antigens found in the subject; combining the set of at least two T cells with the set of at least two B cells and selecting a combination of at least two TCR pairs based upon activation of the at least two T cells via the at least two exogenous neo-antigens; and administering the combination of at least two TCR pairs to the subject, thereby treating the tumor.

In some embodiments, a method of treating a subject is provided. The method comprises: identifying a subject having a tumor; providing a set of at least two T cells, each of which is configured to express at least one different TCR alpha and TCR beta pair, wherein each of the TCR alpha and the TCR beta are from the subject; providing a set of at least two antigen presenting cells, wherein the set of antigen-presenting cells originates from the subject, is configured to express at least two exogenous neo-antigens, and wherein the at least two exogenous neoantigens are the same as those neo-antigens found in the subject; combining the set of at least two T cells with the set of at least two antigen present cells and selecting a combination of at least two TCR pairs based upon activation of the at least two T cells via the at least two exogenous neo-antigens; and administering the combination of at least two TCR pairs to the subject, thereby treating the tumor.

In some embodiments, a pharmaceutical composition is provided. The composition can comprise: a first TCR pair, that binds to a first antigen (or neo-antigen) in a subject's tumor; and a second TCR pair, that binds to a second antigen (or neo-antigen) in the subject's tumor.

In some embodiments, a pharmaceutical composition is provided. The composition can comprise: a first TCR pair, that binds to a first antigen and is MHC-class I restricted; and a second TCR pair, that binds to a second antigen and is MHC-class II restricted.

In some embodiments, a collection of cells is provided. The collection can comprise: a set of at least two T cells, wherein each is configured to express at least one TCR alpha and TCR beta pair, wherein the pair is from a subject, wherein the T cells do not express an endogenous TCR, and wherein the set are configured for activation of one or more T cell activation markers; and a set of at least two antigen present cells (APCs), wherein each of the at least two APCs is configured to express at least one exogenous neo-antigen (or antigen), such that there are at least two exogenous neo-antigens (or antigens) capable of being produced, and wherein the at least two exogenous neo-antigens (or antigens) are the same as those in the subject.

### BRIEF DESCRIPTION OF THE DRAWINGS

FIG. 1 illustrates a method of recovering a TCR repertoire for library generation.

FIG. 2 illustrates a method of recovering a TCR repertoire and identification of antigen specific TCRα and TCRβ sequences for use in cancer therapy.

FIG. 3 shows an exemplary design for a TCR expression cassette.

FIG. 4 illustrates some embodiments for screening of combinatorial TCR libraries.

FIG. 5 shows an exemplary application of processes disclosed herein for the treatment of a cancer patient.

FIG. 6 shows a schematic example of a TCRβ-P2A-TCRα-T2A-Puromycin resistance cassette.

FIG. 7 shows an exemplary strategy to assemble TCR expression cassettes.

FIG. 8 shows a schematic example of a screening method according to embodiments of the present disclosure.

FIG. 9 shows a flow chart of a screening method according to embodiments of the present disclosure.

FIGS. 10A-10J depict the recovery of TCR repertoires from non-viable tumor specimens to identify neo-antigen specific TCR sequences.

FIG. 10A is a schematic of some embodiments of a screening process.

FIG. 10B is a graph depicting the number of TCR clonotypes.

FIG. 10C is a graph depicting the probability density of each of the 10,000 TCR alpha x beta combinations.

FIG. 10D is a plot depicting the resulted cell population after blasticidin selection.

FIG. 10E is a series of FACS results using various indicated markers.

FIG. 10F is a gel depicting various PCR products.

FIG. 10G is a series of plots depiciting the average Rlog-transformed read counts for screens in the presence (x-axis) and absence (y-axis) of TMG expression by B cells, which are represented for the pt1 tumor sample described in 10B)-10F), as well as for three additional MMRp-CRC samples (pt2, pt3 and pt4) processed in an identical manner. Neo-antigen reactive TCR leads are depicted as encircled larger black dots.

FIG. 10H is a plot depicting step 10H.

FIG. 10I is a graph depicting activation as measured as CD69-positivity relative to a positive control (treatment with PMA/Ionomycin). Activation by a control (PARVA-P70R), as well as a AKAP8L-R191W peptide, are shown.

FIG. 10J is a graph depicting activation as measured as CD69-positivity relative to a positive control (treatment with PMA/Ionomycin). Activation by a control (ETS1-R70W), as well as a TP53-R282W peptide, are shown.

FIG. 10K is a graph depicting activation as measured as CD69-positivity relative to a positive control (treatment with PMA/Ionomycin). Activation by control B cells (not expressing a TMG), as well as by B cells expressing MG91 (HSPA9-p.K654RfsX42) or TMG3, are shown.

FIG. 10L is a graph depicting activation as measured as CD69-positivity relative to a positive control (treatment with PMA/Ionomycin). Activation by control B cells (not expressing a TMG), as well as by B cells expressing MG132 (ITPR3-p.L2379M) or TMG4, are shown.

FIG. 11A is bar chart depicting the number of TCR clonotypes.

FIG. 11B is a Schematic representation of the TCR expression plasmid.

FIG. 11C are graphs depicting the probability density of each of the 10,000 alpha x beta combinations for every patient library.

FIG. 11D provides the range of the amount of reads per TCR, the mean coverage, and the percentage of TCRs that fall within a range of the median +/−a ²log-unit.

FIG. 12A is a bar graph representing the number of clonotypes

FIG. 12B are graphs depicting the probability density of each of the 10,000 TCR alpha x beta combinations for two patient libraries.

FIG. 12C is a table providing the coverage of each of the 10,000 alpha x beta combinations.

FIG. 13A is a table providing the dilution of six TCRs with known antigen-specificity among 24 TCRs with unknown antigen-specificity.

FIG. 13B are FACS results showing the frequency of TCR+ Jurkat reporter cells after selection.

FIG. 13C are FACS plots depicting the sorting strategy for Top and Bottom T cell populations based on CD69 activation marker.

FIG. 13D is a gel depicting various PCR products.

FIG. 13E is a graph comparing enrichment to frequency of TCRs with known antigen-specificity and TCRs with unknown antigen-specificity.

FIG. 14A is a flow chart of the design of the 50×50 and 100×100 with spiked in characterized TCR chains.

FIG. 14B is a collection of FACS plots depicting the sorting strategy for Top and Bottom T cell populations based on CD69 activation marker

FIG. 14C is a gel depicting various PCR products

FIG. 14D are plots comparing the fold change of TCR representation in top versus bottom sample relative to average expression in these samples.

FIG. 14E is a table providing the rank order of the most enriched TCR sequences and the base mean, Log(2) fold change and adjusted p-value of such TCRs

FIG. 14F is a plot comparing TCR reactivity in the absence of an antigen to TCR reactivity in the present of an antigen.

FIG. 14G is a graph displaying the probability of antigen-specific TCRs and TCRs with unknown specificity being enriched in the screening

FIG. 15A is a schematic depicting the combinatorial assembly of TCR cassettes

FIG. 15B is a series of graphs depicting the probability densities of TCRα-, TCRβ-chains and TCRαβ combinations in the TCR library.

FIG. 15C and FIG. 15D are graphs depicting alternative strategies to compose higher complexity TCR libraries.

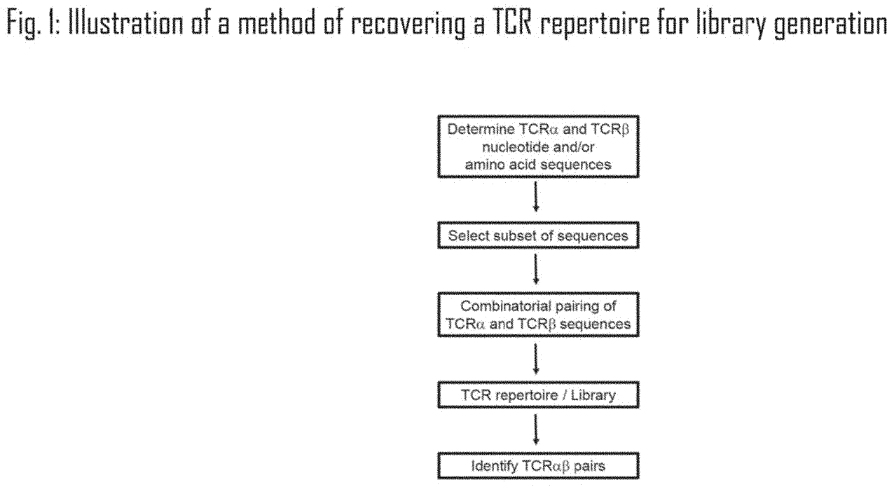

FIG. 1.5E depicts the probability densities of TCR combinations that are present in two 200×200 TCR libraries created by synthesis of four 100×100 libraries, and mixing these in 1:1:1:1 equimolar ratios.

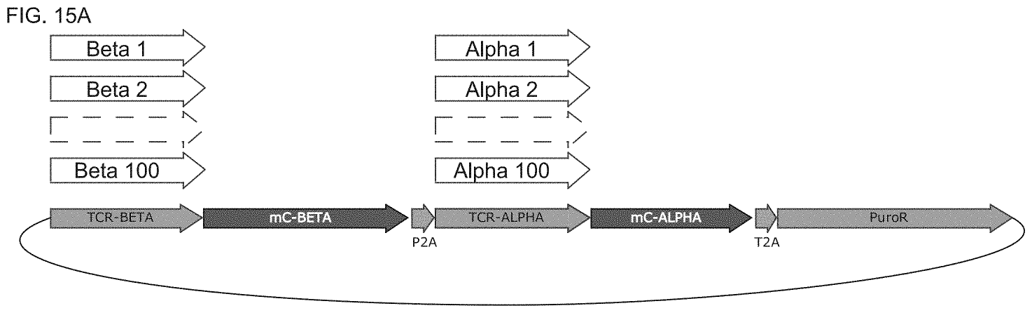

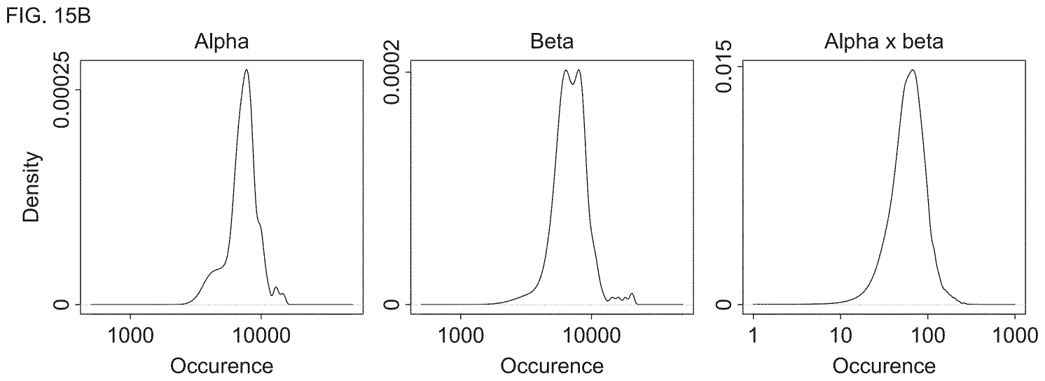

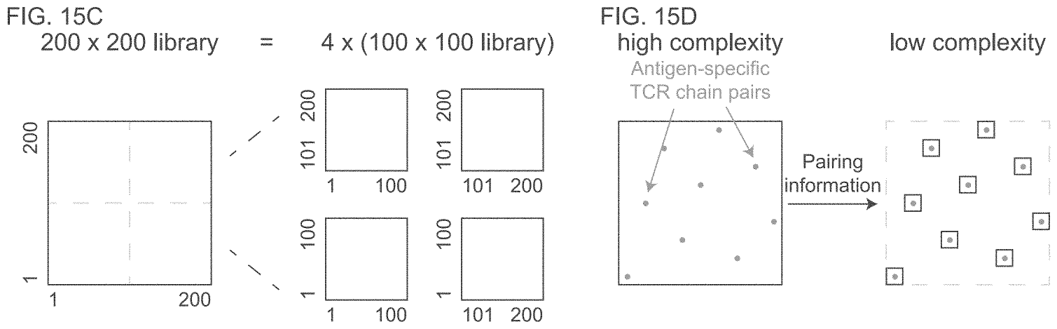

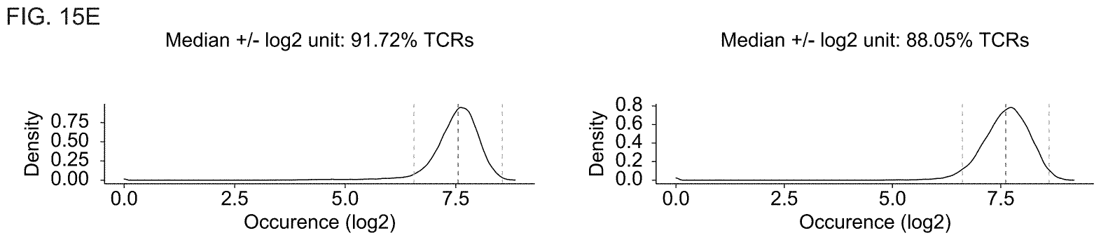

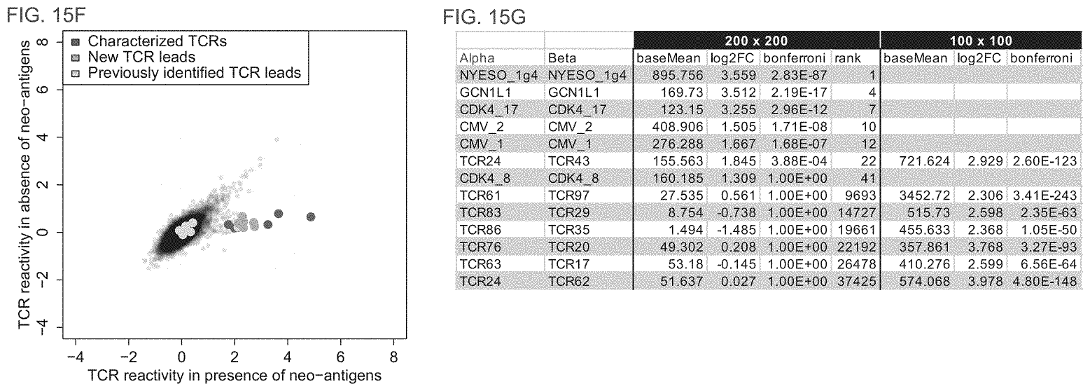

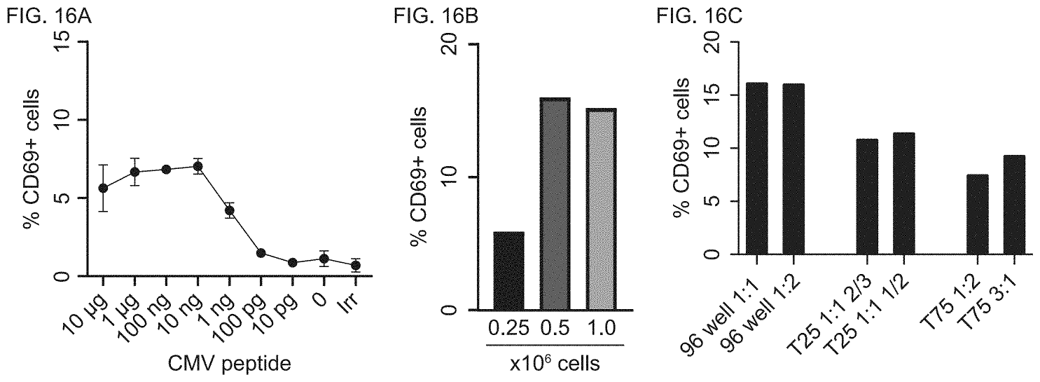

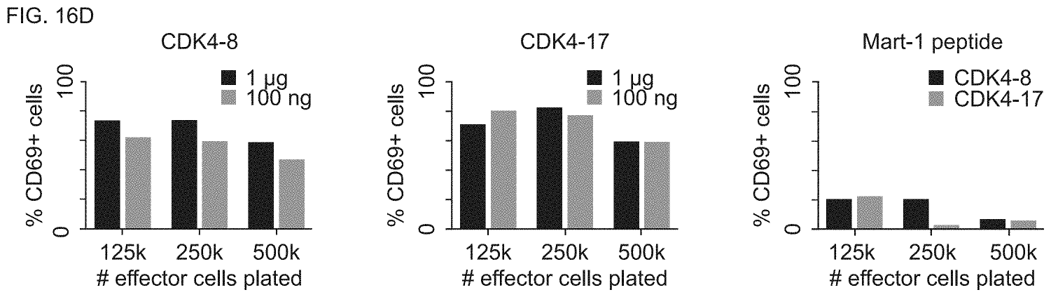

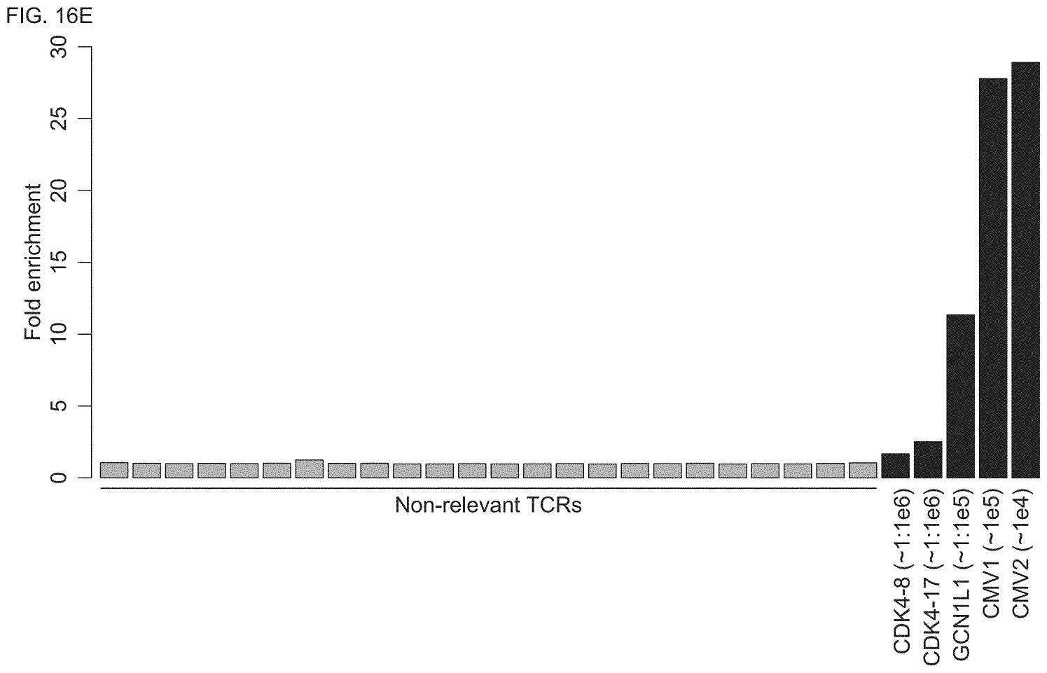

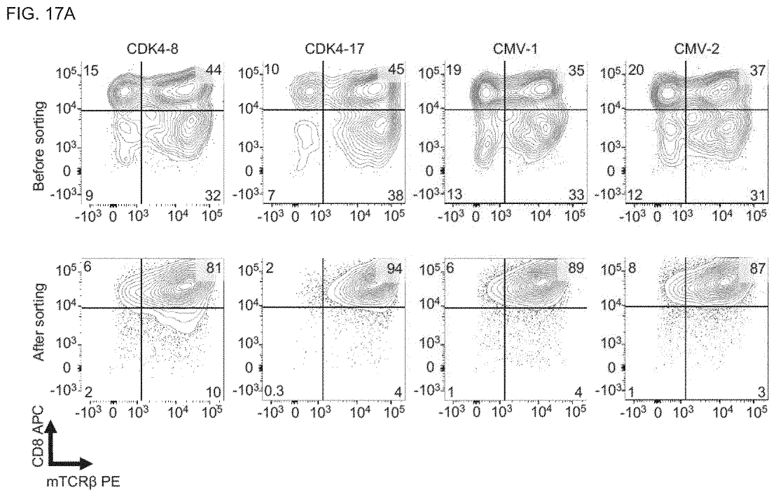

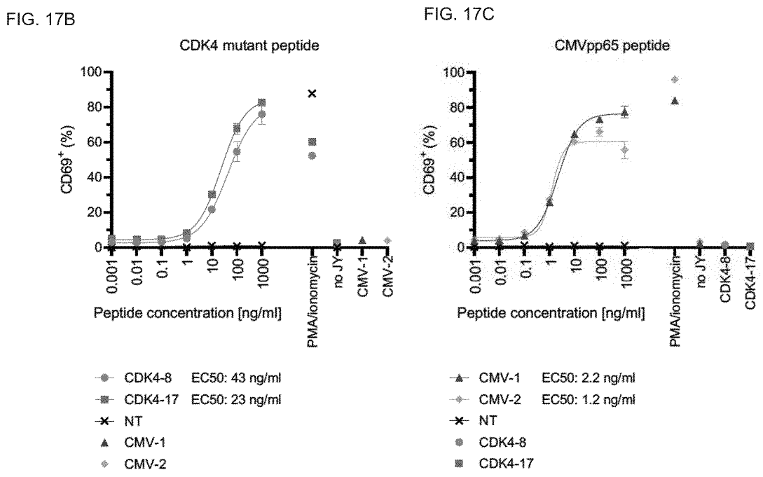

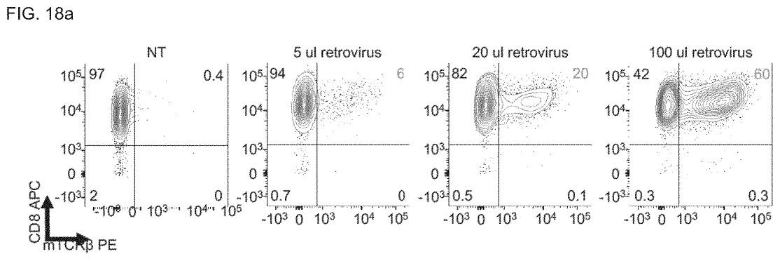

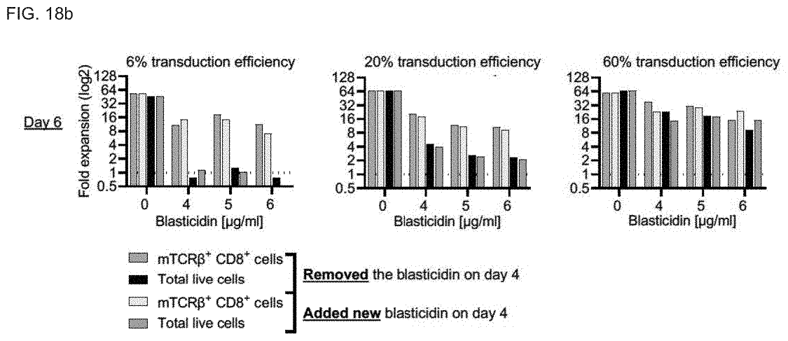

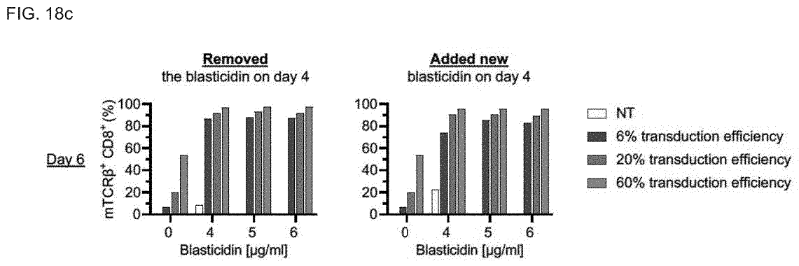

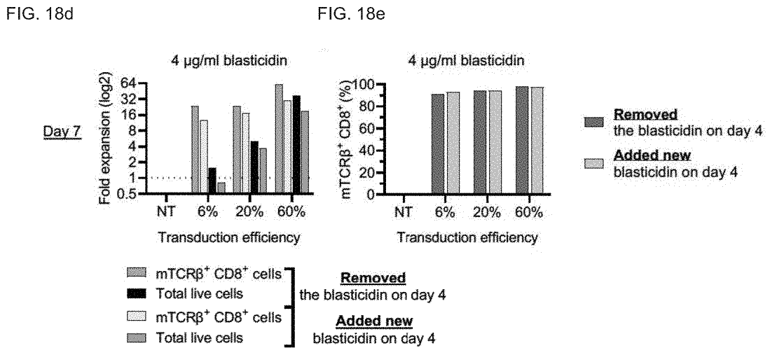

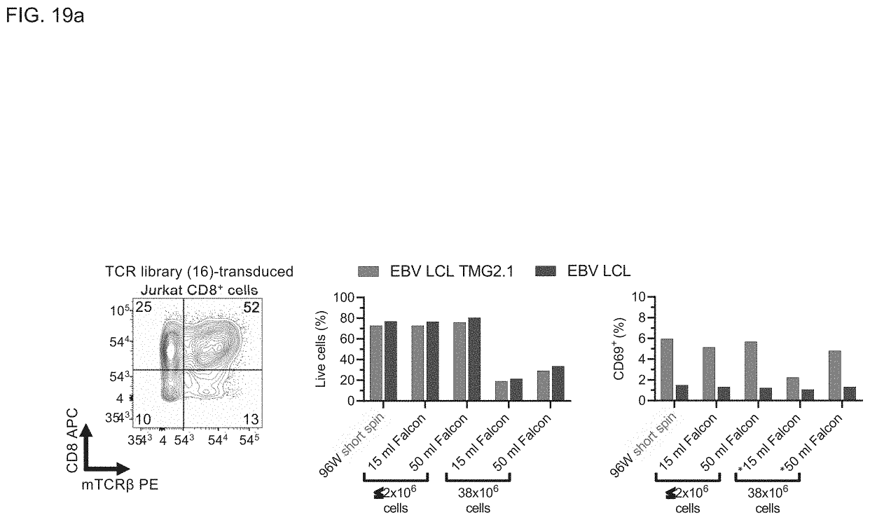

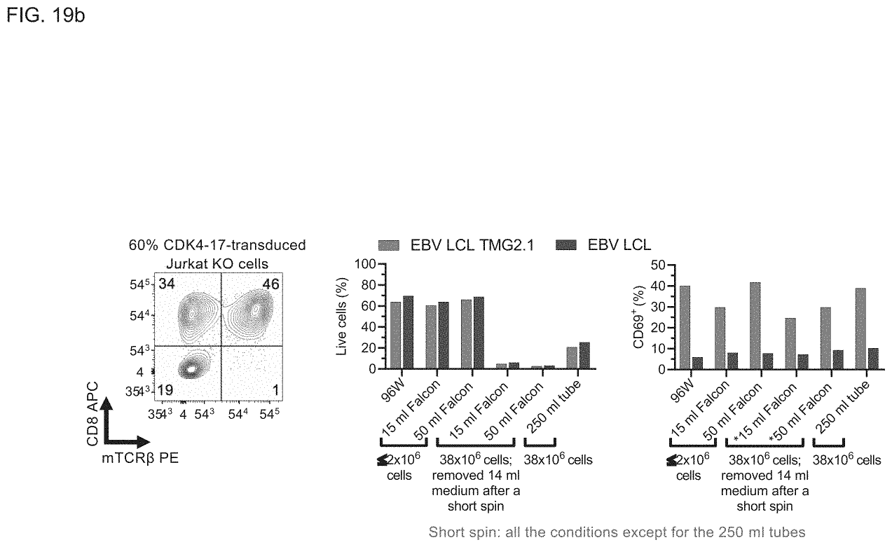

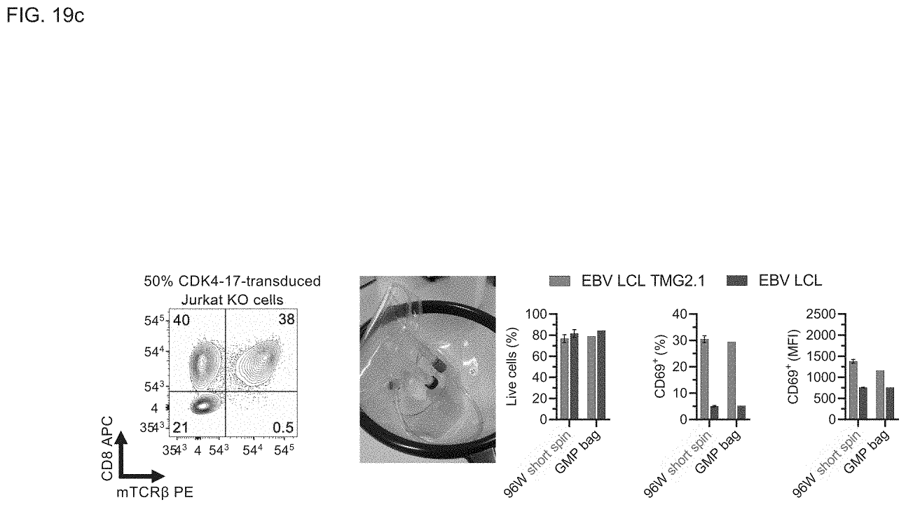

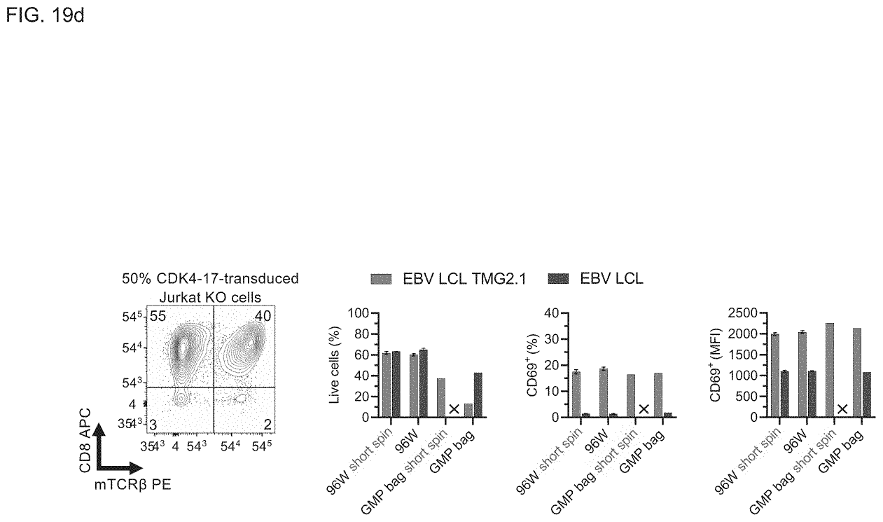

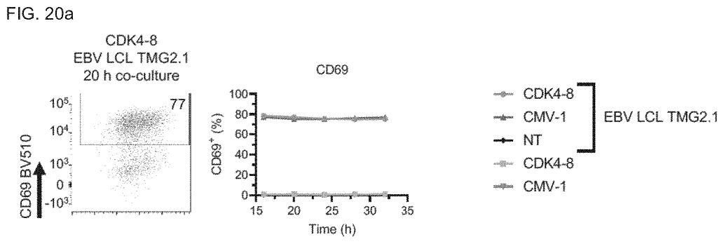

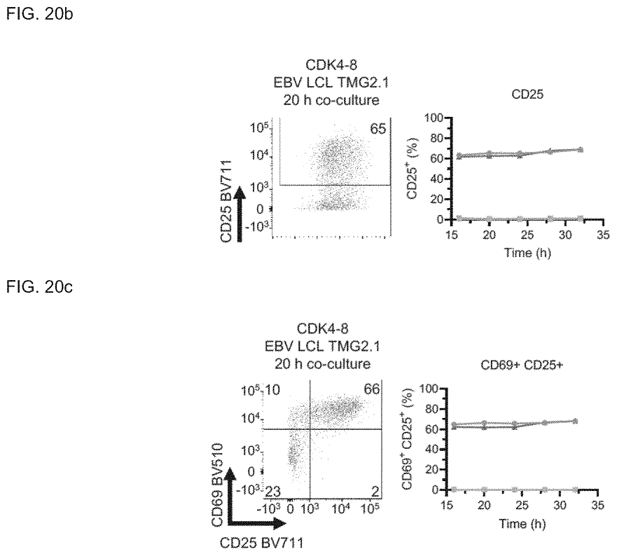

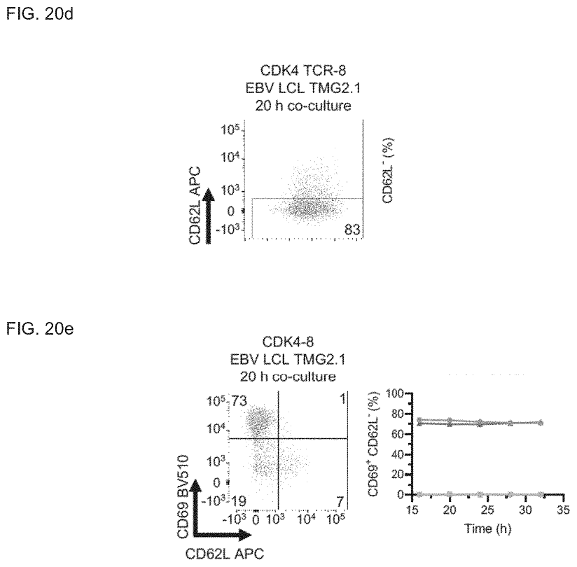

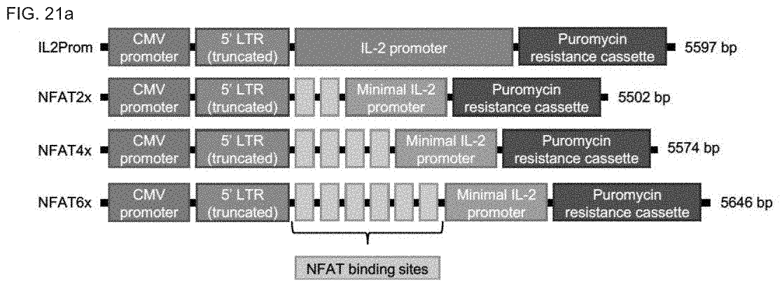

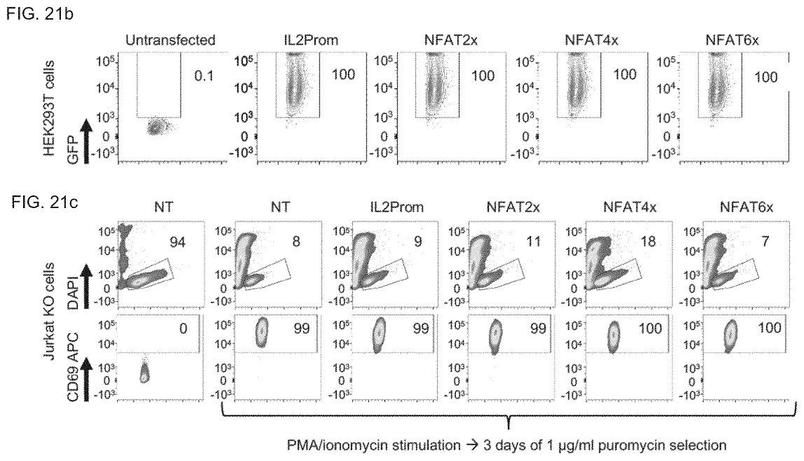

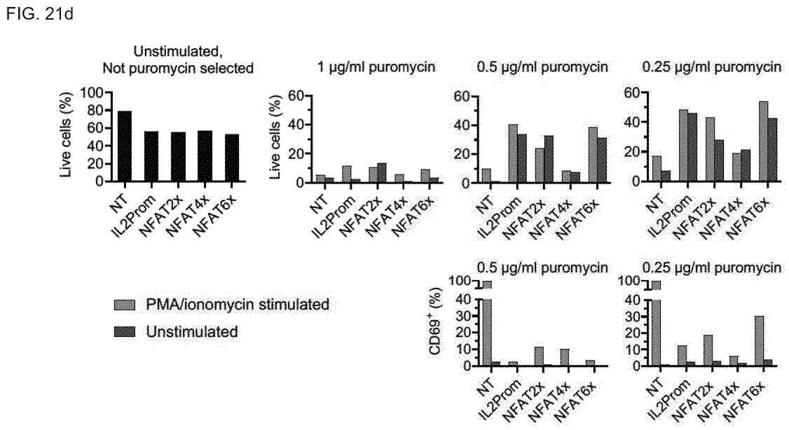

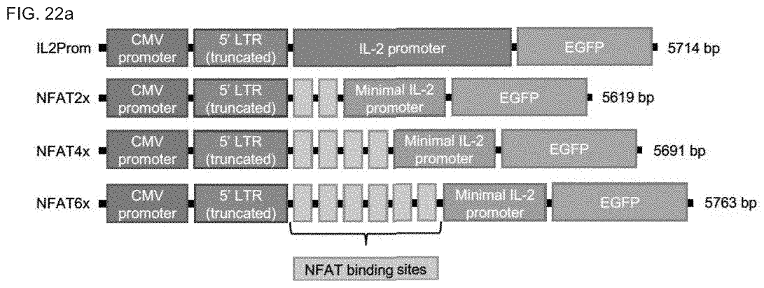

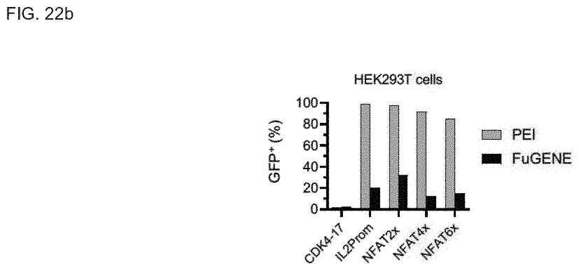

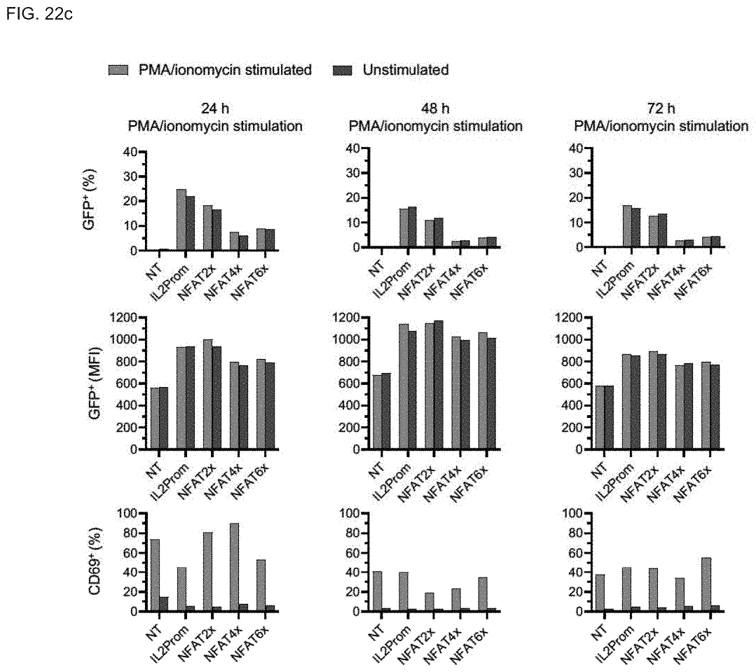

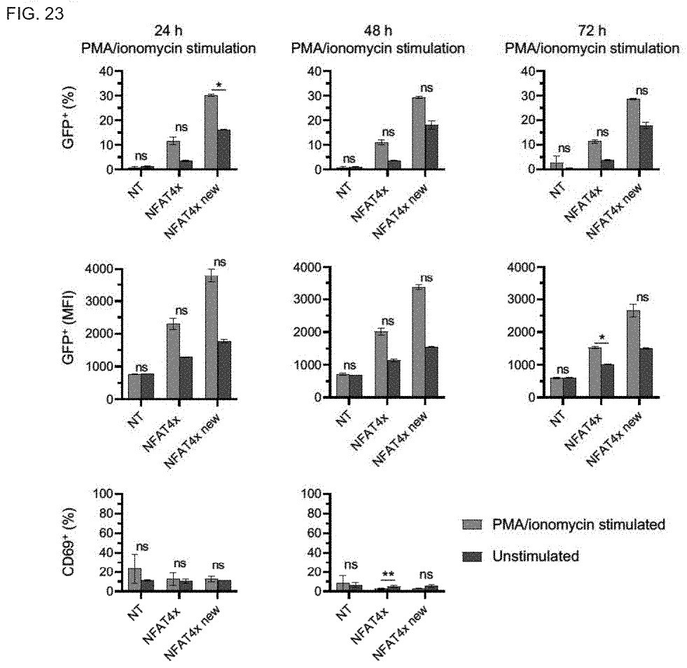

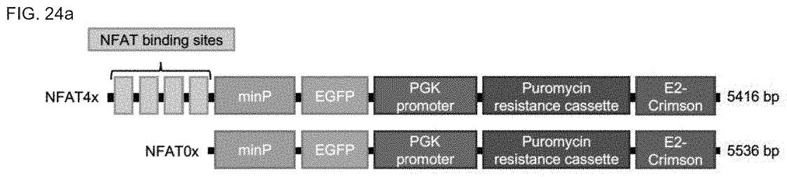

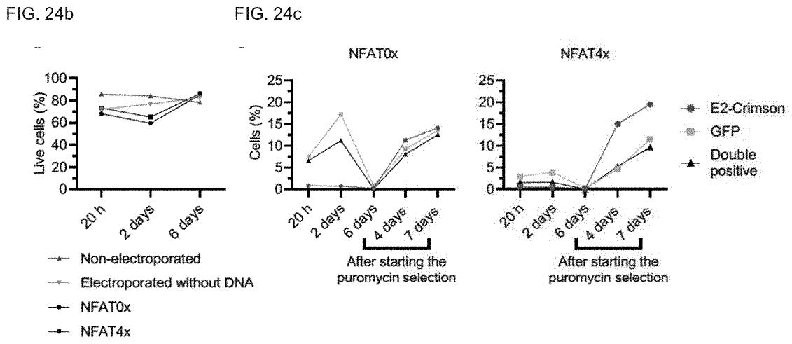

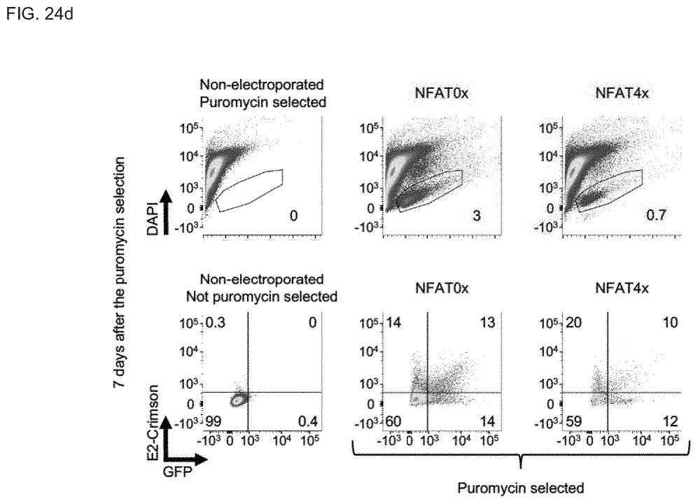

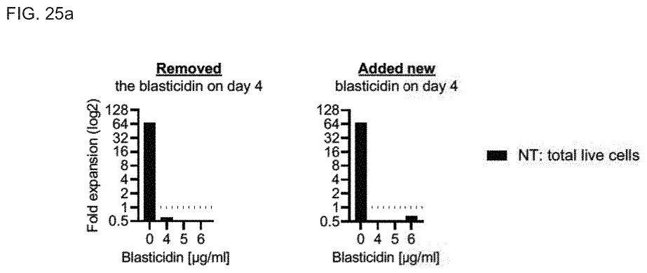

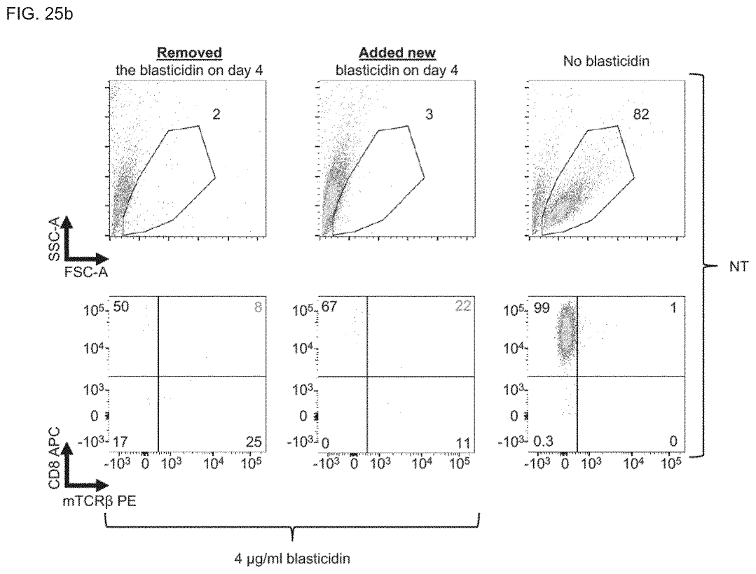

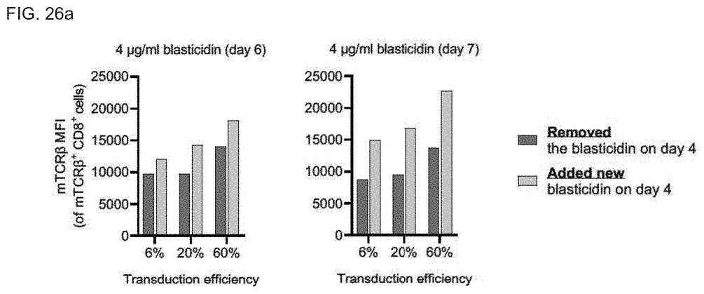

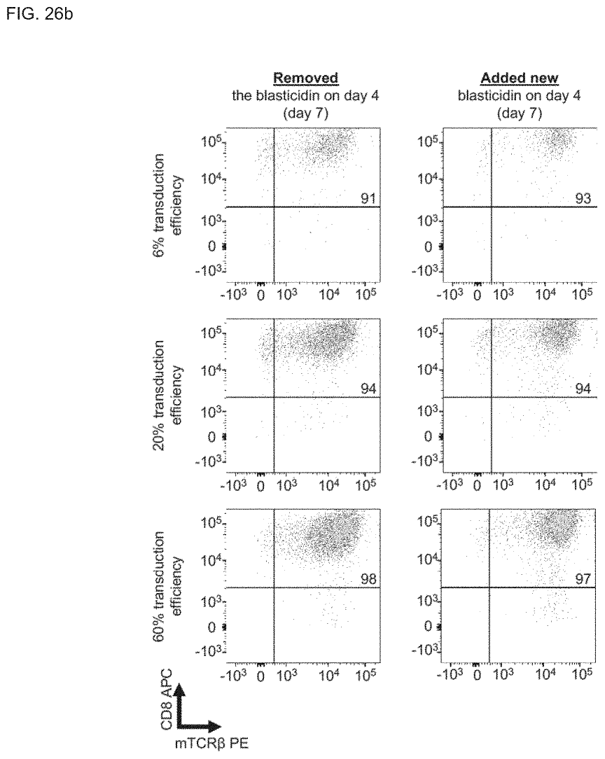

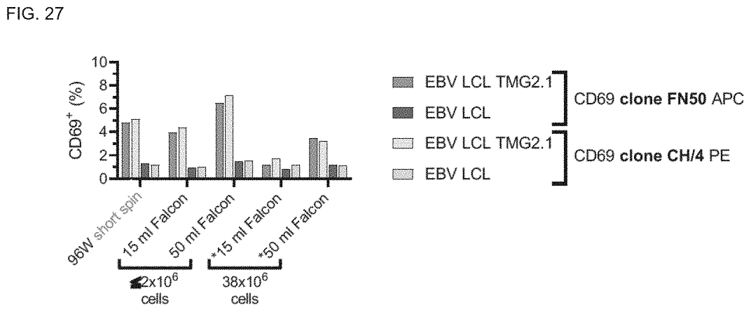

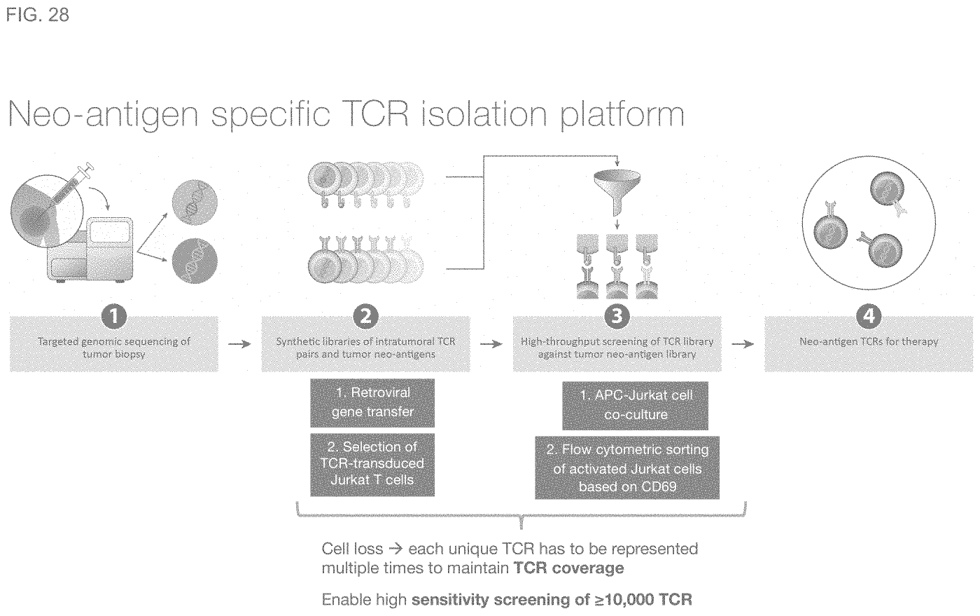

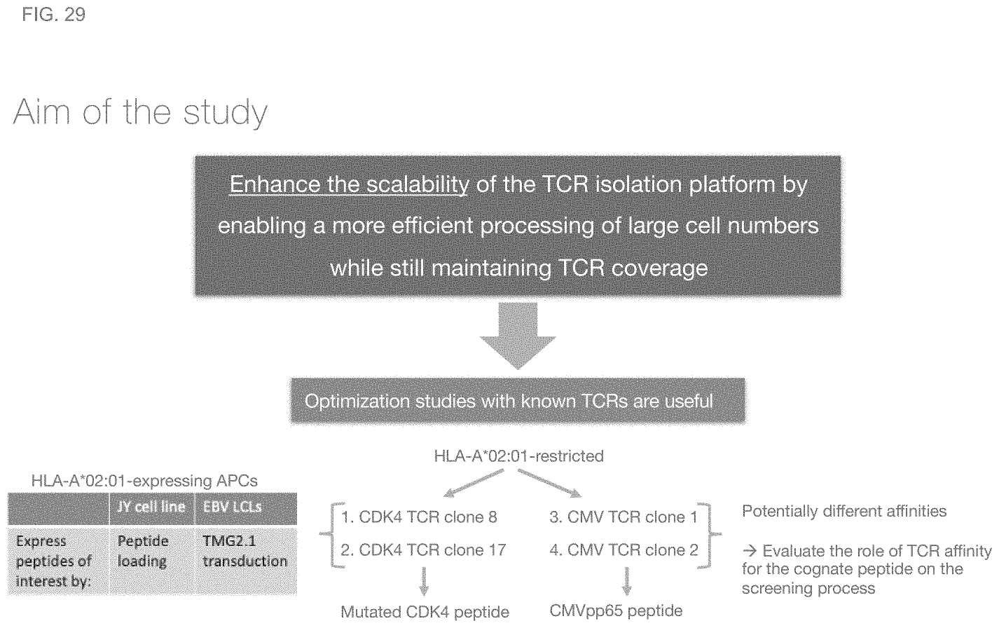

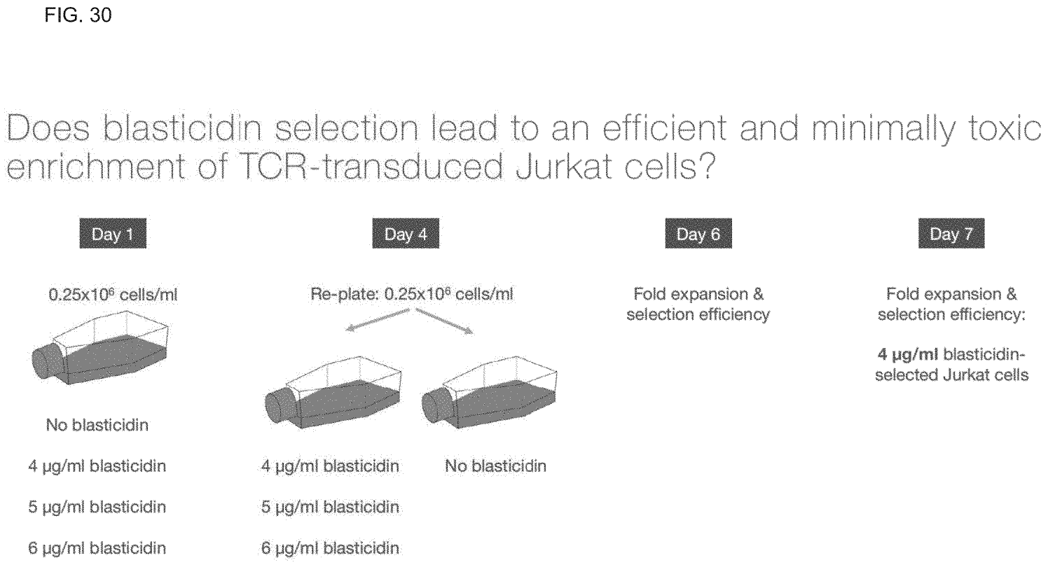

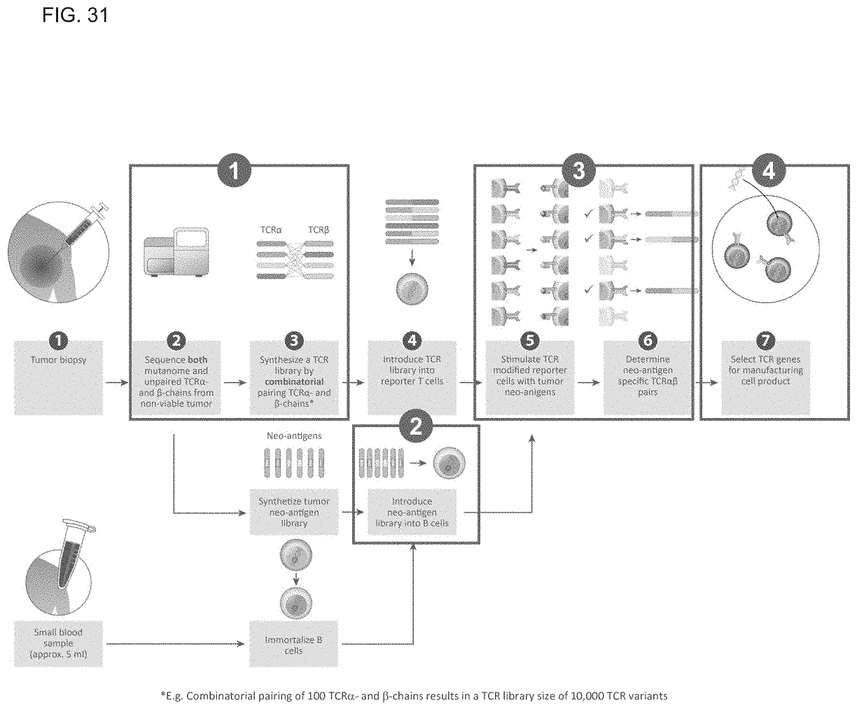

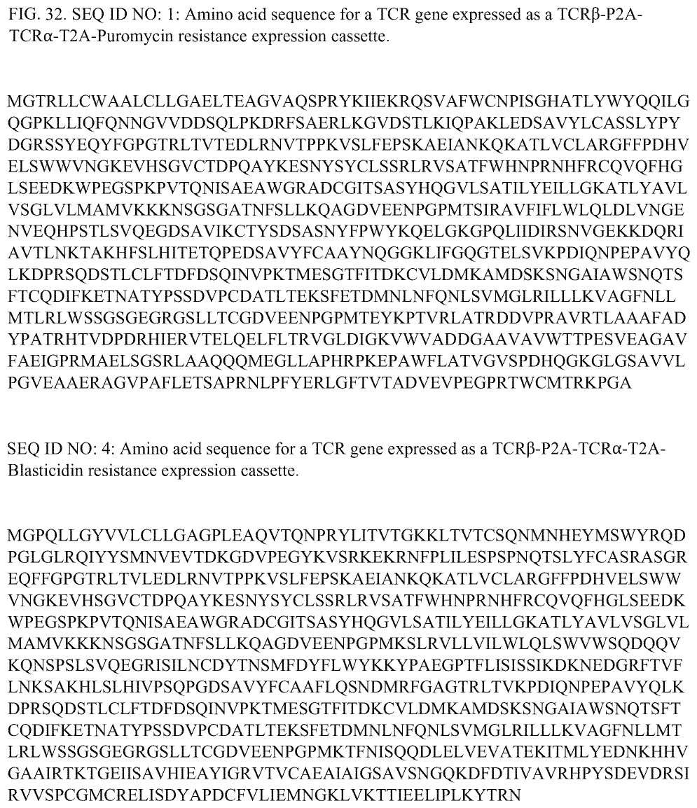

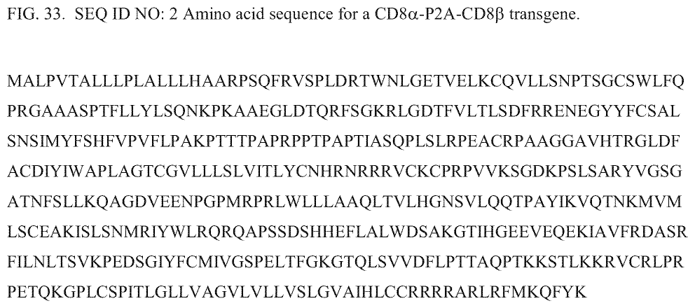

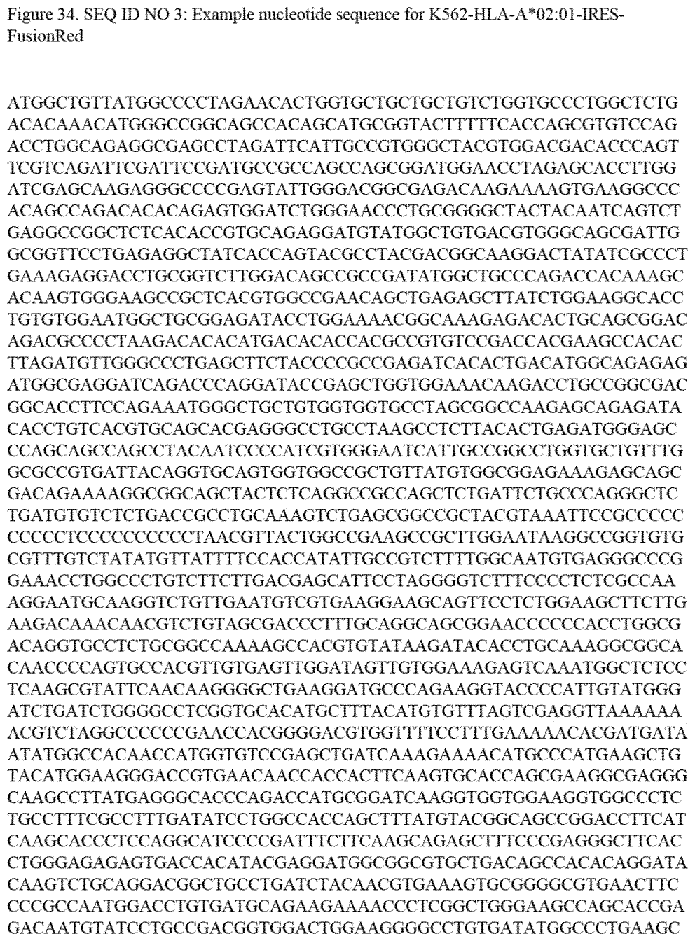

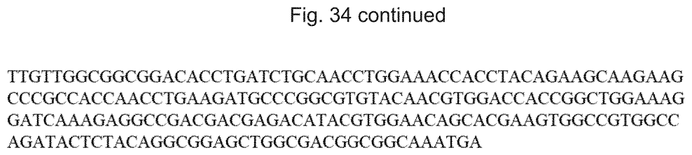

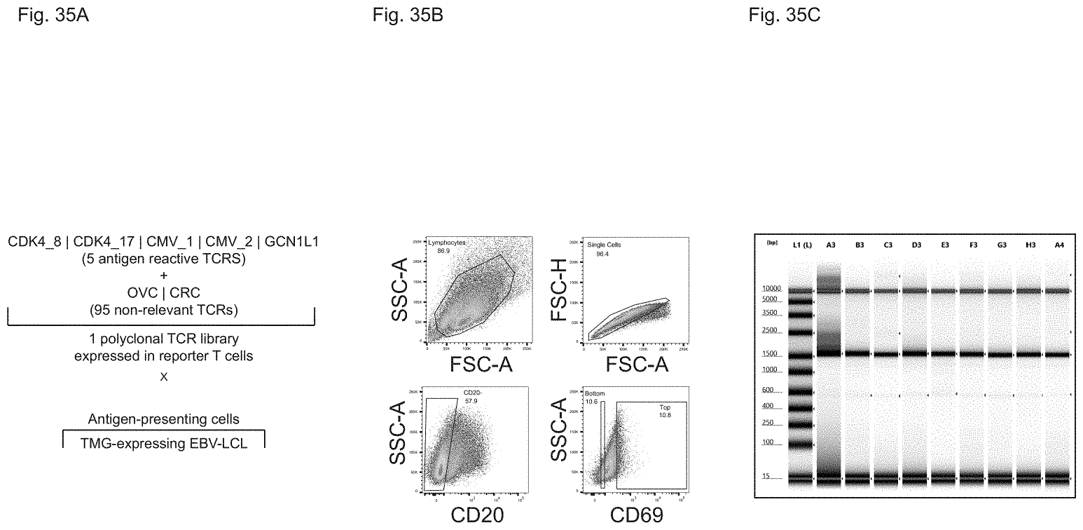

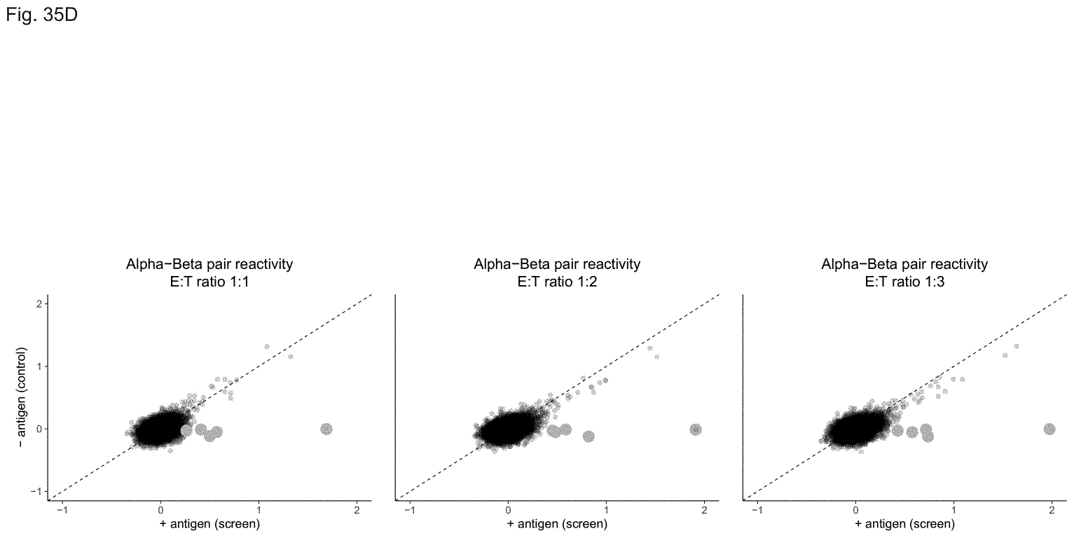

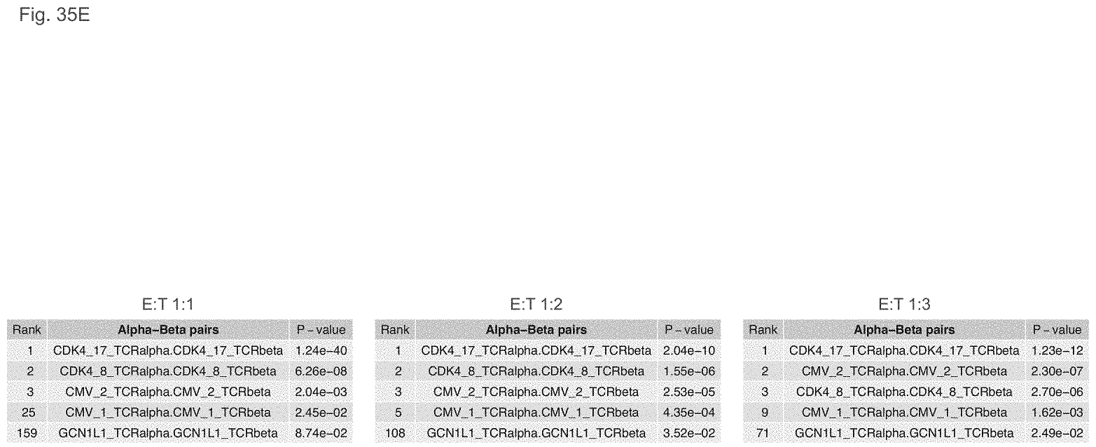

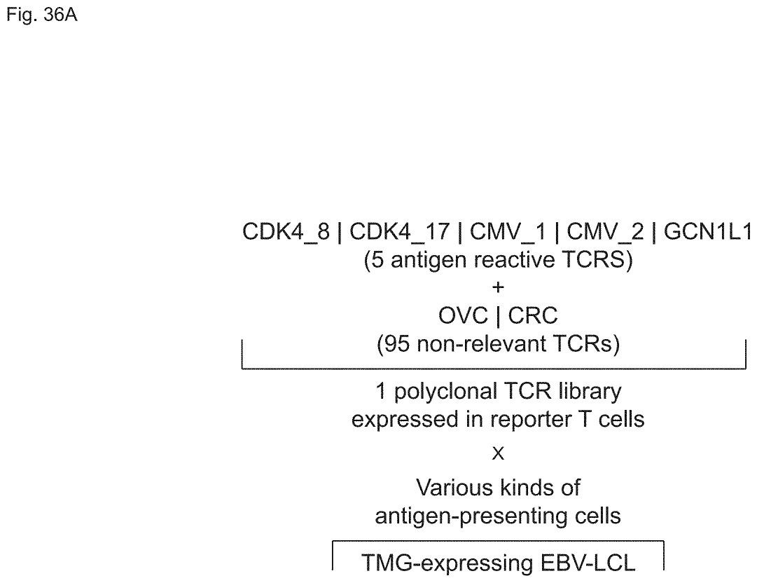

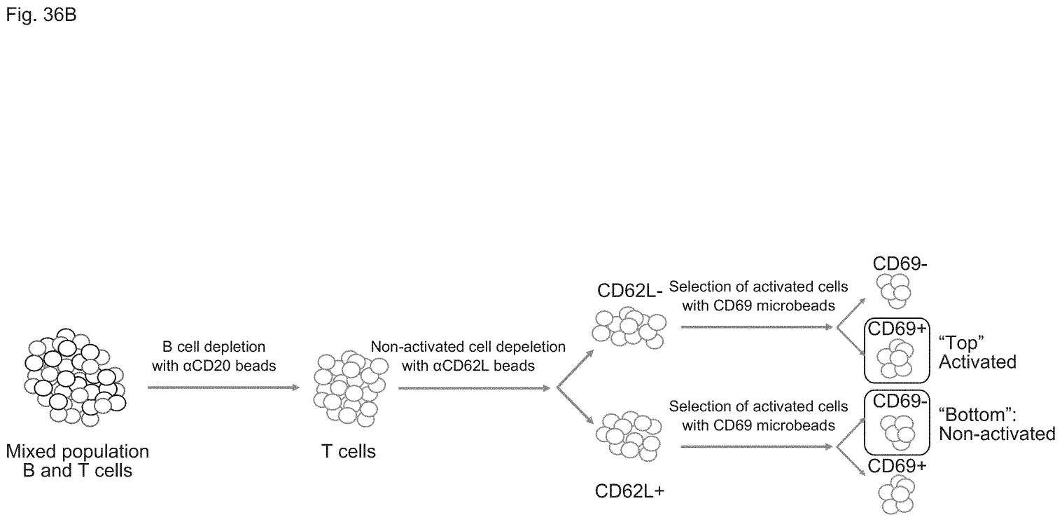

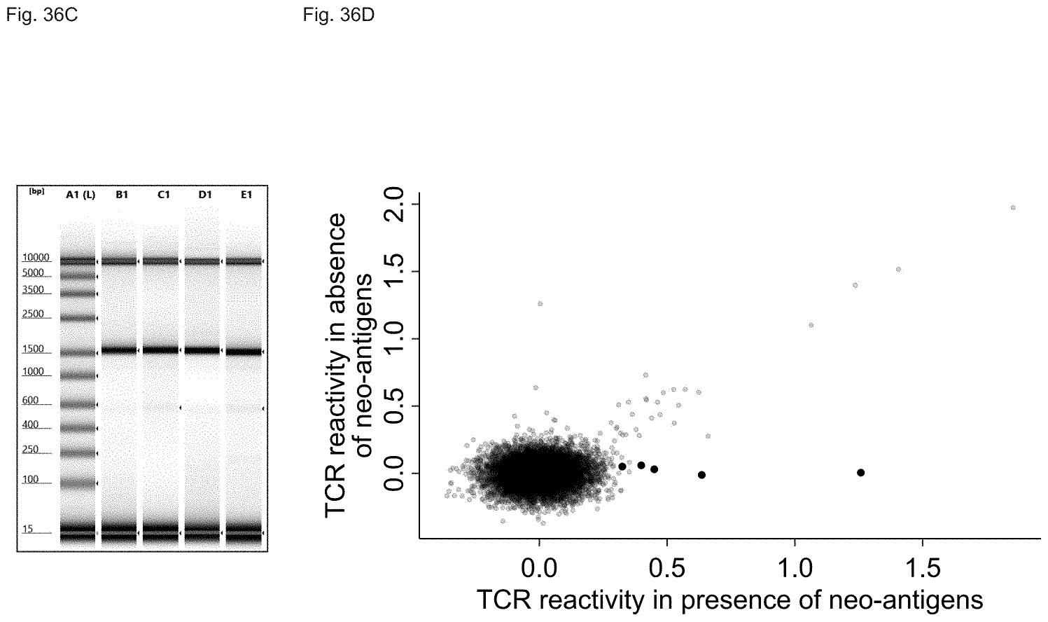

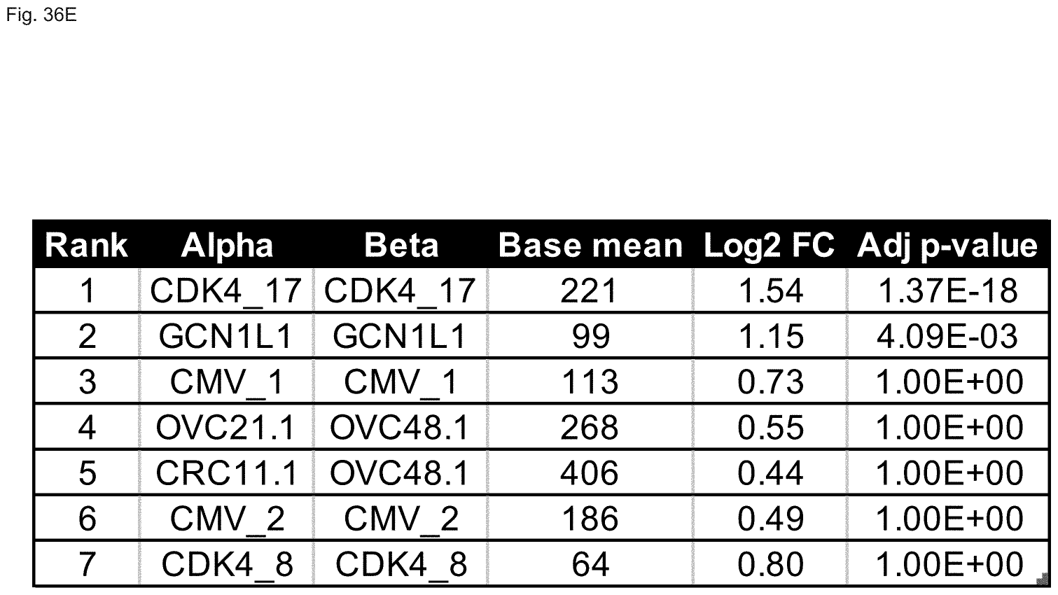

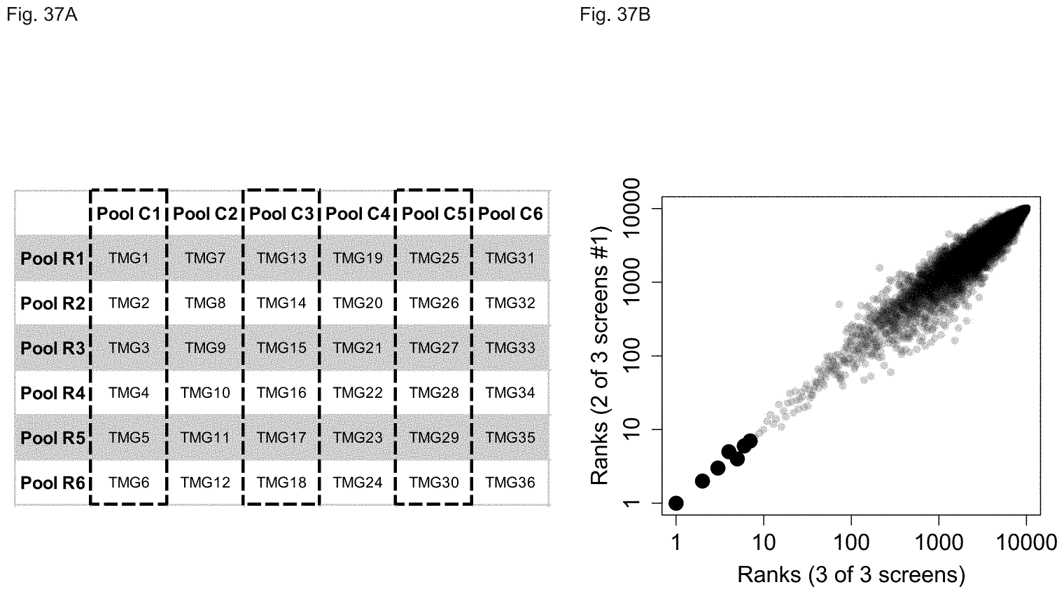

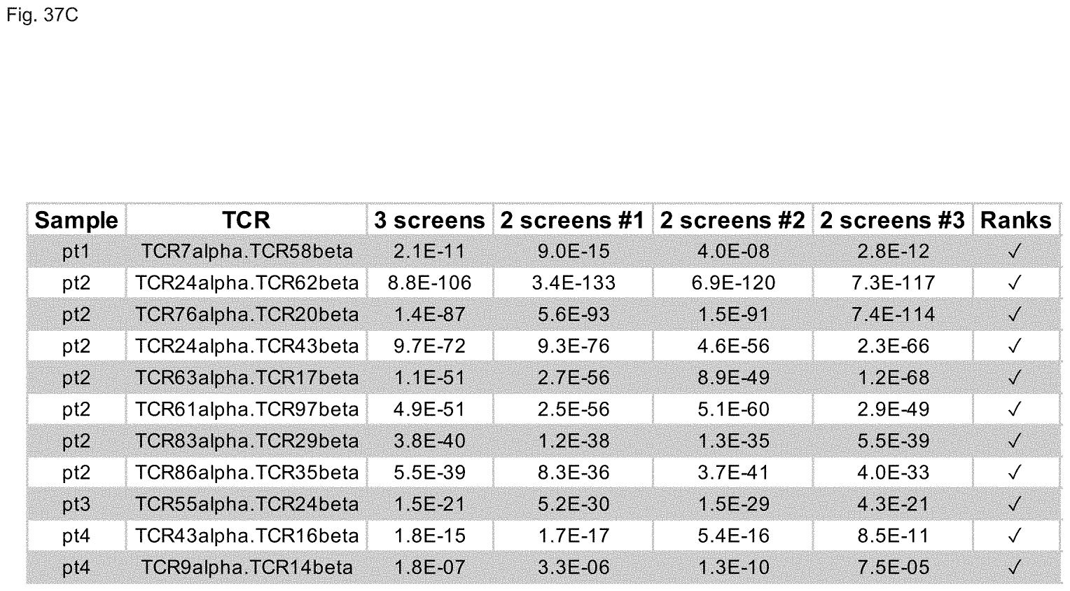

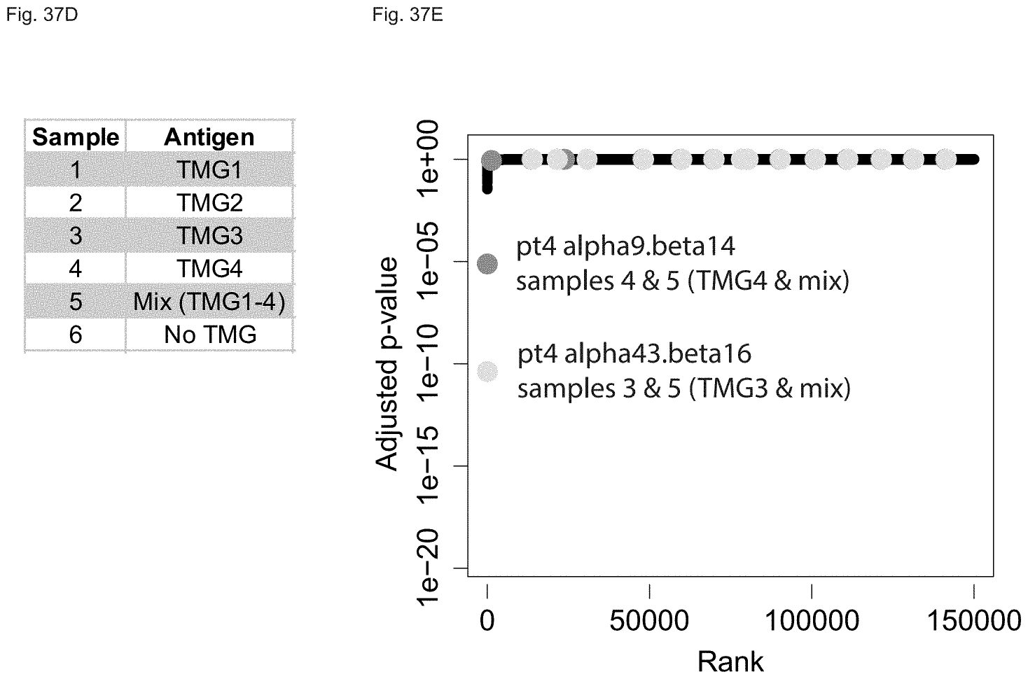

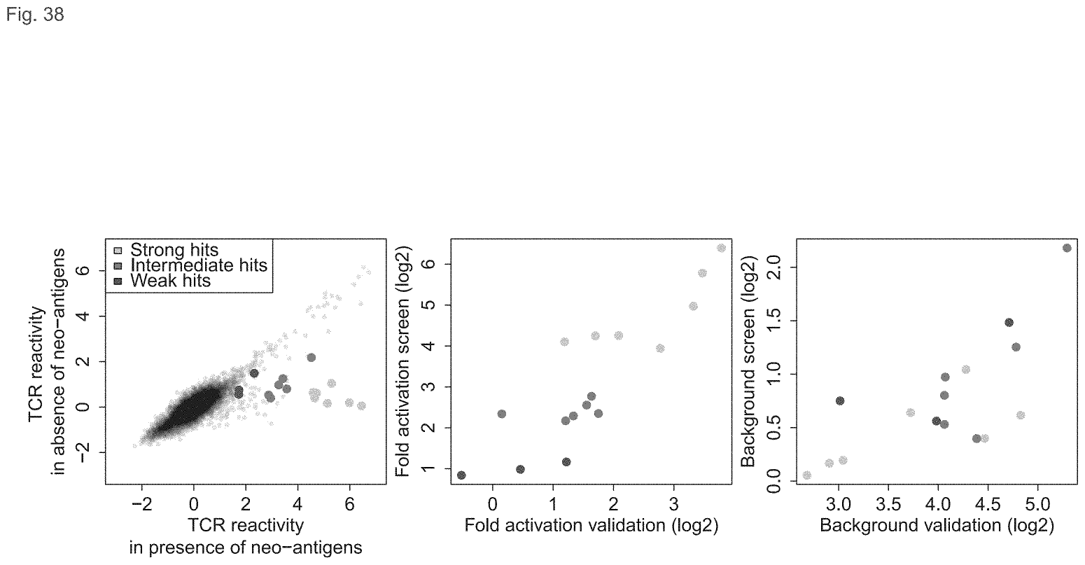

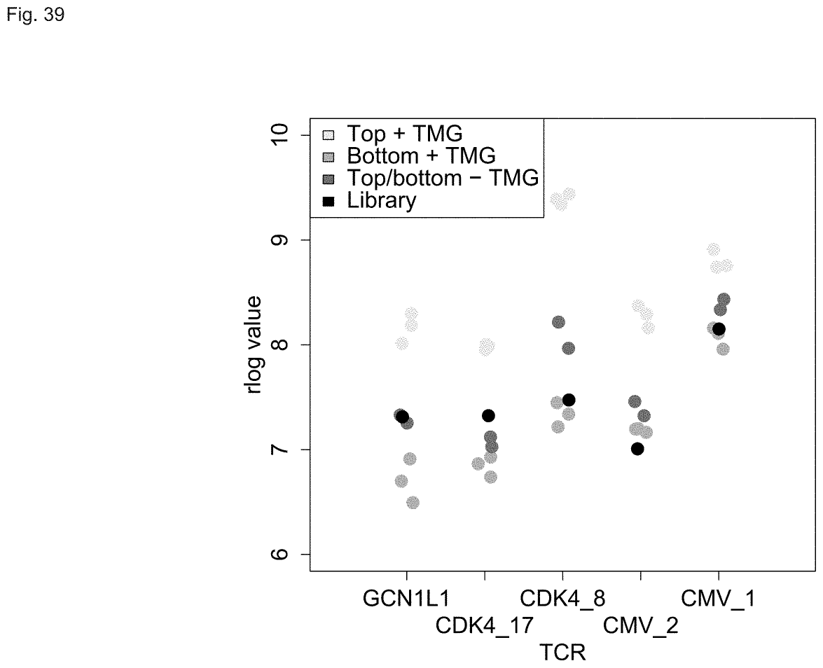

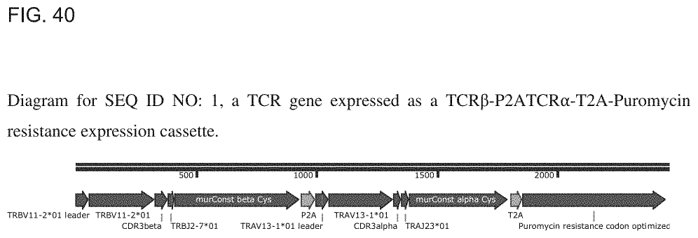

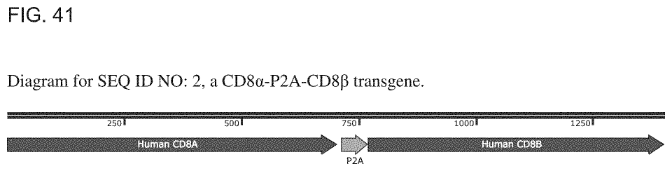

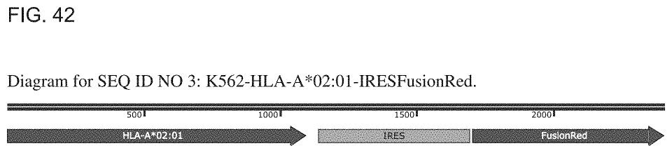

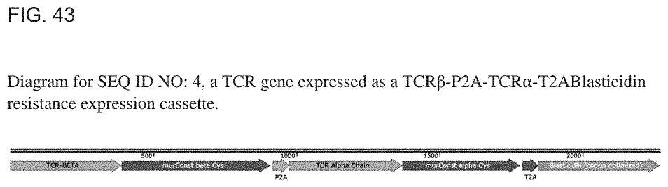

FIG. 15F depicts the TCR identification for pt2 using a 200×200 library screening approach.

FIG. 15G represents a table of the statistical behavior of the pt2 TCRs identified in the 100×100 screen in both 100×100 and 200×200 library screens.

FIG. 16A is a graph showing the relationship of the percent CD69+ cells to the amount of CMV peptide used for pulsing APCs.

FIG. 16B is a bar chart depicting the percent CD69+ cells to cell seeding density.

FIG. 16C is a bar chart depicting percent CD69+ cells in relationship to effector-to-target ratio and culture vessel

FIG. 16D is a series of bar charts depicting the percent CD69+ cells to the number of effector cells plated and the amount of antigenic peptide used.

FIG. 16E is a bar chart showing the degree of enrichment to various TCRs.

FIG. 17A-C. Peptide titration assay with CDK4 and CMV-transduced Jurkat TCR KO cells. (17a) Jurkat TCR KO cells were transduced either with CDK4-8 or 17 TCR or with CMV-1 or 2 TCR retrovirus. The transduced TCRs are comprised of mouse constant regions and human variable regions. Next, the mTCRβ⁺ CD8⁺ population was sorted. The top panel shows the cells before sorting and the bottom panel—after sorting. (17b: CDK4, 17c: CMV) Activation of TCR-transduced or non-transduced Jurkat cells as assessed by CD69 upregulation after a 20-h co-culture with JY cells (E:T ratio was 1:1) loaded with graded concentrations of the cognate peptide. The EC50 values are depicted in the graph legends. PMA/ionomycin stimulation was used as a positive control and culturing without JY cells and with JY cells loaded with the highest concentration (1 μg/ml) of irrelevant peptides were used as negative controls (depicted on the right side of each graph). The assay was performed in triplicate (non-transduced Jurkat cells with CDK4 mutant peptide in duplicate). Data shown as mean±s.d (the error bars are only shown in the peptide titration curves). n=1. NT, non-transduced.

FIGS. 18a-18e. Blasticidin selection of CMV-1-transduced Jurkat TCR KO cells with different transduction efficiencies. (18a) Jurkat TCR KO cells were transduced with different volumes of CMV-1-basticidin retroviral supernatant to achieve different transduction efficiencies. Non-transduced and TCR-transduced Jurkat cells were stained for CD8 and mTCRβ, (18b, 18c, 18d, 18e) Jurkat TCR KO cells with different CMV-1 transduction efficiencies were plated at a concentration of 0.25×10⁶ cells/ml and selected with different blasticidin concentrations. After four days the cells were re-plated at a density of 0.25×10⁶ cells ml by either removing the blasticidin (referred to as ‘removed the blasticidin on day 4’) or by adding new blasticidin with the respective concentration (referred to as ‘added new blasticidin on day 4’). (18b, 18d) Fold expansion of total live and mTCRβ⁺ CD8⁺ cells six or seven days after starting the blasticidin selection, respectively. (18c, 18e) The percentage of mTCRβ⁺ CD8⁺ cells six or seven days after initiating the blasticidin selection, respectively. n=1. NT, non-transduced.

FIGS. 19a-19d. Upscaling the Jurkat cell-APC co-cultures. (a,b,c,d—left) The CD8 and mTCRβ expression of the effector Jurkat cells in each experiment. The Jurkat cells were either transduced with (19a) a TCR library composed of 16 TCRs, four of which are specific for antigens contained within TMG2.1, or with (19b, 19c, 19d) the CDK4-17 TCR. (19b, 19c) The double-negative populations are target cells which were not possible to gate out due to their low CD20 expression. (19a, 19b, 19c, 19d—right) Co-cultures were performed with. TMG2.1-expressing EBV LCLs for 20 h (E:T ratio was 1:1, 2.5×10⁶ cells/ml cell density). Non-transduced EBV LC.Ls were used as a negative control. The percentage of total live cells and. CD69⁺ effector cells are plotted. The text in green indicates which conditions were spun down at 1100 rpm for 1 min (short spin). Total cell numbers used: 0.5×10⁶ cells (96W), ≤2×10⁶ cells (1.3/2×10⁶ cells in 15/50 ml Falcon, respectively), 170×10⁶ cells (GMP bag). The 96W co-cultures were performed in (19c) triplicate or (19d) duplicate. Data shown as mean±s.d. n=1 96W, 96 well round-bottom plate. GMP bag, MACS GMP Cell Differentiation Bag—500. *data from a low number of live cells.

FIGS. 20a-20e, Longitudinal analysis of the T cell activation markers CD69, CD25 and CD62L. Jurkat TCR KO cells transduced with either CDK4-8 or CMV-1 TCR (>80% mTCRβ⁺ CD8⁺ cells) were cultured in the presence (CDK4-8: circles, CMV-1: triangles) or absence (CDK4-8: rectangles, CMV-1: reversed triangles) of TMG2.1-expressing EBV LCLs for 16, 20, 24, 28 and 32 h at an E:T ratio 1:1, followed by a multi-color flow cytometric analysis of the effector cells' CD69, CD25 and CD62L expression. Non-transduced Jurkat TCR KO cells co-cultured with EBV LCLs TMG2.1 were used as a negative control (diamonds). Each representative dot plot is from the 20-h co-culture of CDK4-8-transduced Jurkat cells with EBV :LCLs TMG2.1. (20a) CD69 upregulation, (20b) CD25 upregulation and (20d) CD62L downregulation of the effector cells. Co-expression analysis of CD69 with either (20c) CD25 or (20e) CD62L. The co-cultures were performed in triplicate (shown as mean±s.d), n=1. NT, non-transduced.

FIGS. 21a-21d. Assessment of the efficacy of NFAT-based reporter lentiviral vectors with a puromycin resistance gene in Jurkat TCR KO cells. (21a) Schematic representation of the four NFAT-based reporter lentiviral plasmids with a puromycin resistance cassette. (21b) The four vectors were transfected into HEK293T cells by using PEI as a transfection reagent. An additional plasmid (pniaxGFP) was co-transfected as a measure of the transfection efficiency. The dot plots show the GFP expression of non-transfected and transfected HEK293T cells three days post-transfection. (21c) The lentiviral supernatant was used in combination with polybrene to transduce Jurkat TCR KO cells. The Jurkat cells were stimulated with PMA/ionomycin for 24 h and subsequently selected with 1 μg/ml puromycin for three days. The dot plots display the percentage of live (DAN) and CD69⁺ non-transduced and transduced Jurkat TCR KO cells. (21d) The transduced and puromycin-selected cells were expanded for 20 days in order to undergo a second round of PMA/ionomycin stimulation for 24 h, followed by a 4-day puromycin selection with varying puromycin concentrations. The percentage of live (DAPI⁻) and CD69⁺ cells are plotted. Note that the NT cells are different from the NT cells in (c). n=1. NT, non-transduced.

FIGS. 22a-22c Assessment of the efficacy of EGFP NFAT-based reporter lentiviral vectors in Jurkat TCR KO cells. (22a) Schematic representation of the four NFAT-based reporter lentiviral plasmids with an EGFP gene. (22b) The four NFAT vectors were transfected into HEK293T cells using PEI or FuGENE as a transfection reagent. The percentage of GFP⁺ HEK293T cells three days post-transfection is shown. (22c) The lentiviral supernatant from the PEI transfections was transduced into Jurkat TCR KO cells with the aid of polybrene as a transduction reagent. The NFAT-transduced Jurkat TCR KO cells were stimulated with PMA/ionomycin for 24 h and the GFP expression (shown as percentage and MFI of GFP cells) was measured every 24 h for three days. The activation of the Jurkat cells was assessed by CD69 upregulation. n=1. MFI, mean fluorescence intensity. EGFP, enhanced GFP. NT, non-transduced.

FIG. 23. Evaluation of the efficacy of EGFP NFAT-based reporter lentiviral vectors in primary T cells. Primary T cells were transduced with either the NFAT4x lentiviral vector used in FIG. 23 or an NFAT4x lentiviral vector (NFAT4x new) that contains a different minimal promoter (minP). The NFAT-transduced primary T cells were stimulated with PMA/ionomycin for 24 h and the GFP expression (shown as percentage and MFI of GFP⁺ cells) was measured every 24 h for three days. The activation of the Jurkat cells was assessed by CD69 expression. n=2 biologically independent replicates (shown as mean±s.d). *P≤0.05, **P≤0.01, ns: not significant (two-way ANOVA followed by Sidak's multiple comparisons test). MFI, mean fluorescence intensity. NT, non-transduced.

FIGS. 24a-24d. Non-viral delivery of NFAT-based reporter plasmids in Jurkat TCR KO cells. (24a) Schematic representation of the two NFAT plasmids. NFAT0x does not contain any NFAT binding sites and was used to assess the background signal of the minP alone. (b,c,d) NFAT0x and NFAT4x were electroporated into CDK4-17-transduced Jurkat TCR KO cells (˜90% mTCRβ⁺ CD8⁺ cells). (24b) The cell viability of Jurkat cells that were either non-electroporated or electroporated in the presence or absence of plasmid DNA was assessed 20 h, 2 days and 6 days after the electroporation. (24c) Longitudinal analysis of the percentage of E2-Crimson and GFP single and double-positive populations of NFAT-transfected Jurkat cells. Antibiotic selection was initiated at day 6 with 0.5 μg/ml puromycin for seven days. The puromycin was refreshed four days after starting the selection. (24d) Dot plots of the cell viability (DAPI⁺ cells) and the GFP and E2-Crimson expression seven days after the puromycin selection. n=1. minP, minimal promoter. EGFP, enhanced GFP.

FIGS. 25a, 25b. Blasticidin selection of non-transduced Jurkat TCR KO cells. Non-transduced Jurkat TCR KO cells were plated at a concentration of 0.25×10⁶cells/ml and subjected to selection with different blasticidin concentrations for six days. After four days the cells were re-plated at a concentration of 0.25×10⁶ cells/ml by either removing the blasticidin (referred to as ‘removed the blasticidin on day 4’) or adding new blasticidin with the respective concentration (referred to as ‘added new blasticidin on day 4’). (25a) The fold expansion of total live cells after blasticidin selection. (25b) Flow-cytometry plots showing FSC-A/SSC-A and CD8/mTCRβ expression of non-transduced cells cultured in the presence or absence of 4 μg/ml blasticidin. n=1. NT, non-transduced.

FIGS. 26a, 26b. mTCRβ MFI of 4 μg/ml blasticidin-selected CMV-1-transduced Jurkat TCR KO cells with different transduction efficiencies. (26a, 26b) Jurkat TCR KO cells with different CMV-1 TCR retroviral transduction efficiencies were plated at a concentration of 0.25×10⁶ cells/ml and selected with 4 μg/ml blasticidin. After four days the cells were re-plated at a concentration of 0.25×10⁶ cells/ml by either removing the blasticidin (referred to as: removed the blasticidin on day 4) or adding new 4 μg/ml blasticidin (referred to as: added new blasticidin on day 4). (26a) mTCRβ MFI of mTCRβ⁺ CD8⁺ cells selected with blasticidin for six/seven days. (26b) Representative dot plots showing the CD8 and mTCRβ staining seven days after starting the blasticidin selection, n=1, MFI, mean fluorescence intensity.

FIG. 27. Measurement of T cell activation using two different anti-human CD69 monoclonal antibody, clone FN50 and clone CH/4. The experimental setup is described in the legend of FIG. 19b. Cells were simultaneously stained for CD69 clone FN50 (APC) and CD69 clone CH/4 (PE). n=1. 96W, 96 well round-bottom plate. *data from a low number of live cells.

FIG. 28. Depicts some embodiments of aa neo-antigen specific TCR isolation platform.

FIG. 29. Enhance the scalability of the TCR isolation platform by enabling a more efficient processing of large cell numbers while still maintaining TCR coverage.

FIG. 30. Methods to test the efficiency and toxicity of blasticidin.

FIG. 31. Depicts various embodiments of a TCR platform isolation platform, which can include all of the steps, or each of the boxed steps (e.g., 1, and/or 2, and/or 3, and/or 4), or any one of more of the linear numbered step (e.g., 1-7)

FIG. 32. Depicts SEQ ID NO: 1: Amino acid sequence for a TCR gene expressed as a TCRβ-P2A-TCRα-T2A-Puromycin resistance expression cassette; and SEQ ID NO: 4: Amino acid sequence for a TCR gene expressed as a TCRβ-P2A-TCRα-T2A-Blasticidin resistance expression cassette.

FIG. 33. SEQ ID NO: 2 Amino acid sequence for a CD8α-P2A-CD8β transgene.

FIG. 34. SEQ ID NO 3: Example nucleotide sequence for HLA-A*02:01-IRES-FusionRed.

FIG. 35A shows the schematic of the screen design. Five characterized TCRs and 95 uncharacterized TCRs from ovarian cancer (OVC) or colorectal cancer (CRC) samples were used to create combinatorial TCR libraries of 100×100 design.

FIG. 35B shows cell sorting results for T cell activation by FACS using the CD69 marker.

FIG. 35C shows that the resulting PCR product, where the PCR had a limited number of cycles to amplify part of the TCRβ-P2A-TCRα cassette from the sorted TCR transduced Jurkat T cells, has a size of approx. 1.5 kb.

FIG. 35D shows TCR enrichment analysis of the screen data from FIG. 35C.

FIG. 35E shows characteristics of the five characterized antigen reactive TCRs.

FIG. 36A shows the schematic of the screen design. Five characterized TCRs and 95 uncharacterized TCRs from ovarian cancer (OVC) or colorectal cancer (CRC) samples were used to create combinatorial TCR libraries of 100×100 design.

FIG. 36B shows the sorting strategy for the screen.

FIG. 36C shows the retrieval of TCR expression cassettes.

FIG. 36D shows TCR enrichment analysis of the screen data from FIG. 36C.

FIG. 36E shows characteristics of the top 7 most significantly enriched TCRs in FIG. 36D.

FIG. 37A shows the schematic of a 6×6 combinatorial TMG encoding design.

FIG. 37B shows the analysis of the rank order of all TCR alpha x beta combinations as a function of the number of replicates of the pt2 TCR library screen.

FIG. 37C shows that summary table of the statistical analyses based on 2 or 3 replicates of the CRC TCR library screens.

FIG. 37D shows the table of e pt4 samples used for pairwise TCR enrichment analysis.

FIG. 37E shows that pairwise TCR enrichment analysis results.

FIG. 38 shows the correlation of TCR activation and TCR background activation between screening and validation data.

FIG. 39 denotes a measure of TCR representation. The graph shows that five characterized TCRs which are stimulated with their cognate antigens are enriched in the top samples (lightest grey shade). The bottom samples derived from cocultures with B cells expressing TMG show lower rlog-values.

FIG. 40 depicts a diagram for SEQ :ID NO: 1, a TCR gene expressed as a TCRβ-P2ATCRα-T2A-Puromycin resistance expression cassette.

FIG. 41 depicts a diagram for SEQ ID NO: 2, a CD8α-P2A-CD8β transgene.

FIG. 42 depicts a diagram for SEQ ID NO 3: K562-HLA-A*02:01-IRESFusionRed.

FIG. 43 depicts a diagram for SEQ :ID NO: 4, a TCR gene expressed as a TCRβ-P2A-TCRα-T2ABlasticidin resistance expression cassette.

### DETAILED DESCRIPTION OF VARIOUS EMBODIMENTS

The present disclosure provides methods and compositions for recovering a repertoire of T cell receptors (TCRs) from a diverse T cell populations. Some embodiments of the methods provide for the identification and isolation of antigen-specific TCRs from non-viable material, including human tissue specimens. Some embodiments are according to some or all of FIG. 31. In some embodiments, the method can include one or more of steps (1)-(7) outlined in FIG. 31 and herein. For example, the method can involve (1) Obtaining a sample. The sample can be tissues, blood, or body fluids from a patient suffering infectious diseases, autoimmune diseases, or cancers. The sample can be viable or non-viable. Step (2) Sequencing TCR-α and β chains in the sample. Step (3) Selecting and combinatorial pairing TCRα- and β-chain sequences to create a library of TCRαβ pairs. Step (4) introducing the library of TCRαβ pairs into a pool of reporter cells, for example, Jurkat reporter T cells. Step (5) Stimulating the reporter cells that are modified with the library of TCRαβ pairs with antigen presenting cells presenting at least one antigen of interest. The at least one antigen of interest can be autologous or allogeneic. Step (6) Determining TCRαβ pairs specific to the at least one antigen of interest. Step (7) Introducing the TCRαβ pairs into cells and selecting cells containing the TCRαβ pairs. In some embodiments, the method can involve one or more of the steps (1)-(7) described above. In some embodiments, any of the steps can be omitted, repeated, or substituted by other embodiments provided herein, as appropriate. Additional intervening steps can also be added.

In some embodiments, for any of the methods herein, the TCR pairs and/or the T cells expressing the TCR pairs are selected or identified by binding to an antigen (such as a neoantigen), wherein the antigen is expressed by a B cell or an antigen presenting cell. In some embodiments, for any one of the methods herein, the antigen or neoantigen is from a tumor in a subject, and the TCR alpha and the TCR beta of the TCR pairs are also each from the subject. In some embodiments, there are at least 2, 3, 4, 5, 6, 7, 8, 9, 10, 50, 100, 500, 1000, 10000, 100000, or 1 million TCR pairs (or cells comprising these pairs) and there are at least 2, 3, 4, 5, 6, 7, 8, 9, 10, 50, 100, 500, 1000, 10000, 100000, or 1 million antigens present.

In some embodiments, any of the compositions employed and/or resulting from the above methods are also contemplated as libraries and/or kits and/or compositions and/or for their application in medical applications and/or screening systems. In some embodiments, the composition can be a TCRα- and β-chain pairing that has been selected or generated by any of the methods provided herein. In some embodiments, the composition can be any of the components involved in or products from any of the methods provided herein.

Various cell compositions, libraries, cell and protein related therapeutics are provided as well. In some embodiments, a coculture comprises at least a first and second type of cells for a population of cells. In sonic embodiments, first and second type of cells in a coculture can contact and induce phenotypic changes. In some embodiments, the coculture is maintained in a culture vessel. In some embodiments, the coculture is maintained in a culture bag. In some embodiments, the first and second type of cells are two or more populations of different cell types. In some embodiments, the population of cells are exactly two populations of different cell types. In some embodiments, any of the collections of cells or intermediates or resulting cell populations from any of the methods provided herein are contemplated as specific compositions, libraries, therapeutics, etc. In some embodiments, the coculture comprises a T cell and a B cell.

In some embodiments, a first type of cell in the coculture is a T cell. In some embodiments, the first type comprises a human T cell. In some embodiments, the first type comprises a Jurkat T cell. In some embodiments, the first type comprises a Jurkat T cell that is engineered to express human CD8a and CD8b and that lacks endogenous TCR expression. In some embodiments, the first type comprises a Jurkat T cell that expresses one or more variant nucleic acid molecules. In some embodiments, the first type comprises a jurkat T cell that expresses one or more variant TCRs. In some embodiments, the Jurkat T cells express low background levels of activation markers, including but not limited to, CD69, due to a preculture at low density.

In some embodiments, the second type of cell in the coculture comprises a cell that can present antigens. In some embodiments, the other type of cell comprises a human cell that can present antigens. In some embodiments, the other type of cell comprises a human tumor cell. In some embodiments, the other type of cell comprises a human B cell. In some embodiments, the other type of cell comprises a human autologous B cell. In some embodiments, the other type of cell comprises a human autologous immortalized B cell. In some embodiments, the B cell population is engineered to express an exogenous antigen. In some embodiments, the B cell population is engineered to express multiple exogenous antigens. In some embodiments, the B cell population is engineered to express multiple exogenous neo-antigens. In some embodiments, the B cell population is engineered to express multiple exogenous neo-antigens in the format of multiple minigenes. In some embodiments, the B cell population is engineered to express multiple exogenous neo-antigens in the format of single TMGs. In some embodiments, the B cell population is engineered to express multiple exogenous neo-antigens in the format of multiple TMGs. In some embodiments, individual cells in the B cell population express only a single exogenous minigene or TMG. In some embodiments, individual cells in the B cell population can express multiple exogenous minigenes or TMGs.

In some embodiments, a composition is provided. The compositions comprises: a first population of T cells that are activated as measured by one or more T cell activation markers; and ii) a second population of another selection of T cells as a reference population, expressing the same TCR library or in in the plasmid pool of the same TCR library, wherein one or more TCRs are enriched in the first population of T cells relative to the second population of T cells.

In some embodiments, a collection of cells is provided, the collection comprises a set of T cells that are configured to express at least one TCR alpha and TCR beta pair, wherein the pair is from a subject, wherein the T cells do not express an endogenous TCR, and wherein the set of T cells are configured for activation of one or more T cell activation markers; and a set of B cells, wherein the set of B cells is configured to express at least one exogenous neo-antigen, and wherein the at least one exogenous neoantigen is from a tumor from the subject.

In some embodiments, there are at least two TCR pairs and at least two exogenous neo-antigens present in the collection. In some embodiments, there are at least, 2, 3, 4, 5, 6, 7, 8, 9, 10, 50, 100, 500, 1000, 10000, 100000, or 1 million TCR pairs (or cells comprising the pairs) in the composition. In some embodiments, there are at least 2, 3, 4, 5, 6, 7, 8, 9, 10, 50, 100, 500, 1000, 10000, 100000, or 1 million antigens (or B cells expressing these antigens) present in the collection. In some embodiments, there are there are at least, 2, 3, 4, 5, 6, 7, 8, 9, 10, 50, 100, 500, 1000, 10000, 100000, or 1 million TCR pairs (or cells comprising these pairs) in the composition and there are at least 2, 3, 4, 5, 6, 7, 8, 9, 10, 50, 100, 500, 1000, 10000, 100000, or 1 million antigens present in the collection. In some embodiments, the sequences for each pair or part thereof is different. In some embodiments, each TCR pair is different.

In some embodiments, the ratio in which the two or more populations of cells are present in the coculture is within the range of 1000:1 to 1:1000.

In some embodiments, the ratio in which the two or more populations of cells are present in the coculture is set to allow for inducing a contact-induced phenotype.

In some embodiments, the ratio in which the B and T cell populations are present in the coculture allows for T cell activation.

In some embodiments, the ratio in which the B and T cell populations are present in the coculture allows for T cell activation of a subset of T cells expressing specific TCRs. In some embodiments, the ratio in which the two or more populations of cells are present in the coculture is within the range of 1000:1 to 1:1000. In some embodiments, the ratio is adequate to lead to TCR cell activation.

In some embodiments, a coculture or composition comprises a) Jurkat T cells that have been engineered to express human CD8a, CD8b and a TCR variant library, and that lacks endogenous TCR expression; and b) autologous human B cells that are immortalized and where the autologous human B cells express multiple antigens in the form of single minigenes or TMGs. In some embodiments, the coculture is maintained in a culture vessel or culture bag, and B and T cell populations are present at a ratio that allows T cell activation. Antigen-specific T cell activation is only mediated via specific TCRs in the TCR library.

In some embodiments, there is a number and/or ratio of T cells (expressing TCR pairs) to B cells (providing neo-antigens) of at least 2 to 2, for example, at least 10 to 10, at least 1000 (TCR pairs) to 10 (neo-antigens), at least 10,000 (TCR pairs) to 10 (neo-antigens), at least 10,000 (TCR pairs) to 100 (neo-antigens), at least at least 10,000 (TCR pairs) to 1000 (neo-antigens), at least 100,000 (TCR pairs) to 10, 100, 1000, or 10,000 (antigens), at least 50 to 50, at least 100 to 100, at least 1,000,000 (TCRs) to 10,000 (antigens), including any range defined between any two of the preceding ratios. In some embodiments, a single TCR pair and/or antigen is present in each cell (T or B), such that each of the numbers above can represent cell number as well.

In some embodiments, the composition comprises a coculture of B and T cells, wherein there are at least two different TCR pairs expressed by the T cells and wherein there are at least two different antigens expressed by the B cells.

In some embodiments, there are a multiplicity of TCRs in the T cells and multiplicity of antigens in the B cells. In some embodiments, the composition is configured such that one can induce T cell activation mediated by at least one TCR and one antigen (or at least two or more TCRs and/or two or more antigens). In some embodiments, there are low background levels of CD69 in the composition. In some embodiments, the composition includes autologous APCs (or autologous immortalized B cells). In some embodiments, the T cells are engineered to express a TCR library (and/or lacking endogenous TCR expression). In some embodiments, the T cells are configured such that they are deprived of native TCR expression, but capable of TCR activation via exogenous TCR pairs.

In some embodiments, a collection of cells is provided. The collection comprises: a set of at least two T cells. In some embodiments, each is configured to express at least one TCR alpha and TCR beta pair. In some embodiments, the TCR alpha and the TCR beta are each from a subject, the T cells do not express an endogenous TCR, and the set are configured for activation of one or more T cell activation markers. The collection further comprises a set of at least two B cells. Each of the at least two B cells is configured to express at least one exogenous neo-antigen (or antigen) such that there are at least two exogenous neo-antigens (or antigens) capable of being produced, and the at least two exogenous neo-antigens (or antigens) are the same as those in the subject.

In some embodiments, in the cell composition, the set of at least two B cells comprises: at least a first B cell that produces the exogenous neo-antigen (or antigen); and at least a second B cell that produces the second exogenous neo-antigen (or antigen).

In some embodiments, a library of TCRs (or TCR expressing cells) is provided. The library of comprises: a set of at least three T cells, wherein at least two of the T cells are configured to express at least two TCR alpha and TCR beta pairs (at least two TCR pairs), wherein the at least two TCR pairs are from a subject, wherein the at least three T cells do not express an endogenous TCR, wherein the at least three T cells are configured for activation of one or more T cell activation markers, upon binding to an antigen (or neo-antigen), presented by a B cell, wherein an amount of genomic copies of each TCR pair as reflected in a number of TCR cells is such that one gets a read on every TCR in the sample, and wherein at least one of the TCRs is not distributed equally throughout a composition comprising the library.

In some embodiments, a distribution of at least one T cells is altered by binding to an antigen presented by a B cell. In some embodiments, the at least two TCR pairs are approximately evenly present in the library.

In some embodiments, a collection of cells is provided. The collection comprises: a set of at least two T cells, wherein each is configured to express at least one TCR alpha and TCR beta pair, wherein the pair is from a subject, wherein the T cells do not express an endogenous TCR, and wherein the set are configured for activation of one or more T cell activation markers. The collection of cells can further comprises a set of at least two antigen present cells (APCs), wherein each of the at least two APCs is configured to express at least one exogenous neo-antigen (or antigen), such that there are at least two exogenous neo-antigens (or antigens) capable of being produced, and wherein the at least two exogenous neo-antigens (or antigens) are the same as those in the subject.

In some embodiments, a set or kit comprises a first population of T cells and a second population of T cells. In some embodiments, a first population of T cells is composed of T cells that share a certain phenotype that can be measured. In some embodiments, the first population of T cells comprises T cells that share the phenotype of T cell activation. In some embodiments, the first population of T cells can be a selected population of T cells.

In some embodiments, the first population of T cells comprises T cells that share a certain expression level of one or more marker or markers. In some embodiments, a first population of T cells comprises T cells that share expression of one or more T cell activation marker or markers.

In some embodiments, the T cells express a library of variant nucleic acid molecules. In some embodiments, the T cells express a TCR library.

In some embodiments, a second population of T cells can be a reference population. The reference population can be a selected or unselected population of T cells that express the same TCR library that is expressed by the first selected population of T cells. In some embodiments, a reference is the plasmid pool of the same TCR library that is expressed by the first population of T cells.

In some embodiments, an amount of each TCR is such that it is possible to get a read on every TCR in the sample, and wherein there is at least one TCR pair that is not equally distributed throughout the composition. In some embodiments, an amount of genomic copies of each TCR pair as reflected in a number of TCR cells is such that one gets a read on every TCR in the sample. In some embodiments, at least one of the TCRs is not expressed equally throughout a composition comprising the library. In some embodiments, a majority of TCRs is roughly equally represented among the selected population of T cells and the reference. In some embodiments, more than 90% of all TCRs present in the TCR library are represented in both the first population of T cells and the second population of T cells (e.g., the reference population). In some embodiments, more than 99% of all TCRs present in the TCR library are represented in both the first population of T cells and in the second population of T cells.

In some embodiments, one or more TCRs are enriched in the first population relative to the second population. In some embodiments, one or more TCRs are statistically significantly enriched in the first population relative to the second population.

In some embodiments, the composition comprises the TCRs (or cells expressing these TCRs) that are the top 1, 2, 3, 4, 5, 6, 7, 8, 9, or at least 10% from the screening method, e.g., top-bottom comparison.

In some embodiments, more than 99% of all TCRs are present among the first (selected) population of cells. In some embodiments, the majority of TCRs in a composition are roughly equally distributed among the first population of T cells and the second (e.g., reference in this situation).

In some embodiments, at least one TCRαβ pair with desired features is identified, isolated, and/or provided. In some embodiments, at least one TCRαβ pair with desired features originates from tumor-infiltrating lymphocytes (TIL). In some embodiments, at least one TCRαβ pair with desired features originates from tumor-infiltrating lymphocytes (TIL) and are used for cancer therapy in the same subject. In some embodiments, at least one TCRαβ pair with desired features originates from peripheral blood. In some embodiments, the desired feature is specificity for an antigen. In some embodiments, the desired feature is recognition of a neo-antigen. In some embodiments, the desired feature is recognition of a viral antigen. In some embodiments, the desired feature is recognition of a shared antigen expressed by tumor cells. In some embodiments, the desired feature is restriction to MHC-class I or MHC-Class II. In some embodiments, the desired feature is avidity for an antigen. In some embodiments, the desired feature is absence of reactivity for an antigen. In some embodiments, multiple features are desirable. In some embodiments, that TCR pair is configured for any one or more of these features.

In some embodiments, at least one TCRαβ pair with desired features is used and/or prepared and/or conditioned for therapy. In some embodiments, at least one TCRαβ pair is used and/or prepared and/or conditioned for therapy. In some embodiments, at least one TCRαβ pair is used or configured for use for cancer therapy. In some embodiments, at least one TCRαβ pair is used and/or prepared and/or conditioned for a therapy of infectious disease. In some embodiments, at least one TCRαβ pair is used and/or prepared and/or conditioned for therapy of an autoimmune disease. In some embodiments, at least one TCRαβ pair is used and/or prepared and/or conditioned to engineer a recombinant protein for therapy. In some embodiments, the recombinant protein is administered for therapy.

In some embodiments, at least one TCRαβ pair is used to engineer cells for therapy. In some embodiments, at least two TCRαβ pairs are used to engineer T cells for therapy. In some embodiments, more than two TCRαβ pairs are used to engineer T cells for therapy. In some embodiments, five TCRαβ pairs are used to engineer T cells for therapy. In some embodiments, ten TCRαβ pairs are used to engineer T cells for therapy. In some embodiments, twenty TCRαβ pairs are used to engineer T cells for therapy. In some embodiments, engineered cells are administered for therapy. In some embodiments, a TCRαβ pair is introduced into T cells using virus. In some embodiments, the virus is a lentivirus. In some embodiments, the virus is a retrovirus. In some embodiments, the virus is an adenovirus. In some embodiments, the virus mediates integration of the ICR into the genome of the T cell.

In some embodiments, the virus leads to transient expression of the TCR. In some embodiments, the virus carries the TCR DNA as a repair template of genomic double-strand breaks in T cells by Homology-directed-repair (HDR).

In some embodiments, a TCRαβ pair is introduced into T cells using non-viral gene delivery methods. In some embodiments, the non-viral gene delivery method is based on electroporation. In some embodiments, the non-viral gene delivery method is based on other methods that can introduce temporary perforation of the cell membrane of cells to deliver components into the T cell. In some embodiments, the non-viral gene delivery method involves transposases. In some embodiments, the non-viral gene delivery method involves nucleases.

In some embodiments, the nuclease is a CRISPR/Cas9 complex.

In some embodiments, engineered T cells are modified with a TCR and further genetically modified to control their phenotype and reactivity.

In some embodiments, engineered T cells expressing different TCRαβ pairs with specificity for different antigens are combined into a cell composition for administration. In some embodiments, the combination allows one to target multiple antigens, which can be more effective than monotherapy. In some embodiments, the combination allows for both MHC-Class I and MHC-Class II restricted T cells together which can synergize for the therapy of solid cancer. In some embodiments, the combination allows for truncal and branch tumor mutations to be targeted together. In some embodiments, the combination is based on utilizing equal ratios of each engineered T cell population. In some embodiments, the combination is based on utilizing different cell numbers of each engineered T cell population.

In some embodiments, the composition comprises an engineered T cell product based on using more than one TCR gene. In some embodiments, the engineered cell includes at least one of the following: it is autologous to the patient receiving it; it is TIL-derived; it employs use of at least one Class I and one Class II TCR; and it employs equal ratios.

Some of the embodiments provided herein circumvent the need to recover native combinations of TCRα- and β-chains and can be applied to non-viable cell material and non-viable tissue samples. By the generation of combinatorial TCRαβ libraries, some embodiments of the present disclosure allow identification of antigen-specific TCRαβ pairs from stored or archived samples. For example, embodiments of the present disclosure can solve the problem associated with mixing of TCRα and TCRβ mRNA transcripts from different T cells resulting from loss of cell membrane integrity of non-viable T cells. In such mixtures, information on original TCRαβ pairs is lost. Some embodiments of the methods provided herein solve the low sensitivity of previously described. TCR library screening technologies caused by bias of recovered TCR libraries towards TCR sequences with high frequency. Some embodiments of the methods provided herein eliminate the need to include the complete repertoire of recovered TCR chains in downstream applications, allowing one to e.g. focus TCR discovery to TCR chains with desirable properties. Unlike single-cell approaches, such as Droplet PCR and microfluidic devices, the methods of recovering specific TCRαβ pairs from T cells disclosed herein do not require specific instrumentation and viable cell material that limit scalability. Unlike bulk PCR methods to recover collections of TCRα and TCRβ sequences from a T cell population that a priori do not allow the recovery of native TCRαβ pairs, the methods disclosed herein employ a design to recover a defined part of the identified TCR repertoire to recover TCRαβ pairs of interest.

The methods disclosed herein can be used for therapeutic and diagnostic purposes and/or compositions, and/or medicaments. Recombinant TCR genes can be used to produce T cells with desired specificity for immunotherapy, including cancer immunotherapy, for example. For cancer immunotherapy applications, T cells with desired specificity can be produced by selecting TCR genes to generate antigen-specific T cells by TCR gene transfer. In some embodiments, the approach is based on the observation that antigen-specificity can be transferred between T cells by the transfer of the genes encoding the TCRαβ pair. TCR genes of interest can be introduced into the genome of human T cells by utilizing y-retroviral or lentiviral vectors, transposon-based gene delivery platforms, mRNA delivery (e.g. by electroporation or nanoparticles) or genome-engineering tools, including CRISPR/Cas9. The latter enables the simultaneous knock-out of endogenous TCR chains by site-specific integration of novel TCR chains into the endogenous TCR loci.

In some embodiments, the resulting selected pair of molecules is used for the treatment of a subject and/or a medicament for a subject for any of the disorders provided herein.

In sonic embodiments, a method of treating a subject is provided. The method comprises identifying a subject having a tumor; providing a set of at least two T cells, each of which is configured to express at least one different TCR alpha and TCR beta pair, wherein each of the TCR alpha and the TCR beta are from the subject, providing a set of at least two B cells, wherein the set of B cells is configured to express at least two exogenous neo-antigens, and wherein the at least two exogenous neoantigens are the same as those neo-antigens found in the subject; combining the set of at least two T cells with the set of at least two B cells and selecting a combination of at least two TCR pairs based upon activation of the at least two T cells via the at least two exogenous neo-antigens; and administering the combination of at least two TCR pairs to the subject, thereby treating the tumor. In some embodiments, any of the number of T cell to B cells provided herein can be used in the process. In some embodiments, treating reduces a size of the tumor. In some embodiments, it reduces the size of the tumor by at least 1, 2, 3, 4, 5, 10, 20, 30, 40, 50, 60, 70, 80, 90 95, 99, or 100%, including any range defined between any two of the preceding values.

In some embodiments, a method of treating a subject is provided. The method comprises identifying a subject having a tumor; providing a set of at least two T cells, each of which is configured to express at least one different TCR alpha and TCR beta pair, wherein each of the TCR alpha and the TCR beta are from the subject; providing a set of at least two antigen presenting cells, wherein the set of antigen-presenting cells originates from the subject, is configured to express at least two exogenous neo-antigens, and wherein the at least two exogenous neoantigens are the same as those neo-antigens found in the subject; combining the set of at least two T cells with the set of at least two antigen present cells and selecting a combination of at least two TCR pairs based upon activation of the at least two T cells via the at least two exogenous neo-antigens; and administering the combination of at least two TCR pairs to the subject, thereby treating the tumor. In some embodiments, there are more than two TCR pairs, e.g., 2, 3, 4, 5, 7, or more pairs of TCRs can be employed. In some embodiments, the TCR pairs are administered via a cell therapy. In some embodiments, the pairs have different sequences from other pairs.

In some embodiments, any of the selected TCR pairs or combinations of pairs provided herein by any of the methods can be used in the methods of treatment provided herein.

In some embodiments, a pharmaceutical composition is provided. In some embodiments, the pharmaceutical composition comprises a first TCR pair, that binds to a first antigen (or neo-antigen) in a subject's tumor; and a second. TCR pair, that binds to a second antigen (or neo-antigen) in the subject's tumor. In some embodiments, there are more than two TCR pairs, e.g., 2, 3, 4, 5, 6, 7, or more pairs of TCRs can be employed. In some embodiments, the TCR pairs are administered via a cell therapy. In some embodiments, the pairs have different sequences from other pairs. In some embodiments, the first TCR pair is MHC-class I restricted and wherein the second TCR pair is MHC-class II restricted.

In some embodiments, a pharmaceutical composition is provided. It can include a first TCR pair, that binds to a first antigen and is MHC-class I restricted; and a second TCR pair, that binds to a second antigen and is MHC-class II restricted.

In some embodiments, the composition can further comprise a third TCR pair.

In some embodiments, the first TCR pair binds to a neo-antigen from a tumor, wherein the second TCR pair binds to a neo-antigen from the tumor, and wherein both. the first and second TCR pairs are present in a host of the tumor.

Recombinant TCR genes for therapeutic use in cancer can be obtained from different sources. First, it is possible to detect and isolate T cells with specificity for tumor antigens from viable patient specimens, such as blood or tumor tissue. Technologies described in the art include isolation of MHC-multimer binding T cells as well as the isolation of T cells expressing certain phenotypic markers or secreting certain cytokines after antigen-stimulation by flow cytometry or magnetic bead selection. Subsequently, isolated antigen-specific T cells can be used to determine the sequence of the expressed TCR genes by single cell PCR-based techniques, TCR bulk chain sequencing or microfluidic based PCR techniques. Second, allo-CTL systems or animal models (e.g. HLA-transgenic and/or human TCR transgenic mouse models) provide an alternative source for tumor-antigen specific T cells/TCRs. Third, therapeutic TCR genes can be selected from in vitro mutated TCR chains expressed as recombinant TCR libraries by phage-, yeast- or T cell-display systems.

T cells (or TCRs) for cancer therapy can be selected based on desirable therapeutic criteria; first, TCR genes used for cancer therapy ideally recognize a tumor-specific antigen with low or absent expression in vital tissues. Second, the TCR should recognize its antigen with high sensitivity, e.g. small antigen amounts should trigger effector functions of TCR-modified T cells against tumor cells, for example cytolytic activity. Third, the TCR should have no cross-reactivity against other antigens with expression in vital tissues.

Different tumor-antigens can be targeted by TCR gene transfer, including cell-lineage specific antigens (e.g. MART-1), overexpressed antigens (e.g. WT-1), cancer/testis (C/T) antigens (such as NY-ESO-1, MAGE-A4, MAGE-A10), viral antigens (e.g. HPV E6, E7), and mutated proteins (neo-antigens). Of note, neo-antigen specific TCR sequences can be particularly suitable for the treatment of cancer. For example, neo-antigen specific T cells have been correlated with regression of advanced, metastatic cancer after both immune-checkpoint blockade therapy as well as adoptive T cell therapy. Because the vast majority of genomic mutations in tumors are passenger mutations and are found in only a small fraction of tumors, and because of MHC-restriction, the repertoire of tumor neo-antigens that can be recognized by T cells is largely different between individual patients. Thus, utilizing TCR gene transfer to generate neo-antigen specific T cells for therapy will often require one or more new neo-antigen specific TCR sequences for every patient or tumor. Given the safety requirements for any TCR used for TCR gene transfer a commercially scalable approach ideally relies on autologous tissue as neo-antigen specific TCRs directly derived from the patient can be assumed to be safe. Furthermore, the use of non-viable tissue such as archived tumor samples, for example, is preferred to achieve a commercially scalable process, as it avoids the need to handle viable patient cells for TCR isolation. The methods disclosed herein address the significant need to identify relevant neo-antigen specific TCRs with high-sensitivity on a per patient basis. In addition, neo-antigen specific TCR gene transfer as disclosed in the methods described herein may benefit patients that do not benefit from other therapies such as immune checkpoint blockade, for example.

In some embodiments, a method of identifying nucleotide sequences encoding T cell receptor α (TCRα)- and TCRβ-chains from a combinatorial library of nucleic acids is provided. The method comprises: a) providing a library comprising a plurality of variant nucleic acids, each of the plurality of variant nucleic acids comprising a contiguous portion of at least 600 bp, wherein the contiguous portion comprises: a combination of 1) a first variant nucleotide subsequence encoding a TCRα variant amino acid sequence and defining a first end of the contiguous portion, and 2) a second variant nucleotide subsequence encoding a TCRβ variant amino acid sequence and defining a second end of the contiguous portion opposite the first end. The method further comprises introducing the library into a population of immortalized T cells configured to express TCRα- and TCRβ-chains encoded by a member of the plurality of variant nucleic acids and selecting a subpopulation of the population of immortalized T cells based on an expression of a T cell activation marker above a threshold level in response to contacting the immortalized T cells with immortalized B cells expressing an antigen, wherein the subpopulation comprises a plurality of T cells and/or isolating a subset of the plurality of variant nucleic acids from the subpopulation; and/or determining nucleotide sequences of the contiguous portion of individual members of the subset; and/or identifying at least one combination of the first and second variant nucleotide subsequences based on an enrichment of the at least one combination in the nucleotide sequences of the subset relative to a control.

In some embodiments, a method of identifying a nucleotide sequence encoding a chimeric antigen receptor (CAR) hinge domain, transmembrane domain, and/or an intracellular signaling domain from a combinatorial library of nucleic acids is provided. The method comprises: providing a library comprising a plurality of variant nucleic acids, each of the plurality of variant nucleic acids comprising a contiguous portion of at least 600 bp, wherein the contiguous portion comprises a combination of two or more of: 1) a first variant nucleotide subsequence encoding a CAR hinge domain; 2) a second variant nucleotide subsequence encoding a CAR transmembrane domain; and 3) a third variant nucleotide subsequence encoding a CAR intracellular signaling domain. One of the first, second or third variant nucleotide subsequences define a first end of the contiguous portion, and another one of the first, second or third variant nucleotide subsequences defines a second end of the contiguous portion opposite the first end. The method further comprises introducing the library into a population of cells configured to express a CAR encoded by a member of the plurality of variant nucleic acids, wherein the population of cells comprises a population of immortalized T cells or primary human T cells. The method can further include selecting a subpopulation of the population of cells based on cell proliferation above a threshold level in response to contacting the cells with antigen-presenting cells expressing an antigen specific to an antigen-binding domain of the CAR, wherein the subpopulation comprises a plurality of cells. The method may further include isolating a subset of the plurality of variant nucleic acids from the subpopulation, and/or determining nucleotide sequences of the contiguous portion of individual members of the subset; and/or identifying at least one combination of the first, second, and third variant nucleotide subsequences based on an enrichment of the at least one combination in the nucleotide sequences of the subset relative to a control.

### Definitions

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Unless otherwise specified, the definitions provided herein control when the present definitions may be different from other possible definitions.

Unless explained otherwise, all technical and scientific terms used herein have the same meaning as commonly understood to one of ordinary skill in the art to which this disclosure belongs. All HUGO Gene Nomenclature Committee (HGNC) identifiers (IDs) mentioned herein are incorporated, by reference in their entirety. Although methods and materials similar or equivalent to those described herein can be used in the practice or testing of the present disclosure, suitable methods and materials are described below. The materials, methods, and examples are illustrative only and not intended to be limiting.

“T cell receptor” or “TCR” denotes a molecule found on the surface of T cells or T lymphocytes that recognizes antigen bound as peptides to major histocornpatibility complex (MHC) molecules. MHC molecules include class I, class II, and class III. Both class I and class II MHC molecules play a critical role in immune response. MHC class I molecules are expressed in all nucleated cells and also in platelets—in essence all cells but red blood cells. It presents epitopes to killer T cells, also called cytotoxic T lymphocytes (CTLs). MHC class II can be conditionally expressed by all cell types, but normally occurs only on “professional” antigen-presenting cells (APCs): macrophages, B cells, and especially dendritic cells (I)Cs). An APC takes up an antigenic protein, performs antigen processing, and returns a molecular fraction of it—a fraction termed the epitope—and displays it on the APC's surface coupled within an MHC class II molecule (antigen presentation). On the cell's surface, the epitope can be recognized by immunologic structures like T cell receptors (TCRs). In some embodiments, the TCR comprises two polypeptide chains, TCRα and TCRβ (encoded by TRA and TRB, respectively). In some embodiments, the TCR comprises TCRγ and TCRδ chains (encoded by TRG and TRD, respectively). In sonic embodiments, the TCR comprises an extracellular variable region and an extracellular constant region. In some embodiments, the variable domain of the TCRα and TCRβ chains comprises three hypervariable complementarity determining regions (CDRs), denoted CDR1, CDR2, and CDR3. In some embodiments, CDR3 is the main antigen-recognizing region. In some embodiments, TCRα chain genes comprise V and J, and TCRβ chain genes comprise V, D and J gene segments that contribute to TCR diversity.

As used herein, the term “TCR repertoire” refers to a collection of TCR chains in a sample or library. A collection can comprise at least two or more different TCR chain variants. In some embodiments, “TCR repertoire” refers to a collection of all TCR chains in a sample or library. In some embodiments, “TCR repertoire” refers to a collection of a subset or selection of TCR chains in a sample or library. In some embodiments, “TCR repertoire” refers to a collection of TCRαβ pairs in a sample or library. In sonic embodiments, “TCR repertoire” refers to a collection of a subset or selection of TCRαβ pairs in a sample or library.

A subset or a selection of TCR chains can be based on frequency of the TCR chains, for example. In some embodiments, “frequency of a TCR chain” refers to the absolute number of nucleic acid molecules (RNA and/or DNA) encoding (part of) a particular TCR chain amino acid sequence among the total of all nucleic acids encoding (part of) all TCR chain amino acid sequences. In some embodiments, the absolute number of nucleic acid molecules encoding (part of) a particular TCR chain amino acid sequence may be determined based on the count of unique molecules using a “Unique Molecular Identifier” (UMI) (as a principle for example described in Kivioja et Nat Meth 2011 and Islam et Nat Meth 2013). In some embodiments, TCR chain frequency may be expressed as a percentage. The total number of all TCR chains may include only nucleic acid molecules encoding TCRα-chains, only TCRβ-chains or both TCRα- and TCRβ-chains. In some embodiments, frequency is expressed as a TCR chain having a frequency equal to and above, equal to, above or below 0.001%, 0.01%, 0.1%, 1%, 2%, 3%, 4%, 5%, 6%, 7%, 8%, 9%, 10%, 20%, 30%, 40%, 50%, 60%, 70%, 80%, 90%, 100% or any number or range in between chains in a sample. Using absolute numbers of nucleic acid molecules encoding (part of) a TCR chain amino acid sequence or corresponding percentages, a rank order for TCR chains can be obtained. In some embodiments, frequency is expressed as a TCR chain being among the top 1000, top 900, top 800, top 700, top 600, top 500, top 450, top 400, top 350, top 300, top 250, top 200, top 150, top 140, top 130, top 120, top 110, top 100, top 90, top 80, top 70, top 60, top 50, top 40, top 30, top 20, top 10, top 5, or any number or range in between, chains in a sample in a rank order. In some embodiments, “frequency of a TCR chain” refers to frequency of a TCR chain relative to all TCR chains in the sample. In some embodiments, “frequency of a TCR chain” refers to frequency of a TCR chain relative to fewer than all or relative to a subset of TCR chains in the sample.

As used herein, the term “frequency threshold” refers to a minimum frequency at which a given TCR chain occurs in a sample to be included in a subset or selection of TCR chains. In some embodiments, a frequency threshold comprises the top 1000, top 900, top 800, top 700, top 600, top 500, top 450, top 400, top 350, top 300, top 250, top 200, top 150, top 140, top 130, top 120, top 110, top 100, top 90, top 80, top 70, top 60, top 50, top 40, top 30, top 20, top 10, top 5, or any number or range in between, of TCR chains in a sample in a rank order. In some embodiments, a frequency threshold is expressed as including all TCR chains equal and above, equal and below, equal, above or below 0.001%, 0.01%, 0.1%, 1%, 2%, 3%, 4%, 5%, 6%, 7%, 8%, 9%, 10%, 20%, 30%, 40%, 50%, 60%, 70%, 80%, 90%, 100%, or any number or range in between, chains in a sample. In some embodiments, “frequency threshold” refers to a threshold relative to all TCR chains in the sample. In some embodiments, “frequency threshold” refers to a threshold relative to fewer than all or relative to a subset of TCR chains in the sample.

As used herein, the term “'relative enrichment” refers to a greater abundance or frequency of a TCR chain in one sample as compared to another sample. Samples that can be compared include, for example, a tumor sample and blood, different tumor samples, a tumor sample and a non-tumor sample, samples from different regions of the same tumor, samples from a tumor core and a tumor boundary or margin, samples from T cells with different activation or differentiation state, and others.

As used herein, “switch receptor” is used according to one of skill in the art and includes: switch receptors include but are not limited to receptor molecules that are used to transform extracellular signals usually associated with T cell inhibition or apoptosis into a T cell activating signal. This can be achieved by fusing an extracellular domain (ECD) binding an inhibitory or apoptosis-inducing ligand (for example but not limited to TGFBR2, FAS or TIGIT) with an intracellular signaling domain (ISD) from a T cell activating receptor (such as CD3ϵ, CD28, IL2RB). The skilled artisan may appreciate that a fusion receptor molecule may also be designed to inhibit T cell function by combining ECDs binding T cell activating ligands with ISDs from T cell inhibitory receptors (e.g. PD-1 or CTIA-4). In some embodiments switch receptors may contain but are not limited to a ECD fused with one, two or even more signaling domains. In some embodiments switch receptor molecules include but are not limited to receptor molecules that contain different transmembrane domains (TM) in addition to ECDs and ISDs or any other novel components including but not limited to linker or spacer sequences between different domains, including but not limited to ECD and TM and/or TM and ISD.

As used herein, a “single chain TCR” is used according to one of skill in the art. The term further includes, but is not limited to, covalently linking TCRα and TCRβ Variable chain fragments with a linker. Single chain TCRs include but are not limited to covalently linking TCRα and TCRβ Variable chain fragments with a linker fused to a TCRβ constant domain and are co-expressed with a TCRα constant domain in trans. In some embodiments single chain TCRs include but are not limited to covalently linking TCRα and TCRβ Variable chain fragments with a linker fused to a TCRα constant domain and are co-expressed with a TCRβ constant domain in trans. Some embodiments of single chain TCRs include but are not limited to covalently linking TCRα and TCRβ Variable chain fragments with a linker and fused to CD3ϵ or CD3ζ signaling domains alone or in combination with a CD28 signaling domain.

As used herein, the term “spatial pattern of gene expression” refers to expression of a gene in a particular region or space. In some embodiments, “spatial pattern of gene expression” refers to the expression of a gene within a tissue such as a tumor, i.e., intratumorally. In some embodiments, “spatial pattern of gene expression” refers to enrichment of gene expression in a region or space characterized by expression or absence of expression of one or more phenotypic markers. In some embodiments, a phenotypic marker can be any marker associated with a phenotype, including, but not limited to, one or more surface markers or fragments thereof, one or more proteins or fragments thereof, one or more RNA such as microRNA, siRNA, or any other RNA. In other embodiments, “spatial pattern of gene expression” refers to the expression or absence of expression of one gene in combination with expression or absence of expression of at least one other gene.

As used herein, the term “co-expression pattern” includes expression of one or more genes in the same cell or in the same tissue sample. In some embodiments, the term “co-expression pattern” refers to absence of expression of one or more genes in the same cell or in the same tissue sample.

The term “cancer” denotes a malignant neoplasm that has undergone characteristic anaplasia with loss of differentiation, increased rate of growth, invasion of surrounding tissue, and is capable of metastasis. The term “cancer” shall be taken to include a disease that is characterized by uncontrolled growth of cells within a subject. In some embodiments, the terms “cancer” and “tumor” are used interchangeably. In some embodiments, the term “tumor” refers to a benign or non-malignant growth.

The term “library” refers to a collection of TCR chains. In some embodiments, the library comprises a collection of TCR chains which combine to form TCRαβ pairs. In some embodiments, the library comprises a collection of a subset or selection of TCR chains which combine to form TCRαβ pairs.

As used herein, the term “neo-antigen” refers to an antigen derived from a tumor-specific genomic mutation. For example, a neo-antigen can result from the expression of a mutated protein in a tumor sample due to a non-synonymous single nucleotide mutation or from the expression of alternative open reading frames due to mutation induced frame-shifts. Thus, a neo-antigen may be associated with a pathological condition. In some embodiments, “mutated protein” refers to a protein comprising at least one amino acid that is different from the amino acid in the same position of the canonical amino acid sequence. In some embodiments, a mutated protein comprises insertions, deletions, substitutions, inclusion of amino acids resulting from reading frame shifts, or any combination thereof, relative to the canonical amino acid sequence.

The term “treatment” encompasses its ordinary meaning in the art, and includes alleviation of at least one symptom or other embodiment of a disorder, or reduction of disease severity, and the like. A treatment need not effect a complete cure, or eradicate every symptom or manifestation of a disease, to constitute a viable treatment. As is recognized in the pertinent field, compositions used as therapeutic agent may reduce the severity of a given disease state, but need not abolish every manifestation of the disease to be regarded as useful. Reducing the impact of a disease (for example, by reducing the number or severity of its symptoms, or by increasing the effectiveness of another treatment, or by producing another beneficial effect), or reducing the likelihood that the disease will occur or worsen in a subject, is sufficient.

“Antibody” denotes a polypeptide including at least a light chain or heavy chain immunoglobulin variable region which specifically recognizes and binds an epitope of an antigen. In sonic embodiments, antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. The term antibody includes intact immunoglobulins, as well the variants and portions thereof, such as Fab′ fragments, F(ab)′₂ fragments, and any other molecule derived from an intact immunoglobulin.

As used herein, the phrase “B-cell receptor (BCR)/antibody repertoire” refers to a collection of BCR or antibody chains in a sample. In some embodiments, “BCR/antibody repertoire” refers to a collection of all I3CR or antibody chains in a sample. In some embodiments, “BCR/antibody repertoire” refers to a collection of a subset or selection of BCR or antibody chains in a sample.

As used herein, the terms “fresh-frozen” or “snap-frozen” mean freezing a tissue or cell sample within a short period of time after collection. In some embodiments, the tissue or cell sample is not preserved prior to freezing. The terms “fresh-frozen” or “snap-frozen” can be used interchangeably.

As used herein, the term “TCR isolation” encompasses an evaluation of which specific combinations of TCRα and TCRβ chains mediate the desired functionality. The term “TCR isolation” can refer to the isolation of single-chain TCR molecules. Methods for TCR isolation can differ based on desired functionality and the design of the TCR cassette.

The term “activation marker” encompasses the full scope of the term as understood by one of skill in the art and further denotes one or multiple genes that are differentially regulated within a cell in response to an external stimulus. Genes serving as activation marker can be a natural part of the cell genome or introduced by genetic engineering tools known to a person skilled in the art (e.g. viral gene delivery). Notably, differential regulation may describe increased or decreased expression of a gene as detected on RNA level. In certain instances, such changes in transcript levels can result in detectable changes on protein level. By means of non-limiting example, activation markers in T cells that correlate with T cell receptor triggering may include CD69, CD137, IFN-γ, IL-2, TNF-α, GM-CSF, OX40 as well as artificial reporter genes such as NFAT-GFP or NFAT-puromycin resistance gene.

As used herein, the term “TCR library” refers to a polyclonal collection of plasmids encoding TCRs or cells containing those plasmids. A collection can comprise at least two or more different TCR chain variants. “TCR library” can include a collection of a subset or selection of plasmids encoding TCRs. “TCR library” can refer to a collection of all TCRs that can be expressed from a collection of plasmids encoding TCRs. In some embodiments, “TCR library” refers to a collection of a subset or selection of TCRs that can be expressed from a collection of plasmids encoding TCRs. In some embodiments, “TCR library” refers to a collection of TCRab pairs in polyclonal collection of plasmids encoding TCRs. In some embodiments, “TCR library” refers to a collection of a subset or selection of TCRab pairs in polyclonal collection of plasmids encoding TCRs.

### General Description of Various Embodiments

Some embodiments described herein relate to a method of identifying nucleotide sequences encoding T cell receptor α (TCRα)- and TCRβ-chains from a combinatorial library of nucleic acids. In some embodiments, the method comprises (I) providing a library comprising a plurality of variant nucleic acids encoding TCRα- and TCRβ-chains, (II) introducing the library into a population of cells able to express TCRα- and TCRβ-chains encoded by a member of the plurality of variant nucleic acids, (III) selecting a subpopulation of the population of cells based on an expression of a marker above a threshold level in response to antigen, wherein the subpopulation comprises a plurality of cells, (IV) isolating a subset of the plurality of variant nucleic acids from the subpopulation, (V) determining nucleotide sequences of the variant nucleic acids, and (VI) identifying at least one variant nucleotide sequence based on an enrichment of the nucleotide sequences within the subset relative to a control. In some embodiments, enrichment can be based on statistical enrichment using appropriate analytical software. In some embodiments, the R DESeq2 package can be used to identify significance of enrichment. In some embodiments, the p-value threshold for significance can be defined as 0.2, 0.1, 0.05, 0.01, 0.001, 0.0001, zero or any value in between any of these values.

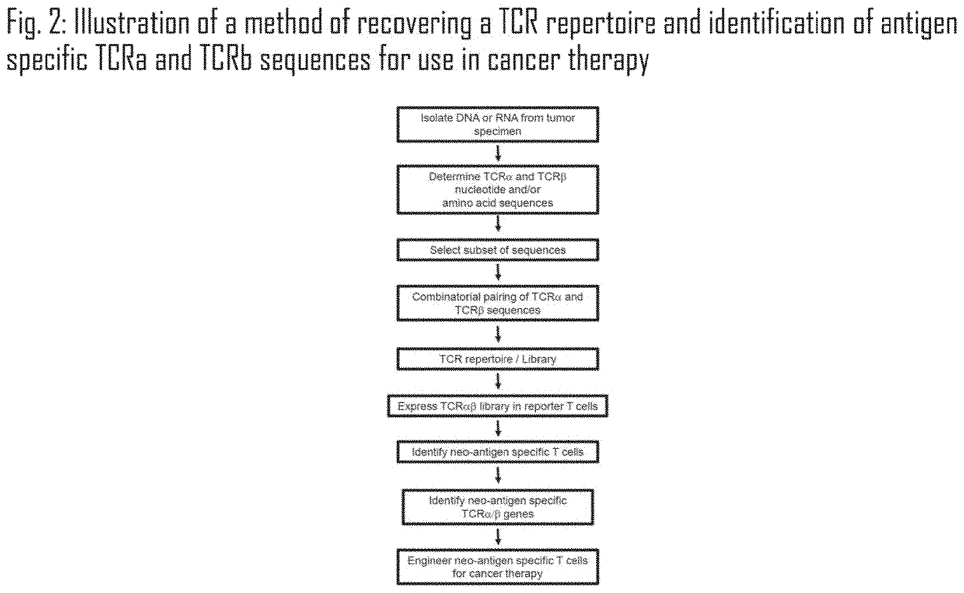

In some embodiments, a method to recover a repertoire of T cell receptors (TCRs) from diverse T cell populations is described. The method can comprise (I) determining TCR-α and β nucleotide sequences within a subject's sample, (II) selecting one or more subsets of TCRα- and β-chain sequences from the total repertoire based on at least one criterion; (III) creating a TCR repertoire by combinatorial pairing of selected TCRα- and β-chain sequences creating a library of TCRαβ pairs; and IV) identifying at least one TCRαβ pair with desired features from the created TCR repertoire. Various embodiments of the methods are provided in FIGS. 1-3.

In some embodiments, TCR chain selection will initially be based on frequency threshold as further described herein. In some embodiments, selection will be based on more than one criterion. More than one criterion for selection may be chosen based on the type of tumor analyzed, for example. In some embodiments, TCR chain selection is based on thresholds of screening efficiency. In some embodiments, selection criteria are chosen based on how many combinatorial TCRαβ chains can be screened efficiently. As an example, if 1×10⁶ TCRαβ pairs can be efficiently screened, up to 1000 TCRα and 1000 TCRβ chains may be selected. In some embodiments, an unequal number of TCRα and TCRβ chains may be selected, e.g. to compensate for the fact that a substantial fraction of T cells carries two in-frame TCRα rearrangements. In some embodiments, the ratio of TCR alpha to TCR beta can be, for example 1 million:1 to 1:1 million. In some embodiments, the ratio is any ratio there between these ranges, including, for example, 100,000:1, 10,000:1, 1,000:1, 100:1, 10:1, 1:1, 1:10, 1:100, 1:1000, 1:10,000, 1:100,000.

In some embodiments, TCR chain sequences identified in libraries generated by the methods described herein are useful for treatment or diagnosis of the patient from whom the TCR chain sequences have been isolated. In some embodiments, TCR chain sequences identified in libraries generated by the methods described herein are useful for the treatment or diagnosis of patients other than the patient from whom the TCR chain sequences have been isolated. For example, TCR chain sequences isolated from one patient may recognize a tumor antigen that is shared by another patient.

In some embodiments, large numbers of libraries are generated by the methods described herein. In some embodiments, screening large numbers of libraries allows for prediction of TCR features, thus allowing for specific selection of TCR chains, for example.

in some embodiments, the TCR libraries can include i) combinatorial TCR libraries; ii) cells expressing such library; and/or iii) TCR amplicon sequencing libraries.

In some embodiments, the TCR libraries are a polyclonal collection of plasmids that express exactly one alpha and one beta TCR chain in a combinatorial fashion. The nature of the library is such that the frequency of a given alpha chain pairing with a given beta chain is proportional to the overall representation of that beta chain. Conversely, the frequency of a given beta chain pairing with a given alpha chain is proportional to the overall representation of that alpha chain (from this it follows that one can control the frequency of individual chains in the library). The percentage of frequencies of the individual combination that are within a range of median frequency +/−1 log2 unit are 25%, 50%, 60%, 70%, 80%, 90%, 95%, 86%, 97%, 98%, 99%, 100% or anything in between any two of the preceding values.

In some embodiments, the library can involve cell expressing relevant nucleic acid sequences. Expression may include stable or temporary approaches and may be conferred by DNA/RNA (or derivatives thereof). The number of cells in a polyclonal pool expressing such library can be 20, 30, 40, 50, 75, 100, 125, 150, 200, 250, 300, 400, 500, 750, 1,000, 5,000, 10,000, 100,000, 1,000,000× the number of TCR variants present in the pool,

In some embodiments, the TCR amplicon sequencing library is a collection of DNA molecules representing the frequency of TCRs in a given sample. The amplicon contains information about both alpha and beta chains that are expressed in a given cell, and the sequence in a stretch of more than 600 contiguous nucleotides is required to identify both V and J region identity, as well as CDR3 sequence for both alpha and beta chains.

In some embodiments, the library is a variant library of approximately 1.5 kb.

Diverse T cell populations used in the methods described herein can comprise T cells of any lineage or mixtures thereof. In some embodiments, diverse T cell populations comprise CD4 or CD8 T cells. In some embodiments, the diverse T cell populations comprise naïve T cells. In some embodiments, the diverse T cell populations comprise effector T cells. Any type of effector T cell may be found in diverse T cell populations, including Th₁, Th₂, Th₁₇ and Cytotoxic T lymphocytes (CTL). In some embodiments, the diverse T cell populations comprise regulatory T cells (T_{reg}). In some embodiments, the diverse T cell populations comprise memory T cells. Any memory subtype may be found in diverse T cell populations, including central memory T cells (T_{CM} cells), effector memory T cells (T_{EM} cells and T_{EMRA} cells), tissue resident memory T cells (T_{RM}), memory stem cell T cells (T_{SCM}) and virtual memory T cells. In some embodiments, virtual memory T cells comprise CD4 positive T cells. In some embodiments, virtual memory cells comprise CD8 positive memory T cells. In some embodiments, diverse T cell populations comprise regulatory cells. In some embodiments, diverse cell populations comprise dysfunctional cells. Dysfunctional T cells are characterized by (1) high levels of inhibitory receptors, (2) loss of classical effector functions (e.g. IFN-γ, IL-2 and TNF-α) while secreting the chernokine CXCL13 and (3) high expression levels for transcriptional profiles associated with cytotoxicity, including high levels of Granzyme B. In some embodiments, diverse T cell populations comprise αβ T cells. In some embodiments, diverse T cell populations comprise γδ T cells. Diverse T cell populations can comprise natural killer T cells (NKT) and mucosal associated invariant T cells (MAIT). A T cell population can be part of a mixture of different cell types or part of a tissue sample, such as blood or tumor tissue, for example. Diverse T cell populations can comprise mixtures of T cells of different lineages or mixtures of T cells and non-T cells.

In some embodiments, the TCR-α and β nucleotide sequences are determined within a subject's sample. TCR-α and β nucleotide sequences can be determined utilizing DNA or RNA obtained from a sample. In some embodiments, determining the TCR-α and β nucleotide sequences comprises use of multiplex PCR. In some embodiments, determining the TCR-α and β nucleotide sequences comprises TCR-sequence recovery by target enrichment. For example, TCR gene capture can be used for target enrichment (Linnemann et al, Nat Med 2013). In some embodiments, TCR-sequence recovery comprises utilizing recovery by 5′RACE and PCR. In some embodiments, TCR-sequence recovery comprises utilizing spatial sequencing.

In some embodiments, DNA or RNA is isolated from viable cells. In some embodiments, DNA or RNA is isolated from preserved cells or preserved tissue samples. Preserved cells and preserved, tissue samples can be viable or non-viable. Preserved, tissue samples can comprise viable or non-viable cells or a combination of both viable and non-viable cells. DNA or RNA can be isolated from a sample or specimen preserved by any preservation method, including snap-frozen cells or tissue and fixed or formalin fixed/paraffin-embedded (FFPE) samples. Preservation methods for cells and tissue samples and DNA and RNA isolation methods are known to a person skilled in the art. In some embodiments, the sample is a tumor sample. In some embodiments, the tumor sample is an FFPE sample. In some embodiments, the tumor sample is a snap-frozen sample. In some embodiments, the T cell population is part of a mixture of different cell types or part of a tissue sample or body fluid, such as blood, urine, draining lymph node or tumor tissue, for example. In some embodiments, the sample is a non-viable tumor specimen. In some embodiments, the non-viable tumor sample is a snap-frozen sample or an FFPE sample.

In some embodiments, one or more subsets of TCRα- and β chain sequences from the total repertoire are selected based on at least one criterion: a) on frequency within the T cell population, b) on relative enrichment compared to a second T cell population, c) on biological properties of the TCR chain, wherein the properties are selected from at least one of: (predicted) antigen-specificity, (predicted) HLA-restriction, affinity, co-receptor dependency or parental T cell lineage (e.g. CD4 or. CD8 T cell), d) on spatial patterns of gene expression, wherein spatial gene expression patterns are derived from at least one of: originating region in the tissue or expression patterns of other genes, including co-expression, for example, e) on co-occurrence or occurrence at a similar frequency in multiple samples, for example occurrence in multiple tumor lesions, f) selection into multiple groups to separately recover specific parts of the TCR repertoire, g) on a combination of multiple criteria as defined in the different embodiments.

In some embodiments, the selection criteria can be used for exclusion instead of inclusion (including, for example, in options b or c in the paragraph above). This can be applied to any of the embodiments provided herein. Thus, they can be applied to not administer or supply to a subject or to exclude their inclusion in a TCR collection.

In some embodiments, TCRα- and β chain sequences are selected from the total repertoire based on relative difference of DNA and RNA copy numbers of a given TCR chain. For example, the ratio between RNA-derived and genomic copy numbers can he obtained based on quantification of genomic DNA and RNA for a given TCR chain. In some embodiments, a TCR chain is selected where the RNA copy number is much higher than the genomic DNA copy number, resulting in a ratio that is greater than 1. In some embodiments the resulting ratio of any given TCR can be ranked and selected relative to all other TCRs in the sample. For example, TCRs with a greater rank based on a greater ratio may be selected compared to TCRs with a lower rank based on a lower ratio, thereby selecting for TCR chains with greater RNA copy numbers. In some embodiments, TCRs with a lower rank based on a lower ratio may be selected compared to TCRs with a greater rank based on a greater ratio, thereby selecting for TCR chains with lower RNA copy numbers. Rank order can be adjusted to any numeric value for the ratio between RNA-derived and genomic copy numbers.

In some embodiments, any number of TCRα and any number of TCRβ chains are selected, up to and including 1000 TCRα and TCRβ chains each. In some embodiments, more than 1000 TCRα and TCRβ chains each are selected. In some embodiments, a number of TCRα and TCRβ chains is selected to result in about 1×10⁶ TCRαβ pairs. In some embodiments, a number of TCRα and TCRβ chains is selected to result in more than 1×10⁶ TCRαβ pairs.

In some embodiments, one or more subsets of TCRα- and β chain sequences from the total repertoire are selected based on frequency within the T cell population. In some embodiments, data on the frequency of TCR sequences is used to create a separate rank order for TCRα- and β-chains. For example, the absolute number of nucleic acid molecules encoding (part of) different TCR chain amino acid sequences may be determined for a T cell containing sample using Multiplex PCR, target enrichment or 5′-RACE and PCR. In some embodiments, DNA is used in the methods described herein. In some embodiments, RNA is used in the methods described herein. The resulting collection of TCR chain sequences is divided into a collection of TCRα- and a collection of TCRβ-chain sequences. Any non-productive TCR chain sequences, in which TCR segments are joined out of frame at the amino acid sequence level, and/or in which stop codons are introduced, and/or in which frameshift mutations are present, and or in which defective splicing sites are present, are removed from the collection. In some embodiments, absolute numbers of nucleic acid molecules encoding (part of) a particular TCR chain amino acid sequence are determined based on the count of unique molecules using a “Unique Molecular Identifier” (UMI), and sorted in descending order to obtain a rank order of TCRα- and β-chains. In some embodiments, each collection is sorted in descending order using either absolute numbers of nucleic acid molecules encoding a particular TCR chain (or corresponding percentage among total TCRα- or TCRβ-chains, respectively) to obtain a rank order for TCRα- and β-chains.

In any of the embodiments provided herein, RNA can be collected or created and the RNA can be sequenced in place of DNA sequencing.

In some embodiments, data on the frequency of TCR sequences is used to create a combined rank order for TCRα- and β-chains. For example, the absolute number of nucleic acid molecules encoding (part of) different TCR chain amino acid sequences may be determined for a T cell containing sample using Multiplex PCR, target enrichment or 5′-RACE and PCR. In sonic embodiments, DNA is used in the methods described herein. In some embodiments, RNA is used in the methods described herein. Any non-productive TCR chain sequences, in which TCR segments are joined out of frame at the amino acid sequence level, and/or in which stop codons are introduced, and/or in which frameshift mutations are present, and/or in which defective splicing sites are present, are removed from the collection. The remaining TCR chain sequences are sorted in descending order using either absolute numbers of nucleic acid molecules, possibly determined by use of a Unique Molecular Identifier” (UMI), encoding a particular TCR chain or corresponding percentage among the total set of TCR chains) to obtain a rank of TCR chains.

In some embodiments, a frequency threshold is defined based on the desired depth for TCR repertoire recovery. For example, the absolute number of nucleic acid molecules encoding (part of) different TCR chain amino acid sequences may be determined for a T cell containing sample using Multiplex PCR, target enrichment or 5′-RACE and PCR and possibly using a Unique Molecular Identifier” (UMI). In some embodiments, DNA is used in the methods described herein. In some embodiments, RNA is used in the methods described herein. The resulting collection of TCR chain sequences will be divided into a collection of TCRα- and a collection of TCRβ-chain sequences. Any non-productive TCR chain sequences, in which TCR segments are joined out of frame at the amino acid sequence level, and/or in which stop codons are introduced, and/or in which frameshift mutations are present, and/or in which defective splicing sites are present, are removed from the collection. Each collection is sorted in descending order using either absolute numbers of nucleic acid molecules encoding a particular TCR chain (or corresponding percentage among total TCRα- or TCRβ-chains, respectively) to obtain a rank order for TCRα- and β-chains. If the intention is to recover the ten most frequent TCRs in the sample, only the Top 10 most frequent TCRα- and β-chains may be selected. In contrast, if it is desirable to recover a larger part of the TCR repertoire in the sample, more than the Top 10 most frequent TCRα- and β-chains may be selected. A lower frequency threshold can lead to greater depth but also higher diversity in the pool of selected TCR chains and the resulting TCR libraries. Importantly, TCRα- or TCRβ-chains may also be selected from a combined rank order as described in one of the disclosed embodiments.

In some embodiments, the lower frequency threshold is used to select collections of TCRα- and β-chains based on frequency. In some embodiments, there is no requirement to select equal numbers of TCRα- and β-chains. For example, the absolute number of nucleic acid molecules encoding (part of) different TCR chain amino acid sequences may be determined for a T cell containing sample using Multiplex PCR, target enrichment or 5′-RACE and PCR and possibly using a Unique Molecular Identifier” (UMI). In some embodiments, DNA is used in the methods described herein. In some embodiments, RNA is used in the methods described herein. The resulting collection of TCR chain sequences will be divided into a collection of TCRα- and a collection of TCRβ-chain sequences. Any non-productive TCR chain sequences, in which TCR segments are joined out of frame at the amino acid sequence level, and/or in which stop codons are introduced, and/or in which frameshift mutations are present, and/or in which defective splicing sites are present, are removed from the collection. Each collection is sorted in descending order using either absolute numbers of nucleic acid molecules encoding a particular TCR chain (or corresponding percentage among total TCRα- or TCRβ-chains, respectively) to obtain a rank order for TCRα- and β-chains. The resulting rank orders may contain diverging numbers of TCRα- or TCRβ-chains preventing the selection of equal numbers of TCRα- and TCRβ-chains or it may be desirable to select more TCR chains from one category than the other, e.g. because of the propensity of both TCRα loci in a cell to undergo a productive rearrangement. Furthermore, if all TCR chains above or below certain frequency (as expressed as an absolute number or percentage) are selected this may lead to the selection of diverging numbers of TCRα- and TCRβ-chains, respectively. Importantly, TCRα- or TCRβ-chains may also be selected from a combined rank order as described in one of the preceding embodiments.

In some embodiments, the top 100 most abundant TCRα- and β-chains are selected based on quantitative frequency data. In some embodiments, more than the top 100 most abundant TCRα- and f3-chains are selected based on quantitative frequency data. In some embodiments, the top 100, top 200, top 300, top 400, top 500, top 600, top 700, top 800, top 900, top 1000 most abundant TCRα- and β-chains, or any number or range in between, are selected based on frequency data. In some embodiments, more than the top 1000 most abundant TCRα- and β-chains are selected based on frequency data. In some embodiments, the top 5%, top 10%, top 20%, top 30%, top 40%, top 50%, top 60%, top 70%, top 80%, top 90%, top 100% of TCRα- and β-chains, or any number or range in between, are selected based on frequency data. In some embodiments, selected chains serve as a building block to assemble a collection of TCRαβ pairs.

In some embodiments, one or more subsets of TCRα- and β chain sequences from the total repertoire are selected based on relative enrichment compared to a second T cell population. In some embodiments, the top 100 TCRα- and β-chains are selected based on highest fold enrichment in a given sample when compared to another sample. In some embodiments, the top 100 most abundant TCRα- and β-chains are selected based on relative enrichment. In some embodiments, the highest fold enrichment is relative to another sample. In some embodiments, more than the top 100 most abundant TCRα- and β-chains are selected based on relative enrichment. In some embodiments, the top 100, top 200, top 300, top 400, top 500, top 600, top 700, top 800, top 900, top 1000 most abundant TCRα- and β-chains, or any number or range in between, are selected based on relative enrichment. In some embodiments, more than the top 1000 most abundant TCRα- and β-chains are selected based on relative enrichment. In some embodiments, the top 5%, top 10%, top 20%, top 30%, top 40%, top 50%, top 60%, top 70%, top 80%, top 90%, top 100% of TCRα- and β-chains, or any number or range in between, are selected based on relative enrichment. In some embodiments, TCR chains from a tumor lesion are selected based on relative enrichment compared to their respective frequency in blood. In some embodiments, TCR chains from a tumor lesion are selected based on relative enrichment compared to second tumor lesion. In some embodiments, quantification of TCR chains is performed on multiple samples in parallel. For example, multiple tumor lesions, a matched tumor lesion and blood sample from the same individual, or multiple discretely sampled sections of a larger tumor lesion can be analyzed in parallel. By analyzing multiple samples or matched samples from the same individual, the biological relevance of TCR chains can be determined. For example, a TCR chain with enriched frequency in the tumor compared to blood or occurrence in multiple tumor lesions is more likely to be associated with recognition of a tumor antigen compared to a TCR chain that also occurs at high frequency in peripheral blood or that occurs in a single tumor lesion. In some embodiments, TCR chains are selected based on TCR chain frequencies in the tumor core as compared to the tumor boundary or the tumor margin, which may include normal tissue surrounding the tumor. In any of the disclosed embodiments, relative frequency differences can be used to create a rank order based on a fold-difference in relative frequency. The fold-difference in relative frequency may be any number between 10⁻⁶ and 10⁶. In some embodiments, TCR chains which are found exclusively in one of at least two compared samples may be preferentially selected or excluded for TCR library generation. In some embodiments, the top 100 ranked TCRα and TCRβ chains are used for TCR library generation. In some embodiments, more than the top 100 ranked TCRα and TCRβ chains are used for TCR library generation. In some embodiments, TCR chain repertoires in different samples are compared for targeted selection of TCGR chains with a high likelihood of neo-antigen specificity. In some embodiments, TCR chain sequences are ordered based on relative enrichment, followed by selection according to rank order based on frequency, tier example, as described above. Any order of criteria and any combination of criteria can be used for TCR chain selection.

In any of the embodiments provided herein, composite metrics can also be used. That is, ranking can be done by a combination of two or more aspects, such as ranking by frequency and by tumor enrichment as well. In some embodiments, the TCR is both high. frequency in the tumor and enriched in the tumor.

In some embodiments, one or more subsets of TCRαand β chain sequences from the total repertoire are selected based on biological properties or sequence features of the TCR chain. In some embodiments, the biological properties or sequence features of the TCR chain are selected from at least one of (predicted) antigen-specificity, (predicted) HLA-restriction, affinity, co-receptor dependency or parental T cell lineage (e.g. CD4 or CD8 T cell). In some embodiments, information on biological properties is obtained. by in silico algorithm-based prediction.

In some embodiments, algorithms are used to identify TCR clusters in the sample. Information on TCR clusters can be used for target selection of TCR chains for subsequent TCR library generation, for example. In some embodiments, information on TCR clusters is used for selection of clusters with defined properties. In some embodiments, information on TCR clusters is used for comparison of clusters against public TCR databases. In some embodiments, information on TCR clusters is used to remove clusters or TCR chains with high probability of irrelevant TCR specificity. In some embodiments, clusters are removed based on comparison of clusters against public TCR databases. Exemplary TCR specificities with a high probability of being irrelevant include, for example, recognition of viral epitopes derived from influenza, CMV, EBV, and other viral and bacterial infectious agents. Generated sets of TCR chains from which irrelevant TCR chains have been removed can be used subsequently for TCR library generation. In some embodiments, information on TCR clusters is used to preferentially include clusters of TCR chains with related amino acid sequence.

In some embodiments, TCR properties are identified based on amino acid sequence of TCR chains. In some embodiments, TCR properties are identified based on structural features of the TCRαβ complex. Exemplary properties include, for example, HLA-restriction, antigen specificity, co-receptor dependency, parental T cell lineage, shared properties among clusters of TCRs, and others.

In some embodiments, one or more subsets of TCRα- and β chain sequences from the total repertoire are selected based on spatial information. In some embodiments, one or more subsets of TCRα- and β chain sequences from the total repertoire are selected, based on spatial patterns of gene or protein expression, wherein spatial gene or protein expression patterns are derived from at least one of: originating region in the tissue or co-expression patterns of other genes or proteins (which for avoidance of doubt includes the possible absence of co-expression). In some embodiments, TCR chains are selected based on intratumoral localization. In some embodiments, TCR chains are selected based on enrichment in spaces showing an overexpression (or absence of expression) of certain phenotypic markers. A phenotypic marker can be any marker associated with a phenotype, including, but not limited to, one or more surface markers or fragments thereof, one or more proteins or fragments thereof, one or more RNA such as microRNA, siRNA, or any other RNA.

In some embodiments, spatial sequencing methods are used to filter for TCR chains. Spatial sequencing enables to recover transcriptomic or genetic information, including TCR sequence information, from cells together with the position of a cell within a tissue. For example, sets of neighboring cells are recovered and labelled to mark their spot of origin within the tissue to link transcriptomic or genetic information with spatial dimension. Cells that are recovered from the same or nearby spatial position in the tissue form a spatial cluster. TCR chains from certain spatial clusters may be preferentially selected based on certain information. Information that can be used includes, for example, anatomical information. For example, clusters of cells located in the center of the tumor or in tertiary lymphoid structures can be of higher interest than clusters at the tumor boundary. Information that can be used includes, for example, transcriptomic and or protein expression information. For example, spatial clusters with high PD-1 and CD39 expression are more likely to be enriched for neo-antigen specific TCR chains than clusters with low expression for such markers. Thus, clusters with high PD-1 and CD39 expression can be selected to filter TCR chains. In some embodiments, a cluster is selected based on overexpression in the center of a tumor as compared to the tumor boundary, for example. Exemplary parameters for selection are shown in Table 1.

TABLE 1
Exemplary Parameters for Selection of Markers
(including any combination thereof)

| | Relevance of expression in |
|---|---|
| Gene marker | spatial cluster |
| PD-1, Tim-3, LAG-3, CD109, CD200, | Overexpression may mark |
| GITR, TNFRSF18, CD39, CD103, IFN-γ, | possible cluster of interest |
| IL-2, TNF-α, Ki67, Granzyme B, | |
| CXCL13 CDS8, CD4, CTLA-4, FoxP3, | |
| RBPJ, ZBED2, ETV1, ID3, MAF, | |
| PRDM1, EOMES, ITM2A, KIR2DL4, | |
| PTMS, FAM3C, AKAP5, CD7, PHLDA1, | |
| ENTPD1, CCL3, CCL4, TNFRSF9, | |
| ZBED2, XCL2, HMGB1P17, ILDR1, | |
| C10orf113, BTNL8, DLGAP5, MIR4298, | |
| CDC25C, KIF20A, E2F2, TSHZ2 | |
| CD45RA, KLRG1, IL7R, TCF7, CCR7 | Overexpression may mark |
| | clusters of lower interest |

Selected sets of TCR chains can be used for TCR library generation. In some embodiments, spatially resolved RNA-or DNA-sequencing is employed. In some embodiments, bulk TCR chain populations are recovered together with additional transcripts relating to T cell phenotype, for example. Exemplary transcripts relating to T cell phenotype include CTLA-4, PD-1, CD103, CD39, FoxP3, IFN-γ, IL-2, CXCL13 and others. In some embodiments, anatomical location and T cell-specific transcriptome recovered from multiple spatial clusters is used to identify clusters of interest.

In some embodiments, one or more subsets of TCRα- and β chain sequences from the total repertoire are selected based on co-occurrence or occurrence at a similar frequency in multiple samples. In some embodiments, one or more subsets of TCRα- and β chain sequences from the total repertoire are selected based on occurrence in multiple tumor lesions. In some embodiments, TCR chain frequency information from multiple tumor lesions is used to filter for TCR chains of interest. In some embodiments, specific information used to filter TCR chains of interest comprises exclusion of TCR chains occurring in only one tumor lesion. In some embodiments, specific information used to filter TCR chains of interest comprises selective inclusion of TCR chains occurring in all tumor lesions tested.

In some embodiments, one or more subsets of TCRα- and β chain sequences from the total repertoire are selected based on selection into multiple groups to separately recover specific parts of the TCR repertoire. In some embodiments, all TCRα- and β-chain sequences with a frequency above a defined threshold are selected together into one group. For example, all TCRα- and βt-chain sequences comprising a certain percentage of total TCRα- and β-chain sequences, for example above and/or equal 10%, 9%, 8%, 7%, 6%, 5%, 4%, 3%, 2%, 1%, 0,5%, 0.1%, 0,05% or 0.01% or any other number between this range may be selected into one group. By similar principle, for example all TCRα- and β-chain sequences with rank position up and equal to 10 (Top 10), 100, (Top 100), 1,000 (Top 1,000) or 10,000 (Top 10,000) may be selected in one group. In some embodiments, all TCRα- and β-chain sequences below the defined threshold or between two defined thresholds are selected into one group. For example, all TCRα- and β-chain sequences below 1% or between 1% and 0.1% may be selected into one group. By similar principle, for example all TCRα- and β-chain sequences below rank position 10 or between rank position 10 and 100 may be selected into one group. In some embodiments, all TCRα- and β-chain sequences with a frequency above a defined threshold are selected together into one group and all TCRα- and β-chain sequences below the defined threshold are selected into another group. For example, all TCRα- and β-chain sequences above and equal to 1% are selected into one group and all TCRα- and β-chain sequences below 1% are selected into a second group. By similar principle, for example all TCRα- and β-chain sequences above or equal to rank position 10 are selected into one group and all TCRα- and β-chain sequences below rank position 10 are selected into a second group.

In some embodiments, larger numbers of TCRs are screened without creating TCR libraries of substantial complexity. As an example, multiple sub-libraries can be generated. In some embodiments, the complexity of TCR libraries is less than the complexity resulting from random pairing of all included TCRα- and β-chain sequences. In some embodiments, the generated TCR library does not contain all possible TCRa and TCRb combinations. In some embodiments, sets of TCR chains are segregated into individual pools to create one or more lower complexity libraries than would be obtained by randomly pairing TCRα- and β-chain sequences. In some embodiments, TCR chains are pooled based on ranking threshold. In some embodiments, all TCRs within a certain position in the rank order form a pool for TCR library generation. For example, the top 50 ranked TCRα and β chains can be included in pool 1; the top 25-top 75 ranked TCRα arid β chains can be included in pool 2; and so forth. As a further example, the top 50-top 100 ranked TCRα and β chains can be included in pool 2; and so forth. Any ranking criteria can be used, including different thresholds for TCRα and β chains. In some embodiments, ranking is based on frequency. In some embodiments, ranking is based on relative enrichment compared to a reference sample. In some embodiments, TCR chains are pooled based on spatial information. For example, all TCRα- and β-chains from a given spatial cluster can form a specific pool. In some embodiments, TCR chains are pooled based on characteristics of the TCRs. Any TCR characteristic can be used to pool TCRs. For example, all TCRs with defined sequence features or a predicted property can form a specific pool. Examples of predicted properties include, for example, co-receptor dependency, originating T cell lineage, HLA-restriction, specificity, and others.

In some embodiments, one or more subsets of TCRα- and β chain sequences from the total repertoire are selected based on a combination of multiple criteria as defined in the different embodiments.

In some embodiments, a TCR repertoire is created by combinatorial pairing of selected TCRα and β-chain sequences. In some embodiments, combinatorial pairing comprises random pairing of all selected. TCRα and β-chain sequences. A library of TCRαβ chains can be created by combinatorial pairing of selected TCRα and β-chain sequences. In some embodiments, selected TCR chain sequences are used to synthesize a library of TCRα- and β-chain DNA or RNA fragments. Using cloning strategies known to the skilled artisan (e.g., including, but not limited to Gibson molecular assembly and Golden Gate assembly), artificial TCR genes can be created by linking exactly one TCRα- and one β-chain DNA or RNA fragment. In some embodiments, combinations of TCRα- and β-chains are generated by directly synthesizing DNA or RNA fragments in which exactly one TCRα- and one β-chain are linked. In some embodiments, combinations of TCRα- and β-chains are created intracellularly by modification of a pool of cells with separate collections of TCRα- and genes in such a way that cells express approximately one TCRα- and one β-chain.

In some embodiments, creating a TCR repertoire by combinatorial pairing of selected TCRα- and β-chain sequences creating a library of TCRαβ pairs is achieved by at least one of the following: a) TCR chain sequences are used to synthesize separate libraries of TCRα- and β-chain DNA or RNA fragments which are subsequently linked into one DNA or RNA fragment in which exactly one TCRα- and one β-chain are linked, b) combinations of TCRα- and β-chains are generated by directly synthesizing DNA or RNA fragments in which exactly one TCRα- and one β-chain are linked, c) combinations of TCRα- and β-chains are created intracellularly by modification of a pool of cells with separate collections of TCRα- and β-genes in such a way that cells will express at least one TCRα- and one β-chain, and/or d) combinations of TCRα- and β-chains are linked in a single-chain TCR construct in which both TCRα and TCRβ Variable chain fragments are fused and in which the single chain TCR construct may be fused to (i) a transmembrane domain alone or (ii) additionally contain intracellular signaling domains, including but not limited to CD3ϵ or CD3ζ signaling domains alone or in combination with a CD28 signaling domain.

For any of the embodiments herein, in some embodiments, Class I and/or Class II restricted TCR sequences are recovered.

For any of the embodiments provided herein, in some embodiments, at least one of: neo-antigen specific TCR sequences, virus-specific TCR sequences, shared tumor-antigen specific TCR sequences, and/or self-antigen specific TCR sequences, are recovered.

In some embodiments, the activation marker can be selected from the group consisting of: CD25, CD69, CD62L, CD137, IFN-γ, IL-2, TNF-α, GM-CSF, OX40.

In some embodiments, the TCR repertoire represents all of the TCRα and β-chain sequences in the sample. In some embodiments, the TCR repertoire represents all of the TCRα and β-chain sequences recovered from the sample. In some embodiments, the TCR repertoire is selected as a subset of TCRα and β-chain sequences from the total repertoire of TCR sequences present in the sample. In some embodiments, the TCR repertoire is selected as a subset of TCRα and β-chain sequences from the total repertoire of TCR sequences recovered from the sample.

In some embodiments, the method comprises identifying at least one TCRαβ pair from the created TCR repertoire. In some embodiments, the TCRαβ pair represents a combination that is newly generated. In some embodiments, the TCRαβ pair represents a combination that is not newly generated. In some embodiments, a pool of reporter cells or T cells is modified with the library of generated TCRαβ pairs. In some embodiments, the pool of modified reporter cells or T cells can be stimulated by antigen presenting cells loaded with at least one antigen of interest. Any stimulation assay for reporter or T cells can be used. Stimulation assays for reporter cells or T cells are known to a person skilled in the art. In some embodiments, antigen-reactive reporter cells or cells are isolated based on at least one activation marker. Any CD4 or CD8 T cell activation marker can be used, for example. In some embodiments, any CD marker can be used. In some embodiments, activation markers can include markers such as CD69, CD137, IFN-γ, IL-2, TNF-α, GM-CSF, for example. In some embodiments, antigen-reactive reporter cells or T cells are isolated based on proliferation. In some embodiments, antigen-reactive reporter cells or T cells are isolated based on resistance to antibiotic selection which is acquired through reporter cell or T cell activation dependent expression of a resistance gene. Any method of reporter cell or T cell isolation can be used, including, but not limited to magnetic bead enrichment or flow cytometry, for example, which are known to the skilled artisan. In some embodiments, no reporter cell or T cell isolation may be necessary. For example, reporter cell or T proliferation or use of antibiotic selection may eliminate the need for selection. In some embodiments, RNA is obtained from bulk antigen-reactive reporter cells or T cells. RNA obtained from bulk antigen-reactive reporter cells or T cells can be used to generate TCRαβ specific cDNA. In some embodiments, TCRαβ specific cDNA is analyzed by DNA sequencing to determine TCRαβ gene sequences of antigen-reactive reporter cells or T cells. In some embodiments, DNA is obtained from bulk antigen-reactive reporter cells or T cells to generate a TCRα/β-specific PCR product which is analyzed by DNA sequencing to determine TCRαβ gene sequences of antigen-reactive reporter cells or T cells. In some embodiments, defined TCRαβ pairs may be associated with a molectilar nucleic acid-based identifier (“barcode”) which can be detected by sequencing of a specific PCR product generated from RNA or DNA.

In some embodiments, TCRαβ pairs are determined using single-cell based approaches. Single-cell based approaches include Droplet-PCR, for example. Using single-cell based approaches, TCR gene sequences of antigen-reactive T cells can be analyzed. In some embodiments, antigen-reactive T cells are identified by one or more activation markers. Any CD marker can be used, including CD4 or CD8 T cell activation marker, for example. In some embodiments, activation markers include CD69, CD137, IFN-γ, TNF-α, GM-CSF, for example. In some embodiments, antigen-reactive T cells are identified by their transcriptional profile.

in some embodiments, TCRαβ pairs are determined by genomic PCR of TCRαβ gene insertions in bulk T cells. In some embodiments, the generated PCR product is subjected to DNA-sequencing analysis.

In some embodiments, activation of TCR-transduced reporter cells or T cells is identified using reporter genes. Reporter genes can report on TCR triggering. Exemplary reporter genes include NFAT-GFP or NFAT-YFP, for example. In some embodiments, antigen-reactive reporter cells or T cells are isolated based on resistance to antibiotic selection which is acquired through T cell activation dependent expression of a resistance gene. Exemplary reporter genes include NFAT-Puromycin resistance or NFAT-Hygromycin, for example. In some embodiments, combinations of reporter genes are used.

In some embodiments, antigen-reactive cells are identified by binding to MHC complexes that carry an antigen of interest.

In any of the above embodiments, at least one TCRαβ pair is identified from the created TCR repertoire. Desired features of a TCRαβ pair can include antigen-specificity, TCR affinity, TCR co-receptor dependency, HLA-restriction, TCR cross-reactivity, TCR anti-tumor reactivity or any combination thereof.

In any of the above embodiments, a recovered TCR-chain sequence can be defined to comprise the CDR3 nucleotide sequence together with sufficient and 3′-nucleotide sequence information to select at least one TCR V- and one TCR J-segment family based on nucleotide sequence alignment to assemble a complete TCR chain sequence. In some embodiments, a J-gene is identified at 2-digit or 4-digit resolution. In some embodiments, nucleotide sequence alignment is based on 65% sequence identity, 70% sequence identity, 75% sequence identity, 80% sequence identity, 85% sequence identity, 90% sequence identity, 95% sequence identity, 96% sequence identity, 97% sequence identity, 98% sequence identity, 99% sequence identity, 100% sequence identity, and any number or range in between. In some embodiments, sufficient sequence information is obtained to identify TCRα- and β-chains from the created TCR library with desired feature(s).

In some embodiments, a recovered TCR chain is defined by the CDR3 nucleotide sequence. In some embodiments, a recovered TCR chain is defined by the CDR3 amino acid sequence.

In some embodiments, a recovered TCR chain is defined by sufficient 5′- and 3′-nucleotide sequence information to select at least one TCR V- and one TCR J-segment family. In some embodiments, a recovered TCR chain is defined by sufficient amino acid sequence information to select at least one TCR V- and one TCR J-segment family. In some embodiments, a recovered TCR chain is defined as sufficient nucleotide or amino acid sequence information to unequivocally identify a TCRαβ pair within a created TCR library. In some embodiments a recovered TCR chain is defined as a unique molecular identifier, such as a nucleotide-based barcode, that unequivocally identifies a TCRαβ pair within a created TCR library.

In some embodiments, a ICR chain sequence is defined based on nucleotide sequence alignment. In some embodiments, a TCR chain sequence is defined based on amino acid sequence alignment. Using nucleotide or amino acid sequence alignment, a complete TCR chain sequence can be assembled.

In some embodiments, a sample from a subject can be used that comprises non-viable starting material as described above. By way of example, non-viable starting material can comprise non-viable cells or non-viable tissue samples. Non-viable starting material can be preserved by any method known in the art.

In any of the foregoing embodiments, a defined part of the identified TCR repertoire can be recovered. In some embodiments, a defined part of the identified TCR repertoire comprises recovering a select part of the TCR repertoire rather than the complete TCR repertoire. A selected TCR repertoire can be defined by any of the criteria set forth above, such as defined frequency within the cell population, relative enrichment compared to a second T cell population, biological properties of the TCR chain, spatial patterns of gene expression, occurrence or co-occurrence at a similar frequency in multiple samples, selection into multiple groups or pools of TCR chains, or any combination thereof. In some embodiments, a selected TCR repertoire is defined by a given antigen-specificity. In some embodiments, the antigen-specificity comprises specificity for a neo-antigen. In some embodiments, antigen-specificity comprises predicted antigen-specificity.

In some embodiments, antigen-specific TCR sequences are recovered. In some embodiments, neo-antigen specific TCR sequences are recovered. By way of example, neo-antigens can be mutated proteins found in a tumor that are recognized by antigen-specific T cells. Thus, antigen-specific T cells directed against a tumor can exist, with TCR sequences that are specific to the tumor or its tumor antigens.

In any of the preceding embodiments, T cells expressing antigen specific TCR sequences can be used to diagnose or treat an infection or autoimmunity disorder.

In any of the preceding embodiments, T cells expressing neo-antigen specific TCR sequences can be administered as cancer therapy. For example, neo-antigen specific T cells can be used to target a tumor that expresses a neo-antigen. In some embodiments, neo-antigen specific T cells are generated by introducing neo-antigen specific TCR chains into the T cells. In some embodiments, the T cells expressing the neo-antigen specific TCR sequences can be autologous or allogeneic.

In any of the preceding embodiments, the method can be used for a diagnostic. For example, presence of antigen-specific TCRs against a certain tissue antigen may be indicative of auto-immune disease. By way of additional example, presence of antigen-specific TCRs against certain pathogens may be indicative of infectious disease.

In some embodiments, the diagnostic is to recover TCR repertoires from pathological sites of infection. In some embodiments, the diagnostic is to recover TCR repertoires from sites of autoimmunity. For example, cells or tissue at sites of infection or autoimmunity may express a particular antigen recognized by certain T cells. By determining the TCR sequences of T cells that can detect a particular antigen at a site of infection or autoimmunity, TCR repertoires associated with or specific to the site of infection or autoimmunity can be recovered. In order to identify TCR sequences against autoimmunity mediating self-antigens or pathogens, the library of combinatorial TCRαβ generated from selected TCRα- and TCRβ-chains can be tested for reactivity against a set of selected self-antigens or pathogen-derived antigens.

In any of the preceding embodiments, the method can be used for recovery of BCR/antibody repertoires. For example, B cells expressing a BCR receptor or producing antibodies specific for a particular antigen can be recovered. Thus, the BCR/antibody repertoire of recovered B cells can be determined by applying any of the methods described above to recover, select and combinatorially pair immunglobulin heavy and light chains to create an antibody repertoire. Antibodies with properties of interest can be selected from the created antibody repertoire.

In any of the above embodiments, the method can comprise isolating nucleic acids from a patient sample that comprises TCR-α and β nucleic acid sequences. The nucleic acid can be DNA or RNA. Nucleic acid can be isolated from any tissue or cell of a subject, including, but not limited to blood, skin, liver, bone marrow, biopsy material, and others. In some embodiments, the subject is a human. In some embodiments, the subject is a mammal. In some embodiments, the subject is an animal.

In any of the herein embodiments, a sample from a subject can comprise cells isolated from a body fluid. In some embodiments, the cells are tumor-specific T cells or tumor-infiltrating lymphocytes. In some embodiments, the body fluid is selected from the group consisting of blood, urine, serum, serosal fluid, plasma, lymph, cerebrospinal fluid, saliva, sputum, mucosal secretion, vaginal fluid, ascites fluid, pleural fluid, pericardial fluid, peritoneal fluid, and abdominal fluid.

In some embodiments, the one or more subsets of TCRα- and β-chain sequences from the total repertoire is selected based on at least one criterion: on frequency within the T cell population, on relative enrichment compared to a second T cell population, on relative difference of DNA and RNA copy numbers of a given TCR chain, on biological properties of the TCR chain, wherein the properties are selected from at least one of: (predicted) antigen-specificity, (predicted) HLA-restriction, affinity, co-receptor dependency, parental T cell lineage (e.g. CD4 or CD8 T cell) or TCR sequence motifs, on spatial patterns of gene expression, wherein spatial gene expression patterns are derived from at least one of: originating region in the tissue or co-expression patterns of other genes, on co-occurrence or occurrence at a similar frequency in multiple samples, for example occurrence in multiple tumor lesions, assignment to multiple groups to separately recover specific parts of the TCR repertoire, on a combination of multiple criteria as defined in the different embodiments.

In some embodiments, determining TCR-α and β sequences is achieved by at least one of: multiplex PCR; TCR-sequence recovery by target enrichment; TCR-sequence recovery by 5′RACE and PCR; TCR-sequence recovery by spatial sequencing; TCR-sequence recovery by RNA-seq, and the use of a Unique Molecular Identifier (UMI).

In some embodiments, step III is achieved by at least one of the following: TCR chain sequences are used to synthesize a library of TCRα- and β-chain DNA or RNA fragments which are linked into one DNA or RNA fragment (optionally, in which exactly one TCRα- and one β-chain are linked), combinations of TCRα- and β-chains are generated by directly synthesizing DNA or RNA fragments in which exactly one TCRα- and one β-chain are linked, or combinations of TCRα- and β-chains are created intracellularly by modification of a pool of cells with separate collections of TCRα- and β-genes in such a way that cells will express one TCRα- and one β-chain, combinations of TCRα- and β-chains are linked in a single-chain TCR construct containing both TCR chain fragments as well as CD3ζ or and CD3ϵ signaling domains alone or in combination with CD28 signaling domains.

In some embodiments, step IV is achieved by at least one of the following: a pool of reporter T cells modified with the library of generated TCRαβ pairs is stimulated by antigen presenting cells presenting at least one antigen of interest and antigen-reactive reporter cells are isolated based on at least one activation marker for TCR isolation; a pool of reporter cells modified with the library of generated TCRαβ pairs is labelled with a fluorescent dye suitable to trace cell proliferation, stimulated by antigen presenting cells expressing at least one antigen of interest, and antigen-reactive reporter cells are isolated based on proliferation for TCR isolation; a pool of reporter cells modified with the library of generated TCRαβ pairs is divided into at least two samples; samples are stimulated by antigen presenting cells expressing at least one antigen of interest or not; after stimulation, both reporter cell populations are incubated for a period of time and subsequently both reporter cell populations are analyzed by TCR isolation; comparison of TCRαβ pairs obtained from both samples will identify TCR genes with higher abundance in the sample exposed to at least one antigen; a pool of reporter cells modified with the library of generated TCRαβ pairs is stimulated by antigen presenting cells presenting at least one antigen of interest and antigen-reactive reporter cells are isolated for TCR isolation based on at least one reporter gene, such as NFAT-GFP or NFAT-YFP that reports on TCR triggering; a pool of reporter cells modified with the library of generated. TCRαβ pairs is stimulated by antigen presenting cells presenting at least one antigen of interest, and antigen-reactive reporter cells are isolated for TCR isolation based on selection of antigen-specific reporter cells based on selective survival, including but not limited to acquired antibiotic resistance, upon TCR signaling, for example by use of a NFAT-puromycin transgene; a pool of reporter cells modified with the library of generated TCRαβ pairs is exposed to one or multiple MHC complexes that carry an antigen of interest; reporter cells binding to an MHC complex are isolated for TCR isolation; a pool of reporter cells modified with the library of generated TCRαβ pairs is stimulated by antigen presenting cells expressing at least one antigen of interest; subsequently, TCRαβ pairs of interest are identified using single-cell based droplet PCR or microfluidic approaches to combine TCR isolation with the detection of transcript levels for at least one activation marker; thereby, single reporter cells within the pool of T cells in which TCRαβ transcripts are co-expressed with increased levels of activation marker are detected.

In some embodiments, the activation marker is selected from the group consisting of CD4 or CD8 T cell activation markers, CD69, CD137, IFN-γ, IL-2, TNF-α, GM-CSF, OX40,

In some embodiments, the activation marker is CD69, and two cell populations are isolated for further analysis, one cell population with high expression of CD69 and the other cell population with low expression of CD69.

In some embodiments, step IV is achieved by at least one of the following: identification or selection based on at least one activation marker; identification or selection based on proliferation in response to antigen; identification or selection based on identification of TCR genes of higher abundance in antigen-stimulated cells as compared to unstimulated cells; identification or selection based on reporter gene activation by TCR triggering; identification or selection based on selective survival, including but not limited to acquired antibiotic-resistance upon TCR signaling; identification or selection based on binding to one or more MHC complexes; identification or selection using single-cell based droplet PCR or microfluidics; or any combination thereof.

In some embodiments, reporter cells are T cells.

In some embodiments, identification or selection using single-cell based droplet PCR or microfluidics; or any combination thereof further comprises determination of co-expression of activation-associated genes.

Described herein, in some embodiments, are methods of creating multiple T cell libraries, the methods comprising: (a) recovering a repertoire of T cell receptors (TCRs) according to the methods described herein; (b) selection of TCRα- and β-chain sequences from the total repertoire into multiple groups to separately recover specific parts of the TCR repertoire, wherein multiple T cell libraries are created that are of smaller complexity or that recover specific parts of the TCR repertoire.

In some embodiments, selection of TCRα- and β-chain sequences is based on frequency range.

In some embodiments, cells are selected or sorted based on gating. In some embodiments, cells are sorted based on the highest 0.1%, 0.5%, 1 5%, 10%, 20%, 30%, 40%, 50%, 60%, 70%, 80%, 90%, 99.9%, or any number or range in between, live, single cells in a sample. In some embodiments, cells are sorted based on the lowest 0.1%, 0.5%, 1%, 5%, 10%, 20%, 30%, 40%, 50%, 60%, 70%, 80%, 90%, 99.9%, or any number or range in between, live, single cells in a sample.

In some embodiments, a library of nucleic acids is introduced into a population of cells. In some embodiments, a population of cells is diploid. In some embodiments, a population of cells is of any ploidy. In some embodiments, a first population of cells is selected from the population of library cells. In some embodiments, enrichment and/or depletion of nucleic acid sequences in a first population of cells is measured to identify nucleic acid sequences of interest. In some embodiments, enrichment and/or depletion of nucleic acid sequences in a first population is measured by comparing the population to a reference. In some embodiments, the reference may be a second population of cells or a library of nucleic acid sequences. In some embodiments, enrichment and/or depletion of nucleic acid sequences in a first population of cells is measured by comparison with more than one reference.

in some embodiments, the first and/or second population is isolated based on flow cytometry sorting. In some embodiments, flow cytometry sorting is carried out based on detecting a change in phenotype. In some embodiments, the change of phenotype is induced by contacting the population of library cells with another population of cells.

In some embodiments, the change of phenotype is detected by binding of a fluorescently labeled probe to the cells. In some embodiments, flow cytometry sorting is carried out based on a threshold. In some embodiments, the threshold is based on the intention to recover a percentage of cells with the highest fluorescent signal from the fraction of the total cells by flow cytometry sorting. In some embodiments, the Top 0.1%, 0.5%, 1%, 5%, 10%, 20%, 30%, 40%, 50%, 60%, 70%, 80%, 90%, 99.9% of cells based on fluorescence signal are isolated. In some embodiments, the threshold is based on the intention to recover a percentage of cells with a low fluorescent signal from the fraction of the total cells by flow cytometry sorting. In some embodiments, the Bottom 0.1%, 0.5%, 1%, 5%, 10%, 20%, 30%, 40%, 50%, 60%, 70%, 80%, 90%, 99.9% of cells based on fluorescence signal are isolated. In some embodiments, multiple thresholds are used to separate a sample into a first and a second population.

In some embodiments, the threshold is based on the intention to recover a minimum number of cells from the total pool of cells by flow cytometry based on fluorescence signal strength, for example, if at least 1×10e6 cells are to be recovered from 10×10e6 cells the Top10% or Bottom10% of cells based on fluorescence signal are isolated.

In some embodiments, the threshold is based on the fluorescence signal strength of a second cell population. In some embodiments, the threshold is based on a fluorescence signal strength that is higher than in the reference population. In some embodiments, the threshold is based on a fluorescence signal strength that is lower than in the reference population. In some embodiments, the fluorescence signal strength in the reference population is based on a subset of the population with secondary marker expression.

In some embodiments, the first and/or second population is isolated based on magnetic bead enrichment. In some embodiments, magnetic bead enrichment is carried out based on a change in phenotype. In some embodiments, the change of phenotype is induced by contacting the population of library cells with another population of cells.

In some embodiments, contacting the population of library cells with another population of cells (which can be referred to as ‘the other population of cells’) is with another population of cells that are genetically engineered to alter the phenotype. In some embodiments, the other population of cells is a polyclonal pool of genetically engineered cells. In some embodiments, the other population of cells are genetically engineered to express variant molecules. In some embodiments, the other population of cells are genetically engineered to express one or more antigens. In some embodiments, the other population of cells are genetically engineered to express one or more antigens in the form of minigenes. In some embodiments, the other population of cells are genetically engineered to express one or more antigens in the form of tandem minigenes.

in some embodiments, the other population of cells are cells that can present antigen (Antigen-Presenting Cells; APCs). In some embodiments, the other population of cells are dendritic cells. In some embodiments, the other population of cells are monocytes. In some embodiments, the other population of cells are cells engineered with MHC-class I and/or Class II alleles. In some embodiments, the other population of cells can be B cells. In some embodiments, the other population of cells can be autologous cells. In some embodiments, the other population of cells can be autologous B cells. In some embodiments, the other population of cells can be immortalized autologous B cells. In some embodiments, the other population of cells can be autologous B cells immortalized by EBV infection.

In some embodiments, contacting the population of library cells with the other population of cells is triggering specific interactions between factors expressed on cells belonging to either of the populations of cells. In some embodiments, the interaction between factors is a receptor—ligand interaction. In some embodiments, the receptor in the receptor—ligand interaction is the T cell receptor (TCR), In sonic embodiments, the ligand in the receptor—ligand interaction is an antigen presented on a major histocompatibility complex (MHC). In some embodiments, the receptor—ligand interactions between the population of library cells and the other population of cells triggers a phenotypic change that can be detected.

In some embodiments, the collection of variants expressed in the population of library cells is a library of plasmids each expressing a combination of a single TCRalpha and a single TCRbeta chain. In some embodiments, the TCR library is constructed by combinatorially joining a collection of TCRalpha and TCRbeta chains. In some embodiments, all combinations of TCRalpha and TCRbeta can be present in the TCR library. In some embodiments, multiple libraries of lesser complexity are used to create a library of higher complexity. In some embodiments, the complexity of the combined higher complexity library is less than a library having all combinations of TCRalpha and TCRbeta that are present in the combined higher complexity library. In some embodiments, pairing information or likelihood of pairing information is used in the design of the multiple libraries of lesser complexity, to maximize the chance of having a these TCRalpha-TCRbeta pair presented in the TCR library. In some embodiments, a library of lesser complexity can contain all combinations of one or more TCRalpha, and one or more TCRbeta chains. In some embodiments, the TCR library is contracted by first generating multiple variants of single nucleotide molecules encoding both TCRalpha and TCRbeta chains, and subsequently mixing two or more different variants of molecules encoding both TCRalpha and TCRbeta chains.

In some embodiments, the change of phenotype is detected by binding of a probe to the cells. In some embodiments, the cells do not need to be treated with any fixative prior to binding of a probe. In some embodiments, the probe allows to couple a magnetic bead to the cell. In some embodiments, magnetic bead enrichment allows to isolate a first and/or a second population of cells from the population of library cells. In some embodiments, the first or the second population of cells are cells retained by a magnet. In some embodiments, the first or the second population of cells are cells not retained by a magnet. In some embodiments, binding of multiple probes is used to isolate a first and/or a second population by sequential magnetic bead enrichment. In some embodiments, the probe binds to CD62L. In some embodiments, the probe binds to CD69. In some embodiments, at least one nucleotide sequence is statistically significantly enriched or depleted in the first population of cells. In some embodiments, statistically significant enrichment or depletion is determined relative to a reference. In some embodiments, at least one nucleotide sequence is statistically enriched in the first population of cells relative to the second population of cells. In some embodiments, flow cytometry sorting and magnetic bead enrichment are combined.

In some embodiments, a method of identifying a nucleotide sequence from a library of nucleic acids is provided. The method comprises introducing the library into a population of cells; contacting the library of cells with a second population of cells, selecting a first population of the library of cells based on expression of at least one marker by magnetic bead enrichment, identifying at least one nucleotide sequence based on a statistically significant enrichment or depletion of the nucleotide sequence within the selected first population relative to a control. In some embodiments, the entity that is enriched or depleted is a nucleotide sequence that is contained within the library of nucleic acids. In some embodiments, at least some of the first population of cells are configured to express one or more polypeptides encoded by a member of the library of nucleic acids. In some embodiments, marker expression is linked to an introduced nucleic acid from the library. In some embodiments, “linked” denotes that the introduced nucleic acid alters marker expression.

In some embodiments, a method of identifying a nucleotide sequence from a library of nucleic acids is provided. The method comprises: introducing the library of nucleic acids into a population of cells to form a library of cells; contacting the library of cells with a first population of cells; selecting a sub-population of the library of cells based on expression of at least one marker by magnetic bead enrichment; and identifying at least one nucleotide sequence based on a statistically significant enrichment or depletion of the nucleotide sequences within the sub-population relative to a control. In some embodiments, at least some of the sub-population of cells are configured to express one or more polypeptides encoded by a member of the library of nucleic acids.

In some embodiments, selecting is based upon an expression of the marker above a first threshold level. In some embodiments, the marker is suitable for magnetic bead enrichment, which may mean, but is not limited to the marker being accessible for labeling by a magnetic bead by extracellular expression (e.g., it must be accessible extracellularly). In some embodiments, the nucleotide sequences encode expressed polypeptides.

In some embodiments, the library of nucleic acids introduced into a population of cells leads to expression of variant molecules. In some embodiments, such variant molecules are T cell receptor sequences. In some embodiments, such variant molecules are switch receptors. In some embodiments, such variant molecules are CAR molecules.

In some embodiments, a method of identifying a nucleotide sequence encoding T cell receptor α(TCRα)- and TCRβ-chains from an (optionally) combinatorial library of nucleic acids is provided. The method comprises: introducing the nucleic acid library into a population of cells able to express TCRα- and TCRβ-chains to make a library of cells; and determining at least one nucleotide sequence or nucleic acid identity of the first population of variant nucleic acids based on an enrichment of the nucleotide sequence within the subset relative to a control. In some embodiments, the at least one nucleic acid is isolated from a first population of cells. In some embodiments, the first population of cells is selected based on an expression of a marker above a first threshold level in response to an antigen.

In some embodiments, any of the methods provided herein further comprise a step of administering T cells expressing the antigen specific TCR sequences to diagnose or treat an infection or autoimmunity.

In some embodiments, for any of the methods or compositions provided herein, the T cells can be autologous or allogeneic.

In some embodiments, for any of the methods or compositions provided herein, the activation marker is CD69.

In some embodiments, for any of the methods or compositions provided herein, two cell populations are isolated, one cell population with high expression of CD69 and the other cell population with low expression of CD69.

In some embodiments, for any of the methods or compositions provided herein, a nucleotide library comprising the repertoire of T cell receptors recovered according to any one of the methods provided herein is provided.

In some embodiments, for any of the methods or compositions provided herein, a nucleotide construct comprising the nucleotide sequence identified according to any of the methods provided herein is provided.

In some embodiments, for any of the methods or compositions provided herein, a cell comprising the nucleotide construct according to the above is provided.

In some embodiments, a method of identifying a nucleotide sequence encoding T cell receptor α (TCRα)- and TCRβ-chains from a sample is provided. The method comprises: a) sequencing TCR-α and β chains in a sample, b) selecting and combinatorial pairing TCRα- and β-chain sequences to create a library of TCRαβ pairs, c) introducing the library of TCRαβ pairs into a pool of reporter cells, d) stimulating the reporter cells that are modified with the library of TCRαβ pairs with antigen presenting cells presenting at least one antigen of interest (which can be done via the exactly two-pool process described herein, in some embodiments), e) determining TCRαβ pairs specific to the at least one antigen of interest, and f) introducing the TCRαβ pairs into cells and selecting cells containing the TCRαβ pairs.

In some embodiments, a method of identifying nucleotide sequences encoding antigen-specific T cell receptor α (TCRα)- and TCRβ-chain pairs from a combinatorial library of nucleic acids is provided. The method comprises: a) introducing a library into a population of cells able to express TCRα- and TCRβ-chains encoded by a member of a plurality of variant nucleic acids, b) selecting a subpopulation of the population of cells based on an expression of a marker above a threshold level in response to antigen (which can optionally be in antigen-presenting cells), wherein the subpopulation comprises a plurality of cells, c) isolating a subset of the plurality of variant nucleic acids from the subpopulation, d) determining nucleotide sequences of the variant nucleic acids, and e) identifying at least one variant nucleotide sequence based on an enrichment of the nucleotide sequences within the subset relative to a control. In some embodiments, the method further comprises providing the library comprising the plurality of variant nucleic acids encoding TCR alpha and TCR beta chains.

In some embodiments the percentage of cells that is sorted is based on comparison of the percentage of T cells with marker expression in control cultures and cultures with neo-antigen expressing cells. Any marker can be used for cell sorting. In some embodiments, markers for sorting cells comprise CD4, CD8 and CD69, for example.

Additional embodiments are shown in FIGS. 35A-35E, which shows the recovery of antigen-specific TCRs from a TCR library generated by gene synthesis. These embodiments show the idea of increasing the sensitivity of the TCR library screening platform by increasing the E:T ratio. These items are also embodied in some of the Examples below.

The number for the “steps” as used herein, merely denotes the embodiment being discussed in that particular section, the letter denotes the step of the process itself. Thus, for example, “step 35” denotes an embodiment entitled 35, while “a” denotes step “a” of that embodiment. The term “step” denotes part of a process, and does not necessarily require that any one step be complete before the another step is started.

Step 35A) Schematic of the screen design. Five characterized TCRs and 95 uncharacterized TCRs from ovarian cancer (©VC) or colorectal cancer (CRC) samples were used to create combinatorial TCR libraries of 100×100 design. The library was assembled by Twist Bioscience using human V, CDR3 and J segments, while the constant (C) region was of murine origin. The library was used for retroviral transduction of Jurkat reporter. T cells. The polyclonal reporter T cells were cocultured with antigen-presenting cells (APCs) that were engineered to present cognate antigens in a TMG format. For this example EBV-LCL cells expressing a TMG, and EBV-LCLs that have not been engineered to present specific antigens were used in the co-cultures.

Step 35B) Sorting strategy for the screen. The Jurkat reporter T cells expressing the 100×100 design TCR library produced as outlined in 35A) were co-cultured for 21 hours at a 1:1/1:2 and 1:3 ratio with the APCs mentioned in 35A). Following the co-culture APCs were depleted using magnetic bead selection based on CD20 expression on the B-cells. After B-cell depletion, cells were then sorted for. T cell activation by FACS using the CD69 marker. In some embodiments, the library can be sorted by any method. The sorting strategy included (from left to right) sequential gating to select lymphocytes, gating to select singlet cells, gating to exclude CD20⁺-cells, and two sorting gates (‘top’ and ‘bottom’) which capture cells expressing high and low CD69, respectively. The results are shown in the graph in FIG. 35B.

Step 35C) Retrieval of TCR expression cassettes. In some embodiments, one can retrieve the relevant TCR expression cassettes by any of a variety of techniques. In some embodiments, TCR expression cassettes of top and bottom samples from FIG. 35B) can be retrieved using the PM strategy described in 10C), followed by a barcoding PM. A control PCR on the plasmid TCR library was included as well. PCR products after the second-round. PCR were analysed using an Agilent TapeStation. The results are shown in FIG. 35C.

Step 35D) TCR enrichment analysis of the screen data. In some embodiments, the PCR product pool from step 35C) can be analysed in any number of ways. For example, the PCR product pool from 35C) was used for library preparation and was sequenced using Nanopore technology. TCR alpha and beta chain identities were recovered. and differentially represented TCR combinations were identified using the DESeq2 R package. Average Rlog-transformed read counts for screens in the presence (x-axis) and absence (y-axis) of TMG expression by B cells are represented for the effector to target (E:T) ratios of 1:1, 1:2 and 1:3. The five characterized antigen reactive TCRs are depicted as larger grey dots in FIG. 35D.

Step 35E) Characteristics of the five characterized antigen reactive TCRs. The rank of the most differentially represented TCR alpha x beta chain combinations from the data in 35D), as well as the concomitant p-value, were identified using the DESeq2 R package. Differential representation analysis is known to the skilled artisan, and is based on a linear model assuming an enriched TM is defined as being enriched in the ‘top’ sample and depleted in the ‘bottom’ sample where antigens were presented, relative to both ‘top’ and ‘bottom’ samples where no antigen was presented. The characteristics are tabulated in FIG. 35E.

Additional embodiments are shown in FIG. 36, which shows the recovery of antigen-specific TCRs from a TCR library generated by gene synthesis. These embodiments show the idea of using bead-based sorting instead of flow-based sorting as a way to separate cells for TCR library screens. The advantage of bead-based cell sorting is its increased scalability over FACS-based cell sorting.

Step 36A) Schematic of the screen design. Five characterized TCRs and 95 uncharacterized TCRs from ovarian cancer (OVC) or colorectal cancer (CRC) samples were used to create combinatorial TCR libraries of 100×100 design. The library was assembled by Twist Bioscience using human V, CDR3 and J segments, while the constant (C) region was of murine origin. The library was used for retroviral transduction of Jurkat reporter T cells. The polyclonal reporter T cells were cocultured with antigen-presenting cells (APCs) that were engineered to present specific antigens in a TMG format.

Step 36B) Sorting strategy for the screen. The Jurkat reporter T cells expressing the 100×100 design TCR library produced as outlined in 36A) were co-cultured for 21 hours at a 1:1 ratio with the APCs mentioned in 36A). Following the co-culture the AutoMACS from Miltenyi was used for sequential cell seperations. First, dead cells were removed using a dead-cell removal kit. With the live cells, APCs were depleted using magnetic bead selection based on CD20 expression on the B-cells. After B-cell depletion, CD20− cells were seperated using CD62L expression, a marker that is expressed on non-activated Jurkat cells. CD62L− and CD62L+ were then separately stained with an anti-CD69-biotin labelled antibody after which the cells were seperated using anti-biotin microbeads. The final fractions that were used to retrieve the TCR cassettes from were the CD20−, CD62L−, CD69+ cells, representing the “top” fraction and the CD20−, CD62L+, CD69− cells, representing the “bottom” fraction. A schematic of the cell separation process is depicted in FIG. 36B),

Step 36C) Retrieval of TCR expression cassettes. In some embodiments, one can retrieve the relevant TCR expression cassettes by any of a variety of techniques. In some embodiments, TCR expression cassettes of top and bottom samples from FIG. 36B) were retrieved using the PCR strategy described in 10C), followed by a barcoding PCR. A control PCR on the plasmid TCR library was included as well. PCR products after the second-round PCR were analysed using an Agilent TapeStation. The results are shown in FIG. 36C.

Step 36D) TCR enrichment analysis of the screen data. In some embodiments, the PCR product pool from step 36C) can be analysed in any number of ways. For example, the PCR product pool from 36C) was used for library preparation and was sequenced using Nanopore technology. TCR alpha and beta chain identities were recovered by alignment to the chains present in the library and differentially expressed TCR combinations were identified using the DESeq2 R package. Average It:log-transformed read counts for screens in the presence (x-axis) and absence (y-axis) of TMG expression by B cells are represented for every TCR in grey, and the five characterized antigen-reactive TCRs are represented as black dots.

Step 36E) Characteristics of the top 7 most significantly enriched TCRs. Differentially represented TCR alpha x beta chain combinations from the data in 36D) were identified using the DESeq2 R package. Differential representation analysis is known to the skilled artisan, and is based on a linear model assuming an enriched TCR is defined as being enriched in the ‘top’ sample where TMGs were expressed, and being depleted in the ‘bottom’ sample where TMGs were expressed, relative to both ‘top’ and ‘bottom’ samples where no TMGs were expressed. The alpha and beta chains of the top 7 most significant hits, as well as their representation, their log2-transformed fold change and the significance of differential representation are tabulated in FIG. 36E).

Additional embodiments are shown in FIG. 37, which shows additional analyses of the genetic screen to identify neo-antigen reactive TCRs from colorectal cancer (CRC) patients 2 and 4 (pt2/pt4). These embodiments show the idea of using combinatorial tandem minigene (TMG) encoding to efficiently screen TCR libraries against a large number of minigenes.

Step 37A) Schematic of a 6×6 combinatorial TMG encoding design. Given a screen where a TCR library needs to be screened against APCs expressing 36 TMG, pools of APCs can be created that each express a unique combination of 6 TINIGs. For instance, pool C1 consists of APCs expressing TMG1, TMG2, TMG3, TMG4, TMG5 and TMG6. Pool R1 consists of APCs expressing TMG1, TMG7, TMG 13, TMG19, TMG25 and TMG 31. Separate TCR library screens against each of the pools of APCs can be performed. From the combination of the two pools that are recognized by a TCR in the screening approach, the TMG that was recognized can be determined as the TMG that is represented in both pools.

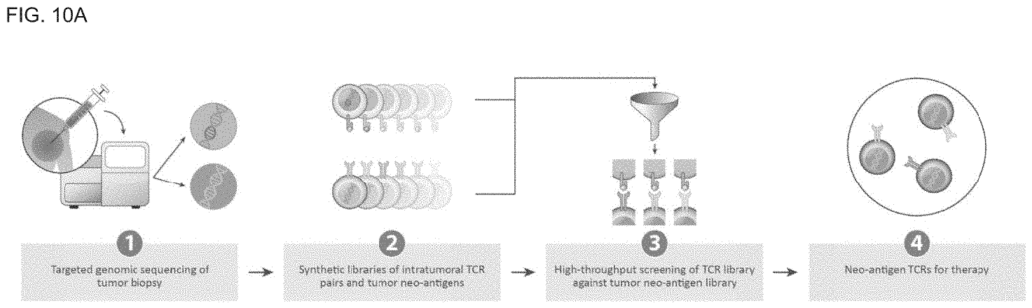

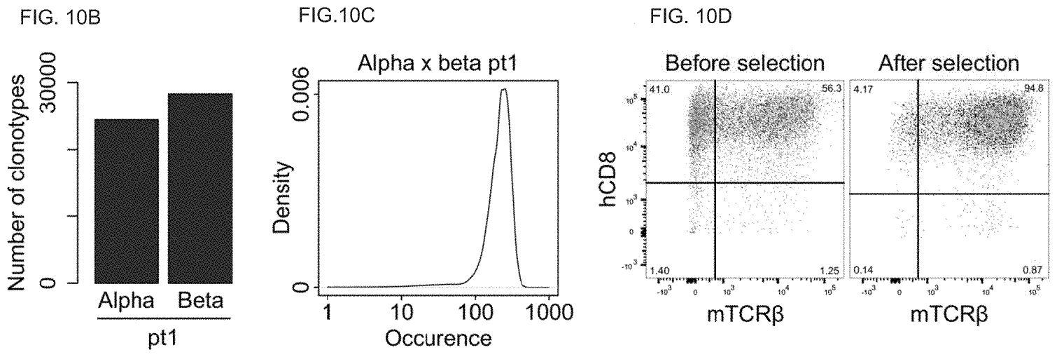

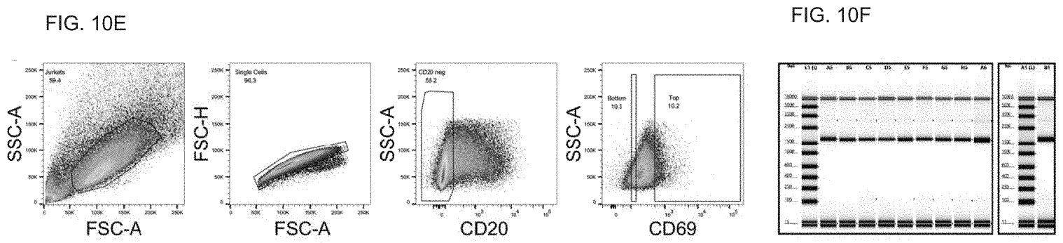

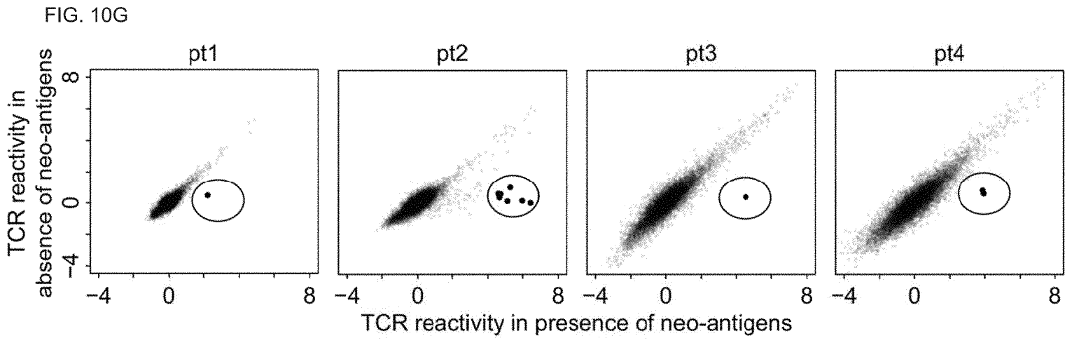

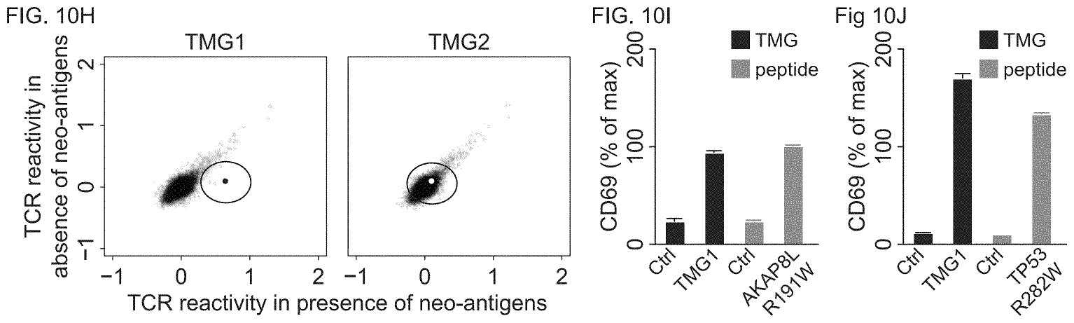

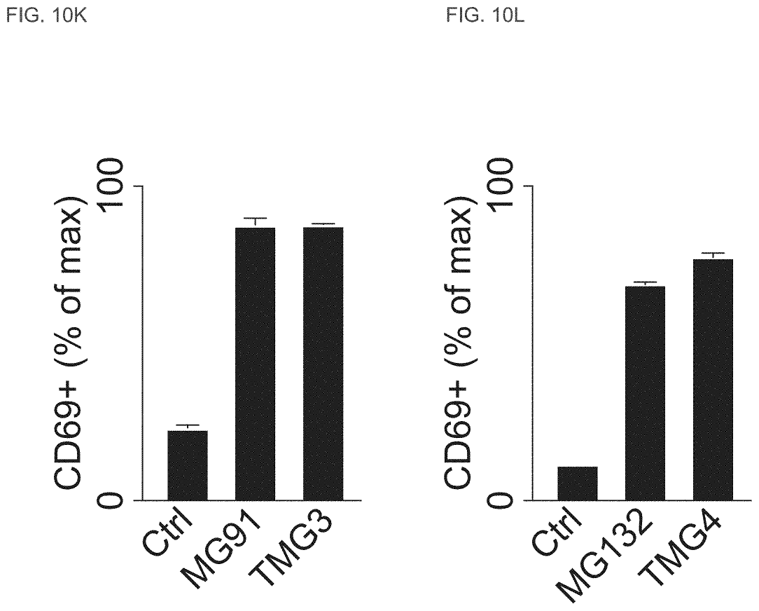

Step 37B) Analysis of the rank order of all TCR alpha x beta combinations as a function of the number of replicates of the pt2 TCR library screen. The pt2 TCR library screen data from FIG. 10G) were analyzed using either all 3 replicates, or 2 out of 3 replicates. Differentially expressed TCR combinations were identified using the DESeq2 R package for both conditions. Ranks for significance of enrichment were based on the Wald statistic as calculated using DESeq2 with the highest rank (rank 1) assigned to the highest Wald statistic value. The ranks based on 2 or 3 replicate-based analyses are represented on the y an x-axis, respectively. The neoantigen-reactive TCR leads identified in FIG. 10G) are represented as bigger black dots.

Step 37C) Summary table of the statistical analyses based on 2 or 3 replicates of the CRC TCR library screens. The analyses from FIG. 37B) were applied to all patient TCR library screens performed in FIG. 10G). All neoantigen-reactive TCR leads identified in FIG. 37B) are tabulated, together with their Bonferroni adjusted p-value for statistical significance of the enrichment of a TCR in the top samples relative to the bottom samples. This p-value is represented for analyses based on 3 screens replicates, or any of the combinations of 2 out of 3 screen replicates. Check marks in the last column represent that the same top-ranked TCR leads would have been picked regardless of whether 2 or 3 screen replicates would have been included in the screen analysis, and also regardless of which of the combinations of 2 out of 3 replicates would have been selected. An example of the is represented in FIG. 37B), where the 7 top-ranked neoantigen-reactive TCRs from pt4 selected for further validation (see FIG. 10G) are the same as the 7 top-ranked TCRs based on an analysis using only 2 replicates.

Step 37D) Table of the pt4 samples used for pairwise TCR enrichment analysis. Six samples (each being represented by both a top and a bottom sample) were included for pairwise TCR enrichment analysis. Samples included cocultures of the TCR library-expressing reporter T cells together with B cell lines that express TMG1, TMG2, TMG3 or TMG4 individually (samples 1-4, respectively), as well as a coculture with a pool of these B cell lines mixed at a 1:1:1:1 ratio (sample 5). For sample 6, the coculture was performed with B cells that were not engineered to express any exogenous antigens.

Step 37E) Pairwise TCR enrichment analysis results. All possible pairs of the samples in FIG. 19D) were analyzed for TCR enrichment in top samples relative to bottom samples using DESeq2. Differential representation analysis is known to the skilled artisan, and is based on a linear model assuming an enriched TCR is defined as being enriched in the ‘top’ sample where TMGs were expressed, and being depleted in the ‘bottom’ sample where TMGs were expressed, relative to both ‘top’ and ‘bottom’ samples where no TMGs were expressed. Bonferroni adjusted p-values were sorted and plotted in increasing order. Light grey dots represent p-values from analyses of the alpha43.beta16 TCR, and dark grey dots represent p-values from analyses involving the alpha9.beta14 TCR. Top outliers were the same TCRs that were identified in FIG. 10G). TCR alpha9.beta14 reactivity could be attributed to an antigen expressed from TMG4, as this was the shared TMG amongst samples 4 and 5. TCR alpha43.beta16 reactivity could be attributed to TMG3, as this was the shared TMG amongst samples 3 and 5.

Additional embodiments are shown in FIG. 38, which shows additional analyses of the genetic screen to identify neo-antigen reactive TCRs from colorectal cancer (CRC) patient 2. These embodiments show the sensitivity of the platform, and supports the notion that TCR characteristics, such as TCR sensitivity, may be determined from genetic screening data.

Step 38A) Correlation of TCR activation and TCR background activation between screening and validation data. Sixteen CRC pt2 TCRs varying in TCR reactivity in the genetic screening approach were identified based on the data from FIG. 10G). First, Rlog-transformed read counts were calculated using the DESeq2 R package, and the Rlog-values of the bottom sample were subtracted from the Rlog-values from the top samples and represented for cocultures that were performed in the presence (x-axis) and absence (y-axis) of TMG expression by B cells (FIG. 38). These TCRs were expressed in reporter T cells, and cocultured with EBV-B cells expressing the relevant TMG (TMG2) in independent validation experiments. T cell activation was measured using the CD69 marker by FACS analysis. To compare validation data with TCR library screening data, the fold activation of a TCR in the screens was defined as the difference between Rlog-values of top samples and bottom samples derived from a coculture with EBV-B cells expressing TMG2 (y-axis; middle panel). The fold activation in the validation experiment is defined as the ratio of CD69+ cells after coculture with TMG2-expressing versus non-engineered EBV-B cells (x-axis; middle panel). The background of the screen is defined as the enrichment of a given TCR in top vs bottom derived from a coculture with non-engineered EBV-B cells (y-axis; right panel). The background of the validation experiment is defined as the percentage CD69+ cells after coculture with non-engineered EBV-B cells (x-axis; right panel).

In some embodiments, the recovery of antigen-reactive TCRs from TCRαβ libraries can be through the isolation of one or more sub-populations based on response to antigen. In some embodiments, this approach entails one or more of the following steps: i) genetic engineering of reporter T cells to allow expression of TCRs of the TCRαβ libraries; ii) performing a coculture of these cells with antigen-presenting cells expressing at least one antigen; iii) cell separation based on a T cell activation markers into a) a ‘top’ population expressing one or multiple markers of T cell activation; and b) a ‘bottom’ population lacking (or having low) expression of one or multiple markers of T cell activation; iv) TCR identification from the top and bottom samples using PCR en genomic DNA and subsequent deep sequencing; and v) identification of at least one antigen-reactive TCRs which is enriched in the top sample relative to the bottom sample. Expression of a marker of T cell activation can be relatively high expression of a marker demarcating activated T cells (for example, CD69), or relatively low levels of expression of a marker demarcating non-activated. T cells (for example, CD62L).

In some embodiments, the top is the top 1, 5, 10, 20, 30, 40, or 50% and the bottom is the bottom 1, 5, 10, 20, 30, 40, or 50%, including any pair of ranges between any two of the noted values for top and bottom. In some embodiments, the top/bottom approach (where one employs both a top population and a bottom population) in any of the embodiments provided herein.

In some embodiments, a method of identifying a nucleotide sequence encoding an antigen-specific T cell receptor α(TCRα)- and TCRβ-chain pairs from a library of nucleic acids is provided. The method comprises a) introducing the nucleic acid library into a population of cells able to express TCRα- and TCRβ-chains to make a library of cells; b) selecting a first population of the library of cells based on an expression of a marker above a first threshold level in response to an antigen; and c) isolating a first population of variant nucleic acids from the first population of the library. In some embodiments, the method further comprises a) determining at least one nucleotide sequences or nucleic acid identity of the first population of variant nucleic acids; and b) identifying at least one variant nucleotide sequence based on an enrichment of the nucleotide sequences within the subset relative to a control. In some embodiments, the threshold level is based on at least one of:
a) recovery of a percentage of the total pool of cells based on expression of a marker; or
b) recovery of a minimal number of cells from the total pool of cells; or
c) recovery of cells retained by magnet based on binding of a magnetic probe to at least one marker expressed in response to an antigen.

In some embodiments, the control is a second population of cells that is below a second threshold. In some embodiments, the control is one or more of: a reference population of cells, the combinatorial library of nucleic acids that was introduced into the population of cells, a population of cells sorted from a same population of cells as the first population based on an expression marker below a second threshold, and/or at least one population of cells obtained from cocultures of reporter T cells expressing the relevant TCR library with B cells that are not engineered to express exogenous antigens. In some embodiments, the bottom (or control) sample is sorted from a same population of cells as the top sample, but having low activation marker expression or wherein the bottom sample is obtained from cocultures of reporter. T cells expressing the relevant TCR library, and B cells that are not engineered to express exogenous antigens. In some embodiments, there is no overlap between the top fraction and the bottom fraction. In some embodiments, the method further comprises adding an antigen to the population of cells. In some embodiments, isolating a first population and/or the control is achieved by at least one of a) magnetic bead enrichment, h) flow cytometry sorting, or c) both. In some embodiments, the control is one or more of: a reference population of cells, the combinatorial library of nucleic acids that was introduced into the population of cells, a population of cells sorted from a same population of cells as the first population based on an expression marker below a second threshold, or at least one population of cells obtained from cocultures of reporter T cells expressing the relevant TCR library with antigen presenting cells such as B cells that are not presenting any exogenous antigens.

In some embodiments, the top-bottom approach is set up so that antigen- reactive TCRs will become activation-marker positive upon antigen stimulation, and therefore such TCRs will be enriched in the top population relative to the bottom population. The top-bottom approach is illustrated by various accompanying figures as described in Example 24.

In some embodiments, the bottom sample (or control) may be any reference population of cells or reference library of TCR plasmids. The bottom sample may be sorted from the same population of cells as the top sample, but having low activation marker expression. The bottom sample may be obtained from cocultures of reporter T cells expressing the relevant TCR library, and B cells that are not engineered to express exogenous antigens. The bottom sample may be the TCR plasmid library that was used to create the reporter T cells from which the top sample was sorted. In some embodiments, the TCR representation in top and bottom samples may be compared to TCR representation in any other additional sample during differential TCR representation analysis. In some embodiments, such additional samples may be the plasmid TCR library. In some embodiments, such additional samples may be derived from cocultures of reporter T cells expressing the relevant TRC library, and B cells that are not engineered to express exogenous antigens.

In some embodiments, the method is one to recover a repertoire of T cell receptors (TCRs) from diverse T cell populations. The method can comprise determining nucleotide or amino acid sequences of paired TCRa and TCRb chains within a subject's sample; selecting TCRab pair sequences from the total repertoire; creating a TCR repertoire by creating a library of the selected TCRalpha/beta pairs; and identifying at least one TCRalpha/beta pair with desired features from the created TCR library.

In some embodiments, the TCRalpha/beta pairs can include TCR sequence motifs.

In some embodiments, provided herein are libraries of TCRαβ pairs. In some embodiments, these libraries can be created by any of the methods provided herein. In some embodiments, the libraries are from a sample that was non-viable.

In some embodiments, provided herein are cell populations and/or libraries that have the library of TCRαβ pairs introduced into a pool of reporter cells. Thus, in some embodiments, a pool of reporter cells is provided that includes the selected and combinatorially paired TCRα- and β-chain sequences (a library of TCRαβ pairs).

In some embodiments, the library can be a stimulated library. In some embodiments, the library can include the reporter cells that are modified with the library of TCRαβ pairs and can further include antigen presenting cells presenting at least one antigen of interest. In some embodiments, the at least one antigen of interest can be autologous or allogeneic.

In some embodiments, provided herein are libraries involving one or more TCRαβ pairs from a sample or characteristic of a non-viable sample.

### Additional Embodiments Regarding Screening

Further embodiments of the present disclosure are disclosed. Any of the present embodiments can be combined with any of the other embodiments provided herein. Provided herein are methods (also described herein as “screening methods”) to identify via high-throughput nucleic acid sequencing, sequences of interest among a library of variant sequences by genetic gain-of-function/loss-of-function screening (also referred to as “genetic variant library screening”). The screening methods can be used to screen libraries of variant nucleotide sequences in which individual nucleic acid sequences can only be unambiguously distinguished by identifying at least 600 by of the variant nucleotide sequence.

In some embodiments, any of the appropriate methods can employ a method of identifying involving pooled antigens. For example, in some embodiments, identifying or stimulating comprises: a) selecting a number of antigens; b) creating antigen-pools in which each antigen is present in exactly two antigen pools; c) evaluating reactivity of reporter cells expressing at least one T cell receptor against each of the antigen pools; and d) determine whether the at least one T cell receptor is reactive towards any of the selected antigens by evaluating for reactivity against exactly two antigen pools. In some embodiments, reactivity against exactly two antigen pools is detected by pairwise enrichment analysis. In some embodiments, the library is a TCR library. In some embodiments, one employs an activation marker. In some embodiments, one employs a top-bottom comparison (as described herein) to evaluate reactivity. In some embodiments, while other approaches may use reactivity against single pools to create unique reactivity patterns, this process can use pairwise analysis to increase signal strength by specifically analyzing replicates.

Screening methods of the present disclosure can be high throughput methods. Polyclonal genetic library screenings can allow screening of large numbers (e.g., several tens of thousands) of protein variants in a single experiment rather than requiring generation and analysis of individual clones. Additionally, the generation of variant gene libraries can be less expensive and time-consuming compared to the synthesis of individual variant genes. Some embodiments of the screening methods allow functional selection of variants, and protein variants can be selected based on one or more functional properties. Screening methods of the present disclosure can be sensitive screening methods. While variants of interest are selected based on functional phenotypes, the sequence identity of the variants of interest is identified based on DNA-sequencing methods which can detect even rare variants with high sensitivity. In some embodiments, the sensitivity of the embodiments provided herein can allow one to distinguish at a desired level, including, 1:1000, 1:10,000, 1:100,000, 1:1,000,000 or even lower. In some embodiments, higher sensitivity is possible provided that: (i) sufficient numbers of cells are analyzed and (ii) enough sequence reads can be generated. In some embodiments, a factor to consider is demultiplexing. However, this can be addressed by, for example: i) not multiplexing or ii) elevating the barcode threshold at the expense of throwing away more reads (e.g., sequencing more).

Some embodiments of the screening methods are methods to perform high-throughput genetic variant library screenings for protein variants where discrimination between variants is based on stretches of >200 amino acids, and on polyclonal population analysis. The present screening methods in some embodiments include a screening protocol and bioinformatic process that overcomes high error rates in some NGS sequencing reads, such as those generated by the Oxford Nanopore platform.

The screening methods of the present disclosure can include identifying any suitable variant proteins, independent of size and without restriction of sequence diversity location. In some embodiments, the method includes the selection of T cell receptor (TCR) sequences of interest from large TCR collections in which the pairing of distinct TCRα and TCRβ chains is either unknown or ambiguous, and includes determining the full sequence of variant gene cassettes that encodes TCRα and TCRβ variable region. In some embodiments, the present screening methods allows screening of TCR libraries with high throughput (e.g., by avoiding generation of clones), based on functional response of reporter cells (e.g. CD69 upregulation) that are mediated by the TCR.

In some embodiments, the method includes the identification of Chimeric antigen receptor (CAR) sequences with enhanced properties from large collections of CAR variants which largely differ in molecule design, for example by combinatorial assembly of up to three different signal domains selected from a pool of several different signaling domains. In some embodiments, the present screening methods allow for comprehensive CAR enhancement with screening variants with mutational diversity throughout the entire CAR molecule.

For any of the embodiments provided herein, where appropriate, there can be more than one CAR intracellular signaling domain. In some embodiments, there are at least two CAR intracellular signaling domains. In some embodiments, the at least two are the at least two CAR intracellular signaling domains: CD3ϵ, CD3ζ ITAM1, CD3ζ ITAM12, CD3ζ ITAM123, CD3ζ with any ITAM of CD3δ, CD3ϵ and CD3γ, CD8α, CD28, ICOS, 4-1BB (CD137), OX40 (CD134), CD27, and CD2.

In some embodiments, the screening methods of the present disclosure includes analyzing polyclonal reporter cell populations without deriving single-cell clones. In some embodiments, the screening methods can be used to screen libraries containing >10,000 variants to identify combinations of interest (e.g., at a coverage of 100×) without generating single cell clones.

In some embodiments, the amount of coverage depends on a number of factors including primary focus of screen (enrichment (lower coverage) or depletion screen (higher coverage)), the spread of representation of individual variants within the library, cell loss during the selection process, etc. Generally, a range of 50-10,000 can be used, including 100-2000 or for example, enrichment screens at 100-400×.

In some embodiments, the screening methods of the present disclosure provides a genetic screening methodology of molecule lead identification and enhancement for larger proteins with mutational diversity throughout the complete protein. Compared to other available methods, the method can in some embodiments provide high throughput (reduced costs and timelines) and high sensitivity in identifying molecule leads.

In general terms, a screening method can include (1) generating a library of variant nucleotide sequences containing at least two variant nucleotide sequences that can (or only can) be unambiguously identified by determining at least 600 bp of their total nucleotide sequence; (2) introducing the library of variant nucleotide sequences into reporter cells; (3) selecting reporter cells based on at least one functional property; (4) isolating variant nucleotide sequences from selected reporter cells; (5) determining at least 600 bp of the total nucleotide sequence of the isolated variant nucleotide sequences; and (6) selecting at least one variant nucleotide sequence of interest.

For any of the embodiments provided herein (that provide a reference to a 600 bp stretch or equivalent length of amino acids), the sequence variability of the library can be present in stretches which total to more than 600 bp. In some embodiments, the library will. contain or consist of or consist essentially of or comprise amplicons that are longer than 1500 bp. In some embodiments, the library will comprise/consist or consist essentially of at least 30, 40, 50, 60, 70, 80, 90, 95, 98, 99, or 100% of amplicons that will be larger than 1500 bp.

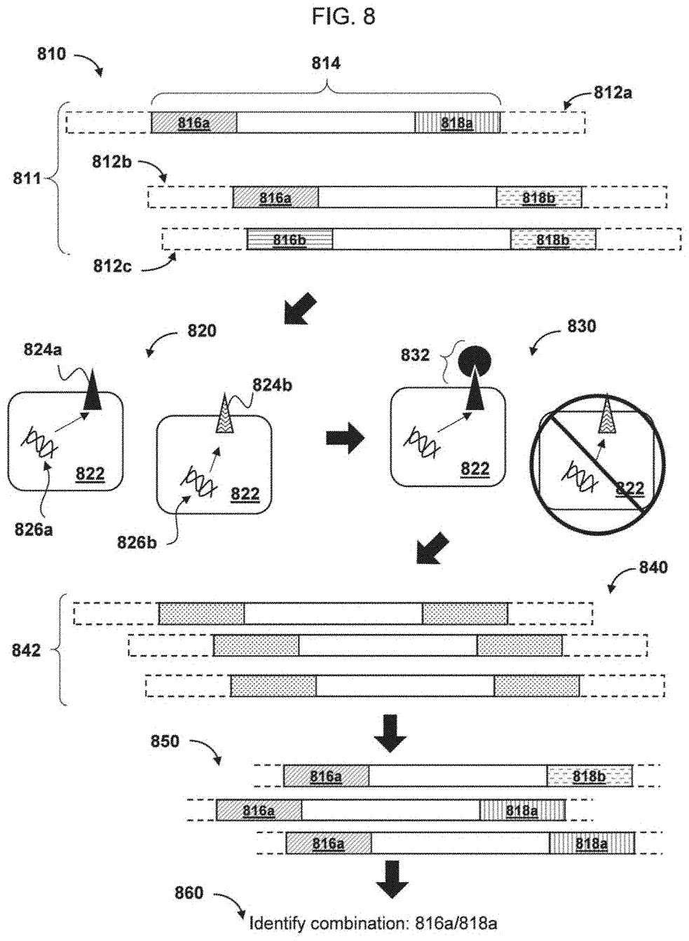

With respect to FIG. 8, some embodiments of the screening method of the present disclosure is provided. The method can include providing 810 a combinatorial library 811 that includes a plurality of variant nucleic acids (exemplified here by 812a, 812b, 812c). Each of the variant nucleic acids in the library can contain a contiguous portion 814 that is at least 600 bp in length.

“Contiguous,” as used herein with reference to a biopolymer (e.g., nucleic acid or polypeptide), refers to a sequence of individual building blocks (e.g., nucleotides or amino acids) of the biopolymer with no intervening sequences (e.g., a sequence of nucleotides with no intervening nucleotides or nucleotide sequence, a sequence of amino acids with no intervening amino acids or amino acid sequences). The contiguous portion 814 can contain two of more variant nucleotide subsequences.

As used herein, a “variant nucleotide subsequence” can include any one of a family of nucleotide sequences that defines a unit of functional activity of the nucleic acid and/or of a polypeptide(s) encoded by the nucleic acid. In some cases, a variant nucleotide subsequence can confer and/or contribute to a discrete functional activity (e.g., binding affinity, specificity) of the nucleic acid and/or of a polypeptide(s) encoded by the nucleic acid. In some cases, the family of nucleotide sequences confers and/or contribute to the discrete functional activity by virtue of: the variant nucleotide subsequence's position within the nucleic acid; having sequence similarity to a consensus sequence; and/or having variable and invariable regions where invariable regions are shared by other members of the same family of nucleotide sequences. In some embodiments, a TCR library includes variant nucleic acids having a variant nucleotide subsequence that corresponds to TCRα-chain, or one or more functional domains thereof (e.g., a TCRα V region, a TCRα complemnentarity determining region 3 (CDR3), a TCRα J-segment, a TCRα constant region), and a variant nucleotide subsequence that corresponds to TCRβ-chain, or a functional domain thereof (e.g., a TCRβ V region, a TCRβ complementarity determining region 3 (CDR3), a TCRβ J-segment, a TCRβ constant region). In some embodiments, a CAR library includes variant nucleic acids having a variant nucleotide subsequence that corresponds to one or more of CAR functional domains (e.g., an antigen-binding domain, a hinge domain, a transmembrane domain and an intracellular signaling domain, which can include 2-3 signaling modules).

Further with reference to FIG. 8, a nucleic acid 812a of the library may contain a variant nucleotide subsequence 816a that is one of multiple possible varieties (816a, 816b) in the library. The nucleic acid 812a may also contain at a different position another variant nucleotide subsequence 818a that is one of multiple possible varieties (818a, 818b). Another nucleic acid 812b in the library may contain a different combination, 816a/818b, of the variant nucleotide subsequences from the first combination, 816a/818a. Yet a third nucleic acid 812c may contain a different combination, 816b/818b, of the variant nucleotide subsequences from the first combination, 816a/818a, or the second combination, 816a/818b. Further, one end (e.g., 5′ or 3′ end) of the contiguous portion may be defined by one of the variant nucleotide subsequences, and the other end (e.g., 3′ or 5′ end, respectively) of the contiguous portion may be defined by another one of the variant nucleotide subsequences.

The contiguous portion may be represented by the formula: 5′-A*-X-B*-3′, where A* and B* represent different families of variant nucleotide subsequences, and X may be absent (in which case the contiguous portion is 5′-A*-B*-3′), or if present, may be any nucleotide sequence of any length. In some embodiments, A* may be any member of the family of variant nucleotide subsequences (e.g., A1, A2, A3, . . . , etc.). In some embodiments, B* may be any member of the family of variant nucleotide subsequences (e.g., B1, B2, B3, . . . , etc.). In some embodiments, X may include members of one of more families of variant nucleotide subsequences (e.g., C, D, . . . , etc.).

The screening method can further include introducing 820 the library into a population of cells 822, which can express one or more gene products (e.g., polypeptides), 824a, 824b, encoded by a member 826a, 826b of the plurality of variant nucleic acids.

The screening method can include selecting 830 a subpopulation of the population of cells based on at least one functional property 832, e.g., binding of the expressed polypeptide(s) to a ligand, where the functional property depends on the combination of the variant nucleotide subsequences in the nucleic acid member 826a, 826b which was introduced into the cell. The subpopulation of cells can include a plurality of cells. In some embodiments, the subpopulation of cells can include a plurality of different members of the plurality of variant nucleic acids, where the different members differ from each other by having different combinations of the variant nucleotide subsequences.

In some embodiments, the screening method can include isolating 840 a subset 842 of the plurality of variant nucleic acids from the subpopulation of cells, e.g., by extracting genomic DNA from the cells.

In some embodiments, the screening method can further include determining 850 the nucleotide sequence of the contiguous portion of individual members of the subset of the plurality of variant nucleic acids, e.g., by high-throughput sequencing of at least the contiguous portion of the variant nucleic acids in the subset 842.

In sonic embodiments, the method can further include identifying 860 the combination of variant nucleotide subsequences, 816a/818a, that was present in the cells of the subpopulation. In some embodiments, identifying includes analyzing whether a combination of variant nucleotide subsequences among two or more combinations found in the subpopulation of cells is enriched compared to a pre-determined threshold level, or compared to the abundance of that particular combination in a control subpopulation of cells. In some embodiments, a combination that is enriched is identified as conferring to the cells the functional property on which basis the subpopulation of cells were selected.

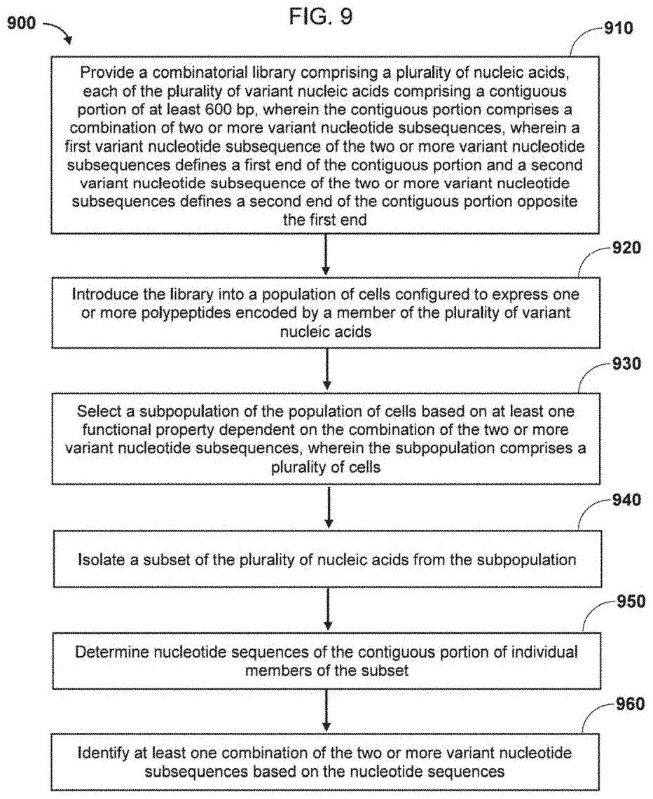

With reference to FIG. 9, some embodiments of the present screening methods are provided. The method 900 can include providing 910 a combinatorial library containing a plurality of variant nucleic acids, each of the plurality of variant nucleic acids having a contiguous portion of at least 600 bp, wherein the contiguous portion comprises a combination of two or more variant nucleotide subsequences, wherein a first variant nucleotide subsequence of the two or more variant nucleotide subsequences defines a first end of the contiguous portion and a second variant nucleotide subsequence of the two or more variant nucleotide subsequences defines a second end of the contiguous portion opposite the first end. The combinatorial library can be introduced 920 into a population of cells configured to express one or more polypeptides encoded by a member of the plurality of variant nucleic acids. Then, the method can include selecting 930 a subpopulation of the population of cells based on at least one functional property dependent on the combination of the two or more variant nucleotide subsequences, wherein the subpopulation comprises a plurality of cells. The method can include isolating 940 a subset (e.g., one or more) of the plurality of variant nucleic acids from the subpopulation, and determining 950 nucleotide sequences of the contiguous portion of individual members of the subset. Then one or more combinations of the two or more variant nucleotide subsequences may be identified 960 based on the determined nucleotide sequences.

In some embodiments, there is a “reference” and a “top” sample, for each screen. The “top” sample contains sorted cells which display the highest CD69 expression, while the “reference” sample contains sorted cells which display low CD69 expression. In some embodiments, samples may be separated on the basis of any other activation marker, including, but not limited to, CD25, CD62L, CD137, IFN-γ, IL-2, TNF-α, GM-CSF, synthetic promoter reporter markers or proliferation markers. For which, one can isolate variants at a minimum of 100-200× coverage for each of the variants in the library with complexity Y. This can total to >100-200*Y variants/cells per sample.

One nucleic acid may be considered “different” or “distinct” from another nucleic acid in the combinatorial library when a variant nucleotide sequence of one nucleic acid encodes for an amino acid sequence that is different from the amino acid sequence encoded by a corresponding variant nucleotide sequence in the other nucleic acid. In some embodiments, one nucleic acid may be considered “different” or “distinct” from another nucleic acid in the combinatorial library based on differences in their nucleotide sequence while they encode the same amino acid sequence.

The combinatorial library can include any suitable number of different (or “variant”) nucleic acids. In sonic embodiments, the combinatorial library includes about 100 or more, e.g., about 200 or more, about 300 or more, about 400 or more, about 500 or more, about 600 or more, about 700 or more, about 800 or more, about 900 or more, about 1,000 or more, about 2,000 or more, about 3,000 or more, about 4,000 or more, about 5,000 or more, about 7,500 or more, about 10,000 or more, about 20,000 or more, about 50,000 or more, about 1×10⁵ or more, about 2×10⁵ or more, about 5×10⁵ or more, about 1×10⁶ or more, about 1×10⁷ or more, about 1×10⁸ or more, about 1×10⁹ or more, about 1×10¹⁰ or more, including about 1×10¹¹ or more different nucleic acids, or a number of different nucleic acids within a range defined by any two of the preceding values. In some embodiments, the combinatorial library includes between about 100 to about 200, about 200 to about 500, about 500 to about 1,000, about 1,000 to about 5,000, about 5,000 to about 1×10⁴, about 1×10⁴ to about 2×10⁴, about 2×10⁴ to about 5×10⁴, about 5×10⁴ to about 1×10⁵, about 1×10⁵ to about 1×10⁶, about 1×10⁶ to about 1×10⁷, about 1×10⁷ to about 1×10⁸, about 1×10⁸ to about 1×10⁹, about 1×10⁹ to about 1×10¹⁰, about 1×10¹⁰ to about 1×10¹¹, or more different nucleic acids. In some embodiments, the library comprises at least 100, e.g., at least 200, at least 300, at least 400, at least 500, at least 600, at least 700, at least 800, at least 900, at least 1,000, at least 2,000, at least 3,000, at least 4,000, at least 5,000, at least 7,500, at least 10,000, at least 20,000, at least 50,000, at least 1×10⁵, at least 2×10⁵, at least 5×10⁵, at least 1×10⁶, at least 1×10⁷, at least 1×10⁸, at least 1×10⁹, at least 1×10¹⁰, including at least 1×10¹¹ different combinations of the two variant nucleotide subsequences.

In some embodiments, the one or more polypeptides (in some embodiments encoded by the variant nucleic acids of the combinatorial library) comprises: T cell receptor a (TCRα)- and TCRβ-chains; a chimeric antigen receptor (CAR); a switch receptor; or one or more chains of an antibody or antigen binding fragment thereof. In some embodiments, the first variant nucleotide subsequence encodes a TCRα variant amino acid sequence and the second variant nucleotide subsequence encodes a TCRβ variant amino acid sequence. In some embodiments, the two or more variant nucleotide subsequences encodes one or more of: a TCR V region, a TCR complementarity determining region 3 (CDR3), a TCR J-segment, and a TCR constant region. In some embodiments each of the two or more variant nucleotide subsequences encodes an antigen binding domain, a hinge domain, a transmembrane domain, or one or more intracellular signaling domains of a CAR.

In some embodiments, the edit distance among contiguous portions of the plurality of variant nucleic acids in the library is maximized. In some embodiments, the edit distance between any two variant nucleic acids of a combinatorial library is maximized, e.g., by controlling codon usage. In some embodiments, this can include codon-optimization. In some embodiments, any of the methods provided herein involving a library, can include the nucleotide sequence(s) in the library (e.g., of the plurality of variant nucleic acids) being optimized based at least one of the following: introduction of preferable codon usage for the host cell, optimization of mRNA structural stability, avoidance of repetitive sequences, avoidance of long stretches of homopolymers, and avoidance of large differences in local GC-content within a given variant nucleic acid sequence.

In some embodiments, the nucleotide sequence of the plurality of variant nucleic acids in the library is optimized based at least one: 1) any method provided herein, where cells of the population of cells are genetically modified, or 2) any method provided. herein where the cells are reconstituted with CD4 and/or CD8 and utilized to screen for Class I and/or Class II restricted TCR sequences.

In some embodiments, the cells employed are T cells.

In some embodiments, the subpopulation and control population of cells are non-overlapping. In some embodiments, non-overlapping denotes that the cells in both populations have a different activation status, but can carry a same variant nucleic acid.

In some embodiments, the one or more polypeptides comprises TCRα- and TCRβ-chains, and wherein the invariant amino acid sequence comprises a TCRβ constant region.

In some embodiments, determining comprises obtaining an average coverage of at least 25, at least 50, 100, at least 200, at least 300, at least 400, at least 500 or at least 1,000 for each of the nucleotide sequences of the contiguous portion.

In some embodiments, any of the methods involving an evaluation of reactivity or that can further comprise an evaluation of reactivity, can employ a top-bottom comparison to evaluate reactivity.

In some embodiments, any of the methods provided herein involving a library, can include the nucleotide sequence(s) in the library (e.g., of the plurality of variant nucleic acids) being optimized based at least one of the following: introduction of preferable codon usage for the host cell, optimization of mRNA structural stability, avoidance of repetitive sequences, avoidance of long stretches of homopolymers, and avoidance of large differences in local GC-content within a given variant nucleic acid sequence.

In some embodiments, the antigen is presented via an antigen-presenting cell.

In some embodiments, the library is a combinatorial library.

In some embodiments, the antigen is provided by a cell.

In some embodiments, the process involves a high degree of antigen diversity and/or complexity.

In some embodiments involving a library, the library is a combinatorial library. In some embodiments, the combinatorial library is a TCR library.

The contiguous portion may have any suitable length of at least 600 bp. In some embodiments, the length of the contiguous portion depends on the read length (e.g., accurate read length) of the sequencing platform used to sequence the contiguous portion after selecting the subpopulation of cells based on a functional property. In some embodiments, the contiguous portion has a length of at least 600 bp, e.g., at least 700 bp, at least 800 bp, at least 900 bp, at least 1,000 bp, at least 1,100 bp, at least 1,200 bp, at least 1,300 bp, at least 1,400 bp, at least 1,500 bp, at least 1,750 bp, at least 2,000 bp, at least 2,500 bp, at least 3,000 bp, at least 4,000 bp, at least 5,000 bp, at least 6,000 bp, at least 7,000 bp, at least 8,000 bp, at least 9,000 bp, at least 10,000 bp, at least 11,000 bp, at least 12,000 bp, at least 13,000 bp, at least 14,000 bp, or at least 15,000 bp, or a length within a range defined by any two of the preceding values. In some embodiments, the contiguous portion has a length of from about 600 bp to about 15,000 bp, e.g., from about 800 bp to about 12,000 bp, from about 1,000 by to about 10,000 bp, from about 1,000 bp to about 8,000 bp, from about 1,000 bp to about 6,000 bp, from about 1,000 by to about 5,000 bp, including from about 1,000 bp to about 4,000 bp.

The variant nucleic acids in the combinatorial library can have any suitable number of variant nucleotide sequences. In some embodiments, all nucleic acids in a combinatorial library have the same number of variant nucleotide sequences. In some embodiments, variant nucleic acids in the combinatorial library have 2, 3, 4, 5, 6, 7, 8, 9, 10, 12, 15, 20 or more variant nucleotide sequences, each of which can have variants which can be assembled in combinatorial fashion to assemble a contiguous portion of the variant nucleic acids.

The combinatorial library may include any suitable number of variants for each variant nucleotide sequence. In some embodiments, the number of variants for a variant nucleotide sequence in the combinatorial library is 2 or more, 3 or more, 4 or more, 5 or more, 6 or more, 7 or more, 8 or more, 9 or more, 10 or more, 12 or more, 14 or more, 16 or more, 18 or more, 20 or more, 25 or more, 30 or more, 35 or more, 40 or more, 45 or more, 50 or more, 75 or more, 100 or more, 125 or more, 150 or more, 175 or more, 200 or more, 250 or more, 300 or more, 350 or more, 400 or more, 450 or more, 500 or more, 600 or more, 700 or more, 800 or more, 900 or more, 1,000 or more, 1,500 or more, 2,500 or more, 3,000 or more, 4,000 or more, 5,000 or more, 7,500 or more, including 10,000 or more, or a number of variants within a range defined by any two of the preceding values. In some embodiments, the number of variants for a variant nucleotide sequence in the combinatorial library is between 2 to 10, between 10 to 20, between 20 to 30, between 30 to 40, between 40 to 50, between 50 to 100, between 100 to 200, between 200 to 500, between 500 to 1,000, between 1,000 to 2,000, between 2,000 to 5,000, or between 5,000 to 10,000. In some embodiments, the distribution of frequencies of individual variants within a library is such that >80% of those variants have a frequency within the range starting from median frequency/8 and ending at median frequency*8.

In some embodiments, for any of the screening and/or library related methods provided herein, the TCR pairs and/or the T cells expressing the TCR pairs are selected or identified by binding to an antigen (such as a neoantigen), wherein the antigen is expressed by a B cell or an antigen presenting cell.

In some embodiments, for any of the screening and/or library related methods provided herein, the antigen or neoantigen is from a tumor in a subject, and a TCR alpha and a TCR beta of the TCR pairs are also each from the subject (meaning that a single subject has both the antigen sequence and both the TCR alpha and TCR beta sequences).

In some embodiments, for any of the screening and/or library related methods provided herein, there are at least 2, 3, 4, 5, 6, 7, 8, 9, 10, 50, 100, 500, 1000, 10000, 100000, or 1 million TCR pairs (or cells comprising these pairs) and there are at least 2, 3, 4, 5, 6, 7, 8, 9, 10, 50, 100, 500, 1000, 10000, 100000, or 1 million antigens present.

In some embodiments, for any of the screening and/or library related methods provided herein, a) the TCR pairs and/or the T cells expressing the TCR pairs are selected or identified by binding to an antigen (such as a neoantigen), wherein the antigen is expressed by a B cell or an antigen presenting cell, b) the antigen or neoantigen is from a tumor in a subject, and a TCR alpha and a TCR beta of the TCR pairs are also each from the subject (meaning that a single subject has both the antigen sequence and both the TCR alpha and TCR beta sequences), and c) there are at least 2, 3, 4, 5, 6, 7, 8, 9, 10, 50, 100, 500, 1000, 10000, 100000, or I million TCR pairs (or cells comprising these pairs) and there are at least 2, 3, 4, 5, 6, 7, 8, 9, 10, 50, 100, 500, 1000, 10000, 100000, or 1 million antigens present.

The combinatorial library may be a library of any suitable biomolecule (e.g., protein, nucleic acid, nucleoprotein, etc.). In some embodiments, a suitable biomolecule are those in which the sequence (e.g., the protein and/or nucleic acid sequence) can be varied over a contiguous portion of sequence units (e.g., amino acids or nucleotides) corresponding to at least 600 nucleotides. A suitable combinatorial library includes, without limitation, a combinatorial library for TCRs, CARs, antibodies, RNA-guided nucleases, etc.

In some embodiments, the combinatorial library includes a repertoire of T cell receptors (TCRs) from diverse T cell populations. In some embodiments, the plurality of nucleic acids of the combinatorial library includes variant nucleotide sequences that encode one or more TCRα functional domains (e.g., a TCRα V region, a TCRα complementarity determining region 3 (CDR3), a TCRα J-segment, a TCRα constant region), and one or more TCRβ functional domains (e.g., a TCRβ V region, a TCRβ complementarity determining region 3 (CDR3), a TCRβ J-segment, a TCRβ constant region). In some embodiments, the contiguous portion of a nucleic acid of a combinatorial library TCRs has a length of between 600 bp to 2,000 bp, e.g., between 800 bp to 1,900 bp, between 1,000 bp to 1,900 bp, between 1,200 bp to 1,900 bp, between 1,400 by to 1,900 bp, between 1,500 by to about 1900 bp, between 1,600 bp to 1900bp, between 1700 bp to 1900 bp, or about 1,800 bp.

In some embodiments, the plurality of nucleic acids of the combinatorial library includes variant nucleotide sequences that encode one or more chimeric antigen receptor (CAR) functional domains (e.g., an antigen-binding domain, a hinge domain, a transmembrane domain and an intracellular signaling domain, which can include 2-3 signaling modules). In some embodiments, the contiguous portion of a nucleic acid of a combinatorial library CARs has a length of between 600 bp to 2,000 bp, e.g., between 800 bp to 1,900 bp, between 1,000 bp to 1,800 bp, between 1,200 bp to 1,800 bp, between 1,400 bp to 1,700 bp, or about 1,500 bp.

In some embodiments, the combinatorial library includes a repertoire of antibody heavy and light chain sequences. In some embodiments, the plurality of nucleic acids of the combinatorial library includes variant nucleotide sequences that encode one or more antibody heavy chain functional domains (e.g., heavy chain variable regions (including one or more CDRs, framework regions), and/or heavy chain constant regions (including one or more of CH1, CH2, CH3 and hinge regions). In some embodiments, the plurality of nucleic acids of the combinatorial library includes variant nucleotide sequences that encode one or more antibody light chain functional domains (e.g., light chain variable regions (including one or more CDRs, framework regions), and/or a light chain constant region.

In some embodiments the nucleic acids of the combinatorial library include a suitable vector that contains the variant nucleic acids. In some embodiments, the nucleic acids of the combinatorial library contain suitable regulatory and/or non-coding sequences. Suitable non-coding sequences include, without limitation, a promoter, signal peptide, splicing site, stop codon, and poly(A) signal sequences. In some embodiments, the nucleic acids contain a selection marker (e.g., an antibiotic resistance gene, a fluorescent molecule or a cell surface marker).

in some embodiments, the screening method includes generating the combinatorial library. The combinatorial library can be made using any suitable options. In some embodiments, generating the combinatorial library includes identifying two or more sets of variant nucleotide subsequences encoding two or more sets of variant amino acid sequences of the one or more polypeptides, wherein the at least one functional property depends on a combination of variant amino acid sequences from each of the two or more sets of variant amino acid sequences; and assembling the contiguous portion by combining a variant nucleotide subsequence from each of the two or more sets of variant nucleotide subsequences to thereby generate the member of the plurality of variant nucleic acids.

In some embodiments, generating a combinatorial library includes identifying multiple variants of variant nucleotide subsequences encoding variant amino acid sequences of the polypeptide that is to be expressed by the reporter cells. Where the polypeptide includes two or more variant nucleotide subsequences, the contiguous portion can be assembled by combining a variant from each of the variant nucleotide subsequences. In some embodiments, generating the combinatorial library includes designing the nucleic acids of the library in silic0, e.g., to maximize the edit distance through control of codon usage. Any suitable algorithm may be used to maximize the edit distance. The combinatorial library may be synthesized using any suitable options based on, e.g., the in silico generated design. In some embodiments, the combinatorial library comprises a repertoire of TCRs from diverse T cell populations.

Any suitable options of introducing the combinatorial library into cells may be used. Suitable options include, without limitation, viral transduction, transposon-mediated gene delivery, transformation, electroporation, nuclease mediated site-specific integration (e.g., CRISPR/Cas9, TALEN). In some embodiments, introducing the combinatorial library into cells includes viral transduction, transposon-based gene delivery, or nuclease-mediated site-specific integration.

The combinatorial library may be introduced into any suitable cells, e.g., reporter cells, configured to express the polypeptide(s) encoded by the nucleic acids of the library. Suitable cells include, without limitation, mammalian cells, insect cells, yeast, and bacteria. In some embodiments, suitable carriers include viruses, yeast, bacteria, and phage. While the present disclosure uses the term “cells” throughout for simplicity, it is contemplated herein that all such disclosures of “cells” herein, includes not just various forms of T cells (such as immortalized T cells), yeast and bacteria, but can also be more generically used with any carrier, including viruses and phage. Accordingly, the disclosure around “cells” as used herein (with reference to cells into which a combinatorial library may be introduced), can include eukaryotic cells, prokaryotic cells, and to denote an option where viruses and phages can also be employed as carriers. The cells can be a cell line, immortalized cells, or primary cells. In some embodiments, the cells are human cells, or are derived from a human cell. In some embodiments, the population of cells comprises immortalized T cells or primary T cells. In some embodiments, the immortalized T cells or primary T cells are human T cells. In some embodiments, the combinatorial library is introduced into immortalized T cells or primary T cells (e.g., by viral transduction). In some embodiments, the cells exhibit none or little of the functional property based on which the cells will be selected to identify the combination of variant nucleotide subsequences of interest. In some embodiments, the cells exhibit none or little of the functional property mediated by the polypeptide encoded by the nucleic acids of the library and dependent on the combination of variant nucleotide subsequences. In some embodiments, the cells of the population of cells are engineered, e.g., genetically modified. In some embodiments, the cells are engineered, e.g., genetically modified, to reduce or eliminate endogenous or background expression of the functional property by the cells. In some embodiments, the cells are engineered, e.g., genetically modified, to enhance the ability of the cells to exhibit the functional property when introduced with the combinatorial library. In some embodiments, the cells are engineered, e.g., genetically modified, to promote growth and/or maintenance of the population in culture. In some embodiments, the cells of the population do not comprise an endogenous polypeptide conferring the at least one functional property to the cells. In some embodiments, the cells are genetically modified to introduce or enhance or eliminate or reduce expression of one or more of CD4, CD8 and CD28. In some embodiments, the genetically modified cells are T cells.

In some embodiments, each cell of the population of cells into which the combinatorial library is introduced includes on average one nucleic acid of the plurality of nucleic acids. In some embodiments, the population of cells is transduced with the combinatorial library at a multiplicity of infection (MCI) of 10 or less, e.g., 7 or less, 5 or less, 3 or less, 2 or less, including 1 or less. In some embodiments, introducing comprises virally transducing the population of cells at a multiplicity of infection (MCI) of 5 or less. In some embodiments, nuclease mediated site-specific integration (e.g., CRISPR/Cas9, TALEN) is used to introduce exactly one or two nucleic acids into each cell of the population of cells.

The size of the population of cells into which the combinatorial library is introduced may include any suitable number of cells. In some embodiments, the number of cells depends on one or more of the size of the library, the relative representation of variant nucleic acids in the library, the desired level of representation of each variant nucleic acid in the population (also referred to as “coverage”), the type of screen that is performed (e.g., whether an ‘enrichment’ screen (primary goal is to identify enriched variants) or a ‘depletion’ screen (primary goal is to identify depleted variants) is executed), the representation and error rate of individual variants within the library and the process steps required to select a subpopulation of the total cell population based on at least one functional property that are associated with cell loss. Examples of such process steps include but are not limited to selection for successfully transduced cells and/or selection for the functional property mediated by the expressed polypeptide by flow cytometry. In some embodiments, the screening method includes adjusting a size of the population of cells based on a number of different combinations of the two or more variant nucleotide subsequences in the library.

In some embodiments, the screening method includes identifying the cells that have been successfully modified by having received a nucleic acid of the library based on a selection marker that is included in the nucleic acids. In some embodiments, the screening method includes using a marker to select or screen for cells in the population of cells expressing at least one of the plurality of variant nucleic acids. In some embodiments, the marker is a cytotoxin resistance marker and/or a cell surface marker. Successfully modified cells may be selected using any suitable method, depending on the selectable marker used. In some embodiments, the cells are selected based on antibiotic resistance, for example, without limitation, resistance to Puromycin or Blasticidin. In some embodiments, the cells are selected based on a detectable marker expression, for example, without limitation, by a cell surface marker or fluorescent molecule that can be used for sorting with flow cytometry. In some embodiments, the cells are selected based on a cell surface marker, for example suited for magnetic bead-based enrichment.

Selecting the subpopulation of the population of cells can be based on any suitable functional property of the polypeptide encoded by the variant nucleic acids. Suitable functional properties include, without limitation, ligand binding (e.g., antigen binding), signal transduction in response to a stimulus (e.g., response to antigen binding). Signal transduction can include, without limitation, phosphorylation, translocation, signaling domain interaction, or transcriptional changes. Selecting the subpopulation of the population of cells based on a functional property dependent on the combination of the variant nucleotides subsequences can be performed using any suitable options. In some embodiments, suitable functional outputs are measured using, without limitation, expression of a marker, or cell proliferation in response to a stimulus.

In some embodiments, selecting comprises selecting the subpopulation based on expression of a detectable marker, wherein the expression depends on the at least one functional property of the one or more polypeptides. In some embodiments, the detectable marker comprises a cell-surface marker, a cytokine marker, a cell proliferation marker, a transcription reporter, a signal transduction reporter, and/or a cytotoxicity reporter. In some embodiments, the cell-surface marker comprises one or more of: CD69, CD62L, CD137; the cytokine marker comprises one or more of: IFN-γ, IL-2, TNF-α, GM-CSF; the transcription reporter comprises one or more of: NF-κB, NFAT, AP-1; the signal transduction reporter comprises one or more of: ZAP70, ERK1/2; and the cytotoxicity reporter comprises one or more of: CD107A, CD107B, Granzyme B.

In some embodiments, selecting the subpopulation of the population of cells includes selecting cells that exhibit the functional property (e.g., respond positively in the functional assay). In some embodiments, selecting the subpopulation of the population of cells includes selecting cells that do not exhibit the functional property (e.g., respond negatively in the functional assay). In some embodiments, the screening method includes selecting a subpopulation of the population of cells that exhibit the functional property, and selecting another subpopulation of the population of cells that do not exhibit the functional property. In some embodiments, selecting the subpopulation of the population of cells includes selecting multiple subpopulation of cells based on stratification of the level or extent of the functional property exhibited by the cells of each subpopulation.

In some embodiments, selecting the subpopulation comprises contacting the population of cells with one or more of: a second population of cells; a ligand for the one or more polypeptides; an agonist or antagonist of the one or more polypeptides; and a small molecule, wherein a change in the subpopulation induced by the contacting depends on the at least one functional property of the one or more polypeptides. In some embodiments, selecting the subpopulation comprises detecting the presence or absence of the change, and/or a magnitude of the change; and selecting the subpopulation based on the detecting. In some embodiments, the second population of cells comprises antigen-presenting cells. In some embodiments, the antigen-presenting cells comprise B-cells and/or dendritic cells. In some embodiments, the second population of cells comprises primary cells or immortalized cells. In some embodiments, the variant nucleotide subsequences of the library are derived from cells expressing a variant polypeptide comprising an amino acid encoded by the variant nucleotide subsequences, wherein the cells are obtained from a subject, and wherein the second population of cells is derived from the subject.

In some embodiments, selecting comprises selecting a first subpopulation of the population of cells based on a measure of the at least one functional property above or below a threshold. In some embodiments, the threshold is determined based on a measure of the functional property in an unselected subpopulation of the population of cells. In some embodiments, selecting further comprises selecting a second subpopulation of the population of cells based on a second measure of the at least one functional property above or below a second threshold, wherein the first and second subpopulations are non-overlapping. In some embodiments, identifying the at least one combination comprises comparing an abundance of the at least one combination between the first and second subpopulations.

In some embodiments, where the combinatorial library is a TCR library containing TCRα and TCRβ variants, the subpopulation of cells is selected based on the ability of the cells to respond to antigen presentation by changes in expression of one or more markers. Suitable markers include, without limitation, CD69, CD62L, CD137, IFN-γ, IL-2, TNF-α and GM-CSF. In some embodiments, the marker is a promoter activity reporter, including, without limitation, NF-κB, NFAT, and AP-1.

In some embodiments, the antigen is presented by antigen-presenting cells including, but not limited to B cells (e.g., immortalized B cells), and dendritic cells. In some embodiments, the identity of the antigen is not known. In some embodiments, the antigen is a neo-antigen.

In some embodiments, where the combinatorial library is a CAR library containing variants of CAR functional domains, the subpopulation of cells is selected based on the ability of the cells to respond to antigen presentation by changes in cell proliferation and/or changes in marker expression. Suitable markers include, without limitation, CD69, CI)62L, CD137, IFN-γ, IL-2, TNF-α, and GM-CSF. In some embodiments, the marker is a signal transduction reporter, including, without limitation, ZAP70 and. ERK1/2 phosphorylation. In some embodiments, the marker is a cytotoxicity reporter, such as, without limitation, CD107A and CD107B. In some embodiments, the marker is a promoter activity reporter, such as, without limitation, NF-κB, NFAT, and AP-1.

In some embodiments, the subpopulation comprises a plurality of cells. In some embodiment, the isolating does not comprise isolating single clones of the subpopulation based on the at least one functional property. In some embodiments, the subpopulation comprises at least 1,000 cells (e.g., 10× coverage on 100 variants).

In some embodiments, the subpopulation selected based on a functional property dependent on the combination of the variant nucleotide subsequences includes about 1,000 or more cells, e.g., about 2,000 or more cells, about 3,000 or more cells, about 4,000 or more cells, about 5,000 or more cells, about 7,500 or more cells, about 10,000 or more cells, about 20,000 or more cells, about 50,000 or more cells, about 1×10³ or more cells, about 2×10⁵ or more cells, about 5×10⁵ or more cells, about 1×10⁶ or more cells, about 1×10⁷ or more cells, about 1×10⁸ or more cells, about 1×10⁹ or more cells, about 1×10¹⁰ or more cells, about 1×10¹¹ or more cells, including about 1×10¹² or more cells, or a number of cells within a range defined by any two of the preceding values. In some embodiments, the subpopulation selected based on a functional property dependent on the combination of the variant nucleotide subsequences includes between about 1,000 to about 1×10¹² cells, e.g., between about 2,000 to about 1×10¹² cells, between about 3,000 to about 1 ×10¹⁰ cells, between about 5,000 to about 1 ×10⁹ cells, between about 5,000 to about 1×10⁹ cells, including between about 1×10⁴ to about 1 ×10⁹ cells.

In some embodiments, the function of the TCR pair is binding to an antigen.

In some embodiments, the subpopulation is a fraction of the initial population, e.g., less than 10⁻¹⁴, 10⁻¹³, 10⁻¹², 10⁻¹¹, 10⁻¹⁰, 10⁻⁹, 10⁻⁸, 0.0000001, 0.000001, 0.0001, 0.001, 0.01, 0.1, 1, 2, 3, 4, 5, 6, 7, 8, 9, 10, 20, 30, 4,0 50, 60, 70, 80 or 90% of the original population (including any range defined between any two of the preceding values)

In some embodiments, the size of the subpopulation selected based on a functional property dependent on the combination of the variant nucleotide subsequences is sufficiently large so that the variant nucleic acids in the library are represented adequately in the subpopulation. In some embodiments, the subpopulation has a size that provides for a fold coverage of about 10 or more, e.g., about 20 or more, about 30 or more, about 40 or more, about 50 or more, about 60 or more, about 70 or more, about 80 or more, about 90 or more, about 100 or more, about 120 or more, about 140 or more, about 160 or more, about 180 or more, about 200 or more, about 250 or more, about 300 or more, about 400 or more, about 500 or more, including about 1,000 or more, or a fold coverage within a range defined by any two of the preceding values, of the total number of variants of variant nucleic acids in the library. In some embodiments, the subpopulation has a size that provides for a fold coverage of between about 10 to about 1,000, e.g., between about 20 to about 1,000, between about 30 to about 750, between about 40 to about 500, between about 50 to about 500, between about 50 to about 400, about 60 to about 300, between about 70 to about 250, including between about 80 to about 200 of the total number of variants of variant nucleic acids in the library.

isolating the variant nucleic acids from the subpopulation can be done using any suitable approaches. In some embodiments, isolating the variant nucleic acids includes extracting genomic DNA from the subpopulation using any suitable method. In some embodiments, isolating the variant nucleic acids includes extracting bulk genomic DNA from the subpopulation. In some embodiments, isolating the variant nucleic acids does not include isolating individual clones from the subpopulation and isolating genomic DNA from the individual clone. In some embodiments, isolating the variant nucleic acids does not include isolating individual clones from the subpopulation and expanding the individual clones, to thereby isolate genomic DNA from the expanded clonal population. In some embodiments, isolating the variant nucleic acids includes extracting bulk genomic DNA from the subpopulation without isolating or expanding individual clones from the subpopulation. In some embodiments, isolating the variant nucleic acids includes extracting RNA from the subpopulation using any suitable method. In some embodiments, isolating the variant nucleic acids includes extracting bulk RNA from the subpopulation. In some embodiments, isolating the variant nucleic acids includes extracting mRNA from the subpopulation. In some embodiments, isolating the variant nucleic acids does not include isolating individual clones from the subpopulation and isolating RNA from the individual clone. In some embodiments, isolating the variant nucleic acids does not include analysis of single cells by single cell PCR methods. In some embodiments, isolating the variant nucleic acids does not include isolating individual clones from the subpopulation and expanding the individual clones, to thereby isolate RNA from the expanded clonal population. In some embodiments, isolating the variant nucleic acids includes extracting RNA from the subpopulation without isolating or expanding individual clones from the subpopulation. The term “RNA” is a genus term and includes natural and artificial versions of RNA, for example. While the present specification often outlines options with respect to “DNA”, it will be understood that all such options and. embodiments can instead be used for RNA.

In some embodiments, the amount of genomic DNA extracted from the subpopulation is sufficient to provide adequate coverage of each variant in the variant nucleic acids of the library. In some embodiments, the amount of genomic DNA extracted from the subpopulation is sufficient to provide for a fold coverage of about 10 or more, e.g., about 20 or more, about 30 or more, about 40 or more, about 50 or more, about 60 or more, about 70 or more, about 80 or more, about 90 or more, about 100 or more, about 120 or more, about 140 or more, about 160 or more, about 180 or more, about 200 or more, about 250 or more, about 300 or more, about 400 or more, about 500 or more, including about 1,000 or more, or a fold. coverage within a range defined by any two of the preceding values, of the total number of variants of variant nucleic acids in the library. In some embodiments, the amount of genomic DNA extracted from the subpopulation is sufficient to provide for a fold coverage of between about 10 to about 1,000, e.g., between about 20 to about 1,000, between about 30 to about 750, between about 40 to about 500, between about 50 to about 500, between about 50 to about 400, about 60 to about 300, between about 70 to about 250, including between about 80 to about 200 of the total number of variants of variant nucleic acids in the library. In some embodiments, the determining comprises obtaining an average coverage of at least 10 for each of the nucleotide sequences of the contiguous portion, before or in the absence of any amplification of the individual members.

In some embodiments, isolating the variant nucleic acids from the subpopulation includes amplifying the extracted variant nucleic acids. Any suitable portion of the variant nucleic acids may be amplified. In some embodiments, substantially only the contiguous portion of the variant nucleic acids is amplified. In some embodiments, the portion of the variant nucleic acids encoding the entire polypeptide is amplified. In some embodiments, the size of the amplification products (or amplicons) is at least 600 bp, e.g., at least 700 bp, at least 800 bp, at least 900 bp, at least 1,000 bp, at least 1,100 bp, at least 1,200 bp, at least 1,300 bp, at least 1,400 bp, at least 1,500 bp, at least 1,750 bp, at least 2,000 bp, at least 2,500 bp, at least 3,000 bp, at least 4,000 bp, at least 5,000 bp, at least 6,000 bp, at least 7,000 bp, at least 8,000 bp, at least 9,000 bp, at least 10,000 bp, at least 11,000 bp, at least 12,000 bp, at least 13,000 bp, at least 14,000 bp, or at least 15,000 bp, or a length within a range defined by any two of the preceding values. In some embodiments, the size of the amplification products (or amplicons) is from about 600 by to about 15,000 bp, e.g., from about 800 bp to about 12,000 bp, from about 1,000 bp to about 10,000 bp, from about 1,000 bp to about 8,000 bp, from about 1,000 bp to about 6,000 bp, from about 1,000 by to about 5,000 bp, from about 1,000 bp to about 4,000 bp, from about 1,000 bp to about 3,000 bp, including from about 1,000 bp to about 2,000 bp.

In some embodiments, determining comprises amplifying at least the contiguous portion of the individual members of the plurality of nucleic acids. In some embodiments, the amplifying comprises using an amplification primer that hybridizes to an invariant nucleotide subsequence, wherein each of the plurality of variant nucleic acids comprises the invariant nucleotide subsequence, and wherein the invariant nucleotide subsequence encodes an invariant amino acid sequence of the one or more polypeptides. In some embodiments, amplifying comprises using an amplification primer that hybridizes to a nucleotide subsequence outside the variant nucleotide sequence, including but not limited to, non-coding nucleotide sequences of the gene vector. In some embodiments, the one or more polypeptides comprises TCRα- and TCRβ-chains, and wherein the invariant amino acid sequence comprises a TCRα constant region.

In some embodiments, the amplification is performed to provide sufficient coverage of each variant in the variant nucleic acids of the combinatorial library in the amplified amplicon library. In sonic embodiments, the isolated variant nucleic acids are amplified to provide a fold coverage of about 1,000 or more, e.g., about 2,000 or more, about 3,000 or more, about 4,000 or more, about 5,000 or more, about 6,000 or more, about 7,000 or more, about 8,000 or more, about 9,000 or more, about 10,000 or more, about 12,000 or more, about 14,000 or more, about 16,000 or more, about 18,000 or more, about 20,000 or more, about 25,000 or more, about 30,000 or more, about 40,000 or more, about 50,000 or more, including about 100,000 or more, or a fold coverage within a range defined by any two of the preceding values, of the total number of variants of variant nucleic acids of the combinatorial library in the resulting amplicon library. In some embodiments, the isolated variant nucleic acids are amplified to provide for a fold coverage of between about 1,000 to about 100,000, e.g., between about 2,000 to about 100,000, between about 3,000 to about 75,000, between about 4,000 to about 50,000, between about 5,000 to about 50,000, between about 5,000 to about 40,000, about 6,000 to about 30,000, between about 7,000 to about 25,000, including between about 8,000 to about 20,000 of the total number of variants of variant nucleic acids of the combinatorial library in the resulting amplicon :library. In some embodiments, the determining comprises obtaining an average coverage of at least 1,000 for each of the nucleotide sequences of the contiguous portion.

In some embodiments, amplification is done in a manner to reduce amplification bias in the resulting amplicon library. In some embodiments, amplification bias is reduced by reducing the number of cycles of amplification. In some embodiments, amplification bias is reduced by having a sufficiently large subpopulation of cells that reduces the number of amplification cycles. In some embodiments, unique molecular identifiers (UMIs) are used to reduce bias in the sequencing data due to amplification bias.

In some embodiments, isolating the variant nucleic acids from the subpopulation does not include amplifying the isolated variant nucleic acids.

In some embodiments, isolating the variant nucleic acids from the subpopulation includes using a CRISPR-based selective library preparation. Any suitable option for CRISPR-based selective library preparation can be used. In some embodiments, the isolating comprises using CRISPR/Cas9-mediated targeted fragmentation of genomic DNA from the subpopulation.. In some embodiments, isolating the variant nucleic acids from the subpopulation includes dephosphorylating genomic DNA extracted from the subpopulation, introducing CRISPR/Cas9-mediated double stranded breaks at positions flanking the sequence of interest (e.g., the contiguous portion of the variant nucleic acid), and ligating adaptors (e.g., sequencing adaptors) at the double-stranded breaks. The adaptor-ligated sequences can then be sequenced using any suitable approaches. In some embodiments, determining the nucleotide sequences of the contiguous portion of individual members of the subset includes harcoding each individual member of the subset.

Determining the nucleotide sequences of the contiguous portion of individual members of the subset can be done using any suitable options. In some embodiments, determining the nucleotide sequences of the contiguous portion involves sequencing the contiguous portion. Any suitable sequencing platform can be used. Suitable sequencing platforms include, without limitation, Sanger sequencing, pyrosequencing, sing-molecule sequencing, ion semiconductor sequencing, sequencing by synthesis, combinatorial probe anchor synthesis sequencing, sequencing by ligation, single molecule real-time (SMRT) sequencing and/or nanopore sequencing. In some embodiments, determining the nucleotide sequences of the contiguous portion involves using a sequencing platform that allows for long sequencing reads. In some embodiments, determining comprises sequencing the individual members by generating sequencing reads of at least 600 by of the contiguous portion. In some embodiments, the sequencing reads are between 600 hp and 15,000 bp long. In some embodiments, determining the nucleotide sequences of the contiguous portion involves generating or Obtaining sequencing reads of at least 600 bp, e.g., at least 700 hp, at least 800 bp, at least 900 bp, at least 1,000 bp, at least 1,100 bp, at least 1,200 bp, at least 1,300 bp, at least 1,400 bp, at least 1,500 bp, at least 1,750 bp, at least 2,000 bp, at least 2,500 bp, at least 3,000 bp, at least 4,000 bp, at least 5,000 bp, at least 6,000 bp, at least 7,000 bp, at least 8,000 bp, at least 9,000 bp, at least 10,000 bp, at least 11,000 bp, at least 12,000 bp, at least 13,000 hp, at least 14,000 bp, or at least 15,000 bp, or a sequence read length within a range defined by any two of the preceding values, of the contiguous portion. In some embodiments, the sequencing reads are from about 600 hp to about 1,000 hp from about 1,000 bp to about 2,000 bp, from about 2,000 by to about 3,000 bp, from about 3,00( )by to about 4,00( )bp, from about 4,000 bp to about 5,000 bp, from about 5,000 bp to about 7,000 bp, from about 7,000 bp to about 10,000 bp, and/or from about 10,000 hp to about 15,000 by of the contiguous portion.

Identifying at least one combination of the two or more variant nucleotide subsequences based on the nucleotide sequences can be done using any suitable option. In some embodiments, a combination of interest is identified by determining that the combination is enriched, or depleted, in the subpopulation selected based on a functional property. Relative abundance of the combination can be based on any suitable comparison of the abundance of the combination in the nucleotide sequences determined from the subpopulation with a reference level of abundance. Suitable reference levels of abundance include, without limitation, the abundance of the combination in another subpopulation of cells selected based on a lack of the functional property, a different level of response, or a different type of response; or the abundance of the combination in a subpopulation that has not been selected. In some embodiments, a combination of interest is identified by determining that the combination is enriched, or depleted, in a positively-selected subpopulation compared to a negatively-selected subpopulation. In some embodiments, a combination of interest is identified by determining that the combination is enriched, or depleted, in the subpopulation selected based on a functional property compared to the abundance of the combination in the combinatorial library.

In some embodiments, identifying the at least one combination comprises measuring an enrichment of the at least one combination in the subpopulation relative to a control population of cells. In some embodiments, the population of cells comprises the control population of cells, and wherein the subpopulation and control population of cells are non-overlapping. In some embodiments, the control population of cells are selected based on a second functional property that is different from the at least one functional property.

In some embodiments, the screening method includes: providing a library comprising a plurality of variant nucleic acids, each of the plurality of variant nucleic acids comprising a contiguous portion of at least 600 bp, wherein the contiguous portion comprises: a combination of a first variant nucleotide subsequence encoding a TCRα variant amino acid sequence and defining a first end of the contiguous portion, and a second variant nucleotide subsequence encoding a TCRβ variant amino acid sequence and defining a second end of the contiguous portion opposite the first end; introducing the library into a population of immortalized T cells configured to express TCRα- and TCRβ-chains encoded by a member of the plurality of variant nucleic acids; selecting a subpopulation of the population of immortalized T cells based on an expression of a T cell activation marker above a threshold level in response to contacting the immortalized T cells with immortalized B cells expressing an antigen, wherein the suhpopulation comprises a plurality of T cells; isolating a subset of the plurality of variant nucleic acids from the subpopulation; determining nucleotide sequences of the contiguous portion of individual members of the subset; and identifying at least one combination of the first and second variant nucleotide subsequences based on an enrichment of the at least one combination in the nucleotide sequences of the subset relative to a control. In some embodiments, the method further includes: selecting a second subpopulation of the population of immortalized cells based on the expression of the cell activation marker below a second threshold level in response to contacting the immortalized T cells with the immortalized B cells, wherein the second subpopulation comprises a second plurality of T cells, and wherein the subpopulation and second suhpopulation are non-overlapping; isolating a second subset of the plurality of variant nucleic acids from the second subpopulation; and determining second nucleotide sequences of the contiguous portion of individual members of the second subset, wherein the at least one combination is identified based on an enrichment of the at least one combination in the subset relative to the at least one combination in the second nucleotide sequences of the second subset.

In some embodiments, the screening method includes: providing a library comprising a plurality of variant nucleic acids, each of the plurality of variant nucleic acids comprising a contiguous portion of at least 600 bp, wherein the contiguous portion comprises a combination of two or more of: a first variant nucleotide subsequence encoding a CAR hinge domain; a second variant nucleotide subsequence encoding a CAR transmembrane domain; and a third variant nucleotide subsequence encoding a CAR intracellular signaling domain, wherein one of the first, second or third variant nucleotide subsequences define a first end of the contiguous portion, and wherein another one of the first, second or third variant nucleotide subsequences defines a second end of the contiguous portion opposite the first end; introducing the library into a population of cells configured to express a CAR encoded by a member of the plurality of variant nucleic acids, wherein the population of cells comprises a population of immortalized T cells or primary human T cells; selecting a subpopulation of the population of cells based on cell proliferation above a threshold level in response to contacting the cells with antigen-presenting cells expressing an antigen specific to an antigen-binding domain of the CAR, wherein the subpopulation comprises a plurality of cells; isolating a subset of the plurality of variant nucleic acids from the subpopulation; determining nucleotide sequences of the contiguous portion of individual members of the subset; and identifying at least one combination of the first, second, and third variant nucleotide subsequences based on an enrichment of the at least one combination in the nucleotide sequences of the subset relative to a control. In some embodiments, the method further includes: selecting a second subpopulation of the population of cells based on cell proliferation below a second threshold level in response to contacting the cells with the antigen-presenting cells, wherein the second subpopulation comprises a second plurality of cells, and wherein the subpopulation and second subpopulation are non-overlapping; isolating a second subset of the plurality of variant nucleic acids from the second subpopulation; determining second nucleotide sequences of the contiguous portion of individual members of the second subset, and wherein the at least one combination is identified based on an enrichment of the at least one combination in the subset relative to the at least one combination in the second nucleotide sequences of the second subset.

Within the present disclosure, various embodiments are often described as involving a contiguous portion that comprises a combination of two or more variant nucleotide subsequences. Furthermore, these embodiments often involve a first variant nucleotide subsequence and a second variant nucleotide and selecting a subpopulation of the population of cells based on at least one functional property dependent on the combination of the two or more variant nucleotide subsequences. However, an alternative embodiment, expressly considered for all such embodiments involving two or more variant nucleotides, is one involving a single functional sequence (i.e., a single variant nucleotide). In such embodiments, the 600 bp sequence would be for a sequence over a single nucleic acid sequence, that could encode, for example, a single protein with a single function. Thus, for all embodiments disclosed herein involving “two or more variant nucleotide subsequences”, it is also envisioned to apply the method(s) to a situation in which there is only “a variant nucleotide subsequence” again, where the sequence itself is 600 bp or larger. For example, in some embodiments, a method of identifying a nucleotide sequence from a combinatorial library of nucleic acids is provided. The method comprises providing a combinatorial library comprising a plurality of variant nucleic acids, each of the plurality of variant nucleic acids comprises a contiguous portion of at least 600 bp. The method further comprises introducing the library into a population of cells configured to express one or more polypeptides encoded by a member of the plurality of variant nucleic acids. The method further comprises selecting a subpopulation of the population of cells based on at least one functional property dependent on the contiguous portion of at least 600 bp, wherein the subpopulation comprises a plurality of cells. The method further comprises isolating a subset of the plurality of variant nucleic acids from the subpopulation. The method further comprises determining nucleotide sequences of the contiguous portion of individual members of the subset. The method further comprises identifying the contiguous portion of at least 600 bp based on the nucleotide sequences. In some embodiments, the method can also be one in which the contiguous portion of at least 600 bp is distributed throughout 600 basepairs.

Further embodiments of the present screening methods are provided. In some embodiments, the screening methods of the present disclosure can be used for any protein variant screening in which (a) the protein sequence is intended to be varied in a consecutive area of 200 amino acids or more; (b) protein variants can be expressed in a reporter cell (including yeast and bacteria) or another functional carrier (e.g. viruses, phages) that can be exposed to a selective pressure; and (c) reporter cells expressing protein variants of interest can be selected on at least one functional property after selective pressure (e.g. antigen-binding, gene expression in response to antigen, etc.).

Generation of a Library of Variant Nucleotide Sequences Containing at Least Two Variant Nucleotide Subsequences that can Only be Unambiguously Identfied by Determining at Least 600 bp of their Total Nucleotide Sequence

In some embodiments, the library of variant nucleotide sequences is generated by any suitable in silica design and protein engineering approaches. Each variant can be encoded in a gene vector that will lead to expression of the variant nucleotide sequence within cells. Depending on the exact screening to be conducted, variant nucleotide sequences can be combined with appropriate promotor, signal peptide, splice donor/acceptor, stop codon and poly(A) signal sequences. The expression construct may also contain a selection marker, e.g., an antibiotic resistance gene, a fluorescent molecule or a cell surface marker.

In some embodiments, in order to enhance the ability to identify variants with high confidence, in silica algorithms can be utilized to control codon usage, thereby maximizing the edit distance (which may be the number of nucleotide changes involved to transform a given nucleotide sequence into any other variant nucleotide sequence in the library) between any two variants and enhancing the ability to distinguish variant nucleotide sequences. The variant sequence library can be generated by any suitable options, such as, but not limited to DNA synthesis.

Screening methods of the present disclosure in some embodiments can he used to screen for any suitable protein in which variants can only be confidently identified by determining more than 200 amino acids. Examples include, but are not limited to, TCRs, CARs, antibodies, RNA-directed nucleases, synthetic switch receptors and directed protein evolution.

In some embodiments, the library is a TCR library containing TCRα and TCRβ variants. The TCR library can be any suitable library in which any given TCRα and TCRβ chain may either occur with more than one complimentary dimerization partner or the dimerization partner is unknown. In both cases, the TCR variant can only be unambiguously identified by sequencing both TCRα and TCRβ variable sequences.

In some embodiments, the library is a CAR library containing CAR variants. The CAR library can be any suitable library in which more than 200 amino acids need to be sequenced to identify any given CAR variant with confidence. Examples include any CAR libraries with highly diverse antigen-binding domains or any other combinatorial construction using some or all of the CAR protein domains, e.g., a library in which variants of hinge domains, transmembrane domains and two signaling domains are combined to create a library with 500 or more variants.

In some embodiments, the library is an antibody library containing antibody variants. Variant nucleotide sequences can include nucleic acids encoding antibody heavy and light chains that are paired in one expression construct which can only be unambiguously identified by sequencing both heavy and light chain sequences. In some embodiments, libraries of antibody variants can be introduced into reporter cells (or another functional vehicle) and selected based on at least one functional property (such as antigen binding).

Introducing the Library of Variant Nucleotide Sequences into Reporter Cells

The library of variant nucleotide sequences can be introduced into reporter cells by any suitable approach. In some embodiments, retro- or lentiviral gene delivery is used to introduce the library into reporter cells (or, as noted herein any carrier). Any suitable approach may be used to introduce the library into reporter cells. The reporter cell may be any suitable cell (or carrier). In some embodiments, the reporter cell can be selected based on several criteria: (1) its ability to demonstrate a measurable gain- or loss-of-function in dependency to the introduced variant nucleotide sequence and in response to an external selection pressure; (2) minimal background expression of the marker molecule used to measure response to the selection pressure and (3) proliferation and cell survival of reporter cells in culture. Suitable reporter cells include, without limitation, immortalized Jurkat T cells or primary human Tcells for screening, e.g., immune receptor (TCR/CAR) libraries. Other carrier options are also noted herein.

In some embodiments, on average each reporter cell is transduced with only one variant nucleotide sequence, hence viral transductions are performed with a low MOI. In some embodiments, in order to maintain a level of representation of each variant nucleotide sequence in the polyclonal reporter cell pool (hereafter: coverage), the number of reporter cells to be transduced is directly related to the number of library variants.

In some embodiments, after modification with the library, successfully modified reporter cells may be selected, for example based on antibiotic resistance (e.g. Puromycin or Biasticidin). Such selection can reduce overall cell numbers by eliminating non-transduced cells from the population after low MOI transduction. In some embodiments, the strength of selection depends on the diversity of the variant sequence library. In some embodiments, the library is introduced into a larger population of reporter cells where the library has higher diversity to maintain library coverage within the population. In some embodiments, a sufficient number of polyclonal reporter cells are used to maintain a specified level of coverage after selection.

In some embodiments, the library can be introduced into reporter cells by transposon-mediated gene delivery. In some embodiments, the library can be introduced into reporter cells by DNA-nuclease mediated site-specific integration (e.g. using CRISPR/Cas9 and TALEN).

In some embodiments, modified reporter cells may be selected based on bead-based enrichment for a cell surface marker by the modified reporter cells. In some embodiments, modified reporter cells may be selected by flow cytometry sorting for a cell surface marker or a fluorescent molecule.

in some embodiments, reporter cells are genetically modified in order to (1) enhance their ability to demonstrate a gain- or loss-of-function in response to an external selection pressure; (2) reduce background expression of the marker molecule used to measure response to the selection pressure; and/or (3) enhance §the ability to maintain the reporter cell population in cell culture.

### Selection of Reporter Cells Based on at Least One Functional Property

In some embodiments, a selective pressure is applied towards the polyclonal population of reporter cells in order to measure a gain- or loss-of-function by the reporter cells depending on the expressed protein variant. For example, reporter cells can be stimulated with antigen-expressing cells. Subsequently, in response to the stimulus reporter cells can he isolated based on a suitable marker. For example, CD69 upregulation on TCR-transduced Jurkat cells in response to antigen-expressing cells can be used to perform flow cytometry sorting of reporter cells. Both responding and non-responding population can be isolated with sufficient coverage and analyzed separately. Thereby, relative fold enrichment of a given variant nucleotide sequence can be measured by determining enrichment in the positive and depletion in the negative population.

In some embodiments, selective pressure on reporter cells is applied in one or more of the following manners: stimulation with receptor agonists or antagonists; exposure to small molecules; exposure to more than one simultaneous stimulus. In some embodiments, reporter cells are isolated based on protein marker upregulation or downregulation, e.g. used for flow cytometry or bead-based sorting of marker-positive and negative reporter cells. In some embodiments, reporter cells are isolated based on drug resistance/sensitivity, which leads to selective survival or cell death of reporter cells. In some embodiments, reporter cells are isolated based on multiple marker molecules.

In some embodiments, only one population is isolated instead of isolating both positively and negatively responding reporter cells. The fold enrichment of a given variant nucleotide sequence can be established by comparison to polyclonal reporter cells that were not exposed to a selective pressure.

Any one or combination of the above-described approaches of selecting the reporter cells may be used.

Isolation of Variant Nucleotide Sequences from Selected Reporter Cells

In some embodiments, in order to analyze the isolated polyclonal reporter cell populations on a bulk level, genomic DNA (gDNA) is isolated using any suitable approaches. Subsequently, the variant nucleotide sequences are amplified by PCR. Such amplification may be performed for the complete variant nucleotide sequence or only the region in which the variants exhibit mutational diversity. PCR amplicons can be prepared for NGS-analysis on a platform that can provide sufficient read length and total number of sequencing reads, such as, but not limited to Oxford Nanopore technology. In some embodiments, the PCR protocol can be improved to avoid any biased amplification of defined variant nucleotide sequences, using any suitable options (e.g. use of Unique molecular identifiers (UMI) and minimal numbers of PCR cycles).

In some embodiments, as a means to prevent biased amplification of variant nucleotide sequences, gDNA from selected reporter cells is isolated and subjected to CRISPR-based selective library preparation. Genomic DNA can be dephosphorylated, and CRISPR/Cas9-mediated double-stranded breaks can be introduced in the sequences flanking the sequences of interest. The nucleotides directly adjacent to the double-stranded break can remain phosphorylated following this treatment, which can allow for phosphorylation-dependent adapter ligation in a subsequent library preparation step. Depending on the orientation of the Protospacer adjacent motif (PAM) and the protospacer sequence that is used, the insert can be specifically sequenced using a suitable sequencing options (e.g., Oxford Nanopore technology).

### Determine at Least 600 bp of the Total Nucleotide Sequence of the Isolated Variant Nucleotide Sequences

In some embodiments, PCR amplicons are sequenced utilizing a suitable NGS-platform. Any suitable sequencing platform may be used to sequence the amplicons, depending on the genetic variant library properties. Suitable sequencing platforms include, without limitation, Oxford Nanopore technology.

### Selection of at Least One Variant Nucleotide Sequence of Interest

In some embodiments, a variant nucleotide sequence of interest, e.g., TCRs and CARs, is selected based on relative enrichment by determining variant enrichment in the positively selected (marker molecule positive) reporter cell population and variant depletion in the negatively selected (marker molecule negative) reporter cell population as determined by variant read counts in both cell populations. In some embodiments, variant nucleotide sequence of interest is selected based on one or more of: relative enrichment; relative enrichment occurring under different selective pressures, e.g. different antigen doses; relative enrichment in multiple cell populations isolated based on different marker molecules; relative enrichment in cell populations that are isolated based on a combination of marker molecules; relative enrichment occurring in multiple cell populations, such cell populations being comprised of different types of reporter cells; enrichment of variant nucleotide sequences, e.g., TCRs or CARs, in an isolated population of reporter cells relative to the original gene variant library.

In some embodiments, any of the following arrangements or subparts thereof can be part of or combined with the embodiments provided herein. Arrangements are numbered 1-145 as follows:

1. A method to recover a repertoire of T cell receptors (TCRs) from diverse T cell populations, the method comprising:
determining TCR-α and β nucleotide or amino acid sequences within a subject's sample;
selecting one or more subsets of TCRα- and β-chain sequences from the total repertoire;
creating a TCR repertoire by combinatorial pairing of selected TCRα- and β-chain sequences creating a library of TCRαβ pairs; and
identifying at least one TCRαβ pair with desired features from the created TCR repertoire.

2. The method of arrangement 1, wherein the one or more subsets of TCR α- and β-chain sequences from the total repertoire is selected based on at least one criterion:
on frequency within the T cell population,
on relative enrichment compared to a second T cell population,
on relative difference of DNA and RNA copy numbers of a given TCR chain
on biological properties of the TCR chain, wherein the properties are selected from at least one of: (predicted) antigen-specificity, (predicted) HLA-restriction, antigen-affinity, co-receptor dependency, parental T cell lineage (e.g. CD4 or CD8 T cell) or TCR sequence motifs,
on spatial patterns of gene expression, wherein spatial gene expression patterns are derived from at least one of: originating region in the tissue or co-expression patterns of other genes,
on co-occurrence or occurrence at a similar frequency in multiple samples, for example occurrence in multiple tumor lesions,
assignment to multiple groups to separately recover specific parts of the TCR repertoire,
on a combination of multiple criteria as defined in the different embodiments.

3. The method of arrangement 2, wherein selection based on frequency within the T cell population is based upon data of the frequency of TCR sequences, which is used to create a separate rank order for TCRα- and β-chains or a combined rank order for TCRα- and β-chains.

4. The method of any one of arrangements 1-3, further comprising determining a frequency threshold that is defined based on the desired depth for TCR repertoire recovery and used to select collections of TCRα- and β-chains based on frequency.

5. The method of any one of arrangements 1-4, wherein determining TCR-α and β sequences is achieved by at least one of:
multiplex PCR;
TCR-sequence recovery by target enrichment;
TCR-sequence recovery by 5′RACE and PCR;
TCR-sequence recovery by spatial sequencing;
TCR-sequence recovery by RNA-seg;
and the use of a Unique Molecular Identifier (UMI)

6. The method of any one of arrangements 1-5, wherein a recovered TCR-chain sequence is defined as the CDR3 nucleotide sequence together with sufficient 5′- and 3′-nucleotide sequence information to select at least one TCR V- and one TCR J-segment family based on nucleotide sequence alignment to assemble a complete TCR chain sequence.

7. The method of any one of arrangements 1-6, wherein creating a TCR repertoire by combinatorial pairing of selected TCRα- and β-chain sequences creating a library of TCRαβ pairs is achieved by at least one of the following:
TCR chain sequences are used to synthesize separate libraries of TCRα- and β-chain DNA or RNA fragments which are subsequently linked into one DNA or RNA fragment in which exactly one TCRα- and one β-chain are linked,
combinations of TCRα- and β-chains are generated by directly synthesizing DNA or RNA fragments in which exactly one TCRα- and one β-chain are linked,
combinations of TCRα- and β-chains are created intracellularly by modification of a pool of cells with separate collections of TCRα- and β-genes in such a way that cells will express at least one TCRα- and one β-chain, and/or
combinations of TCRα- and β-chains are linked in a single-chain TCR construct in which both TCRα and TCRβ Variable chain fragments are fused and in which the single chain TCR construct may be fused to (i) a transmembrane domain alone or (ii) additionally contain intracellular signaling domains, including but not limited to CD3ϵ or CD3ζ signaling domains alone or in combination with a CD28 signaling domain.

8. The method of any one of arrangements 1-7, wherein identifying at least one TCRαβ pair with desired features from the created TCR repertoire is achieved by at least one of the following:
a pool of reporter cells modified with the library of generated TCRαβ pairs is stimulated by antigen presenting cells presenting at least one antigen of interest and antigen-reactive reporter cells are isolated based on at least one activation marker for TCR isolation;
a pool of reporter cells modified with the library of generated TCRαβ pairs is labelled with a fluorescent dye suitable to trace cell proliferation, stimulated by antigen presenting cells expressing at least one antigen of interest, and antigen-reactive reporter cells are isolated based on proliferation for TCR isolation;
a pool of reporter cells modified with the library of generated TCRαβ pairs is divided into at least two samples; samples are stimulated by antigen presenting cells expressing at least one antigen of interest or not; after stimulation, both reporter cell populations are incubated for a period of time and subsequently both reporter cell populations are analyzed by TCR isolation; comparison of TCRαβ pairs obtained from both samples is used to identify TCR genes with higher abundance in the sample exposed to at least one antigen;
a pool of reporter cells modified with the library of generated TCRαβ pairs is stimulated by antigen presenting cells presenting at least one antigen of interest and antigen-reactive reporter cells are isolated for TCR isolation based on at least one reporter gene, such as NFAT-GFP or NFAT-YFP that reports on TCR triggering;
a pool of reporter cells modified with the library of generated TCRαβ pairs is stimulated by antigen presenting cells presenting at least one antigen of interest, and antigen-reactive reporter cells are isolated for TCR isolation based on selection of antigen-specific reporter cells based on selective survival, including but not limited to acquired antibiotic resistance, upon TCR signaling, for example by use of an NFAT-puromycin transgene;
a pool of reporter cells modified with the library of generated TCRαβ pairs is exposed to one or multiple MHC complexes that carry an antigen of interest; reporter cells binding to an MHC complex are isolated for TCR isolation;
a pool of reporter cells modified with the library of generated TCRαβ pairs is stimulated by antigen presenting cells expressing at least one antigen of interest; subsequently, TCRαβ pairs of interest are identified using single-cell based droplet PCR or microfluidic approaches to combine TCR isolation with the detection of transcript levels for at least one activation marker; thereby, single reporter cells within the pool of T cells in which TCRαβ transcripts are co-expressed with increased levels of activation marker are detected.

9. The method of any one of arrangements 1-8, wherein the subject's sample comprises non-viable starting material.

10. The method of any one of arrangements 1-9, wherein a defined part of the identified TCR repertoire is recovered.

11. The method of any one of arrangements 1-10, wherein antigen-specific TCR sequences are recovered.

12. The method of any one of arrangements 1-11, wherein Class I and/or Class II restricted TCR sequences are recovered.

13. The method of any one of arrangements 1-12, wherein at least one of:
neo-antigen specific TCR sequences
virus-specific TCR sequences
shared tumor-antigen specific TCR sequences
self-antigen specific TCR sequences
are recovered.

14. The method of arrangement 12, further comprising the step of administering T cells expressing the neo-antigen specific TCR sequences as a cancer therapy.

15. The method of any one of arrangements 1-13, wherein the method is for a diagnostic.

16. The method of arrangement 15, wherein the diagnostic is to recover TCR repertoires from pathological sites of infection or autoimmunity.

17. The method of any one of arrangements 1-15, wherein the method is for the recovery of BCR/antibody repertoires.

18. The method of any one of arrangements 1-16, further comprising isolating nucleic acids from a subject that comprise the TCR-α and β nucleotide sequences.

19. The method of arrangement 8, wherein the activation marker is selected from the group consisting of: CD25, CD69, CD62L, CD137, IFN-γ, IL-2, TNF-α, GM-CSF, OX40.

20. The method of any one of arrangements 1-18, wherein DNA and RNA isolation is from a T cell population that is a mixture of different cell types or part of a tissue sample (such as blood or tumor tissue).

21. The method of any one of arrangements 1-19, wherein the subject's sample comprises cells isolated from a body fluid.

22. The method of arrangement 20, wherein the cells are tumor-specific T cells or tumor-infiltrating lymphocytes.

23. The method of arrangements 20-21, wherein the body fluid is selected from the group consisting of blood, urine, serum, serosal fluid, plasma, lymph, cerebrospinal fluid, saliva, sputum, mucosal secretion, vaginal fluid, ascites fluid, pleural fluid, pericardial fluid, peritoneal fluid, and abdominal fluid.

24. The method of arrangement 1, further comprising using the TCRαβ chain sequences to treat a subject suffering from cancer, an immunological disorder, an autoimmune disease, or an infectious disease.

25. The method of any one of arrangements 1-7, wherein identifying at least one TCRαβ pair with desired features from the created TCR repertoire is achieved by at least one of the following:
identification or selection based on at least one activation marker;
identification or selection based on proliferation in response to antigen;
identification or selection based on identification of TCR genes of higher abundance in antigen-stimulated cells as compared to unstimulated cells;
identification or selection based on reporter gene activation by TCR triggering;
identification or selection based on selective survival, including but not limited to acquired antibiotic-resistance upon TCR signaling;
identification or selection based on binding to one or more MIX complexes;
identification or selection using single-cell based droplet PCR or microfluidics;
or any combination thereof.

26. The method of arrangement 8, wherein TCR isolation is achieved by DNA or RNA isolation from bulk antigen-reactive T cells to generate TCRαβ specific PCR product which is analyzed by DNA-sequencing or RNA sequencing to determine TCRαβ gene sequences of antigen-reactive T cells or single-cell based droplet PCR or microfluidic approaches to analyze the TCRαβ gene sequences expressed in analyzed single T cells.

27. The method of arrangement 8, wherein the reporter cells are T cells.

28. The method of arrangement 24, wherein identification or selection using single-cell based droplet PCR or microfluidics further comprises determination of co-expression of activation-associated genes.

29. A method of creating multiple T cell libraries, the method comprising: recovering a repertoire of T cell receptors (TCRs) according to the method of arrangement 1; selection of TCRα- and β-chain sequences from the total repertoire into multiple groups to separately recover specific parts of the TCR repertoire, wherein multiple T cell libraries are created that are of smaller complexity or that recover specific parts of the TCR repertoire.

30. The method of arrangement 29, wherein selection of TCRα- and β-chain sequences is based on frequency range.

31. A method of identifying a nucleotide sequence from a combinatorial library of nucleic acids, comprising:
providing a combinatorial library comprising a plurality of variant nucleic acids, each of the plurality of variant nucleic acids comprising a contiguous portion of at least 600 bp, wherein the contiguous portion comprises a combination of two or more variant nucleotide subsequences, wherein a first variant nucleotide subsequence of the two or more variant nucleotide subsequences defines a first end of the contiguous portion and a second variant nucleotide subsequence of the two or more variant nucleotide subsequences defines a second end of the contiguous portion opposite the first end;
introducing the library into a population of cells configured to express one or more polypeptides encoded by a member of the plurality of variant nucleic acids;
selecting a subpopulation of the population of cells based on at least one functional property dependent on the combination of the two or more variant nucleotide subsequences, wherein the subpopulation comprises a plurality of cells;
isolating a subset of the plurality of variant nucleic acids from the subpopulation;
determining nucleotide sequences of the contiguous portion of individual members of the subset; and
identifying at least one combination of the two or more variant nucleotide subsequences based on the nucleotide sequences.

32. The method of arrangement 1, wherein the one or more polypeptides comprises:
T cell receptor α (TCRα)- and TCRβ-chains;
a chimeric antigen receptor (CAR);
a switch receptor; or
one or more chains of an antibody or antigen binding fragment thereof

33. The method of arrangement 31 or 32, wherein the first variant nucleotide subsequence encodes a TCRα variant amino acid sequence and the second variant nucleotide subsequence encodes a TCRβ variant amino acid sequence.

34. The method of any one of arrangements 31 to 33, wherein the two or more variant nucleotide subsequences encode one or more of: a TCR V region, a TCR complementarity determining region 3 (CDR3), a TCR J-segment, and a TCR constant region.

35. The method of arrangement 31 or 32, wherein each of the two or more variant nucleotide subsequences encodes an antigen binding domain, a hinge domain, a transmembrane domain, or one or more intracellular signaling domains of a CAR.

36. The method of any one of arrangements 31-35, wherein the contiguous portions is from 600 bp to 15,000 bp long.

37. The method of any one of arrangements 31-36 wherein providing comprises generating the library.

38. The method of arrangement 37, wherein generating the library comprises:
identifying two or more sets of variant nucleotide subsequences encoding two or more sets of variant amino acid sequences of the one or more polypeptides, wherein the at least one functional property depends on a combination of variant amino acid sequences from each of the two or more sets of variant amino acid sequences; and
assembling the contiguous portion by combining a variant nucleotide subsequence from each of the two or more sets of variant nucleotide subsequences to thereby generate the member of the plurality of variant nucleic acids.

39. The method of any one of arrangements 31-38, wherein an edit distance among contiguous portions of the plurality of variant nucleic acids in the library is maximized.

40. The method of any one of arrangements 31-39, wherein the library comprises at least 100 different combinations of the two or more variant nucleotide subsequences.

41. The method of any one of arrangements 31-40, wherein introducing comprises introducing the library via viral transduction, transposon-based gene delivery, or nuclease-mediated site-specific integration.

42. The method of arrangement 41, wherein introducing comprises virally transducing the population of cells at a multiplicity of infection (MOI) of 5 or less.

43. The method of any one of arrangements 31-42, comprising adjusting a size of the population of cells based on a number of different combinations of the two or more variant nucleotide subsequences in the library.

44. The method of any one of arrangements 31-43, wherein the population of cells comprises immortalized T cells or primary T cells.

45. The method of arrangement 44, wherein the immortalized T cells or primary T cells are human T cells.

46. The method of any one of arrangements 31-45, further comprising using a marker to select or screen for cells in the population of cells expressing at least one of the plurality of variant nucleic acids.

47. The method of arrangement 46, wherein the marker is a cytotoxin resistance marker and/or a cell surface marker.

48. The method of any one of arrangements 31-47, wherein cells of the population of cells are genetically modified.

49. The method of any one of arrangements 31-47, wherein cells of the population do not comprise an endogenous polypeptide conferring the at least one functional property to the cells.

50. The method of any one of arrangements 1 to 49, wherein the cells are genetically modified to eliminate or reduce expression of one or more of CD4, CD8 and CD28.

51. The method of arrangement 48 or 49, wherein the cells are reconstituted with CD4 and/or CD8 and utilized to screen for Class I and/or Class II restricted TCR sequences.

52. The method of arrangement 50 or 51, wherein the cells are T cells.

53. The method of any one of arrangements 31-52, wherein selecting comprises selecting the subpopulation based on expression of a detectable marker, wherein the expression depends on the at least one functional property of the one or more polypeptides.

54. The method of arrangement 53, wherein the detectable marker comprises a cell-surface marker, a cytokine marker, a cell proliferation marker, a transcription reporter, a signal transduction reporter, and/or a cytotoxicity reporter.

55. The method of arrangement 54, wherein
the cell-surface marker comprises one or more of: CD25, CD69, CD62L, CD137, OX40;
the cytokine marker comprises one or more of: IFN-γ, IL-2, TNF-α, IL-8, GM-CSF;
the transcription reporter comprises one or more of: NF-κB, NFAT, AP-1;
the signal transduction reporter comprises one or more of: ZAP70, ERK1/2; and
the cytotoxicity reporter comprises one or more of: CD107A, CD107B.

56. The method of any one of arrangements 31-55, wherein selecting comprises contacting the population of cells with one or more of:
a second population of cells;
a ligand for the one or more polypeptides;
an agonist or antagonist of the one or more polypeptides; and
a small molecule,
wherein a change in the subpopulation induced by the contacting depends on the at least one functional property of the one or more polypeptides.

57. The method of arrangement 56, wherein selecting further comprises:
detecting the presence or absence of the change, and/or a magnitude of the change; and
selecting the subpopulation based on the detecting.

58. The method of arrangement 56 or 57, wherein the second population of cells comprises antigen-presenting cells.

59. The method of arrangement 58, wherein the antigen-presenting cells comprise B-cells and/or dendritic cells.

60. The method of any one of arrangements 56-59, wherein the second population of cells comprises primary cells or immortalized cells.

61. The method of any one of arrangements 56-60, wherein variant nucleotide sequences of the library are derived from cells expressing a variant polypeptide comprising an amino acid encoded by the variant nucleotide subsequences, wherein the cells are obtained from a subject, and wherein the second population of cells is derived from the subject.

62. The method of any one of arrangements 31-61, wherein selecting comprises selecting a first subpopulation of the population of cells based on a measure of the at least one functional property above or below a threshold.

63. The method of arrangement 62, wherein the threshold is determined based on a measure of the functional property in an unselected subpopulation of the population of cells.

64. The method of arrangement 62, wherein selecting further comprises selecting a second subpopulation of the population of cells based on a second measure of the at least one functional property above or below a second threshold, wherein the first and second subpopulations are non-overlapping.

65. The method of arrangement 64, wherein identifying the at least one combination comprises comparing an abundance of the at least one combination between the first and second subpopulations.

66. The method of any one of arrangements 31-64, wherein identifying the at least one combination comprises measuring an enrichment of the at least one combination in the subpopulation relative to a control population of cells.

67. The method of arrangement 66, wherein the population of cells comprises the control population of cells

68. The method of arrangement 67, wherein the subpopulation and control population of cells are non-overlapping, wherein non-overlapping denotes that the cells in both populations have a different activation status, but can carry a same variant nucleic acid.

69. The method of arrangement 66, wherein the control population of cells are selected based on a second functional property that is different from the at least one functional property.

70. The method of any one of arrangements 31-69, wherein the subpopulation comprises a plurality of cells.

71. The method of any one of arrangements 31-70, wherein the isolating does not comprise isolating single clones of the subpopulation based on the at least one functional property.

72. The method of any one of arrangements 31-71, wherein the subpopulation comprises at least 1,000 cells.

73. The method of any one of arrangements 31-72, wherein determining comprises sequencing the individual members by generating sequencing reads of at least 600 bp of the contiguous portion.

74. The method of arrangement 73, wherein the sequencing reads are between 600 bp and 15,000 bp long.

The method of any one of arrangements 31-74, wherein determining comprises amplifying at least the contiguous portion of the individual members.

76. The method of arrangement 75, wherein amplifying comprises using an amplification primer that hybridizes to an invariant nucleotide subsequence, wherein each of the plurality of variant nucleic acids comprises the invariant nucleotide subsequence, and wherein the invariant nucleotide subsequence encodes an invariant amino acid sequence of the one or more polypeptides.

77. The method of arrangement 76, wherein the one or more polypeptides comprises TCRα- and TCRβ-chains, and wherein the invariant amino acid sequence comprises a TCRα constant region.

78. The method of arrangement 76, wherein the one or more polypeptides comprises TCRα- and TCRβ-chains, and wherein the invariant amino acid sequence comprises a TCRβ constant region.

79. The method of any one of arrangements 31-78, wherein the isolating comprises using CRISPR/Cas9-mediated targeted fragmentation of genomic DNA from the subpopulation.

80. The method of any one of arrangements 31-78, wherein the determining comprises obtaining an average coverage of at least 10 for each of the nucleotide sequences of the contiguous portion, before or in the absence of any amplification of the individual members.

81. The method of any one of arrangements 31-80, wherein the determining comprises obtaining an average coverage of at least 25, at least 50, at least 100, at least 200, at least 300, at least 400, at least 500 or at least 1,000 for each of the nucleotide sequences of the contiguous portion.

82. A method of identifying nucleotide sequences encoding T cell receptor a (TCRα)- and TCRβ-chains from a combinatorial library of nucleic acids, comprising:
providing a library comprising a plurality of variant nucleic acids, each of the plurality of variant nucleic acids comprising a contiguous portion of at least 600 bp, wherein the contiguous portion comprises:
   a combination of
      a first variant nucleotide subsequence encoding a TCRα variant amino acid sequence and defining a first end of the contiguous portion,
   and
      a second variant nucleotide subsequence encoding a TCRβ variant amino acid sequence and defining a second end of the contiguous portion opposite the first end;
introducing the library into a population of immortalized T cells configured to express TCRα- and TCRβ-chains encoded by a member of the plurality of variant nucleic acids;
selecting a subpopulation of the population of immortalized T cells based on an expression of a T cell activation marker above a threshold level in response to contacting the immortalized T cells with immortalized B cells expressing an antigen, wherein the subpopulation comprises a plurality of T cells;
isolating a subset of the plurality of variant nucleic acids from the subpopulation;
determining nucleotide sequences of the contiguous portion of individual members of the subset; and
identifying at least one combination of the first and second variant nucleotide subsequences based on an enrichment of the at least one combination in the nucleotide sequences of the subset relative to a control.

83. The method of arrangement 82, further comprising:
selecting a second subpopulation of the population of immortalized T cells based on the expression of the T cell activation marker below a second threshold level in response to contacting the immortalized T cells with the immortalized B cells, wherein the second subpopulation comprises a second plurality of T cells, and wherein the subpopulation and second subpopulation are non-overlapping;
isolating a second subset of the plurality of variant nucleic acids from the second subpopulation; and
determining second nucleotide sequences of the contiguous portion of individual members of the second subset,
wherein the at least one combination is identified based on an enrichment of the at least one combination in the subset relative to the at least one combination in the second nucleotide sequences of the second subset.

84. A method of identifying a nucleotide sequence encoding a chimeric antigen receptor (CAR) hinge domain, transmembrane domain, and/or an intracellular signaling domain from a combinatorial library of nucleic acids, comprising:
providing a library comprising a plurality of variant nucleic acids, each of the plurality of variant nucleic acids comprising a contiguous portion of at least 600 bp, wherein the contiguous portion comprises a combination of two or more of:
   a first variant nucleotide subsequence encoding a CAR hinge domain;
   a second variant nucleotide subsequence encoding a CAR transmembrane domain; and
   a third variant nucleotide subsequence encoding a CAR intracellular signaling domain,
   wherein one of the first, second or third variant nucleotide subsequences define a first end of the contiguous portion, and wherein another one of the first, second or third variant nucleotide subsequences defines a second end of the contiguous portion opposite the first end;
introducing the library into a population of cells configured to express a CAR encoded by a member of the plurality of variant nucleic acids, wherein the population of cells comprises a population of immortalized T cells or primary human T cells;
selecting a subpopulation of the population of cells based on cell proliferation. above a threshold level in response to contacting the cells with antigen-presenting cells expressing an antigen specific to an antigen-binding domain of the CAR, wherein the subpopulation comprises a plurality of cells;
isolating a subset of the plurality of variant nucleic acids from the subpopulation;
determining nucleotide sequences of the contiguous portion of individual members of the subset; and
identifying at least one combination of the first, second, and third variant nucleotide subsequences based on an enrichment of the at least one combination in the nucleotide sequences of the subset relative to a control.

85. The method of arrangement 84, wherein there is more than one CAR intracellular signaling domain.

86. The method of arrangement 85, wherein there are at east two CAR intracellular signaling domains.

87. The method of arrangement 84, wherein at least two of the following are the at least two CAR intracellular signaling domains: CD3ϵ, CD3ζ ITAM1, CD3ζ ITAM12, CD3ζ ITAM123, CD3ζ with any ITAM of CD3δ, CD3ϵ and CD3γ, CD8α, CD28, ICOS, 4-1BB (CD137), OX40 (CD134), CD27, and CD2

88. The method of arrangement 84, comprising:
selecting a second subpopulation of the population of cells based on cell proliferation below a second threshold level in response to contacting the cells with the antigen-presenting cells, wherein the second subpopulation comprises a second plurality of cells, and wherein the subpopulation and second subpopulation are non-overlapping;
isolating a second subset of the plurality of variant nucleic acids from the second subpopulation;
determining second nucleotide sequences of the contiguous portion of individual members of the second subset, and
wherein the at least one combination is identified based on an enrichment of the at least one combination in the subset relative to the at least one combination in the second nucleotide sequences of the second subset.

89. A method of identifying a nucleotide sequence from a combinatorial library of nucleic acids, comprising:
providing a combinatorial library comprising a plurality of variant nucleic acids, each of the plurality of variant nucleic acids comprises a contiguous portion of at least 600 bp;
introducing the library into a population of cells configured to express one or more polypeptides encoded by a member of the plurality of variant nucleic acids;
selecting a subpopulation of the population of cells based on at least one functional property dependent on the contiguous portion of at least 600 bp, wherein the subpopulation comprises a plurality of cells;
isolating a subset of the plurality of variant nucleic acids from the subpopulation;
determining nucleotide sequences of the contiguous portion of individual members of the subset; and
identifying the contiguous portion of at least 600 bp based on the nucleotide sequences.

90. The method of arrangement 89, wherein variability in the contiguous portion of at least 600 bp is distributed throughout 600 basepairs.

91. A method of identifying nucleotide sequences encoding antigen-specific T cell receptor α (TCRα)- and TCRβ-chain pairs from a library of nucleic acids, comprising:
introducing a library into a population of cells able to express TCRα- and TCRβ-chains encoded by a member of a plurality of variant nucleic acids,
selecting a subpopulation of the population of cells based on an expression of a marker above a threshold level in response to antigen, wherein the subpopulation comprises a plurality of cells,
isolating a subset of the plurality of variant nucleic acids from the subpopulation,
determining nucleotide sequences of the variant nucleic acids, and.
identifying at least one variant nucleotide sequence based on an enrichment of the nucleotide sequences within the subset relative to a control.

92. The method of arrangement 90, further comprising providing the library comprising the plurality of variant nucleic acids encoding TCR alpha and TCR beta chains.

93. A method of identifying nucleotide sequences encoding T cell receptor α (TCRα)- and TCRβ-chains from a sample, comprising:
sequencing TCR-αand β chains in a sample,
selecting and combinatorial pairing TCRα- and β-chain sequences to create a library of TCRαβ pairs,
introducing the library of TCRαβ pairs into a pool of reporter cells,
stimulating the reporter cells that are modified with the library of TCRαβ pairs with antigen presenting cells presenting at least one antigen of interest,
determining TCRαβ pairs specific to the at least one antigen of interest, and
introducing the TCRαβ pairs into cells and selecting cells containing the TCRαβ pairs.

94. The method of arrangement 14, further comprising a step of administering T cells expressing the antigen specific TCR sequences to diagnose or treat an infection or autoimmunity.

95. The method of arrangement 14, wherein the T cells can be autologous or allogeneic.

96. The method of arrangement 8, wherein the activation marker is CD69, and wherein two cell populations are isolated, one cell population with high expression of CD69 and the other cell population with low expression of CD69.

97. A nucleotide library comprising the repertoire of T cell receptors recovered according to any one of arrangements 1-30 and 95, 96.

98. A nucleotide construct comprising the nucleotide sequence identified according to any one of arrangements 1-96.

99. A cell comprising the nucleotide construct according to arrangement 98.

100. A method of identifying a nucleotide sequence encoding an antigen-specific T cell receptor α (TCRα)- and TCRβ-chain pair from a library of nucleic acids, the method comprising:
introducing the nucleic acid library into a population of cells able to express TCRα- and TCRβ-chains to make a library of cells;
selecting a first population of the library of cells based on an expression of a marker above a first threshold level in response to an antigen; and
isolating a first population of variant nucleic acids from the first population of the library.

101. The method of arrangement 100, further comprising:
determining at least one nucleotide sequences or nucleic acid identity of the first population of variant nucleic acids; and
identifying at least one variant nucleotide sequence based on an enrichment of the nucleotide sequences within the subset relative to a control.

102. The method of arrangement 100 or 101, wherein the threshold level is based on at least one of:
a) recovery of a percentage of the total pool of cells based on expression of a marker; or
b) recovery of a minimal number of cells from the total pool of cells; or
c) recovery of cells retained by a magnet based on binding of a magnetic probe to at least one marker expressed in response to an antigen

103. The method of arrangement 66-69, 82, 84, 91, or 101, wherein the control is a second population of cells that is below a second threshold.

104. The method of arrangement 66-69, 82, 84, 91, or 101, wherein the control is one or more of:
a reference population of cells,
the combinatorial library of nucleic acids that was introduced into the population of cells
a population of cells sorted from a same population of cells as the first population based on an expression marker below a second threshold,
at least one population of cells obtained from cocultures of reporter T cells expressing the relevant TCR library with antigen presenting cells such as B cells that are not presenting any exogenous antigens,

105. The method of arrangements 104, wherein the control (or bottom sample) is sorted from a same population of cells as the top sample, but having low activation marker expression or wherein the bottom sample is obtained from cocultures of reporter T cells expressing the relevant TCR library, and B cells that are not engineered to express exogenous antigens.

106. The method of any one of arrangements 100-105, further comprising adding an antigen to the population of cells.

107. The method of any one of arrangements 100-106, wherein the isolating a first population and/or the control is achieved by at least one of a) magnetic bead enrichment, b) flow cytometry sorting, or c) both.

108. A method of identifying a nucleotide sequence encoding a T cell receptor α (TCRα)- and TCRβ-chain from a library of nucleic acids, the method comprising:
introducing the nucleic acid library into a population of cells able to express TCRα- and TCRβ-chains to make a library of cells; and
determining at least one nucleotide sequence or nucleic acid identity of the first population of variant nucleic acids based on an enrichment of the nucleotide sequence within the subset relative to a control.

109. The method of arrangement 108, wherein the at least one nucleic acid is isolated from a first population of cells.

110. The method of arrangement 109, wherein the first population of cells is selected based on an expression of a marker above a first threshold level in response to an antigen.

111. A method of identifying a nucleotide sequence from a library of nucleic acids, comprising:
introducing the library of nucleic acids into a population of cells to form a library of cells;
contacting the library of cells with a first population of cells;
selecting a sub-population of the library of cells based on expression of at least one marker by magnetic bead enrichment; and
identifying at least one nucleotide sequence based on a statistically significant enrichment or depletion of the nucleotide sequences within the sub-population relative to a control.

112. The method of arrangement 111, wherein at least some of the sub-population of cells are configured to express one or more polypeptides encoded by a member of the library of nucleic acids.

113. The method of arrangement 111, wherein marker expression is linked to an introduced nucleic acid from the library, wherein linked denotes that the introduced nucleic acid alters marker expression.

114. The method of arrangement 111, wherein selecting is based upon marker expression above a threshold level.

115. The method of arrangement 111, wherein selecting is based upon an expression of at least two markers above a threshold level.

116. The method of any one of arrangements 8, 82, 91, 93 or 100, wherein identifying or stimulating or providing antigen comprises one or more of:
a) selecting a number of antigens;
b) creating antigen-pools in which each antigen is present in exactly two antigen pools
c) evaluating reactivity of reporter cells expressing at least one T cell receptor against each of the antigen pools; and
d) determine whether the at least one T cell receptor is reactive towards any of the selected antigens by evaluating for reactivity against exactly two antigen pools

117. The method of arrangement 116, wherein reactivity against exactly two antigen pools is detected by pairwise enrichment analysis.

118. The method of arrangement 111, wherein the library is a TCR library.

119. The method of arrangement 111, wherein one employs an activation marker.

120. The method of arrangement 111, wherein one employs a top-bottom comparison to evaluate reactivity.

121. The method of any one of the preceding arrangements involving an evaluation of reactivity or further comprising an evaluation of reactivity, wherein one employs a top-bottom comparison to evaluate reactivity.

122. The method of any one of the preceding arrangements involving a library, wherein the nucleotide sequence of the plurality of variant nucleic acids in the library is optimized based at least one of the following:
introduction of preferable codon usage for the host cell,
optimization of mRNA structural stability,
avoidance of repetitive sequences,
avoidance of long stretches of homopolymers, and
avoidance of large differences in local GC-content within a given variant nucleic acid sequence

123. The method of arrangement 91, wherein the antigen is presented via an antigen-presenting cell.

124. The method of arrangement 91, wherein the library is a combinatorial library.

125. The method of any one of the preceding arrangements, wherein the antigen is provided by a cell.

126. The method of any one of the preceding arrangements, wherein the process involves a high degree of antigen diversity and/or complexity.

127. The method of any of the preceding methods involving a library, wherein the library is a combinatorial library.

128. The method above, wherein the combinatorial library is a TCR library.

129. A collection of cells, the collection comprising:
a set of at least two T cells, wherein each is configured to express at least one TCR alpha and TCR beta pair, wherein the TCR alpha and the TCR beta are each from a subject, wherein the T cells do not express an endogenous TCR, and wherein the set are configured for activation of one or more T cell activation markers; and
a set of at least two B cells, wherein each of the at least two B cells is configured to express at least one exogenous neo-antigen (or antigen), such that there are at least two exogenous neo-antigens (or antigens) capable of being produced, and wherein the at least two exogenous neo-antigens (or antigens) are the same as those in the subject.

130. The composition of arrangement 129, wherein the set of at least two B cells comprises:
at least a first B cell that produces the exogenous neo-antigen (or antigen); and
at least a second B cell that produces the a second exogenous neo-antigen (or antigen).

131. A library of TCR expressing cells, the library of TCR expressing cells comprising: a set of at least three T cells,
wherein at least two of the T cells are configured to express at least two TCR alpha and TCR beta pairs (at least two TCR pairs),
wherein the at least two TCR pairs are from a subject,
wherein the at least three T cells do not express an endogenous TCR,
wherein the at least three T cells are configured for activation of one or more T cell activation markers, upon binding to an antigen (or neo-antigen), presented by a B cell,
wherein an amount of genomic copies of each TCR pair as reflected in a number of TCR cells is such that one gets a read on every TCR in the sample, and
wherein at least one of the TCRs is not distributed equally throughout a composition comprising the library.

132. The library of TCR expressing cells of arrangement 131, wherein a distribution of at least one T cells is altered by binding to an antigen presented by a B cell.

133. The library of TCR expressing cells of arrangement 131, wherein the at least two TCR pairs are approximately evenly present in the library of TCR expressing cells.

134. A method of treating a subject, the method comprising:
identifying a subject having a tumor;
providing a set of at least two T cells, each of which is configured to express at least one different TCR alpha and TCR beta pair, wherein each of the TCR alpha and the TCR beta are from the subject,
providing a set of at least two B cells, wherein the set of B cells is configured to express at least two exogenous neo-antigens, and wherein the at least two exogenous neoantigens are the same as those neo-antigens found in the subject;
combining the set of at least two T cells with the set of at least two B cells and selecting a combination of at least two TCR pairs based upon activation of the at least two T cells via the at least two exogenous neo-antigens; and
administering the combination of at least two TCR pairs to the subject, thereby treating the tumor.

135. The method of arrangement 134, wherein treating reduces a size of the tumor.

136. A method of treating a subject, the method comprising:
identifying a subject having a tumor;
providing a set of at least two T cells, each of which is configured to express at least one different TCR alpha and TCR beta pair, wherein each of the TCR alpha and the TCR beta are from the subject,
providing a set of at least two antigen presenting cells, wherein the set of antigen-presenting cells originates from the subject, is configured to express at least two exogenous neo-antigens, and wherein the at least two exogenous neoantigens are the same as those neo-antigens found in the subject;
combining the set of at least two T cells with the set of at least two antigen present cells and selecting a combination of at least two TCR pairs based upon activation of the at least two T cells via the at least two exogenous neo-antigens; and
administering the combination of at least two TCR pairs to the subject, thereby treating the tumor.

137 A pharmaceutical composition comprising:
a first TCR pair, that binds to a first antigen (or neo-antigen) in a subject's tumor; and
a second TCR pair, that hinds to a second antigen (or neo-antigen) in the subject's tumor.

138. The pharmaceutical composition of arrangement 137, wherein the first TCR pair is MHC-class I restricted and wherein the second TCR pair is MHC-class II restricted.

139. A pharmaceutical composition comprising:
a first TCR pair, that binds to a first antigen and is MHC-class I restricted; and
a second TCR pair, that binds to a second antigen and is MHC-class II restricted.

140. The pharmaceutical composition of any one of arrangements 137-139, further comprising a third TCR pair.

141. The pharmaceutical composition of any one of arrangements 137-140, wherein the first TCR pair binds to a neo-antigen from a tumor, wherein the second TCR pair binds to a neo-antigen from the tumor, and wherein both the first and second TCR pairs are present in a host of the tumor.

142. A collection of cells, the collection comprising:
a set of at least two I cells, wherein each is configured to express at least one TCR alpha and TCR beta pair, wherein the pair is from a subject, wherein the T cells do not express an endogenous TCR, and wherein the set are configured for activation of one or more T cell activation markers; and
a set of at least two antigen present cells (APCs), wherein each of the at least two APCs is configured to express at least one exogenous neo-antigen (or antigen), such that there are at least two exogenous neo-antigens (or antigens) capable of being produced, and wherein the at least two exogenous neo-antigens (or antigens) are the same as those in the subject.

143. The method of any one of the methods above, wherein, the TCR pairs and/or the T cells expressing the TCR pairs are selected or identified by binding to an antigen (such as a neoantigen), wherein the antigen is expressed by a B cell or an antigen presenting cell.

144. The method of any one of the methods above, wherein the antigen or neoantigen is from a tumor in a subject, and wherein a TCR alpha and a TCR beta of the TCR pairs are also each from the subject.

145. The method of any one of the methods above, wherein there are at least 2, 3, 4, 5, 6, 7 8, 9, 10, 50, 100, 500, 1000, 10000, 100000, or 1 million TCR pairs (or cells comprising these pairs) and there are at least 2, 3, 4, 5, 6, 7, 8, 9, 10, 50, 100, 500, 1000, 10000, 100000, or 1 million antigens present.

### Additional Embodiments

Various embodiments of various methods are also presented in FIGS. 10A-10J. In some embodiments, the panels in FIGS. 10A-10J show a method of, and that it is possible to, isolate neoantigen-reactive TCRs from mismatch-proficient colorectal cancer (MMRp-CRC) tumors as exemplary of other tumors. These tumors generally have a low mutational burden. There is ample evidence to suggest that mutational burden is well correlated with response to immuno-oncology therapies, such as anti-PD1/PD-L1 checkpoint inhibition therapies. Indeed, poor response rates are observed in MMRp-CRC cohorts treated with immune checkpoint inhibitors.

Similar to immune checkpoint inhibitor therapies, T cell therapies are driven by the recognition of tumor cells by the immune system. Given the low mutational burden in MMRp-CRC, one would expect to find limited reactivity of T cells against tumor neoantigens. Unexpectedly, it was found (as outlined below and demonstrated in Example 13 below) TCRs that recognize tumor neoantigens for all four MMRp-CRC patient samples that were screened, thereby enabling the use of TCR T cell therapies for this patient group (which otherwise would have limited therapeutic options).

In some embodiments, the method can involve one or more of the steps outlines as process 10A-10J below (and accompanying figures of some of said embodiments).

Any of the steps can be repeated or substituted by other embodiments provided herein, as appropriate. Additional intervening steps can also be added.

Step 10A) Schematic of the screening process. In some embodiments, mismatch-proficient colorectal cancer (MMRp-CRC, for example) patient samples are subjected to the TCR identification platform, starting with obtaining genetic information from routine non-viable tumor biopsies. Bulk TCR sequencing information was retrieved from tumor-infiltrating lymphocytes (TIL) and used to assembled a combinatorially paired library of alpha and beta chain expression cassettes. These were expressed in Jurkat reporter T cells, and screened against autologous B cells expressing tumor neo-antigens as minigenes in a tandem minigene (TMG) format. By retrieving activated reporter T cells and isolation of their TCRs, neo-antigen-reactive TCRs can be identified.

Step 10B) Bulk TCR sequencing of infiltrating lymphocytes in a human MMRp-CRC sample. In some embodiments, the product from 10A can be subject to bulk sequencing of the TCRs. In sonic embodiments, human MMRp-CRC tumor sample pt1 was subjected to bulk TCR sequencing by Milaboratory. After alignment and TCR identification, clonotypes were collapsed based on their CDR3 amino acid sequence and their. V and J identity. The number of unique clonotypes are represented for both alpha and beta chains. The results are shown in the graph in FIG. 10B.

Step 10C) Quality control of TCR library. In some embodiments, an optional quality control check can be employed. In some embodiments, the 100 most prevalent alpha and beta chains from the tumor sample in 10A) were selected and used for creating a combinatorial library. The library was assembled by Twist Bioscience using human V, CDR3 and J segments, while the constant (C) region was of murine origin. A primer pair flanking the variable TCR alpha and beta chain domains was used to amplify both chains, and Nanopore sequencing was used to unveil the identity of both chains. The representation of each of the 10,000 alpha x beta combinations is represented. The results are shown in the graph in FIG. 10C10C, where the probability density is represented on the y-axis.

Step 10D) Library expression in Jurkat reporter T cells. In some embodiments, the library from above can be expressed in a reporter T cell, such as Jurkat. The library from 10C) was transfected into Phoenix cells for virus production, and the resulting viral supernatant was used for retroviral transduction of CD8+ TCR-KO Jurkat reporter cells. Cells were selected using blasticidin, and positivity for TCR expression was tested using an antibody directed against the murine TCR-beta constant region. The results are shown in the graph in FIG. 10D.

Step 10E) Sorting strategy for the screen. In some embodiments, the library can be sorted by any method. The Jurkat reporter. T cells from 10D) were co-cultured for 21 hours at a 1:1 ratio with B cells expressing the pt1 mutanome in the form of multiple tandem minigenes (TMGs). Cells were then sorted for T cell activation by FACS using the CD69 marker. The sorting strategy included (from left to right) sequential gating to select lymphocytes, gating to select singlet cells, gating to exclude CD20⁺-cells, and two sorting gates (‘top’ and ‘bottom’) which capture cells expressing high and low CD69, respectively. The results are shown in the graph in FIG. 10E.

Step 10F) Retrieval of TCR expression cassettes. In some embodiments, one can retrieve the relevant TCR expression cassettes by any of a variety of techniques. In some embodiments, TCR expression cassettes of top and bottom samples from 10E) (three replicates, and including three control screens on a coculture of Jurkat reporter T cells with B cells that do not express TMGs) were retrieved using the PCR strategy described in 10C), followed by a barcoding PCR. A control PCR on the plasmid TCR library was included as well. PCR products were analysed using an Agilent TapeStation (left). PCR products were pooled in a 1:1 ratio and analysed on the TapeStation (right). The results are shown in FIG. 10F.

Step 10G) Screen analysis. In some embodiments, the PCR product pool from step F can be analysed in any number of ways. For example, the PCR product pool from 10F) was used for library preparation and was sequenced. TCR alpha and beta chain identities were recovered and differentially expressed TCR combinations were identified using the DESeq2 R package. Average Rlog-transformed read counts for screens in the presence (x-axis) and absence (y-axis) of TMG expression by I3 cells are represented for the pt1 tumor sample described in 10B)-10F), as well as for three additional MMRp-CRC samples (pt2, pt3 and pt4) processed in an identical manner. Neo-antigen reactive TCR leads are depicted as encircled larger black dots in FIG. 10G.

Step 10H) Deconvolution of relevant TMGs. In some embodiments, the relevant TMGs can be deconvoluted in any number of ways. For example, the neo-antigen reactive TCRs identified in 10G) were re-screened (single replicate) using B cells expressing a single TMG construct. As an example, the demultiplexing screens for the pt1 sample are represented. The pt1 TCR lead recognizes pt1-TMG1 and not pt1-TMG2. The results are shown in the plot in FIG. 10H.

Step 10I) Identification of the pt1 TCR Lead antigen. The TCR lead antigen (e.g., pt1) can be identified in any number of ways. For example, the neo-antigen recognized by the pt1 TCR lead was identified by loading B cells with peptides of the single minigenes represented in pt1-TMG1. As a positive control, pt1-TMG1 expressing B cells were used. APCs were cocultures with Jurkat reporter T cells, which express the pt1 neo-antigen reactive TCR lead identified in 10G). Activation was measured as CD69-positivity relative to a positive control ( ).treatment with PMA/Ionomycin). Activation by a control (PARVA-P70R), as well as a AKAP8L-R191W peptide, are shown in FIG, 10I.

Step 10J) Identification of the pt3 TCR lead antigen. The TCR lead antigen (e.g., pt3) can be identified in any number of ways. For example, the neo-antigen recognized by the pt3 TCR lead was identified by loading B cells with peptides of the single minigenes represented in pt3-TMG1. As a positive control, pt3-TMG1 expressing B cells were used. APCs were cocultures with Jurkat reporter T cells, which express the pt3 neo-antigen reactive TCR lead identified in 10G). Activation was measured as CD69-positivity relative to a positive control (treatment with PMA!Ionomycin). Activation by a control (ETS1-R70W), as well as a TP53-R282W peptide, are shown in FIG. 10J.

Step 10K) Identification of the first pt4 TCR lead antigen. The first TCR lead antigen (e.g., pt4) can be identified in any number of ways. For example, the neo-antigen recognized by the first pt4 TCR lead was identified by expression of a minigene (MG91 encoding HSPA9-p.K654RfsX42) in B cells. As a positive control, pt4-TMG3 expressing B cells were used. APCs were cocultured with Jurkat reporter T cells, which express the first pt4 neo-antigen reactive TCR lead identified in 10G). Activation was measured as CD69-positivity relative to a positive control (treatment with PMA/Ionomycin). Activation by a control (B cells that do not express a MG or TMG), as well as by the MG91/TMG3 samples, are shown in FIG. 10K.

Step 10L) Identification of the second pt4 TCR lead antigen. The second TCR lead antigen (e.g., pt4) can be identified in any number of ways. For example, the neo-antigen recognized by the second pt4 TCR lead was identified by expression of a minigene (MG132 encoding ITPR3-p.L2379M) in B cells. As a positive control, pt4-TMG4 expressing B cells were used. APCs were cocultured with Jurkat reporter T cells, which express the second pt4 neo-antigen reactive TCR lead identified in 10G). Activation was measured as CD69-positivity relative to a positive control (treatment with PMA/Ionomycin). Activation by a control (B cells that do not express a MG or TMG), as well as by the MG132/TMG4 samples, are shown in FIG. 10L.

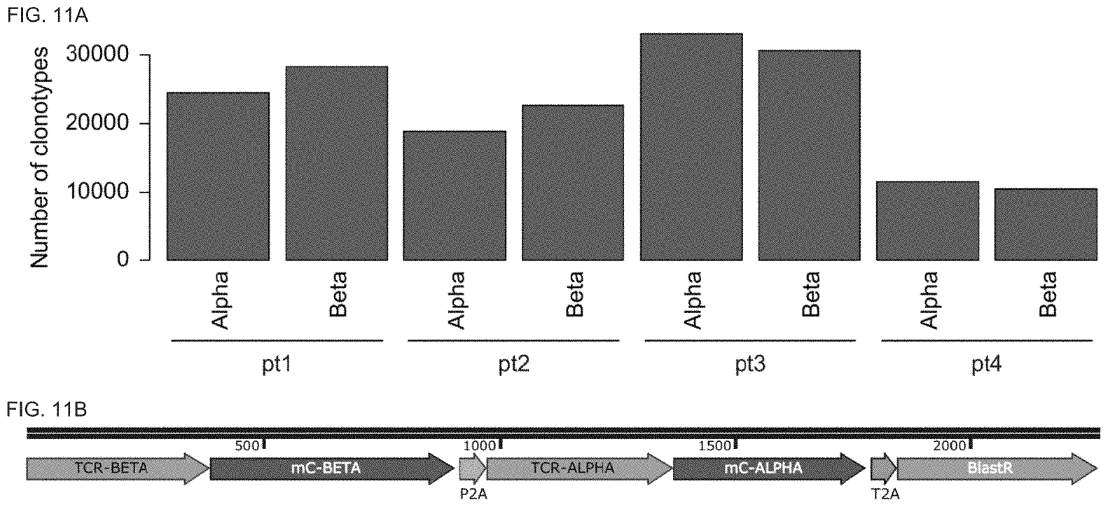

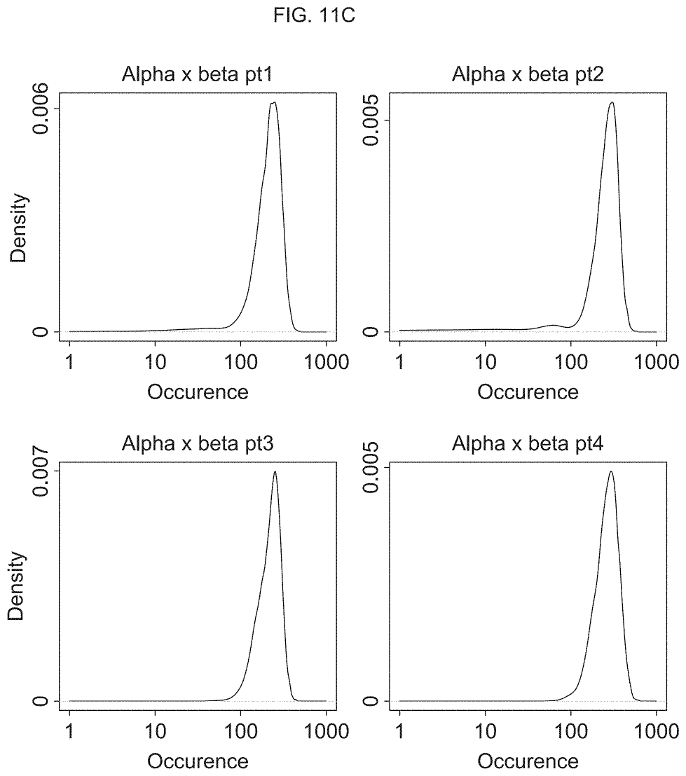

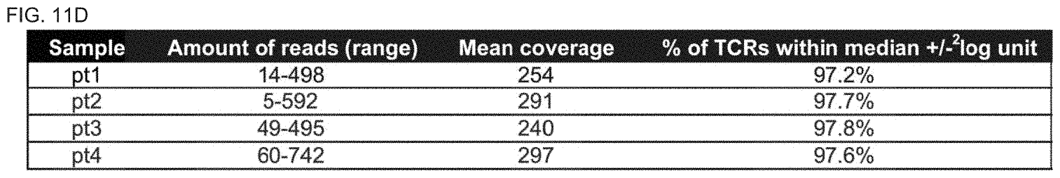

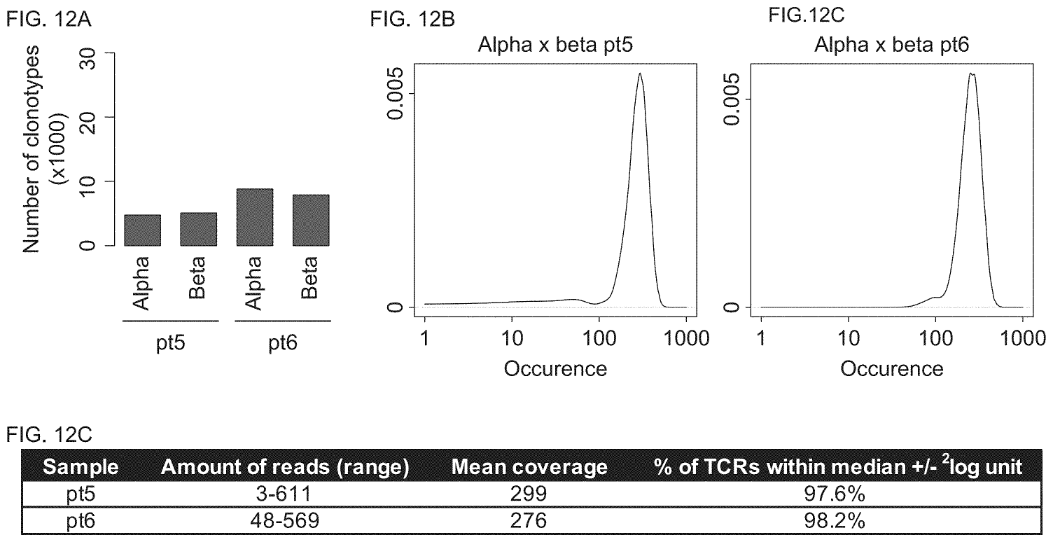

Some additional embodiments are depicted in FIGS. 12A-12C: Recovery of TCR repertoires from melanoma for the generation of TCRαβ libraries. FIG. 12A depicts bulk TCR sequencing of infiltrating lymphocytes in human melanoma samples. Two human tumor samples (pt5 and pt6) were subjected to bulk TCR sequencing by Milaboratory (Moscow/Russia). After alignment and. TCR identification, clonotypes were collapsed based on their CDR3 amino acid sequence and their V and J identity. The number of unique clonotypes are represented for both alpha and beta chains for each tumor sample. In FIG. 12B quality control of 100×100 libraries is depicted. The 100 most prevalent alpha and beta chains for each sample from A) were selected and used for creating a combinatorial library. The TCR-beta and TCR-alpha, regions in B) were synthesized and inserted into the retroviral construct represented in FIG. 11B. In a combinatorial cloning approach executed by Twist Bioscience. A primer pair flanking the variable TCR alpha and beta chain domains was used to amplify both chains, and Nanopore sequencing was used to identify the identity of both chains. The representation of each of the 10,000 alpha x beta combinations is represented for every patient library. In FIG. 12C) characteristics of the TCR representations of the patient libraries are provided. For each patient library in 12B) the range of the amount of reads per TCR, the mean coverage, and the percentage of TCRs that fall within a range of the median +/−a ²log-unit are represented.

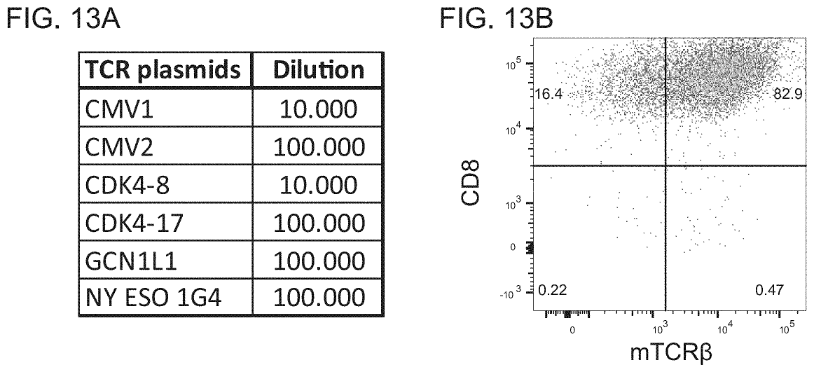

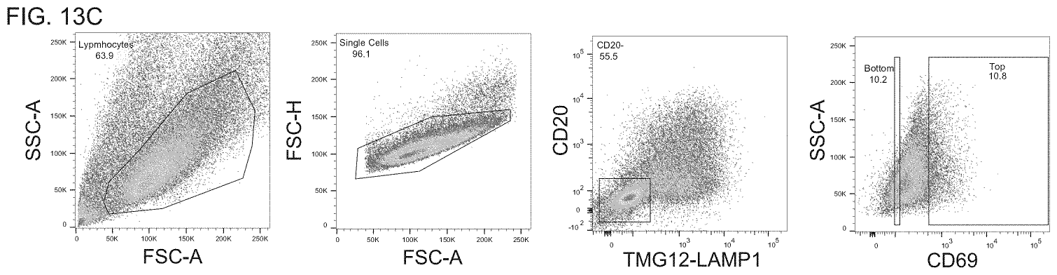

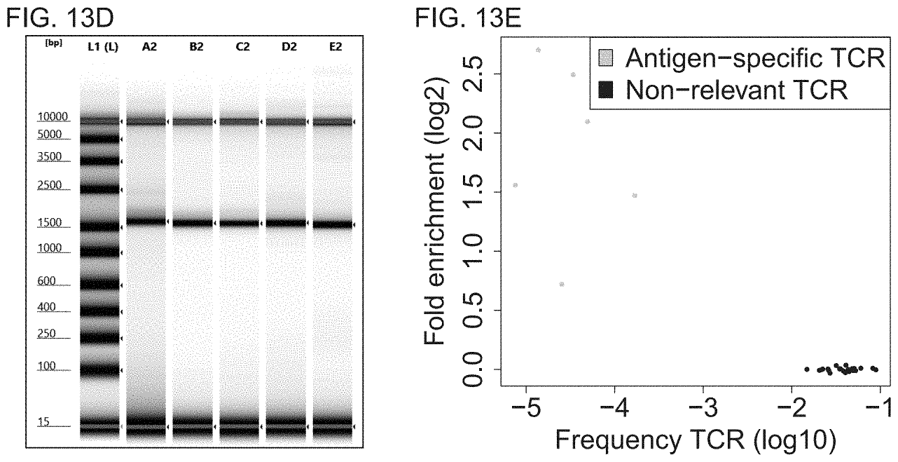

Sonic additional embodiments are provided in FIGS. 13A-13E (as steps for some embodiments. FIG. 13(A-E) shows the recovery of antigen-specific TCRs from a TCR library generated by artificial mixing of plasmids. In Step 13A) Library generation by artifical mixing of TCR plasmids. A TCR library was generated by artifical mixing of plasmids. Six plasmids each expressing a single characterized TCR were mixed into a pool of 11 plasmids each expressing a single uncharacterized ovarian cancer (OW) TCR and 13 plasmids each expressing a single uncharacterized colorectal cancer (CRC) TCR. Of the characterized TCRs, CMV1 and CDK4-8 TCR plasmids were mixed into this pool at 1:10.000 molar ratio and CMV2, CDK4-17, GCN1L1 and NY-ESO 1G4 TCR plasmids were mixed into this pool at 1:100.000 molar ratio. The frequencies are shown in FIG. 13A. In Step 13B) Library expression in Jurkat reporter T cells. In some embodiments, the mix of TCRs from step 13A) can be expressed in a reporter T cell, such as Jurkat. The pool of TCR plasmids from step 13A) was transfected into Phoenix cells for virus production, and the resulting viral supernatant was used for retroviral transduction of TCR-KO Jurkat reporter T cells. Cells were selected using puromycin, and positivity for TCR expression was tested using an antibody directed against the murine TCR-beta constant region. The results are shown in the graph in FIG. 13B. In Step 13C) a sorting strategy for the screen is provided. In some embodiments, the library can be sorted by any method. The Jurkat reporter T cells from step 13B) were co-cultured for 21 hours at a 1:1 ratio with B cells expressing each of the cognate antigens for the characterized TCRs in the form of multiple tandem minigenes (TMGs). Cells were then sorted for' cell activation by FACS using the CD69 marker. The sorting strategy included (from left to right) sequential gating to select lymphocytes, gating to select singlet cells, gating to exclude CD20⁺-cells, and two sorting gates (‘top’ and ‘bottom’) which capture cells expressing high and low CD69, respectively. The results are shown in the graph in FIG. 13C.

In step 13D) information regarding TCR expression cassettes is provided. In some embodiments, one can retrieve the relevant TCR expression cassettes by any of a variety of techniques. In some embodiments, TCR expression cassettes of top and bottom samples from step 13C) (one replicate of a screen with B cells expressing a TMG and one replicate of a screen with B cells that do not express exogenous antigens) were retrieved using the PCR strategy described in 10C), followed by a barcoding PCR. A control PCR on the plasmid TCR library was included as well. PCR products after the second-round PCR were analysed using an Agilent TapeStation. The results are shown in FIG. 13D.

In step 13E) an analysis of screen data is provided. In some embodiments, the PCR product pool from step 13D) can be analysed in any number of ways. For example, the PCR product pool from 13D) was used for library preparation and was sequenced using Nanopore technology. TCR alpha and beta chain identities were recovered and differentially expressed TCR alpha x beta chain combinations were identified using the DESeq2 R package. The log2-transformed ratio between normalized read counts in the top versus the bottom sample are represented (y-axis) relative to the measured frequency of the TCR (x-axis). The characterized (antigen-specific) TCRs are represented in grey, while the uncharacterized (non-relevant) TCRs are represented in black in FIG. 13E,

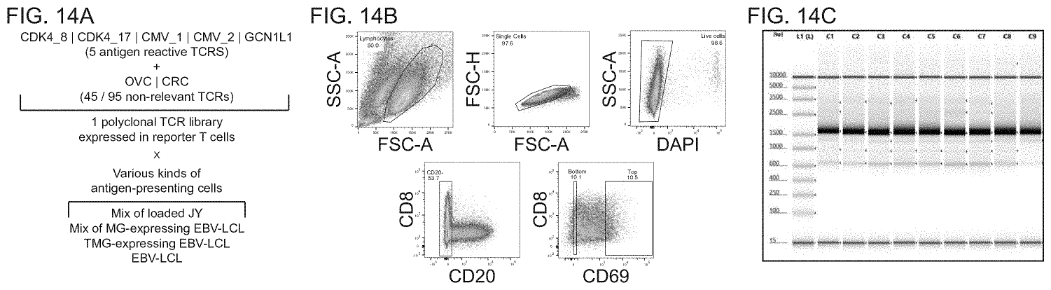

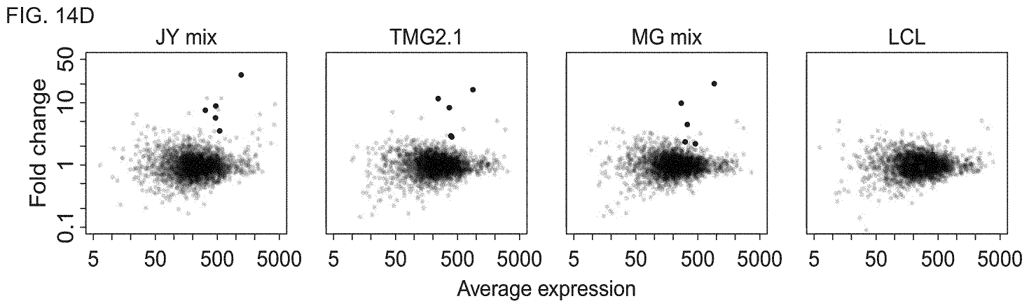

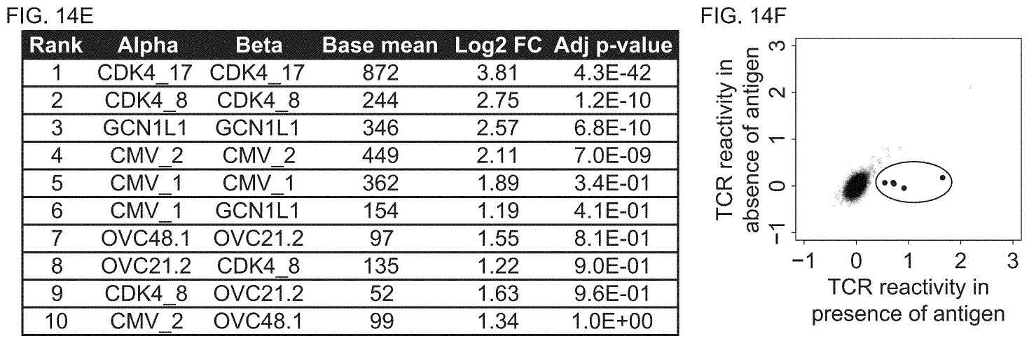

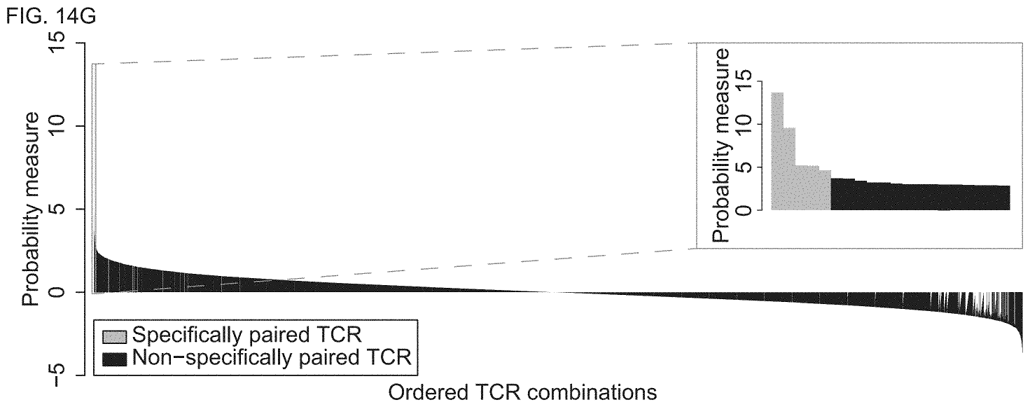

Additional embodiments are shown in FIG. 14, which shows the recovery of antigen-specific TCRs from a TCR library generated by gene synthesis. In Step 14A (FIG. 14A)a schematic of the screen design is provided. Five characterized TCRs and 45 or 95 uncharacterized TCRs from ovarian cancer (OVC) or colorectal cancer (CRC) samples were used to create combinatorial TCR libraries of 50×150 and 100×100 design, respectively. The library was assembled by Twist Bioscience using human V, CDR3 and J segments, while the constant I region was of murine origin. The library was used for retroviral transduction of Jurkat reporter T cells. The polyclonal reporter T cells were cocultured with antigen-presenting cells (APCs) that were engineered to present antigens in various ways. APCs included JY cells loaded with peptide, a mix of EBV-LCL cell lines each expressing a different minigene, EBV-LCL cells expressing a TMG, and EBV-LCLs that have not been engineered to present specific antigens.

In step 14B, a sorting strategy for the screen is provided. The Jurkat reporter T cells expressing the 50×50 design TCR library produced as outlined in 14A) were co-cultured for 21 hours at a 1:1 ratio with the APCs mentioned in 14A). Cells were then sorted for T cell activation by FACS using the CD69 marker. In some embodiments, the library can be sorted by any method. The sorting strategy included (from left to right) sequential gating to select lymphocytes, gating to select singlet cells, gating to select live cells, gating to exclude CD20⁺-cells, and two sorting gates (‘top’ and ‘bottom’) which capture cells expressing high and low CD69, respectively. The results are shown in the graph in FIG. 14B.

Step 14C (FIG. 14C) shows the retrieval of TCR expression cassettes. In some embodiments, one can retrieve the relevant TCR expression cassettes by any of a variety of techniques. In some embodiments, TCR expression cassettes of top and bottom samples from FIG. 14B) were retrieved using the PCR strategy described in 10C), followed by a barcoding PCR. A control PCR on the plasmid TCR library was included as well. PCR products after the second-round PCR were analysed using an Agilent TapeStation. The results are shown in FIG. 14C.

Step 14D) shows the analysis of a 50×50 screen data. In some embodiments, the PCR product pool from step 14C) can be analysed in any number of ways. For example, the PCR product pool from 14C) was used for library preparation and was sequenced using Nanopore technology. TCR alpha and beta chain identities were recovered and the fold change between TCR representation in top and bottom samples (y-axis) is represented as a function of the mean representation of the TCR (x-axis) for every TCR.

Step 14E) shows the characteristics of the top 10 most significantly enriched TCRs. Differentially represented TCR alpha x beta chain combinations from the data in 14D) were identified using the DESeq2 R package. Differential representation analysis is known to the skilled artisan, and is based on a linear model assuming an enriched TCR is defined being enriched in the ‘top’ sample where antigens were presented, and being depleted in the ‘bottom’ sample where antigens were represented, relative to both ‘top’ and ‘bottom’ samples where no antigen was presented. The alpha and beta chains of the top 10 most significant hits, as well as their representation, their log2-transformed fold change and the significance of differential representation are tabulated in FIG. 14E.

Step 14F) shows the analysis of 100×100 screen data. The 100×100 library was screened analogous to the 50×50 library screen described in 14)-)-14E). After TCR alpha and beta chain identification, differentially expressed TCR combinations were identified using the DESeq2 R package. Differential representation analysis is known to the skilled artisan. Average Rlog-transformed read counts for the 100×100 library screen in the presence (x-axis) and absence (y-axis) of TMG expression by B cells is represented in FIG. 14F. The 5 spiked-in characterized TCRs are depicted as encircled larger black dots.

Step 14G shows a rank ordering of the characterized TCRs. Rank order of the significance of enrichment for all TCR combinations represented in the 100×100 library, where statistical analyses were performed as described in 14E). The Wald statistic was calculated using the DESeq2 R package and represented as an ordered plot with decreasing Wald statistic (probability measure; y-axis). The spiked-in characterized TCRs are represented in gray shades. Inset: magnification of the top 20 most statistically significantly enriched TCRs.

FIGS. 15A-15D despict some embodiments for the creation of a TCR repertoire using gene synthesis. In step 15A) (FIG. 15A) a schematic layout of a TCR library is provided. A retroviral construct containing both beta and alpha TCR chains, as well as a puromycin selection marker was used as a scaffold for creating the library. The variable regions of either alpha or beta TCR chains (V-CDR3-J) were synthesized as a pool of 100 oligonucleotide pools each. Subsequently, alpha and beta chain pools were used in a combinatorial cloning reaction to obtain a library of the total complexity of 10,000. Both TCR chain synthesis and combinatorial cloning were conducted by Twist Bioscience (San Francisco/USA). Retroviral transduction using this construct ultimately leads to expression of a single transcript, which results in translation of the TCR beta and alpha chains, as well as a puro resistance marker, due to peptide cleavage at the 2A sites.

In Step 15B) a quality control of a combinatorial TCR library of 100 alpha and 100 beta chains is provided. A primer pair flanking the variable TCR alpha and beta chain domains was used to amplify both chains, and Nanopore sequencing was used to identify the identity of both chains. The representation of each of the 100 alpha chains (left plot), 100 beta chains (middle plot) or 10,000 alpha x beta combinations is represented.

In Step 15C) there is a creation of higher complexity libraries from multiple libraries of lesser complexity. This schematic depicts the idea of creating a more complex library from multiple libraries of lesser complexity. In some embodiments, the libraries of lesser complexity do not contain any possible overlapping combination of alpha and beta chains. In some other embodiments, the libraries of lesser complexity do contain possible overlapping combination of alpha and beta chains. In this example, a combinatorial library of 200 alpha and 200 beta chains (200×200 library) is created by mixing four 100×100 libraries in equimolar ratios. In this example, the four sublibraries are generated as combinatorial libraries of i) TCR alpha numbers 1-100 and TCR beta number 1-100; ii) TCR alpha number 101-200 and TCR beta number 1-100; iii) TCR alpha number 1-100 and TCR beta number 101-200; and iv) TCR alpha number 101-200 and TCR beta number 101-200. In some embodiments, the complexity of the library can vary between 50×50 to 2000×2000.

Step 15D) (as FIG. 15D depicts) TCR library reduction by design based on pairing information or pairing likelihood. This schematic depicts the idea of reducing the complexity of a TCR library without affecting, or with limited effect on, the number of antigen-specific TCR chain pairs present in the library. As an example, the complexity of 10,000 for a 100×100 library may be reduced by equimolar mixing of 10 combinatorial sublibraries of 10×10 design. This leads to a 10-fold TCR library complexity reduction. Information on the pairing of TCR alpha and beta chains, or pairing likelihood information between alpha and beta chains, can be included in the design of this library, in such a way that all the alpha and the beta chains of experimentally identified or otherwise known TCR alpha-beta pairs, or those that are likely to pair, are represented within a single combinatorial sublibrary. In some embodiments, this principle can be applied to composite libraries of higher or lower complexities. In some embodiments, the complexity of combinatorial sublibraries can be higher or lower. In some embodiments, the combinatorial sublibraries can contain overlapping TCR chain combinations. In some embodiments, a composite library can have a range of 100 (10×10) 90,000 (300×300). In some embodiments, the size range of the sublibraries can be 1 (1×1)−100 (50×50).

Additional embodiments are shown in FIGS. 16A-16E, providing for the coculture conditions used for identification of TCRalpha/beta pairs from the TCR repertoire.

FIG. 16A) depicts the results of the use of CD69 as a selection marker of activated Jurkat cells. Jurkat cells expressing hCD8 were transduced with the CMV#1 TCR with a transduction efficiency of 21%. JY cells were pulsed with varying amounts of CMVₚₚ₆₅ peptide (range from 0-10 ug/ml as indicated) or with 1 ug/ml. MART-1 irrelevant peptide (IRR) for 1 hour at 37° C. After 1 hour the cells were washed and 1×10⁵ cells were co-cultured with 1×10⁵ transduced Jurkat cells/well in a 96 U bottom well plate at 37° C. for 20 hours. Cells were harvested, stained with anti-human CD69 (Clone: FN50) and analysed by FACS,

FIG. 16B) depicts CD69 background expression depending on seeding density. Jurkat cells expressing hCD8 and the CMV#1 TCR were cultured for 2 days at different densities (0.25×10⁶/ml, 0.5×10⁶/ml and 1×10⁶/ml). Cells were harvested, stained with anti-human CD69 (Clone: FN50) and analysed by FACS.

FIG. 16C depicts CD69 expression of activated. Jurkat cells in different culture vessels and at various effector to target ratios. Jurkat cells expressing hCD8 and a TCR library (4×4 combinatorial library consisting of alpha and beta chains of 4 characterized antigen-specific TCRs) were co-cultured with target B cells expressing the cognate minigenes (TMG2.1) while maintaining 2.5×10⁵ effector cells per 0.32 CM² culture area. Cells were cultured in 96 U bottom well plate using 1:1 or 1:2 effector to target cell ratios. Alternatively, cells were cultured in a T25 culture flask at ⅓ or ½ of the total amount of cells otherwise used for this surface-area while maintaining a 1:1 ratio of effector to target cells. In addition, cells were cultured in a T75 culture flask at 1:2 and 3:1 effector to target cell ratios. After 20 hours of incubation cells were harvested, stained with anti-human CD69 (Clone: FN50) and analysed by FACS.

FIG. 16D depicts CD69 expression at various coculture densities. Jurkat cells expressing hCD8 and the CDK4-17 or CDK4-8 TCR were co-cultured in a 96 U bottom well plate at a 1:1 ratio with JY cells pulsed with the indicated amount of CDK4_{23-32(24L)} peptide or with the irrelevant MART-1_{26-35(27L)} peptide. The seeding density of effector cells was either 125×10³, 250×10³ or 500×10³ per well. After 20 h cells were harvested, stained with anti-human CD69(Clone: FN50) and analysed by FACS.

FIG. 16E depicts the use of CD69 as a marker for T cell activation in a genetic screen to identify reactive TCRs. A pool of jurkat hCD8+ cells expressing TCRs of unknown specificity at equal ratios was mixed with Jurkat hCD8+cells expressing either CDK4-17, CDK4-8, V#1, CMV#2 or GCN1L1 TCR at the indicated frequencies. This mix of cells was co-cultured with target B cells expressing all the cognate minigenes. After 20 hours cells were harvested, stained with anti-human CD69 (Clone: FN50) and sorted into a ‘top’ and a ‘bottom’ population of cells expressing high and low CD69 levels, respectively. Each of these samples contained about 10% of the total amount of sorted cells. Genomic DNA was retrieved from these cells, and the TCRB variable domains were amplified by PCR. Samples were subjected to Illumina sequencing, and the resulting reads were mapped onto the reference TCRB sequence. The fold enrichment is calculated as the normalized number of reads in the top versus the bottom sample.

Additional embodiments are shown in FIGS. 17-27.

FIG. 17 and FIG. 27 describe the use of CI)69 for detection of antigen-activated T cells. Knowledge of CD69 expression patterns allow detection and selection of activated Tcells in T cell receptor (TCR) library screenings.

FIG. 18, FIGS. 25a and 25b and FIG. 26 describe the use of Blasticidin for the selection of Jurkat reporter T cells transduced with TCR genes thereby providing for the efficient selection of reporter T cells after introduction of TCR libraries.

FIG. 19 describes the stimulation of jurkat reporter T cells in cell culture bags allowing for the stimulation of large numbers of reporter T cells transduced with TCR libraries during the TCR library screening process. The use of large cell numbers can increase sensitivity of the TCR library screening by maintaining sufficient coverage.

FIG. 20 describes the longitudinal analysis of CD69, CD25 and. CD62L expression on Jurkat reporter T cells transduced with different TCRs. Understanding of longitudinal expression of expression markers allows the selection of single (or multiple) activation markers to specifically detect antigen-activated T cells and perform two-step selection procedures, for example by magnetic bead enrichment.

FIG. 21 to FIG. 24 describe the use of different NFAT reporter systems for the detection of antigen-activated T cells providing an understanding that the design of NFAT reporter gene cassettes, type of delivery and their genomic insertion site controls the functionality and the level of antigen-independent background expression of the reporter gene. The data demonstrate that different viral delivery vectors, clonal reporter T cell populations, different reporter T cells or different reporter systems may lead to more optimal reporter function.

In some embodiments, the method can include steps (1)-(7) described below (and inf FIG. 31). Step (1) Obtaining a sample. The sample can be tissues, blood, or body fluids from a patient suffering infectious diseases, autoimmune diseases, or cancers. The sample can be viable or non-viable. Step (2) Sequencing TCR-α and β chains in the sample. Step (3) Selecting and combinatorial pairing TCRα- and β-chain sequences to create a library of TCRαβ pairs. Step (4) introducing the library of TCRαβ pairs into a pool of reporter cells, for example, Jurkat reporter T cells. Step (5) Stimulating the reporter cells that are modified with the library of TCRαβ pairs with antigen presenting cells presenting at least one antigen of interest. The at least one antigen of interest can be autologous or allogeneic. Step (6) Determining TCRαβ pairs specific to the at least one antigen of interest. Step (7) Introducing the TCRαβ pairs into cells and selecting cells containing the TCRαβ pairs.

In some embodiments, the method can involve one or more of the steps (1)-(7) described above. Any of the steps can be omitted, repeated, or substituted by other embodiments provided herein, as appropriate. Additional intervening steps can also be added. For example, some embodiments include steps (2) and (3). Other embodiments include steps (5) and (6). Still others include step (7). Some embodiments include steps (1)-(7), and further include administering the cells containing the TCRαβ pairs into patients for treatment. In some embodiments, the antigen presenting cells can be obtained by introducing neo-antigen library into B cells. The neo-antigen library can be autologous or allogeneic. Some embodiments relate to creating TCR repertoires by selection of TCR chain subsets. Some embodiments relate to a B cell comprising any neo-antigen from the neo-antigen library. Some embodiments relate to an application of genetic screening based on enrichment/depletion. Some embodiments relate to performing genetic screening with large size amplicons. Some embodiments relate to a method for detection of TCR modified T cells. Some embodiments combine any one of more of the preceding embodiments.

Some embodiments relate to a nucleotide library that comprises the repertoire of T cell receptors recovered according to any one of the above embodiments.

In some embodiments, a nucleotide construct comprising the nucleotide sequence identified according to any one of the above embodiments. In some embodiments, a cell comprises the nucleotide construct described herein.

Sonic embodiments are according to FIG. 28. In some embodiments, neo-antigen specific TCR identification is achieved by applying a genetic screening approach which is scalable and minimally invasive. A small amount of non-viable archival tumor tissue is used as a source of intratumoral TCR sequences instead of TILs. Next, retroviral gene transfer is used to introduce the identified library of intratumoral TCRs into an immortalized T lymphocyte cell line, called a Jurkat reporter T cell line. The TCR library-expressing Jurkat cells (effector cells, E in short) are enriched and subsequently screened for their reactivity against mutanome-expressing patient-derived APCs (target cells, T in short). Following flow cytometric sorting, Jurkat cells are selected based on their expression of the early T cell activation marker CD69 (as an example) which is involved in cell proliferation and downstream signal transduction³². In some embodiments, samples may be separated on the basis of any other activation marker, including, but not limited to, CD25, CD62L, CD13, IFN-γ, Il-2, TNF-α, GM-CSF, synthetic promoter reporter markers or proliferation markers. As a final step, the neo-antigen specific TCRs are identified by next generation sequencing. The current TCR isolation platform provides for the screening of a library of 10,000 TCRs. In order to be able to identify neo-antigen specific TCR(s) from such a library with high sensitivity and ensure that TCRs are not lost during the different processing steps, each unique TCR has to be represented multiple times during the screening process to maintain the TCR coverage. Therefore, a large number of TCR-transduced Jurkat cells and APCs have to be screened³³.

Following the retroviral TCR library transduction into Jurkat TCR KO cells, an efficient and high throughput selection procedure is useful to enrich successfully TCR-transduced Jurkat cells (>80% mTCRβ⁺ CD8⁺ cells) without causing toxicity and losing TCR coverage. In some embodiments, 96 well round-bottom plates can be used for APC-Jurkat co-culture. In some embodiments, GMP bags can be used for APC-Jurkat co-culture. In some embodiments, the co-culture can be carried out in a closed system. In some embodiments, a co-culture with 168×10⁶ Jurkat cells and APCs can be set up in a GMP bag. In some embodiments, the co-culture can be 16, 20, 24, 48 or 32 hours. In some embodiments, the readout can be with respect to CD69, CD25, or CD62L. In some embodiments, the readout can be the combination of CD69 and CD25. In some embodiments, the readout can be the combination of CD69 and CD62L. Some embodiments relate to selection of CD25⁺ CD62L⁻ Jurkat cells in combination with CD69. In some embodiments, a GMP bag can be employed.

Antibiotic selection is an attractive strategy to enrich for TCR-transduced Jurkat T cells. In some embodiments, blasticidin selection can be used. Some embodiments are according to FIG. 30. In some embodiments, on day 4 of the selection the cells are re-plated at their starting density either in medium with or without the respective concentration of blasticidin. In some embodiments, the concentration of blasticidin is 4 ug/ml. Some embodiments are a 7-day selection with the starting cell density at 0.25×10⁶ cells/ml, the concentration of blasticidin at 4 ug/ml, removing the antibiotic on day 4. In some embodiments, one can plate at 0.5e6/ml cells and add 6 ug/mL blasticidin and re-plate the cells at 0.5e6/ml on day 4 without adding Blastblasticidin.

In some embodiments, the reporter systems can be AP-1 or NFkB signaling pathways.

Some embodiments are according to FIG. 29. In some embodiments, one goal is to upscale the number of TCRs in a library to allow high sensitivity screening of greater than 10,000 TCRs. This highlights the need to enhance the scalability of the TCR discovery platform by optimizing various process steps to allow a more efficient processing of a large number of cells while still maintaining TCR coverage. In some embodiments, four known HLA-A*02:01-restricted TCRs-CDK4 TCR clone 8 and 17 (CDK4-8 and 17 in short) and CMV TCR clone 1 and 2 (CMV-1 and 2 in short) were used. The two CDK4 TCRs are specific for a mutated cyclin-dependent kinase 4 (CDK4_{R24C}) peptide. This mutation-derived neo-antigen epitope was identified in multiple melanoma patients³⁴. Furthermore, the two CMV TCRs target a peptide encoded by a component of the human cytomegalovirus (CMV), pp65³⁵. For both the CDK4 and the CMV epitopes, two distinct TCR clones with potentially different affinities in the studies were used. This allows one to evaluate the role of TCR affinity for the cognate peptide on the screening process.

### Additional Embodiments—Results in Example 19

TCR gene therapy involves engineering autologous T cells to express TCRs of desired specificity against cancer antigens. One class of cancer antigens are the nonsynonymous somatic mutation-derived neo-antigens which are solely expressed on malignant cells and are thus an attractive target for TCR gene therapies. However, most neo-antigens are unique to a given patient's tumor and targeting them necessitates a personalized approach. To overcome this challenge, a fully personalized neo-antigen specific TCR gene therapy is provided by incorporating a genetic screening approach to identify such TCRs from a library of a patient's TCR genes isolated from a tumor biopsy. The current TCR isolation platform allows the screening of 10,000 TCRs which involves the processing of a large amount of TCR library-transduced reporter Jurkat T cells and neo-antigen-expressing antigen-presenting cells (APCs) to maximize the screening sensitivity. However, the handling of a large number of cells might affect the scalability of the platform. Therefore, this study aims at enhancing the scalability of the screening platform while maintaining its sensitivity by examining alternative methods for the processing of large cell numbers. First, it was shown that blasticidin selection leads to an efficient and minimally toxic enrichment of TCR-expressing Jurkat cells. Next, upscaling the Jurkat cell-APC co-cultures from the established 96 well plate format to a more high throughput MACS GMP Cell Differentiation Bag setup resulted in a comparable activation of the Jurkat cells. Furthermore, different methods were examined to replace flow cytometric sorting which is currently used in the screening process with a more scalable bead-based selection. Therefore, in addition to CD69, the expression profiles of two additional T cell activation markers CD25 and CD62L were examined, and they were found to be potentially suitable candidates for a two-step bead-based enrichment in combination with CD69. Additionally, an NEAT (family of nuclear factor of activated T cells)-based reporter system was assessed which would circumvent the usage of flow cytometric sorting by utilizing a reporter gene such as an antibiotic resistant cassette or a cell surface marker suitable for bead-based selection. However, the NFAT-reporter system displayed a high level of background signal.

Within the tumor microenvironment cytotoxic T lymphocytes (CD8⁺ T cells) are mainly responsible for tumor regression¹. T cells express unique T cell receptors (TCRs) which are heterodimers consisting of α and β chains. Each α and β chain is made up of a constant and variable region². The variable regions confer the specificity and affinity of a given T cell for a cognate peptide presented by an antigen-presenting cell (APC) on its major histocompatibility complex (MHC). The MHC molecules are also referred to as human leukocyte antigens (HLAs) in humans^{2,3}. CD8/CD4 and CD28 are examples of T cell co-receptors which stabilize the TCR-HLA complex and together with CD3ζ initiate downstream signaling involving protein tyrosine phosphorylation and cytoplasmic calcium release. This downstream signaling induces the nuclear translocation of the transcription factors NFAT (family of nuclear factor of activated T cells), AP-1. and NF-κB and the subsequent transcription of genes specific for T cell activation^{4,5}. Activated CD8⁺ T cells are able to kill target cells expressing viral, bacterial or cancer antigens by producing a variety of inflammatory cytokines such as interleukin-2 (IL-2), IFN-γ and TNF-α. Secreted IL-2 binds to the IL-2 receptor on T cells, resulting in a positive feedback loop by stimulating the production of more IL-2 and enhancing the proliferation of T cells^{5,6}.

To date cancer immunotherapy has been shown to be one of the most effective methods to treat advanced tumors. Many immunotherapy approaches exploit the killing properties of cytotoxic T cells^{7,8}. One example is the blockage of the immune checkpoint molecules CTLA-4 and PD-1, which suppress the cytotoxic activity of CD8⁺ T cells in the tumor microenvironment^{9,10}. Checkpoint blockade is a scalable routinely administered therapy that has been most effective in cancers with a high rate of nonsynonymous mutations¹¹ such as melanoma^{12,13} and non-small-cell lung cancer (NSCLC)^{14,15}. Furthermore, the adoptive cell transfer of autologous tumor infiltrating lymphocytes (TILs), expanded in vitro and supplemented with IL-2, has demonstrated curative potential in melanoma^{16,17} and cervical cancer^{18,19}. However, the tumor antigen specific TILs are frequently terminally differentiated T cells and may be short-lived and lost during the in vitro expansion process^{7,20}.

None of the above-mentioned immunotherapies identifies the tumor antigen reactive TCRs involved in the tumor regression. The detection of those TCRs is useful to genetically engineer T cells with TCRs against the tumor antigens. Such a therapy is called TCR gene therapy and presents an advantage over other immunotherapeutic strategies since it allows the generation of a great number of ‘fitter’ T cells with a desired antigen specificity²¹. Tumor antigens can be non-self-antigens or self-antigens^{7,21}. Self-antigens have been the main focus of cancer vaccine trials but possibly due to central tolerance against self-antigens those trials have been ineffective²². However, some TCR gene therapy trials targeting aberrantly expressed self-antigens have shown clinical efficacy. For instance, targeting of the cancer germline NY-ESO-1 epitope in patients with synovial cell sarcoma and melanoma has shown curative potential²³. Nevertheless, targeting tumor-associated self-antigens has also frequently resulted in severe on-target toxicities^{24,25}, underlying the need for immunotherapies to target non-self-antigens. One such type are the mutation-derived neo-antigens^{7,8,21}.

Neo-antigens arise from nonsynonymous somatic mutations and result in the generation of novel polypeptides absent in healthy tissue. This makes neo-antigens useful targets for immunotherapies as their complete absence in healthy tissue would prevent on-target toxicity. Additionally, the discovery of neo-antigen specific T cells would not be influenced by central tolerance against high affinity self-antigen reactive T cells. Neo-antigens mostly occur from mutations in passenger genes which do not confer any survival advantage to the malignant cells. These mutations are normally unique to each patient and therefore the targeting of neo-antigens requires a personalized treatment involving a genome sequencing approach^{8,26}.

With the recently developed whole-exome sequencing (WES) and RNA sequencing it has become apparent that neo-antigen reactive T cells are found in TILs and can. mediate tumor regression^{27,28}. For instance, WES coupled with highly specific and sensitive peptide-MHC (pMHC) multimers²⁹ has led to the identification of neo-antigen specific cells from TIL material in melanoma patients^{13,30}. Even though this method is scalable, it is dependent on a limited HLA allele coverage and requires algorithms to predict the pMHC binding. These restrictions might result in some neo-antigen reactive TCRs being overlooked. To circumvent the limits of pMHC multimers, studies have used mass spectrometry to identify MHC-bound neo-antigens. Combining this approach with WES and transcriptome sequencing as a reference has led to the discovery of neo-antigens in murine tumor cell lines³¹. However, this setup is low throughput and frequently results in false negatives⁸. Despite the possibility to simultaneously identify multiple neo-antigen specific TCRs with the above-mentioned methods, there is still a need for a more scalable and sensitive platform for the discovery of such TCRs.

Neo-antigen specific TCR identification is achieved by applying a genetic screening approach which is scalable and minimally invasive. A small amount of non-viable archival tumor tissue is used as a source of intratumoral TCR sequences instead of TILs. Next, retroviral gene transfer is used to introduce the identified library of intratumoral TCRs into an immortalized T lymphocyte cell line, called a Jurkat reporter T cell line. The TCR library-expressing Jurkat cells (effector cells, E in short) are enriched and subsequently screened for their reactivity against mutanome-expressing patient-derived APCs (target cells, T in short). Following flow cytometric sorting, Jurkat cells are selected based on their expression of the early T cell activation marker CD69 which is involved in cell proliferation and downstream signal transduction³². As a final step, the neo-antigen specific TCRs are identified by next generation sequencing. In some embodiments, the current TCR isolation platform provides for the screening of a library of 10,000 TCRs. In line with the above, Example 19 provides further results and evidence to support this approach.

Selection of TCRs can be achieved in a number of ways. Variant enrichment may be determined using suitable analytical tools, including but not limited to the DESeq2 R package. Variant enrichment may include contrasting top-bottom pairs where reporter T cells were contacted with B cells that express TMGs with top-bottom pairs where reporter T cells were contacted with B cells that were not engineered to express TMGs. Variant enrichment may include ranking TCR combinations based on the DI Seq2 Wald test statistic in decreasing order. Variant enrichment may include determining statistical significance based on Bonferroni adjusted p-values for the higher ranked TCR combinations. Selection of at least one TCR combination may be based on the adjusted p-values and other statistical metrics. The procedure in this example may be executed as a single replicate, or in duplicate, triplicate or more than three replicates to increase sensitivity of TCR reactivity. The number of replicates may be varied for samples that were derived from cocultures with APCs expressing TMGs, and for samples that were derived from cocultures with APCs that were not engineered to express TMGs. Any of these options can be combined with any of the methods provided herein.

Embodiments of the present disclosure are further illustrated in the following Examples, which are given for illustration purposes only and are not intended to limit the scope of the claimed subject matter in any way.

### EXAMPLES

### Example 1

This example describes the recovery of TCR repertoires from non-viable tumor specimens to identify neo-antigen specific TCR sequences.

DNA or RNA is isolated from fresh-frozen or fixed or formalin fixed/paraffin-embedded (FFPE) tumor specimen and used to perform bulk TCRα- and β-chain sequencing. Absolute numbers of nucleic acid molecules encoding (part of) a particular TCR chain amino acid sequence are determined based on the count of unique molecules using a “Unique Molecular Identifier” (UMI), In the alternative, UMIs are not included in the the TCRα- and β-chain sequencing, and frequency of TCR chains is measured based on next generation sequencing read counts rather than UMI count. By applying criteria such as intratumoral TCR chain abundance, for example, a defined set of TCRα- and β-chains is selected from the total set of identified TCR sequences. Subsequently, DNA or RNA fragments of the selected TCRα- and β-chains are generated by DNA or RNA synthesis, respectively. RNA fragments can be converted to cDNA by standard techniques. Through combinatorial pairing of all selected TCRα- and β-chains into single expression constructs encoding TCRαβ genes, a defined part of the original repertoire of intratumoral TCRαβ pairs is recreated. A single expression construct can be used for expression of a given combination of a single TCRα and TCRβ chain. Alternatively, TCRα and TCRβ chains can be expressed from separate expression constructs. Any suitable expression vector can be used, including viral vectors. For stable expression, retroviral or lentiviral vectors or particles are used. The resulting library of TCRαβ genes is expressed in a pool of reporter T cells. Library-expressing T cells are activated by neo-antigen stimulation, and neo-antigen specific T cells are enriched based on T cell activation markers. Subsequently, the expressed neo-antigen-specific TCRαβ genes are identified by enrichment in antigen-stimulated samples relative to samples which were not antigen-stimulated. The identified TCR gene(s) or set of TCR genes are utilized to engineer neo-antigen specific T cells for cancer therapy.

### Example 2

This example describes the recovery of TCR repertoires for the generation of TCRαβ libraries.

DNA or RNA is isolated from a fresh-frozen or fixed or formalin fixed/paraffin-embedded (FFPE) specimen and used to perform bulk TCRα- and 62 -chain sequencing. Absolute numbers of nucleic acid molecules encoding (part of) a particular TCR chain amino acid sequence are determined based on the count of unique molecules using a “Unique Molecular Identifier” (UMI). By applying criteria such as TCR chain abundance, for example, a defined set of TCRα- and β-chains is selected from the total set of identified TCR sequences. Subsequently, DNA or RNA fragments of the selected TCRα- and β-chains are generated by DNA or RNA synthesis, respectively. RNA fragments can be converted to cDNA by standard techniques. Through combinatorial pairing of all selected TCRα- and β-chains into TCRαβ genes, a defined part of the original repertoire of TCRαβ pairs is recreated. The paired. TCRα and TCRβ chains represent a TCRαβ library comprising a selected TCR repertoire.

### Example 3

This example describes treating cancer patients with immunotherapy utilizing libraries of recovered TCR repertoires.

TCR repertoires are recovered and TCRαβ libraries are generated by the methods outlined in Examples 1 and 2. The library of TCRαβ genes is expressed in a pool of reporter T cells. Neo-antigen specific T cells are activated by antigen stimulation and are isolated based on T cell activation. Subsequently, the expressed neo-antigen-specific TCRαβ genes are identified. The identified TCR gene(s) or set of TCR genes are utilized to engineer neo-antigen specific T cells for cancer therapy by expressing the TCR genes in the T cells. Engineered neo-antigen specific T cells are infused into a cancer patient as immunotherapy to treat the cancer. The cancer patient may be the patient whose TCRαβ repertoire was sequenced or a patient whose cancer harbors or expresses the same neo-antigen. The cells that are used for therapy may be autologous or allogeneic.

### Example 4

This example describes the recovery of TCR repertoires from sites of infection or autoimmunity.

DNA or RNA is isolated from a fresh-frozen or fixed or formalin fixed/paraffin-embedded (FFPE) specimen obtained from a site of infection or autoimmunity and used to perform bulk TCRα- and β-chain sequencing. By applying criteria such as TCR chain abundance, for example, a defined set of TCRα- and β-chains is selected from the total set of identified TCR sequences. Subsequently, DNA or RNA fragments of the selected TCRα- and β-chains are generated by DNA or RNA synthesis, respectively. RNA fragments can be converted to cDNA by standard techniques. Through combinatorial pairing of all selected TCRα- and β-chains into TCRαβ genes, a defined part of the original repertoire of TCRαβ pairs is recreated. By determining the TCR sequences of T cells that can detect a particular antigen at a site of infection or autoimmunity, TCR repertoires associated with or specific to the site of infection or autoimmunity can be recovered.

The resulting library of TCRαβ genes is expressed in a pool of reporter T cells. Antigen specific T cells are activated by antigen stimulation and isolated based on T cell activation. Subsequently, the expressed antigen-specific TCRαβ genes are identified. The identified TCR gene(s) or set of TCR genes are utilized to diagnose or treat an infection or autoimmunity.

### Example 5

This example describes the recovery of antigen-specific TCRs from a TCR library generated by artificial mixing of TCR plasmids.

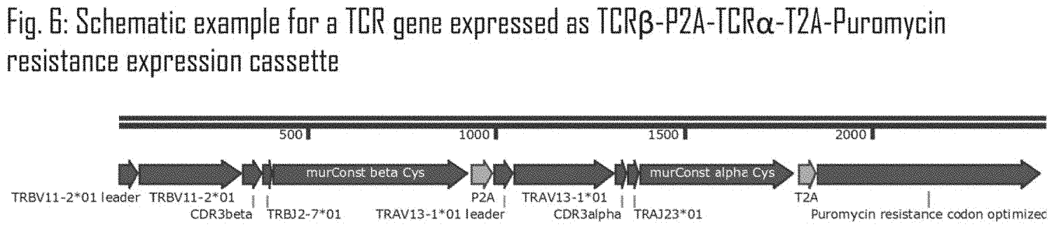

TCR expression cassettes are generated in the format of TCRβ-P2A-TCRα-T2A-Puromycin resistance (FIG. 6 shows a schematic example of a TCRβ-P2A-TCRα-T2A-Puromycin resistance cassette; Example of a TCR expressed in such a format is given SEQ ID NO: 1, FIG. 32, FIG. 40). Multiple TCR libraries are generated by mixing one neo-antigen specific TCR with various non-related TCRs in several ratios. The resulting TCR libraries contain one HLA-A*02:01 restricted, neo-antigen specific TCR in a frequency of 1:10, 1:100, 1:1,000 and 1:10,000. To create a TCR library containing the neo-antigen specific TCR at a frequency of 1:10, one plasmid copy encoding the neo-antigen specific TCR in a retroviral expression vector is mixed with one plasmid copy for each of nine other retroviral vectors each encoding a distinct TCR of other specificity. Accordingly, one plasmid copy encoding the neo-antigen specific TCR in a retroviral expression vector is mixed with 1, 11, 111 and 1,111 plasmid copies for each of nine other retroviral vectors each encoding a distinct TCR of other specificity to obtain TCR libraries containing the neo-antigen specific TCR at a frequency of 1:10, 1:100, 1:1,000 and 1:10,000, respectively.

In some embodiments, any one or more nucleic acid encoding for SEQ ID NO: 1 can be employed. In some embodiments, one can alter the above by mixing in 5 characterized TCRs and at 1:16, 1:100, 1:2500 and 1:10,000 frequencies. Finally one can use 24 rather than 9 other TCR plasmids for mixing.

Each TCR library is separately transfected into amphotropic virus producer cells such as Phoenix-Arnpho (ATCC CRL-3213) by methods known to the skilled artisan. The resulting retroviral virions are used to transduce a Jurkat reporter T cell line. The Jurkat reporter T cell line lacks endogenous TCR expression (for example described in Mezzadra et al Nature 2017) and is modified to express human CD8α and CD8β after transduction with a CD8α-P2A-CD8β transgene (SEQ ID NO: 2) using methods known to the skilled artisan. Jurkat reporter T cells are transduced with the individual TCR libraries using a low MOI in order to limit the frequency of TCR transduced. Jurkat T cells to 25-30% of total T cells. Jurkat T cells modified to express a TCRβ-P2A-TCRα-T2A-Puromycin resistance transgene are positively selected by the addition of Puromycin to the cell culture media after transduction. Antibiotic selection of genetically modified cells is known to an ordinary person skilled in the art.

In some embodiments, any one or more nucleic acid encoding for SEQ ID NO: 2 (FIG. 33) can be employed. FIG. 41 depicts a diagram for SEQ ID NO: 2, a CD8α-P2A-CD8β transgene

An ordinary person skilled in the art will further appreciate that the methods described herein can include selection without puromycin, such as sorting of TCR-transduced cells by FACS or magnetic bead-based selection, for example. Thus, a TCR cassette may lack the puromycin selection gene.

TCR transduced Jurkat T cells are stimulated with antigen-loaded K562 cells expressing a recombinant HLA-A*02:01-IBES-FusionRed transgene (K562-HLA-A*02:01-IRES-FusionRed; SEQ ID NO: 3; FIG. 34, FIG. 42). The generation of transgene-expressing K562 cells has been described (for example, Hirano et al. Clin Canc Res 2006; Butler et al Int Immunol 2010; Butler et al Clin Cane Res 2007; Lorenz et al. Hum Gene Ther 2017) and is known to the skilled artisan. Peptide-loaded K562-HLA-A2 cells are obtained by pulsing with the peptide of interest for 90 minutes at 37° C. and subsequent washing. The skilled artisan will appreciate that peptide-presenting HLA-A*02:01. positive K562 cell line mentioned herein may be substituted with other HLA-A*02:01 positive antigen-presenting cells. In the alternative, a variant (expression of TMG in HLA-A02*01-transduced K562 cells) can be used.

The following stimulation conditions will be used for each TCR library respectively:
1. 4e⁷ TCR transduced Jurkat T cells+4e⁷ K562-HLA-A2 loaded with 1 ug of neo-antigen peptide
2. 4e⁷ TCR transduced Jurkat T cells+8e⁷ K562-HLA-A2 loaded with 1 ug of neo-antigen peptide

Subsequently, co-cultures are harvested and stained with fluorochrome-labeled antibodies for CD4, CD8 and CD69. Using a BD Biosciences ARIAFusion flow cytometry sorter the following populations are sorted for each stimulation condition:
1. Live, single cell, CD4⁺, CD8⁺, CD69^{hi} (CD69^{hi} includes the highest 5-50% of live, single cell, CD4⁺, CD8⁺ cells based on CD69 fluorescence signal)
2. Live, single cell, CD4⁺, CD8⁺, CD69^{lo} (CD69^{lo} includes 5-50% of the live, single cell, CD4⁺, CD8⁺ cells based on a low CD69 fluorescence signal)

Alternatively, the following stimulation conditions can be used for each TCR library respectively:
1. 4e⁷ TCR transduced Jurkat T cells+4e⁷ K562-HLA-A2 loaded with 1 ug of neo-antigen peptide
2. 4e⁷ TCR transduced Jurkat T cells+8e⁷ K562-HLA-A2 loaded with 1 ug of neo-antigen peptide
3. 4e⁷ TCR transduced Jurkat T cells+4e⁷ K562-HLA-A2 loaded with 1 ug of irrelevant control peptide
4. 4e⁷ TCR transduced Jurkat T cells+8e⁷ K562-HLA-A2 loaded with 1 ug of irrelevant control peptide

Subsequently, co-cultures are harvested and stained with fluorochrome-labeled antibodies for CD4, CD8 and CD69. Using a BD Biosciences ARIAFusion flow cytometry sorter live, single cell, CD4⁺, CD8⁺, CD69⁺ cells are sorted for each sample.

Genomic DNA is isolated from the sorted TCR transduced Jurkat T cells and used as template for a PCR to amplify part of the TCRβ-P2A-TCRα cassette. The resulting PCR product has a size of approx. 1.5 kb and can be sequenced using an Oxford. Nanopore MinIon sequencer to compare the relative abundance of neo-antigen specific TCR sequences in the sorted T cell populations (either CD69^{lo} and CD69^{hi} or CD69⁺).

In some embodiments, the Oxford Nanopore Minion sequencer may be replaced by other sequencing instruments or other sequencing strategies can be employed.

### Example 6

This example describes the recovery of antigen-specific TCRs from a TCR library generated by gene synthesis.

Multiple TCR libraries containing two FILA-A*02:01 restricted, neo-antigen specific TCR are generated by gene synthesis. For this, two TCRα and two TCR chain fragments derived from the neo-antigen specific TCRs and 98 TCRα and 98 TCRβ chain fragments derived from TCRs with other specificity are synthesized. In the alternative, 5+95 TCRs can be employed. Subsequently, the resulting fragments are used to generate TCR libraries containing TCR expression cassettes in a TCRβ-P2A-TCRα format. For library generation all selected TCRα and TCRβ fragments are mixed and joined to continuous nucleic acid molecules encoding a TCRβ-P2A-TCRα cassettes. Importantly, only one TCRβ- and TCRα-fragment can be joined per TCR cassette. By selection and mixing of different numbers of TCRα and TCRβ fragments, TCR libraries of different complexity can be created:
1. two TCRα and two TCRβ chain fragments derived from the neo-antigen specific TCRs will create a TCR library with complexity 4 (4 different TCRαβ pairs)
2. two TCRα and two TCRβ chain fragments derived from the neo-antigen specific TCRs AND eight TCRα and eight TCRβ chain fragments derived from TCRs with other specificity will create a TCR library with complexity 100 (100 different TCRαβ pairs)
3. two TCRα and two TCRβ chain fragments derived from the neo-antigen specific TCRs AND 48 TCRα and 48 TCRβ chain fragments derived from TCRs with other specificity will create a TCR library with complexity 2,500 (2,500 different TCRαβ pairs)
4. two TCRα and two TCRβ chain fragments derived from the neo-antigen specific TCRs AND 98 TCRα and 98 TCRβ chain fragments derived from TCRs with other specificity will create a TCR library with complexity 10,000 (10,000 different TCRαβ pairs)

In the alternative, options can be: 4+0; 5+5; 5+45 and 5+95 designs for TCRα and TCRβ chain fragments

The resulting TCR libraries will contain TCR expression cassettes in the format of TCRβ-P2A-TCRα-T2A-Puromycin resistance (SEQ ID NO: 1, FIG. 40).

Each TCR library is separately transfected into amphotropic virus producer cells such as Phoenix-Ampho (ATCC CRL-3213) by methods known to the skilled artisan. The resulting retroviral virions are used to transduce a Jurkat reporter T cell line. The Jurkat reporter T cell line lacks endogenous TCR expression (for example described in Mezzadra et al Nature 2017) and is modified to express human CD8α and CD8β after transduction with a CD8α-P2A-CD8β transgene (SEQ ID NO: 2) using methods known to the skilled artisan. Jurkat reporter T cells are transduced with the individual TCR libraries using a low MOI in order to limit the frequency of TCR transduced Jurkat T cells to 25-30% of total ‘I’ cells. Jurkat T cells modified to express a TCRβ-P2A-TCRα-T2A-Puromycin resistance transgene are positively selected by the addition of Puromycin to the cell culture media after transduction. Antibiotic selection of genetically modified cells is known to an ordinary person skilled in the art.

An ordinary person skilled in the art will further appreciate that the methods described herein can include selection without puromycin, such as sorting of TCR-transduced cells by FACS or magnetic bead-based selection, for example. Thus, a TCR cassette may lack the puromycin selection gene.

TCR transduced Jurkat T cells are stimulated with antigen-loaded K562 cells expressing a recombinant HLA-A*02:01-IRES-FusionRed transgene (K562-HLA-A*02:01-IRES-FusionRed; SEQ ID NO: 3) for 6 hours. The generation of transgene-expressing K562 cells has been described (for example, Hirano et al. Clin Canc Res 2006; Butler et al Int Immunol 2010; Butler et al Clin Cane Res 2007; Lorenz et al. Hum Gene Ther 2017) and is known to the skilled artisan. Peptide-loaded K562-HLA-A2 cells are obtained by pulsing with the peptide of interest for 90 minutes at 37° C. and subsequent washing. The skilled artisan will appreciate that peptide-presenting HLA-A*02:01 positive K562 cell line mentioned herein may be substituted with other HLA-A*02:01 positive antigen-presenting cells.

The following stimulation conditions will be used:
1. 4e⁷ TCR transduced Jurkat T cells+4e⁷ K562-HLA-A2 loaded with 1 ug of neo-antigen peptide
2. 4e⁷ TCR transduced Jurkat T cells+8e⁷ K562-HLA-A2 loaded with 1 ug of neo-antigen peptide

Subsequently, co-cultures are harvested and stained with fluorochrome-labeled antibodies for. CD4, CD8 and CD69. Using a BD Biosciences ARIAFusion flow cytometry sorter the following populations are sorted for each stimulation condition:
1. Live, single cell, CD4⁺, CD8⁺, CD69^{hi} (CD69^{hi} includes the highest 5-60% of five, single cell, CD4⁺, CD8⁺ cells based on CD69 fluorescence signal)
2. Live, single cell, CD4⁺, CD8⁺, CD69^{lo} (CD69^{lo} includes 5-50% of the live, single cell, CD4⁺, CD8⁺ cells based on a low CD69 fluorescence signal)

Alternatively, the following stimulation conditions can be used for each TCR library respectively:
1. 4e⁷ TCR transduced Jurkat T cells+4e⁷ K562-HLA-A2 loaded with 1 ug of neo-antigen peptide
2. 4e⁷ TCR transduced Jurkat T cells+8e⁷ K562-HLA-A2 loaded with 1 ug of neo-antigen peptide
3. 4e⁷ TCR transduced Jurkat T cells+4e⁷ K562-HLA-A2 loaded with 1 ug of irrelevant control peptide
4. 4e⁷ TCR transduced Jurkat T cells+8e⁷ K562-HLA-A2 loaded with 1 ug of irrelevant control peptide

Subsequently, co-cultures are harvested and stained with fluorochrome-labeled antibodies for CD4, CD8 and CD69. Using a BI) Biosciences ARIAFusion flow cytometry sorter live, single cell, CD4⁺, CD8⁺, CD69⁺ cells are sorted for each sample.

Genomic DNA is isolated from the sorted TCR transduced Jurkat T cells and used as template for a PCR to amplify part of the TCRβ-P2A-TCRα cassette. The resulting PCR product has a size of approx. 1.5 kb and can be sequenced using an Oxford Nanopore Minion sequencer to compare the relative abundance of neo-antigen specific TCR sequences in the sorted T cell populations (either CD69^{lo} and CD69^{hi} or CD69⁺).

In some embodiments, the Oxford Nanopore Minion sequencer may be replaced by other sequencing instruments or other sequencing strategies may be employed.

### Example 7

This example describes the recovery of neo-antigen specific TCRs from a TCR library generated from a fresh-frozen melanoma lesion.

DNA and RNA are isolated from a fresh-frozen melanoma specimen and used two-fold:

First, DNA and/or RNA is utilized to perform bulk TCRα- and β-chain sequencing. Absolute numbers of nucleic acid molecules encoding (part of) a particular TCR chain amino acid sequence are determined based on the count of unique molecules using a “Unique Molecular Identifier” (UMI). In the alternative, read counts can be used. The resulting collection of TCR chain sequences is divided into a collection of TCRα-and a collection of TCRβ-chain sequences. Any non-productive TCR chain sequences, in which TCR segments are joined out of frame at the amino acid sequence level, and/or in which stop codons are introduced, and/or in which frameshift mutations are present, and/or in which defective splicing sites are present, are removed from the collection. Each collection is sorted in descending order using either absolute numbers of nucleic acid molecules encoding a particular TCR chain (or corresponding percentage among total TCRα- or TCRβ-chains, respectively) to obtain a rank order for TCRα- and β-chains. The Top 100 most abundant TCRα- and β-chains are selected and generated as fragments by DNA synthesis. For library generation all selected TCRα and TCRβ fragments are mixed and joined to continuous nucleic acid molecules encoding TCRβ-P2A-TCRα cassettes. Importantly, only one TCRβ- and TCRα-fragment can be joined per cassette. The resulting TCR libraries will contain approximately 10,000 TCR expression cassettes in the format of TCRβ-P2A-TCRα-T2A-Puromycin resistance (SEQ ID NO: 1).

Second, tumor-derived as well as healthy tissue DNA and RNA is used to determine the set of tumor-specific mutations using Whole-exome-sequencing (WES) and to establish the set of expressed mutated genes by utilizing RNA-seq. Tandem-minigene (TMG) constructs can be generated that encode multiple tumor-derived mutated peptides in tandem arrays. TMG constructs are used to generate in vitro transcribed mRNA (for example, Stevanovié et al. Science 2017). In the alternative, TMG expression constructs can be used for virus production/transduction of B cells (APCs).

In parallel, matched autologous blood from the melanoma patient is used to generate immortalized B cells. EBV-immortalization of human B cells is known to the skilled artisan (for example, Traggiai et al Methods Mol Biol 2012). Immortalized, autologous B cells are used to generate antigen-expressing B cells by electroporation of B cells with TMG-mRNA. Electroporation of antigen-presenting cells (APCs) has been described previously and is known to the skilled artisan. An ordinary person skilled in the art will appreciate that also other methods will allow to immortalize autologous B cells and to induce antigen expression by such autologous, immortalized B cells, for example pulsing with peptide, transfection with TMG-encoding DNA plasmids or transduction with TMG-encoding viral particles.

The TCR library is transfected into amphotropic virus producer cells such as Phoenix-Ampho (ATCC CRL-3213) by methods known to the skilled artisan. The resulting retroviral virions are used to transduce a Jurkat reporter T cell line. The Jurkat reporter T cell line lacks endogenous TCR expression (for example described in Mezzadra et al Nature 2017) and is modified to express human CD8α and CD8β after transduction with a CD8α-P2A-CD8β transgene (SEQ ID NO: 2) using methods known to the skilled artisan. Jurkat reporter T cells are transduced with the TCR library using a low MOI in order to limit the frequency of TCR transduced Jurkat T cells to 25-30% of total T cells. Jurkat T cells modified to express a TCRβ-P2A-TCRα-T2A-Puromycin resistance transgene are positively selected by addition of Puromycin to the cell culture media. Antibiotic selection of genetically modified cells is known to an ordinary person skilled in the art. An ordinary person skilled in the art will further appreciate that the methods described herein can include selection without puromycin, such as sorting of TCR-transduced cells by FACS or magnetic bead-based selection, for example. Thus, a TCR cassette may lack the puromycin selection gene.

TCR transduced Jurkat T cells are stimulated by antigen-loaded B cells for 6 hours using the following conditions:
1. 4e⁷ TCR transduced Jurkat T cells+4e⁷ antigen-loaded autologous B cells
2. 4e⁷ TCR transduced Jurkat T cells+4e⁷ autologous B cells

Subsequently, co-cultures are harvested and stained with fluorochrome-labeled antibodies for CD4, CD8 and CD69. Using a BD Biosciences ARIAFusion flow cytometry sorter the following populations are sorted for each stimulation condition:
1. Live, single cell, CD4⁺, CD8⁺, CD69^{hi} (CD69^{hi} includes the highest 5-50% of live, single cell, CD4⁺, CD8⁺ cells based on CD69 fluorescence signal)
2. Live, single cell, CD4⁺, CD8⁺, CD69^{lo} (CD69^{lo} includes 5-50% of the live, single cell, CD4⁺, CD8⁺ cells based on a low CD69 fluorescence signal)

Alternatively, the following stimulation conditions can be used for each TCR library respectively:
1. 4e⁷ TCR transduced Jurkat T cells+4e⁷ K562-HLA-A2 loaded with 1 ug of neo-antigen peptide
2. 4e⁷ TCR transduced Jurkat T cells+8e⁷ K562-HLA-A2 loaded with 1 ug of neo-antigen peptide
3. 4e⁷ TCR transduced Jurkat T cells+4e⁷ K562-HLA-A2 loaded with 1 ug of irrelevant control peptide
4. 4e⁷ TCR transduced Jurkat T cells+8e⁷ K562-HLA-A2 loaded with 1 ug of irrelevant control peptide

Subsequently, co-cultures are harvested and stained with fluorochrome-labeled antibodies for CD4, CD8 and CD69. Using a BD Biosciences ARIAFusion flow cytometry sorter live, single cell, CD4⁺, CD8⁺, CD69⁺ cells are sorted for each sample.

Genomic DNA is isolated from the sorted TCR transduced. Jurkat T cells and used as template for a PCR to amplify part of the TCRβ-P2A-TCRα cassette. The resulting PCR product has a size of approx. 1.5 kb and can be sequenced using an Oxford Nanopore Minton sequencer to compare the relative abundance of neo-antigen specific TCR sequences in the sorted T cell populations (either. CD69^{lo} and CD69^{hi} or CD69⁺).

In some embodiments, the Oxford Nanopore Minion sequencer may be replaced with some other sequencing instrument or other sequencing strategies may be employed.

### Example 8

This Example describes creating a TCR repertoire using gene synthesis.

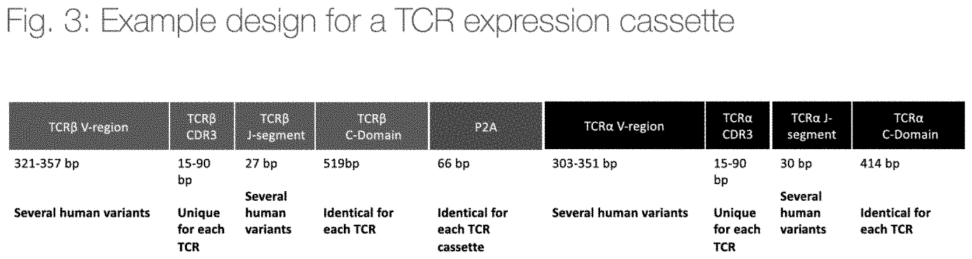

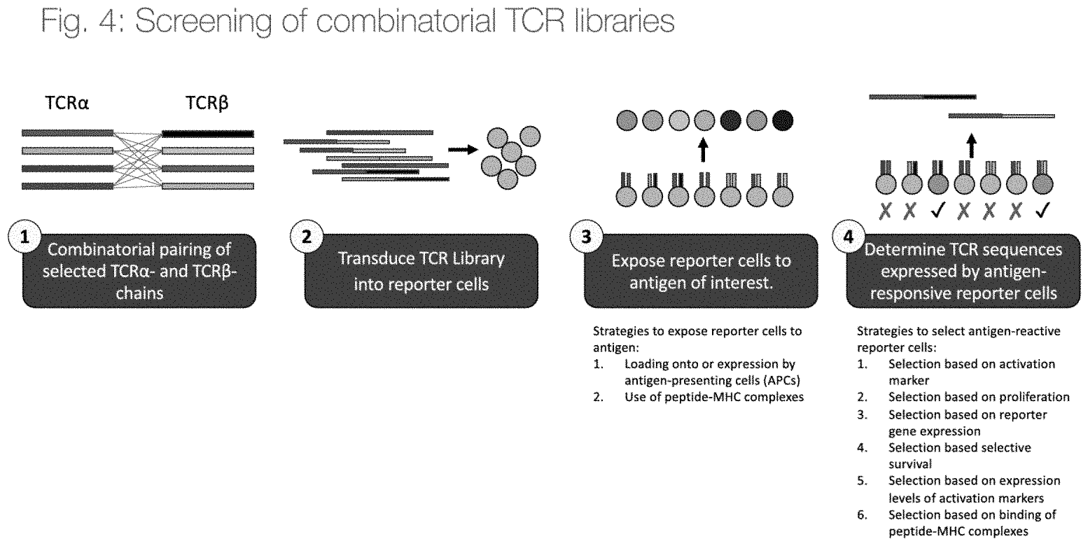

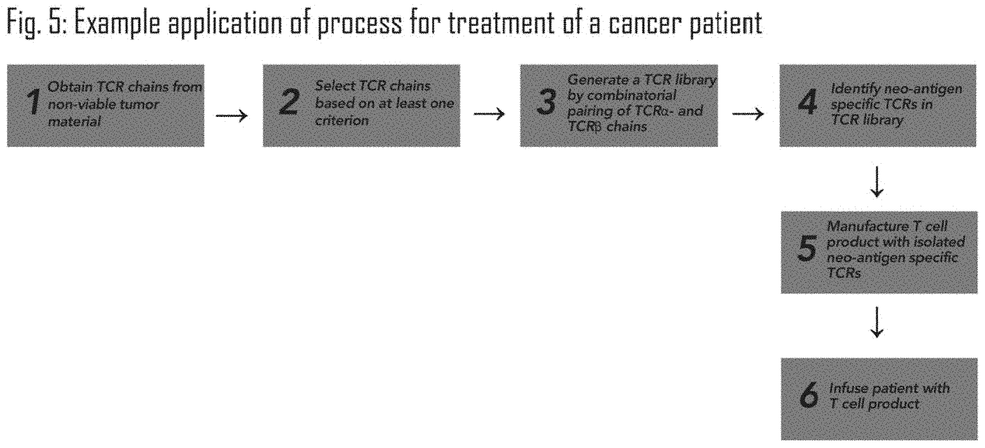

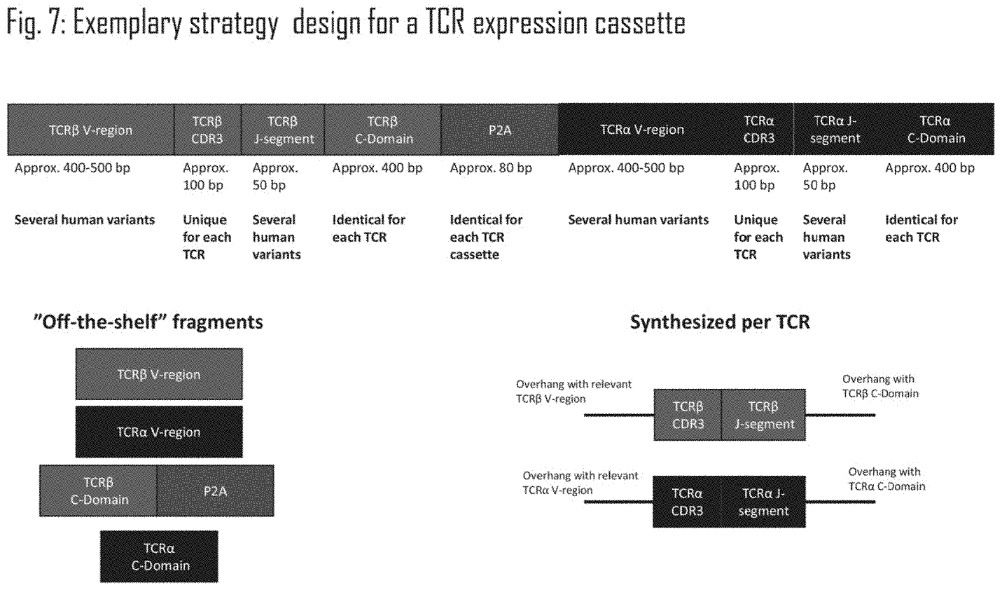

A TCR can be expressed in a cassette design as provided in FIG. 3. In order to synthesize high numbers of such TCR cassettes highly variable parts of each FOR chain are synthesized as fragments, such as CDR3-J-segments, for example. Other components can be “off-the-shelf” building blocks. In order to recreate all TCR chains of interest, the variable/unique fragments are synthesized and mixed with “off-the-shelf” building blocks. The principle is depicted in FIG. 7. Subsequently, all components are mixed together and assembly will create a TCR cassette as depicted in FIG. 3. The assembly is controlled by specific sequence overhangs: specific CDR3-J-segments will only fuse to certain V-segments and because of overlap between the different fragments every TCR cassette will ultimately be in the format TCRβ-P2A-TCRα. Every TCRα can pair with every TCRβ fragment. Finally, all TCRβ-P2A-TCRα cassettes will be cloned into an expression vector. In some situations, V-CDR3-J (both for alpha and beta chains) ,s the variable parts and Cbeta-P2A as the off the shelf building block can be used.

Alternatively, any and all possible TCRβ-P2A-TCRα combinations are generated fully by gene synthesis. In this way, it is possible to combinatorially pair all TCR and TCRβ fragments.

Yet another method includes generation of TCRα and TCRβ fragments by combinatorial synthesis or by gene synthesis, as described above. However, instead of generating a TCRβ-P2A-TCRα cassette, the resulting collections of TCRα and TCRβ chains are cloned into separate expression vectors. Cells are modified with the vector collections in such a way that every T cell on average expresses one TCRα and one TCRβ, resembling combinatorial pairing as described above.

As an alternative method, modified TCRs, such as single-chain TCR constructs fused with CD3ϵ or CD3ζ signaling domains alone or in combination with a CD28 signaling domain, can be employed, instead of just TCRα and TCRβ.

### Example 9

This example describes identification of TCRαβ pairs from the TCR repertoire.

A pool of T cells modified with the library of generated TCRαβ pairs is stimulated by antigen presenting cells presenting at least one antigen of interest and antigen-reactive T cells are isolated based on at least one activation marker for TCR isolation. Alternatively, the pool of T cells is labelled with a fluorescent dye suitable to trace cell proliferation, stimulated by antigen presenting cells expressing at least one antigen of interest, and antigen-reactive T cells are isolated based on proliferation for TCR isolation. Purification of activated T cells can be achieved by antibody-labelling and subsequent isolation based on flow cytometry sorting, magnetic bead based selection or any other antibody-binding based selection method.

In yet another method, a pool of T cells modified with the library of generated TCRαβ pairs is divided into at least two samples. Samples are stimulated by antigen presenting cells expressing at least one antigen of interest or not. After stimulation, both T cell populations are incubated for a period of time and subsequently both T cell populations are analyzed by TCR isolation. Comparison of TCRαβ pairs obtained from both samples will identify TCR genes with higher abundance in the sample exposed to at least one antigen. Detection of proliferation can be based on detection of dilution of a fluorescent dye such as CFSE or Cell Tracer Violet. Proliferating cells are sorted based on a diluted fluorescence signal by flow cytometry.

In a further method, a pool of T cells modified with the library of generated TCRαβ pairs is stimulated by antigen presenting cells presenting at least one antigen of interest and antigen-reactive T cells are isolated based on at least one reporter gene, such as NFAT-GFP or NFAT-YFP that reports on TCR triggering.

In yet another method, a pool of T cells modified with the library of generated TCRαβ pairs is stimulated by antigen presenting cells presenting at least one antigen of interest, and antigen-reactive T cells are isolated for TCR isolation using selection of antigen-specific T cells based on acquired antibiotic resistance upon TCR signaling, for example by use of a NFAT-puromycin transgene. Alternatively, a pool of modified T cells is exposed to one or multiple MHC complexes that carry an antigen of interest. T cells that bind to a MHC complex are isolated for TCR isolation. Isolation based on MHC-complex binding may be performed by flow cytometry sorting or magnetic bead enrichment.

TCR isolation in any of the above methods can be achieved by (i) DNA or isolation from bulk antigen-reactive T cells to generate TCRαβ specific PCR products which are analyzed by DNA-sequencing or RNA sequencing to determine TCRαβ gene sequences of antigen-reactive T cells or (ii) single-cell based droplet PCR or microfluidic approaches to analyze the TCRαβ gene sequences expressed in analyzed single T cells. In this manner, single T cells within the pool of T cells in which TCRαβ transcripts are co-expressed with increased levels of activation marker are detected.

In a further method, a pool of T cells modified with the library of generated TCRαβ pairs is stimulated by antigen presenting cells expressing at least one antigen of interest. Subsequently, TCRαβ pairs of interest are identified using single-cell based droplet PCR or microfluidic approaches to combine TCR isolation with the detection of transcript levels for at least one activation marker. Thereby, single T cells within the pool of T cells in which TCRαβ transcripts are co-expressed with increased levels of one or more activation markers are detected.

### Example 10

This example describes a screening method for identifying a functional TCRα/TCRβ combination from a combinatorial library of nucleic acid sequences encoding variant TCRα and TCRβ polypeptides.

In order to identify neo-antigen specific TCRs from non-viable tumors, a process to generate TCR libraries by combinatorial assembly of tumor-derived TCRα and TCRβ chains was developed. These TCR chains are identified by TCR bulk chain sequencing of DNA or RNA isolated from tumor tissue. TCRαβ pairs are encoded as transgenes of approx. 1.5 kb and introduced into reporter cells. By stimulation with antigen-expressing cells, reporter cells expressing antigen-reactive TCRαβ combinations can be selected in a genetic variant library screening. Given the combinatorial assembly, each TCR variant can only be unambiguously identified by determining both TCRα and TCRβ variable sequences. Hence, transgenes encoded in reporter cells isolated during the genetic screen are recovered as PCR amplicons of approx. 1.5 kb, sequenced in full length using Oxford Nanopore sequencing and analyzed using a bioinfoiniatic analysis pipeline.

This process can identify TCR leads that can be further evaluated for potential use in cancer therapy.

### 1. Generation of a Library of Variant Nucleotide Sequences

TCRα and TCRβ chains are selected as described in one or more of the examples herein, and combinatorially paired, thereby generating a total set of TCR variants. Because of the combinatorial pairing, any given TCR variant can only be unambiguously identified by determining the TCRα and TCRβ variable sequences. This may involve sequencing a PCR amplicon of about 1.5 kb.

2. Introducing the Library of Variant Nucleotide Sequences into Reporter Cell

A combinatorial TCR library described in step 1) is transfected into virus-producing Phoenix-ampho cells to produce retrovirus encoding all TCR variants present in the library. The virus is subsequently used to transduce Jurkat T cells lacking endogenous TCR expression. A blasticidin selection marker is co-encoded within the TCR library, which allows antibiotic selection of transduced reporter T cells expressing a pool of TCRs.

### 3. Selection of Reporter Cells Based on at Least One Functional Property

A polyclonal mix of reporter T cells expressing a variety of TCRs are seeded at low density, and subsequently co-cultured with immortalized B cells expressing potential neo-antigens. The amount of reporter T cells is such that all TCR variants are represented at an average coverage of at least 100. Subsequently, cocultures are harvested and subjected to FACS-sorting based on either high or low expression of the T cell activation marker CD69. These respective ‘top’ and ‘bottom’ populations are harvested and further analyzed. Other activation markers may be used for FACS-sorting, where CD62L, CD137, IFN-γ, IL-2, TNF-α and GM-CSF may either replace or be combined with CD69 to select for activated reporter T cells. In addition, various promoter activity reporters may be used to select activated cells.

4. Isolation of Variant Nucleotide Sequences from Selected Reporter Cells

Genomic DNA is isolated and subjected to PCR-amplification of the retroviral insert encoding a TCR. PCR primers are in the retroviral vector and in the constant region of the TCR alpha chain, yielding an arnplicon of about 1500 bases. Sufficient genomic DNA is used to represent all TCR variants at an average coverage of at least 100. Amplification is minimized to prevent biases in amplification of specific TCR variants, but should yield an average coverage of at least 10000 for each TCR represented in the library. TCR amplification from genomic DNA is performed for both top and bottom samples.

### 5. Determine at Least 600 bp of the Total Nucleotide Sequence of the Isolated Variant Nucleotide Sequences

Amplified TCR sequences are further processed for Oxford Nanopore sequencing. In a first PCR-reaction, tailed primers are used. These contain a new binding site for a second PCR with barcoded outer primers modified with rapid attachment chemistry. Distinct barcodes are used for the PCRs on top and bottom samples. In all PCR steps, amplification is minimized to prevent biases in amplification of specific TCR variants. However, sufficient PCR product is used to represent all TCR variants at an average coverage of at least 10000.

In the alternative to the above, amplified TCR sequences are further processed for Oxford Nanopore sequencing. In a first PCR-reaction, TCR cassettes are amplified with untailed primers. In a subsequent PCR round, tailed primers are used. These contain a new binding site for a third PCR with barcoded outer primers modified with rapid attachment chemistry. Distinct barcodes are used for the PCRs on top and bottom samples. In all PCR steps, amplification is minimized to prevent biases in amplification of specific TCR variants. However, sufficient PCR product is used to represent all TCR variants at an average coverage of at least 10000. An ordinary person skilled in the art will recognize that alternative PCR amplification strategies may be employed.

Barcoded PCR products from top and bottom samples are pooled in equimolar ratios and in a final step rapid 1D sequencing adapters are ligated onto this pool to yield a library preparation that is ready for sequencing. This library is loaded onto an Oxford Nanopore R9.4.1 flow cell and sequenced up to an average coverage of at least 100 reads for every TCR encoded in the library.

For bioinformatic analyses, the Oxford Nanopore guppy toolkit is used. Sequence reads are retrieved from raw data using guppy_basecaller. Samples are demultiplexed using guppy_barcoder. Alternatively, for GridIon-based sequencing, demultiplexed sequence reads are obtained using the MinKnow software package. Sequence reads are aligned to a reference consisting of individual alpha and beta chain sequences using guppy_aligner, and alpha and beta chain identity for each read is extracted from the resulting barn alignment files. In a final step, the frequency of occurrence of each TCR is calculated and used for further analysis.

### 6. Selection of at Least One Variant Nucleotide Sequence of Interest

In order to identify a variant nucleotide sequence of interest, TCRs are selected based on relative enrichment by determining variant enrichment in the positively selected (marker molecule positive) reporter cell population and variant depletion in the negatively selected (marker molecule negative) reporter cell population as determined by variant read counts in both cell populations.

### Example 11

This example describes a screening method for identifying a functional CAR variant from a combinatorial library of nucleic acid sequences encoding variant CAR protein domains.

in order to identify CAR designs with optimal functional properties, the CAR molecule domains can be assembled in combinatorial fashion. A CAR molecule is comprised of (i) an antigen-binding domain, (ii) a hinge domain, (iii) a transmembrane domain and (iv) an intracellular signaling domain (usually comprised of 2-3 signaling modules) creating a synthetic molecule of approx. 1.5 kb. The library of CAR variants can be introduced into reporter cells and by stimulation with antigen-expressing cells, reporter cells expressing a CAR variant leading to the desired activation phenotype can be selected in a genetic screening. Given the combinatorial assembly of several molecule domains, each variant can only be unambiguously identified by determining the sequence of all variable molecule parts. Hence, transgenes encoded in reporter cells isolated during the genetic screen are recovered as PCR amplicons of approx. 1.5 kb, sequenced in full length using Oxford Nanopore sequencing and analyzed using a customized bioinformatic analysis pipeline.

This process can identify CAR leads that can be further evaluated for potential therapeutic use, e.g. in cancer.

### 1. Generation of a Library of Variant Nucleotide Sequences

A library of CAR variants is generated by combinatorial assembly of several CAR protein domains: 2 hinge domains, 12 transmembrane domains and 13 signaling domains (with 3 signaling domains incorporated in each variant) generating a library with more than 50,000 protein variants. In order to unambiguously identify any given CAR variant a PCR amplicon of 1.3 kb can be sequenced.

2. Introducing the Library of Variant Nucleotide Sequences into Reporter Cell

A CAR variant library described in step 1 is transfected into virus-producing Phoenix-ampho or 293T cells to produce retro- or lentivirus, respectively, encoding all CAR variants present in the library. The virus is subsequently used to transduce immortalized Jurkat T cells or in vitro-activated primary human T cells. A cell surface marker and/or antibiotic selection marker is co-encoded within the CAR variant library, which allows selection of transduced reporter T cells expressing a pool of CAR variants.

### 3. Selection of Reporter Cells Based on at Least One Functional Property

A polyclonal mix of reporter T cells expressing a library of CAR variants is labeled with a cell proliferation dye, seeded at low density, and co-cultured with antigen-presenting cells expressing the cognate ligand of the CAR antigen-binding domain. The amount of reporter T cells used is such that all CAR variants are represented at an average coverage of at least 100. Subsequently, cocultures are harvested and subjected to flow cytometry sorting based on T cells that have divided at least once or that have not divided. These respective ‘top’ and ‘bottom’ populations are harvested and further analyzed. Other activation markers may be used for flow cytometry-based sorting of responding and non-responding T cells, such as CD69, CD137, IFN-γ, IL-2, TNF-α and GM-CSF, either alone or in combination. In addition, various transcription factor activity reporters (NF-κB, NFAT, AP-1), signal transduction reporters (ZAP70, ERK1/2 phosphorylation) or cytotoxicity reporters (CD107A expression) may be used to select responding and non-responding T cells.

4. Isolation of Variant Nucleotide Sequences from Selected Reporter Cells

From selected CAR variant-expressing reporter T cells, genomic DNA is isolated and subjected to PCR-amplification of the retro- or lentiviral inserts encoding a CAR. PCR primers bind to an invariable region of the CAR insert, yielding an average amplicon size of about 1300 bases.

Alternatively, a similar Unique Molecular Identifiers (UMI)-based approach may be applied. Sufficient genomic DNA is used to represent all CAR variants at an average coverage of at least 100. The amount of PCR cycles is minimized to prevent biases in amplification of specific CAR variants, but should yield an average coverage of at least 10000 for each CAR represented in the library. CAR variant amplification from genomic DNA is performed for both responding and non-responding reporter T cells (top and bottom samples).

### 5. Determine at Least 600 bp of the Total Nucleotide Sequence of the Isolated Variant Nucleotide Sequences

Amplified CAR sequences are further processed for Oxford Nanopore sequencing. In a first PCR-reaction, tailed primers are used. These contain a new binding site for a second PCR with barcoded outer primers modified with rapid attachment chemistry. Distinct barcodes are used for the PCRs on top and bottom samples. In all PCR steps, amplification is minimized to prevent biases in amplification of specific CAR variants. However, sufficient PCR product is used to represent all CAR variants at an average coverage of at least 10000.

Barcoded PCR products from top and bottom samples are pooled in equimolar ratios and in a final step rapid 1D sequencing adapters are ligated onto this pool to yield a library preparation that is ready for sequencing. This library is loaded onto an Oxford Nanopore R9.4.1 flow cell and sequenced up to an average coverage of at east 100 reads for every CAR encoded in the library.

For bioinformatic analyses, the Oxford Nanopore guppy toolkit is used. Sequence reads are retrieved from raw data using guppy_basecaller. Samples are demultiplexed using guppy_barcoder. Sequence reads are aligned to a reference consisting of individual CAR variant sequences using guppy_aligner, and CAR variant identity for each read is extracted from the resulting bam alignment files. In a final step, the frequency of occurrence of each CAR variant is calculated and used for further analysis. As an option to circumvent amplification bias from PCRs, UMI-based counting of CAR variants may be applied.

### 6. Selection of at Least One Variant Nucleotide Sequence of Interest

In order to identify a variant nucleotide sequence of interest, CARs are selected based on relative enrichment by determining variant enrichment in the positively selected (marker molecule positive) reporter cell population and variant depletion in the negatively selected (marker molecule negative) reporter cell population as determined by variant read counts in both cell populations.

### Example 12

This example describes calculation of the number of cells that are screened by the present screening methods as described in Examples 10 and 11.

For a combinatorial library having a size of 100 combinations, 100× coverage can be achieved by recovering 10,000 cells upon selection for positive responders, and recovering 10,000 negative responders. Thus, the population of cells before selection can be greater than 20,000.

For a combinatorial library having a size of 1000 combinations, 100× coverage can be achieved by recovering 100,000 cells upon selection for positive responders, and recovering 100,000 negative responders. Thus, the population of cells before selection can be greater than 200,000.

For a combinatorial library having a size of 1×10⁹ combinations, 100× coverage can be achieved by recovering 1×10¹¹ cells upon selection for positive responders, and recovering 1×10¹¹ negative responders. Thus, the population of cells before selection can be greater than 2×10¹¹.

### Example 13

This example describes the recovery of neo-antigen specific T cell receptor sequences from Mismatch-Repair-proficient colorectal cancer (MMRp-CRC) tumors (FIG. 10A).

DNA and RNA were isolated from four fresh-frozen MMRp-CRC tumor specimens and used in the following two ways:

First, DNA and/or RNA was utilized to perform bulk TCRα- and β-chain sequencing (performed by MiLaboratory; Moscow/Russia). The resulting collection of TCR chain sequences was divided into a collection of TCRα- and a collection of TCRβ-chain sequences leading to collections of approx. 10,000-30,000 TCRα- and TCRβ-chains per sample (FIGS. 10B and 11A).

FIG. 10B and FIG. 11A depict bulk TCR sequencing of infiltrating lymphocytes in human tumor samples. Four human MMRp-CRC tumor samples were subjected to bulk TCR sequencing by Milaboratory. After alignment and TCR identification, clonotypes were collapsed based on their CDR3 amino acid sequence and their V and J identity. The number of unique clonotypes are represented for both alpha and beta chains for each tumor sample.

Any non-productive TCR chain sequences, in which TCR segments (also known as TCR gene elements) are joined out of frame at the amino acid sequence level, and/or in which stop codons are introduced, and/or in which frameshift mutations are present, and or in which defective splicing sites are present, were removed from the collection. Each collection was sorted in descending order using either read counts or unique molecular identifier (UMI) counts of nucleic acid molecules encoding a particular TCR chain (or corresponding percentage among total TCRα- or TCRβ-chains, respectively) to obtain a rank order for TCRα- and β-chains.

The Top 100 most abundant TCRα- and β-chains were selected for TCR library generation (performed by Twist Bioscience; San Francisco/USA). In brief, the selected TCRα- and β-chains were generated as fragments by DNA synthesis. For library generation all selected TCRα and TCRβ fragments were combinatorially joined to continuous nucleic acid molecules encoding TCRβ-P2A-TCRα cassettes (FIG. 11B Schematic representation of the TCR expression plasmid. A retroviral construct containing both beta and alpha TCR chains, as well as a blasticidin selection marker can be used as a scaffold for creating the library). Retroviral transduction using this construct ultimately lead to expression of a single transcript, which resulted in translation of the TCR beta and alpha chains, as well as a blasticidin resistance marker, due to peptide cleavage at the 2A sites). One TCRβ- and TCRα-fragment can be joined per cassette in the format of TCRβ-P2A-TCRα-T2A-Blasticidin resistance (FIG. 11B) (SEQ ID NO: 4) (FIG. 43). The resulting TCR libraries generally contained 10,000 TCR variants without drop-outs in a very narrow frequency range (FIG. 10C; FIG. 11C and 11D) Quality control of 100×100 libraries. The 100 most prevalent alpha and beta chains for each sample from 11A) were selected and used for creating a combinatorial library. The TCR-beta, and TCR-alpha regions in 11B) were synthesized and inserted into the vector in 11B) in a combinatorial cloning approach executed by Twist Bioscience. A primer pair flanking the variable TCR alpha and beta chain domains was used to amplify both chains, and Nanopore sequencing was used to identify the identity of both chains.

The representation of each of the 10,000 alpha x beta combinations was represented for every patient library (FIG. 11D). Characteristics of the TCR representations of the patient libraries. For each patient library in 11C) the range of the amount of reads per TCR, the mean coverage, and the percentage of TCRs that fall within a range of the median +/″ a ²log-unit are represented. Second, tumor-derived as well as healthy tissue DNA and RNA was used to determine the set of tumor-specific mutations using Whole-exome-sequencing (WES) and RNA sequencing to establish the set of expressed mutated genes. A pipeline to identify and select tumor-specific mutations can provide options. Methods to identify such mutations based on WES and RNA-seq have been described previously and are known to a person skilled in the art. Subsequently, tandem-minigene (TMG) constructs were generated that encode multiple tumor-derived mutated peptides in tandem arrays. TMG constructs encode 12 individual tumor mutations as 25 mer polypeptide minigenes which were concatenated and included LAMP-1 signaling and transmembrane domains (e.g. described in Gros et. Nat Med 2016; except for pt4) and a puromycin resistance marker fused to the LAMP-1 cytoplasmic domain using a 2A-element. For Pt4, tandem-minigene designs encoding 33 or 34 different concatenated minigenes without LAMP-1 signaling and transmembrane domains but containing a puromycin resistance marker are used. In parallel, matched autologous Hood from the MMRp-CRC patient was used to generate immortalized B cells. EBV-immortalization of human B cells is known to the skilled artisan (for example, Traggiai et al Methods Mol. Biol 2012). Immortalized, autologous B cells were used to generate antigen-expressing B cells by retroviral transduction with TMG-encoding viral particles. Protocols for retroviral transduction of primary human B cells with other genes than TMGs have been described in the literature (e.g. Kwakkenbos et al. Nat Med 2010) and are known to the skilled artisan. TMG-transduced B cells can be selected based on puromycin resistance. The process for puromycin selection is known to the skilled artisan. The TCR library was transfected into Phoenix-Ampho virus producer cells (ATCC CRL-3213) using Eugene transfection reagent and protocols known to the skilled artisan. The resulting retroviral virions were used to transduce a Jurkat reporter T cell line. The Jurkat reporter T cell line lacks endogenous TCR expression (generation of such a genetic knock-out being described for example in Mezzadra et al Nature 2017) and is modified to express human CD8α and CD8β after transduction with a CD8α-P2A-CD8β transgene (SEQ ID NO: 2) using methods known to the skilled artisan. Jurkat reporter T cells were transduced with the TCR libraries resulting in 10-60% TCR-modified T cells of total live T cells (FIG. 10D). The use of murine TCR constant domain sequences in the TCR library allowed for the detection of TCR-modified Jurkat T cells by flow cytometry using a murine TCRβ constant domain specific antibody. Jurkat T cells modified to express a TCRβ-P2A-TCRα-T2A-Blasticidin resistance transgene were positively selected to high purity by addition of Blasticidin to the cell culture media (FIG. 10D). Antibiotic selection of genetically modified cells is known to an ordinary person skilled in the art.

Next, TCR transduced Jurkat T cells were stimulated by co-culturing with TMG-transduced B cells. Three days prior to the co-culture Jurkat reporter T cells were seeded at a low density (0.1×10⁶ cells per ml). B cells expressing various TMG were all mixed at an equal (1:1) ratio. 90×10⁶ pooled B cells (or control B cells that lack TMG expression) were mixed with 90×10⁶ Jurkat reporter T cells (1:1 ratio) in 72 ml total volume of medium. 200 ul (0.5×10⁶ cells per well) of this mix was distributed over ˜360 wells of U-bottom TC-treated 96-well plates. Plates were centrifuged at 1000 rpm for 1 minute, and incubated for 20-22 hours at 37° C.

Subsequently, co-cultures were harvested and stained with fluorochrome-labeled antibodies for CD20 and CD69. Next, the cells were fixed using a fixation buffer containing 4% formaldehyde. Using a BD Biosciences ARIAFusion flow cytometry sorter the following populations were sorted for each stimulation condition (FIG. 10E):

Lymphocyte, single cell, CD20⁻, CD69^{hi} (CD69^{hi} includes the highest 10-15% of single cell, CD20⁻ cells based on CD69 fluorescence signal)

Lymphocyte, single cell, CD20⁻, CD69^{lo} (CD69^{lo} includes 10-15% of the single cell, CD20⁻ cells based on a low CD69 fluorescence signal)

Genomic DNA was isolated from the sorted TCR transduced Jurkat T cells and used as template for multiple rounds of PCR with a limited amount of cycles to amplify part of the TCRβ-P2A-TCRα cassette using PCR methods known to the skilled artisan. The resulting PCR product has a size of approx. 1.5 kb (FIG. 10F) and were sequenced using an Oxford Nanopore MinIon or GridIon sequencing instrument. The entire screening procedure up to this point is carried out at a minimum coverage of 100×, and for three replicates in the context of TMG expression, and for three replicates in the absence of TMG expression. TCR alpha and beta chain identities are recovered and differentially expressed TCR combinations are identified using the DESeq2 R package. Average Rlog-transformed read counts in the presence and absence of TMG-expression in B cells can be used to depict neo-antigen reactive TCR leads. Neo-antigen reactive TCR leads are depicted as encircled larger black dots (FIG. 10G).

In order to deconvolute the TMG recognized by the TCR leads, TCR libraries were screened using B cells expressing a single TMG construct (rather than mixtures of TMG-expressing B cells) in a single replicate (FIG. 10H). Subsequently, to determine the exact neo-antigen recognized by the TCR lead, B cells loaded with single 25mer peptides that are encoded in the TMG were used (FIGS. 10I and 10J). The exact neo-antigen recognized by the TCR lead was determined using B cells expressing single minigenes that are encoded in the TMG (FIG. 10K and 10L).

Taken together, this example shows that the described platform can successfully identify neo-antigen specific TCRs from fresh-frozen tumor material.

### Example 14

This example describes the recovery of TCR sequences from melanoma tumor samples for the generation of patient-specific TCRαβ libraries (FIG. 12A-12C).

DNA and RNA were isolated from two fresh-frozen melanoma tumor specimens and used in the following two ways:

First, DNA and/or RNA were utilized to perform bulk TCRα- and β-chain sequencing (performed by MiLaboratory; Moscow/Russia) of infiltrating lymphocytes in the tumor samples. The resulting collection of TCR chain sequences was divided into a collection of TCRα- and a collection of TCRβ-chain sequences leading to collections of approx. 5,000-10,000 TCRα- and TCRβ-chains per sample (FIG. 12A). Two human melanoma tumor samples were subjected to bulk TCR sequencing by Milaboratory. After alignment and TCR identification, clonotypes were collapsed based on their CDR3 amino acid sequence and their V and J identity. The number of unique clonotypes are represented for both alpha and beta chains for each tumor sample.

Any non-productive TCR chain sequences, in which TCR segments (also known as TCR gene elements) are joined out of frame at the amino acid sequence level, and/or in which stop codons are introduced, and or in which frameshift mutations are present, and/or in which defective splicing sites are present, were removed from the collection. Each collection was sorted in descending order using either read counts or unique molecular identifier (UMI) counts of nucleic acid molecules encoding a particular TCR chain (or corresponding percentage among total TCRα- or TCRβ-chains, respectively) to obtain a rank order for TCRα- and β-chains. The Top 100 most abundant TCRα- and β-chains are selected for TCR library generation (performed by Twist Bioscience; San Francisco/USA). In brief, the selected TCRα- and β-chains are generated as fragments by DNA synthesis. For library generation all selected TCRα and TCRβ fragments are combinatorially joined to continuous nucleic acid molecules encoding TCRβ-P2A-TCRα cassettes (FIG. 11B Schematic representation of the TCR expression plasmid. A retroviral construct containing both beta and alpha TCR chains, as well as a blasticidin selection marker can be used as a scaffold for creating the library. Retroviral transduction using this construct ultimately leads to expression of a single transcript, which results in translation of the TCR beta and alpha chains, as well as a blasticidin resistance marker, due to peptide cleavage at the 2A sites). One TCRβ- and TCRα-fragment can be joined per cassette in the format of TCRβ-P2A-TCRα-T2A-Blasticidin resistance (FIG. 11B) (SEQ ID NO: 4). The resulting TCR libraries will generally contain 10,000 TCR variants without drop-outs in a very narrow frequency range (FIG. 12B). Quality control of 100×100 libraries. The 100 most prevalent alpha and beta chains for each sample from 12A) can be selected and used for creating a combinatorial library. The TCR-beta and TCR-alpha regions in 12A) can be synthesized and inserted into the vector in 11B) in a combinatorial cloning approach executed by Twist Bioscience. A primer pair flanking the variable TCR alpha and beta chain domains can be used to amplify both chains, and Nanopore sequencing was used to identify the identity of both chains. The representation of each of the 10,000 alpha x beta combinations is represented for every patient library. 12C) Characteristics of the TCR representations of the patient libraries. For each patient library in 12B) the range of the amount of reads per TCR, the mean coverage, and the percentage of TCRs that fall within a range of the median +/− a ²log-unit are represented.

Taken together, this example shows that combinatorial TCR libraries can be synthesized and cloned based on bulk TCR sequencing.

### Example 15

This example describes Recovery of antigen-specific TCRs from a TCR library generated by mixing TCR plasmids.

A TCR library was generated by mixing 6 plasmids each encoding a single characterized TCR with 24 plasmids each encoding a single uncharacterized TCR each (FIG. 13A). TCR expression plasmid design is depicted in FIG. 15A. This TCR library was transfected into Phoenix-Ampho virus producer cells (ATCC CRL-3213) using Fugene transfection reagent and protocols known to the skilled artisan. The resulting retroviral virions were used to transduce a Jurkat reporter T cell line. The Jurkat reporter T cell line lacks endogenous TCR expression (generation of such a genetic knock-out being described for example in Mezzadra et al Nature 2017) and is modified to express human CD8α and CD8β after transduction with a CD8α-P2A-CD8β transgene (SEQ ID NO: 2) using methods known to the skilled artisan. Jurkat reporter T cells were transduced with the TCR library resulting in 80% TCR-modified T cells of total live T cells. The use of murine TCR constant domain sequences in the TCR library (FIG. 15A) allows for the detection of TCR-modified Jurkat cells by flow cytometry using a murine TCRI3 constant domain specific antibody. Jurkat T cells modified to express a TCRβ-P2A-TCRα-T2A-Puromycin resistance transgene were positively selected to high purity by addition of Puromycin to the cell culture media (FIG. 13B). Antibiotic selection of genetically modified cells is known to an ordinary person skilled in the art.

Next, TCR transduced Jurkat T cells were stimulated by co-culturing with TMG-transduced B cells. Three days prior to the co-culture Jurkat reporter. T cells were seeded at a low density (0.1×10⁶ cells per ml). 90×10⁶ pooled B cells expressing TMG (or control B cells that lack TMG expression) were mixed with 90×10⁶ Jurkat reporter T cells (1:1 ratio) in 72 ml total volume of medium. 200 ul (0.5×10⁶ cells per well) of this mix was distributed over ˜360 wells of U-bottom TC-treated 96-well plates. Plates were centrifuged at 1000 rpm for 1 minute, and incubated for 20-22 hours at 37° C.

Subsequently, co-cultures were harvested and stained with fluorochrome-labeled antibodies for CD2β and CD69. The cells were then fixed using a fixation buffer containing 4% formaldehyde. Using a BD Biosciences ARIAFusion flow cytometry sorter the following populations were sorted for each stimulation condition (FIG. 13C):
1. Lymphocyte, single cell, CD20⁻, CD69^{hi} (CD69^{hi} includes the highest 10-15% of single cell, CD20⁻ cells based on CD69 fluorescence signal)
2. Lymphocyte, single cell, CD20⁻, CD69^{lo} (CD69^{lo} includes 10-15% of the single cell, CD20⁻ cells based on a low CD69 fluorescence signal)

Genomic DNA was isolated from the sorted TCR transduced Jurkat T cells and used as template for multiple rounds of PCR with a limited number of cycles to amplify part of the TCRβ-P2A-TCRα cassette using PCR methods known to the skilled artisan. The resulting PCR product has a size of approx. 1.5 kb (FIG. 13D) and can be sequenced using an Oxford Nanopore Minion or Gridlon sequencing instrument using techniques known to a person skilled in the art. TCR identities were recovered using alignment techniques known to the skilled artisan, and for each TCR the log2-transformed fold enrichment of normalized read counts in the top versus bottom samples is represented as a function of the log10-transformed TCR frequency (FIG. 13E; single replicate in the context of TMG expression; single replicate in the absence of TMG expression).

Taken together, this example shows that antigen-reactive TCRs can be isolated from a mix of TCR plasmids.

### Example 16

This example describes recovery of antigen-specific TCRs from a TCR library generated by gene synthesis.

Five characterized TCRs of known antigen reactivity, as well as 45 or 95 uncharacterized TCRs (for the 50×50 and 100×100 libraries, respectively; FIG. 14A) were selected for TCR library generation (performed by Twist Bioscience; San Francisco/USA). In brief, the TCRα- and β-chains from these TCRs were synthesized as fragments by DNA synthesis. For library generation all selected TCRα and TCRβ fragments were combinatorially joined to continuous nucleic acid molecules encoding TCRβ-P2A-TCRα cassettes (SEQ ID 1). A retroviral construct containing both beta and alpha. TCR chains, as well as a puromycin selection marker were used as a scaffold for creating the library. Retroviral transduction using this construct ultimately leads to expression of a single transcript, which results in translation of the TCR beta and alpha chains, as well as a puromycin resistance marker, due to peptide cleavage at the 2A sites. One TCRβ- and TCRα-fragment were joined per cassette in the format of TCRβ-P2A-TCRα-T2A-Puromycin resistance (SEQ ID NO: 1). The TCR-beta and TCR-alpha regions of each of the 50 or 100 TCRs were synthesized and inserted into this vector in a combinatorial cloning approach executed by Twist Bioscience.

The 50×50 library was transfected into Phoenix-Ampho virus producer cells (ATCC CRL-3213) using Fugene transfection reagent and protocols known to the skilled artisan. The resulting retroviral virions were used to transduce a Jurkat reporter T cell line. The Jurkat reporter T cell is modified to express human CD8α and CD8β after transduction with a CD8α-P2A-CD8β transgene (SEQ ID NO: 2) using methods known to the skilled artisan. Jurkat reporter T cells were transduced with the TCR library resulting in 15% TCR-modified T cells of total live T cells (based on staining 4 days after transduction—after puro selection and on the day of the assay the purity was >60%). The use of murine TCR constant domain sequences in the TCR library (SEQ ID NO: 1) allows for the detection of TCR-modified Jurkat T cells by flow cytometry using a murine TCRβ constant domain specific antibody. Jurkat T cells modified to express a TCRβ-P2A-TCRα-T2A-Puromycin resistance transgene were positively selected to high purity by addition of Puromycin to the cell culture media. Antibiotic selection of genetically modified cells is known to an ordinary person skilled in the art.

Next, TCR transduced Jurkat T cells were stimulated with APCs that were engineered to present antigens in various ways. APCs included JY cells loaded with peptide, a mix of EBV-LCL cell lines each expressing a different minigene, EBV-LCL cells expressing a TMG, and EBV-LCLs that were not engineered to express specific antigens. Three days prior to the co-culture Jurkat reporter T cells were seeded at a low density (0.1×10⁶ cells per ml). APCs were mixed with Jurkat reporter T cells in a 1:1 ratio at a concentration of 2.5×10⁶ cells per ml. 200 uL (0.5×10⁶ cells per well) was distributed over ˜40 wells of a U-bottom TC-treated 96-well plate. Plates were centrifuged at 1000 rpm for 1 minute, and incubated for 20-22 hours at 37° C.

Subsequently, co-cultures were harvested and stained with fluorochrome-labeled antibodies for CD8, CD20 and CD69. Using a BD Biosciences ARIAFusion flow cytometry sorter the following populations were sorted for each stimulation condition (FIG. 14B):
1. Lymphocyte, single cell, live cell, CD20⁻, CD8⁺, CD69^{hi} (CD69^{hi} includes the highest 10-15% of single cell, live cell, CD20⁻, CD8⁺ cells based on CD69 fluorescence signal)
2. Lymphocyte, single cell, live cell, CD20⁻, CD8⁺, CD69^{lo} (CD69^{lo} includes 10-15% of the single cell, live cell, CD20⁻, CD8⁺ cells based on a low CD69 fluorescence signal)

Genomic DNA was isolated from the sorted TCR transduced. Jurkat T cells and used as template for multiple rounds of PCR with a limited number of cycles to amplify part of the TCRβ-P2A-TCRα cassette using PCR methods known to the skilled artisan. The resulting PCR product had a size of approx. 1.5 kb (FIG. 14C) and were sequenced using Oxford Nanopore Minion sequencing instruments using techniques known to a person skilled in the art. TCR identities were recovered using alignment techniques known to the skilled artisan, and for each TCR the fold enrichment of normalized read counts in the top versus bottom samples (y-axis) is represented as a function of the average TCR representation (x-axis; FIG. 14D). 14D; single replicate for each condition in presence of exogenous antigen; single replicate in absence of exogenous antigen expression). TCR combinations that were enriched in the ‘top’ versus the ‘bottom’ sample were identified using the DESeq2 R package as described using a linear model assuming an enriched TCR was defined as being enriched in the ‘top’ sample where antigens were presented, and being depleted in the ‘bottom’ sample where antigens were presented, relative to both ‘top’ and ‘bottom’ samples where no antigen was presented. TCR alpha and beta chain identity, as well as key statistical metrics are represented (FIG. 14E).

The 100×100 library screen was conducted in an analogous manner to the 50×50 screen, except that Jurkat reporter T cells were engineered to lack endogenous TCR expression and to exogenously express human CD8α and CD8β. In contrast to the 50×50 library screen, three replicates of the screen were performed. In the context of TMG-expressing B cells, and one replicate in the context of B cells that were not engineered to express TMG. Jurkat reporter T cells were transduced with the TCR library resulting in 14% TCR-modified. T cells of total live T cells. Rlog-transformed read counts were calculated using the DESeq2 R package, and the average Rlog-value for each TCR over all replicates of bottom samples were subtracted from the average Rlog-value for each TCR over all replicates of top samples and represented for cocultures that were performed in the presence (x-axis) and absence (y-axis) of TMG expression by B cells (FIG. 14F). The 5 spiked-in characterized TCRs are depicted as encircled larger black dots. The Wald statistic was used as a metric for the sorted probability measure plot (FIG. 14G).

Taken together, this example shows that combinatorial libraries of 50×50 and 100×100 design can be screened to identify antigen-reactive TCRs.

### Example 17

This example describes creation of a TCR repertoire using gene synthesis.

Five characterized TCRs of known antigen reactivity, as well as 95 uncharacterized TCRs are selected for TCR library generation (performed by Twist Bioscience; San Francisco/USA). In brief, the selected TCRα- and β-chains are generated as fragments by DNA synthesis. For library generation all selected TCRα and β fragments are combinatorially joined to continuous nucleic acid molecules encoding TCRβ-P2A-TCRα cassettes (SEQ ID NO: 1; A retroviral construct containing both beta and alpha TCR chains, as well as a puromycin selection marker can be used as a scaffold for creating the library. Retroviral transduction using this construct ultimately leads to expression of a single transcript, which results in translation of the TCR beta and alpha chains, as well as a puromycin resistance marker, due to peptide cleavage at the 2A sites). One TCRβ- and TCRα-fragment can be joined per cassette in the format of TCRβ-P2A-TCRα-T2A-Puromycin resistance (SEQ II) NO: 1; FIG. 15A). A primer pair flanking the variable TCR alpha and beta chain domains can be used to amplify both chains, and Nanopore sequencing was used to identify the identity of both chains. TCR chain identification is known to the person skilled in the art. The representation of each of the 10,000 alpha x beta combinations is represented for the 100×100 library. The resulting TCR library contained all 10,000 TCR variants without drop-outs in a very narrow frequency range (FIG. 15B).

Alternative ways of composing combinatorial libraries are envisioned, where a higher complexity library can be created by multiple overlapping or non-overlapping combinatorial sublibraries, collectively but not individually representing all the TCR combinations that are required to be present in the composite library (FIG. 15C). Alternatively, the complexity of libraries may be reduced by composing a bigger library of smaller combinatorial sublibraries in such a way that not all possible combinations of all alpha and all. beta chains in the composite library are represented. Using pairing information, or pairing likelihood information, a person skilled in the art can design the combinatorial sublibraries iii such a way that the paired chains, or the chains that likely constitute a pair, are all contained within one of the combinatorial sublibraries (FIG. 15D).

FIG. 15E depicts the identification of TCR combinations that are present in two 200×200 TCR libraries created by synthesis of four 100×100 libraries, and mixing these in 1:1:1:1 ratios. The occurrence of each possible TCR combination (x-axis) is represented as its density (y-axis). The number of TCR combinations that fall into a range of median +/− one log2-unit are 92% and 88%, respectively. The principle of creating a 200×200 library from mixing four 100×100 libraries in 1:1:1:1 ratios as depicted in FIG. 15C) is tested for two patient libraries (FIG. 15E). This shows that combinatorial TCR libraries of higher complexity can be created from mixing of multiple combinatorial TCR libraries with lower complexity.

FIG. 15F represents the pt2 TCR reactivity in the absence and presence of neo-antigens in a 200×200 library screen. The 200×200 library is created by combinatorially joining the 193 most frequently expressed TCRalpha and TCRbeta chains as measured using bulk TCR sequencing data from FIG. 10A)-10H). In addition, 7 previously characterized TCRalpha and TCRbeta chains are included. The 200×200 library is screened analogous to the 100×100 library screen described in 10A)-10H), except that coverage after gDNA isolation is in the range of 57-133, and that two replicates were performed in the context of expression of TMGs, and two replicates were performed in a context without TMGs. Another difference is that B cells are depleted using anti-CD20 or -Ly6G microbeads prior to FACS-sorting. After TCR alpha and beta chain identification, differentially expressed TCR combinations are identified using the DESeq2 R package. Differential representation analysis is known to the skilled artisan. Average Rlog-transformed read counts for the 200×200 library screen in the presence (x-axis) and absence (y-axis) of TMG expression by B cells is represented. In FIG. 15F. Six spiked-in characterized TCRs are depicted as larger dark grey dots. One characterized TCR is not represented because it is restricted to an HLA-allele that is not expressed in pt2 EBV-LCLs. TCRs that were identified in 10A)-10H) using a 100×100 library screen are represented as larger light grey dots. Additional TCR leads that are i) identified in the 200×200 library screen and ii) are not represented in the 100×100 library are represented as larger, intermediate shade grey dots.

This shows that six TCRs that are spiked into the library are identified in the 200×200 screening approach. In addition, this shows that new TCR leads that are not represented in the 100×100 library, can be identified using a 200×200 library screen. Thus, more neo-antigen reactive TCRs may be identified from 200×200, or otherwise more complex, TCR libraries in saturated library screens than from screens using 100×100 TCR libraries.

FIG. 15G represents the statistical metrics of six characterized TCRs, as well as TCRs that were identified in 10A)-10H) using a 100×100 library screen, in both. 100×100 and 200×200 library screens. The representation of TCRs that were identified in 10A)-10H) using a 100×100 library screen, as measured by the DESeq2 baseMean metric, is lower by a factor of more than 6 in the 200×200 screen than in the 100×100 screen. This is a likely explanation for the failure to identify these TCRs, and stresses the importance of equal representation of TCR variants in a TCR library.

Taken together, this example shows how TCR libraries can be created using gene synthesis, and how library complexity can be increased or reduced based on the idea of combining multiple combinatorial sublibraries.

### Example 18

This example describes optimizing coculture conditions for the identification of TCRalpha/beta pairs from a TCR repertoire.

In this example, co-culture conditions were adjusted and used to identify TCRαβ pairs from a TCR repertoire of highly diluted antigen-reactive TCRs. To test the suitability of CD69 as a T cell activation marker for screening purposes, Jurkat T cells expressing hCD8 and CMV#1 TCR were co-cultured with JY cells loaded with varying amounts of CMV peptide. Peptide loading of APCs is known to a person skilled in the art. CD69 positivity as measured by FACS increases depending on the concentration of the antigenic peptide (FIG. 16A).

To test whether seeding density influences the background of CD69 staining of Jurkat T cells, cells were seeded at various densities. Low-density seeding decreases CD69 background expression as measured by FACS (FIG. 16B). To test what the optimal coculture plates are, TCR+Jurkat reporter T cells were co-cultured with B cells expressing the relevant antigens in T75 or T25 flasks or 96 U bottom well plates. Activation of Jurkat T cells is most prominent when the co-culture is performed in a 96 U bottom well plate (FIG. 16C). To test the effect of the co-culture density on T cell activation, TCR+Jurkat T cells were co-cultured at various densities. Jurkat T cell activation is most efficient when 250,000 or 125,000 effector cells are seeded in the co-culture (FIG. 16D). To test the co-culture conditions determined in FIGS. 16A-16D in a genetic screening setting, a polyclonal pool of Jurkat reporter T cells was created by mixing Jurkat T cell lines that each express one of five characterized or one of twenty-four uncharacterized (non-relevant) TCRs. Mixing was performed as such that cells expressing a characterized TCR were present at frequencies between 1:10,000 and 1;1,000,000. Cells were seeded at low density (100,000 cells/ml) 3 days prior to co-culture, and co-cultured at a seeding density of 250,000 cells in 96 U well plates.

After 20 hours, co-cultures were harvested and stained with fluorochrome-labeled antibodies for CD20 and CD69. Next, the cells were fixed using a fixation buffer containing 4% formaldehyde. Using a BD Biosciences ARIAFusion flow cytometry sorter the following populations were sorted for each stimulation condition:
1. Lymphocyte, single cell, CD20⁻, CD69^{hi} (CD69^{hi} includes the highest 10-15% of single cell, CD20⁻ cells based on CD69 fluorescence signal)
2. Lymphocyte, single cell, CD20⁻, CD69^{lo} (CD69^{lo} includes 10-15% of the single cell, CD20⁻ cells based on a low CD69 fluorescence signal)

Genomic DNA was isolated from the sorted TCR transduced Jurkat cells and used as template for multiple rounds of PCR with a limited number of cycles to amplify the TCRβ cassette using PCR methods known to the skilled artisan. The resulting PCR product has a size of approx. 0.5 kb and can be sequenced using an Illumina sequencing instrument using techniques known to a person skilled in the art. TCR identities were recovered using alignment techniques known to the skilled artisan, and for each TCR the fold enrichment of normalized read counts in the top versus bottom samples is represented (FIG. 16E).

Taken together, this example shows that genetic screening using the described coculture conditions can be used to identify antigen-reactive TCRs that have frequencies of 1:1,000,000 or higher.

### Example 19

In order to be able to identify neo-antigen specific TCR(s) from such a library with high sensitivity and ensure that TCRs are not lost during the different processing steps, each unique TCR has to be represented multiple times during the screening process to maintain the TCR coverage. Therefore, a large number of TCR-transduced Jurkat cells and APCs have to be screened³³. In addition, one goal is to upscale the number of TCRs in a library to allow high sensitivity screening of greater than 10,000 TCRs. This highlights the need to enhance the scalability of the TCR discovery platform by optimizing various process steps to allow a more efficient processing of a large number of cells while still maintaining TCR coverage. Therefore, the aim of this study was to further optimize a number of these process steps to enhance scalability while maintaining the sensitivity of the TCR isolation platform.

In these studies four known HLA-A*02:01-restricted TCRs-CDK4 TCR clone 8 and 17 (CDK4-8 and 17 in short) and CMV TCR clone 1 and 2 (CMV-1 and 2 in short) were used. The two CDK4 TCRs are specific for a mutated cyclin-dependent kinase 4 (CDK4_{R24C}) peptide. This mutation-derived neo-antigen epitope was identified in multiple melanoma patients³⁴. Furthermore, the two CMV TCRs target a peptide encoded by a component of the human cytomegalovirus (CMV), pp65³⁵. For both the CDK4 and the CMV epitopes, two distinct TCR clones with potentially different affinities in the studies were used. This would possibly allow one to evaluate the role of TCR affinity for the cognate peptide on the screening process.

As a first step in the processing of large cell numbers, blasticidin selection as a method to enrich for TCR-expressing Jurkat cells following retroviral transduction was assessed. It was observed that blasticidin selection led to an efficient enrichment of transduced Jurkat cells and resulted in minimal toxicity. In addition, it was found that upscaling the co-culture format from 96 well (96W) round-bottom plates to GMP bags allowed a high throughput processing of at least 170×10⁶ effector and target cells, while maintaining a comparable activation of the effector cells.

Next, different methods to replace the flow cytometric sorting step in the TCR library screening process with a bead-based selection step were explored. In addition to CD69, the T cell activation markers CD25 and CD62L were assessed in a longitudinal co-culture assay. CD25 functions as the IL-2. receptor a chain and is known to be expressed by T cells following activation³², while CD62L is expressed on nave T cells and is downregulated upon T cell activation, enabling effector T cells to re-enter the bloodstream³⁶. CD25 and CD62L showed promising expression profiles and are thus candidates for a two-step bead-based selection in combination with CD69.

Finally, the efficacy of an NFAT-based reporter system was assessed. Vectors containing multiple human NFAT binding sites followed by a minimal promoter and a reporter gene have been established to study antigen-specific responses of T cells³⁷⁻⁴⁰. The minimal promoter does not contain any NFAT binding sites and its sole purpose is to initiate transcription of the reporter gene upon binding of transcription factors to the NFAT binding site(s). Jurkat cells transduced with these constructs would express the reporter gene only upon T cell activation. A cell surface marker suitable for bead-based selection or an antibiotic resistance gene can be introduced as a reporter gene to circumvent the flow cytometric selection step. However, the results indicated that using this reporter system leads to a high background and therefore, is unlikely to be incorporated into the screening platform.

### Results

Peptide titration assays suggest that there is no or minimal difference in the affinities between CDK4 TCR clone 8 and 17 and between CMV TCR clone 1 and 2.

The affinity of the four model TCRs (CDK4 TCR clone 8 and 17 and CMV TCR clone 1 and 2) for their cognate peptide was characterized prior to their use in the studies. This was achieved by performing peptide titrations in a co-culture assay.

The four TCRs were introduced into a Jurkat TCR knockout (KO) CD8⁺ cell line by retroviral gene transfer. The transduced Jurkat TCR KO cells were 35-45% mTCRβ⁺ CD8⁺ and displayed a CD8⁻ population (FIG. 17a, top panel). In order to avoid correcting for the transduction efficiency during the co-culture assay, the TCR-transduced Jurkat cells were sorted to obtain a population of ˜90% mTCRβ⁺ CD8⁺ cells (FIG. 17a, bottom panel).

Using CD69 as readout, 20 hours co-culture of CDK4-8 and 17-transduced Jurkat TCR KO cells with FILA-A*02:01-expressing JY cells loaded with graded concentrations of the CDK4 mutant peptide revealed that the CDK4-17 TCR has a slightly higher affinity for its cognate peptide than the CDK4-8 TCR (FIG. 17b, EC50 of CDK4-8: 43 ng/ml, EC50 of CDK4-17: 23 ng/ml). In the case of the CMV TCRs, CMV-1 and 2 TCRs displayed a comparable affinity for the CMVpp65 peptide when Jurkat TCR KO cells expressing these TCRs were co-cultured with HLA-A*02:01-expressing JY cells loaded with graded concentrations of the CMVpp65 peptide (FIG. 17c, EC50 of CMV-1: 2.2 ng/m, EC50 of CMV-2: 1.2 ng/ml).

Therefore, the data suggest that CDK4-8 and 17 have similar (or minimal different) affinities for the CDK4 mutant peptide and CMV-1 and 2 have comparable (or minimal different) affinities for the CMVpp65 peptide. However, these different TCR clones are of limited value as tools for studying the effect of TCR affinity on the screening process. Therefore, one of the CDK4 TCRs and one of the CMV TCRs were standardly included in the studies before moving forward with testing a library of TCRs.

Jurkat cells with different TCR transduction efficiencies are efficiently selected with blasticidin.

Following the retroviral TCR library transduction into Jurkat TCR KO cells, an efficient and high throughput selection procedure is useful to enrich successfully TCR-transduced Jurkat cells (>80% mTCRβ⁺ CD8⁺ cells) without causing toxicity and losing TCR coverage. Antibiotic selection is an attractive strategy to enrich for TCR-transduced Jurkat T cells and was assessed.

For this purpose, blasticidin selection was initially assessed by using the CDK4-17 TCR, Since the transduction efficiencies of TCR libraries vary (usually 10-56% mTCRβ⁺ CD8⁺ cells), blasticidin selection was evaluated by Jurkat TCR KO cells with different CDK4-17 transduction efficiencies (10 and 30% mTCRβ⁺ CD8⁺ cells). Cells plated at a density of 0.25×10⁶ cells/ml and selected for seven days with 6 μg/ml blasticidin resulted in the highest fold expansion as compared to using lower antibiotic concentrations (2 and 4 μg/ml) and higher starting cell densities (0.5 and 1×10⁶ cells/ml). Additionally, the above-mentioned selection condition led to ˜90% mTCRβ⁺ CD8⁺ cells (data not shown).

In order to validate if the most optimal blasticidin selection condition for CDK4-17 can also be applied to other TCRs, one of the CMV TCRs, CMV-1, was examined. In addition, if removing the blasticidin at an earlier time point allows the selection to continue and improves the fold expansion was assessed. To achieve that, on day 4 of the selection the cells were re-plated at their starting density either in medium with or without the respective concentration of blasticidin (referred to as ‘removed the blasticidin on day 4’ and ‘added new blasticidin on day 4’, respectively).

Jurkat TCR KO cells were transduced with different volumes of retroviral supernatant in order to achieve different transduction efficiencies (6, 20 and 60% mTCRβ⁺ CD8⁺ cells, FIG. 18a).

The TCR-transduced cells were plated at a concentration of 0.25×10⁶ cells/ml and selected with different concentrations of blasticidin (0, 4, 5 and 6 μg/ml). Six days after the selection there was no noticeable difference in terms of fold expansion between cells with or without the removal of blasticidin on day 4. Contrary to the data with CDK4-17 TCR, differentially transduced Jurkat cells selected with lower blasticidin concentrations (4 and 5 μg/ml) appeared to have a slightly higher fold expansion than cells selected with 6 μg/ml of this antibiotic (FIG. 18b, FIG. 25a shows the fold expansion of non-transduced cells). Despite these differences in fold expansion, varying blasticidin concentrations consistently resulted in a selection efficiency of 80-90% mTCRβ⁺ CD8⁺ cells (FIG. 18c, FIG. 25b shows that the percentage of NT cells in the 4 μg/ml conditions was due to very few surviving cells being present, some of which appeared in the mTCRβ⁺ CD8⁺ gate). Finally, removing the blasticidin on day 4 did not have any effect on the fold expansion and the selection efficiency (FIG. 18b,c). Based on this data the measurements on day 7 only with the Jurkat cells selected with 4 μg/ml blasticidin was continued because this antibiotic concentration resulted in a high fold expansion and 80-90% mTCRβ⁺ CD8⁺ cells. Furthermore, it was reasoned that using 4 μg/ml blasticidin would cause the least toxicity from the three tested concentrations.

Seven days after the selection, TCR-transduced Jurkat cells for which blasticidin was removed on day 4 revealed a slightly higher fold expansion than cells for which blasticidin was added on day 4 with respect to both total live cells and mTCRβ⁺ CD8⁺ cells (FIG. 18d). However, removing blasticidin on day 4 did not have an effect on the selection efficiency which led to >90% mTCRβ⁺ CD8⁺ cells in all the tested conditions (FIG. 18e).

Of note, differences were observed in the mTCRβ MFI of CD8 and mTCRβ double-positive Jurkat cells depending on if the blasticidin was removed or added on day 4. Cells with added blasticidin displayed a higher MFI than cells with removed blasticidin on day 4. This difference was more apparent on day 7 after the selection than on day 6 (FIG. 26).

In conclusion, Jurkat TCR KO cells with varying CMV-1 transduction efficiencies were efficiently selected with three different blasticidin concentrations (4, 5 and 6 μg/ml). In terms of fold expansion, the lower blasticidin concentrations appeared to be less toxic even though the differences between the three tested antibiotic concentrations were minor. Furthermore, it has been shown that removing the blasticidin at an earlier time point allowed for the selection to continue and resulted. In a slightly better fold expansion than exposing the cells to the antibiotic during the full 7-day selection.

Jurkat cell-APC co-cultures in GMP bags lead to a comparable expression of the CD69 activation marker to co-cultures in 96W round-bottom plates.

Enhancing the scalability of the screening platform also necessitates optimizing the established 96W round-bottom plate co-culture format to a setup that allows a more efficient processing of a large number of effector and target cells while maintaining TCR coverage. Previous studies carried out demonstrated that performing co-cultures in flat-bottom cell culture vessels with larger surface areas than the well of a 96W plate caused a decrease in the CD69 expression of Jutkat cells. In addition, varying the E:T ratio and the starting cell density of such co-cultures did not lead to an increased. CD69 upregulation of the effector cells (data not shown). This data suggests that using round-bottom culture vessels, such as the 96W round-bottom plate, results in more efficient effector-target contact. Therefore, it was decided to identify a larger scale co-culture system which would enhance effector-target contact and allows the processing of high cell numbers in a single culture vessel.

For the comparison of different co-culture systems, CD8⁺ Jurkat cells of which 40-50% were TCR-positive were used as effector cells in order to obtain clear CD69 positive and negative populations (FIG. 19a, 19b, 19c, 19d—left). As target cells, HLA-A*02:01-expressing EBV-immortalized B cells (EBV LCLs) were utilized that had been engineered, to express the antigens recognized by the TCR-expressing Jurkat cells (EBV LCL TMG2.1).

Enhancing the effector-target contact was first examined, by performing co-cultures in 15 and 50 ml Falcon tubes positioned on a rack. Since Flacon tubes are not cell culture-treated, a small amount of cells (≤2×10⁶ cells, upscaled based on the diameter differences between a well of a 96W plate and 15/50 nil Falcon tube) was used to assess the cell viability and activation of effector cells after 20 h of co-culture. The percentage of live and CD69⁺ cells was comparable between co-cultures in a 96W plate and a Falcon tube (FIG. 19a). However, increasing the cell number to 38×10⁶ caused a sharp decrease in cell viability (FIG. 19a).

Of note, since CD69 upregulation was the only measure of Jurkat cell activation, if the anti-human CD69 antibody (clone FN50) provides an accurate representation of CD69 expression was verified. Co-staining with an anti-human. CD69 antibody against a different epitope (clone CH/4) revealed a comparable level of staining between the two different antibody clones (FIG. 27).

Next, it was examined if the cell loss in the Falcon tube co-culture was due to the small surface area of the medium in contact with the atmosphere preventing efficient gas exchange. To assess that, Falcon tubes were spun down and 14 ml of medium was taken off from the tubes with 38×10⁶ cells resulting in a total volume of 1 ml. However, a sharp decrease in cell viability was observed in the Falcon tube co-culture with 38×10⁶ cells (FIG. 19b). Additionally, co-cultures in a 250 ml tube were further tested since these containers have a wider diameter and might allow for more efficient gas exchange to occur (not spun down because the appropriate centrifuge rotor was not available). Even though the 250 ml tube co-culture resulted in a similar CD69 upregulation as the 96W plate co-culture, the cell death was increased (FIG. 19b).

It was concluded that performing co-cultures of large number of cells in (Falcon) tubes was not viable due to the increase in cell death. This effect on cell viability is possibly due to inefficient gas exchange and the large cell numbers placed in (Falcon) tubes. Therefore, the cell viability and effector-target contact was assessed in MACS GMP Cell Differentiation Bags—500 (GMP bags in short), a culture vessel that can accommodate large numbers of cells and allows for gas exchange to occur across the entire surface area of the bag.

The current TCR library screening platform allows the screening of 10,000 TCRs and requires the co-culture of 170×10⁶ effector and target cells in order to maintain the TCR coverage. Therefore, a co-culture with 170×10⁶ Jurkat cells and APCs was set up in a GMP bag.

In the alternative a screening platform can include 80×10̂6 effector cells and 80×10̂6 target cells, so the value can be 160×10̂6. In the alternative, co-cultures can be set up with 90×10̂6 cells since one may oselose ˜10% due to the use of multichannels and one may wish to make sure one keeps at least 80×10̂6 cells.

To enhance the effector-target contact and allow efficient gas exchange, the GMP bags were placed horizontally into a round colander (FIG. 19c). After a 20-h co-culture, comparable cell viability and CD69 upregulation was observed between the GMP bag and 96W plate co-cultures. However, the CD69 MFI was slightly decreased in the GMP bag condition (FIG. 19c). Therefore, it was assessed if spinning down the GMP bag would increase the CD69 MFI. In order to be able to spin down the GMP bags, they were placed vertically in the centrifuge rotor and moved to the colander without disturbing the cells. GMP bags that were not spun down were also placed in an upright position. The percentage and MFI of CD69⁺ Jurkat cells was comparable between co-cultures in a GMP bag and 96W plate. In addition, no major difference was observed in terms of CD69 expression depending on if the effector and target cells were spun down before the 20-h co-culture (FIG. 19d). However, the cell viability decreased after 20 h of co-culture in the GMP bags. Surprisingly, the amount of cell loss varied between the different GMP bags (FIG. 19d).

Altogether, the data suggests that co-cultures in GMP bags result in a similar activation of the effector cells as the already established 96W plate co-culture format. However, further studies—for example, different way of placing the bag—are required in order to examine the cause of the increased cell death in FIG. 19d.

CD25 and CD62L show promising expression profiles for a two-step bead-based selection in combination with CD69.

To date, CD69 has been used as an activation marker for selecting activated Jurkat cells in the TCR library screening platform following a 20-h co-culture and flow cytometric sorting. In order to assess a more scalable bead-based selection approach, in addition to CD69 that was explored the expression profiles of two supplementary T cell activation markers CD25 and CD62L. For that purpose, a longitudinal analysis of CD69, CD25 and CD62L expression of TCR-transduced Jurkat cells following co-culture with cognate antigen-expressing target cells was carried out.

The CD69 upregulation of Jurkat cells appeared to remain stable 16-32 h after starting the co-culture (FIG. 20a), In comparison, the expression profiles of CD25⁺ and CD69⁺CD25⁺ effector cells showed a slight upregulation after the 24-h time point (FIGS. 20b, 20c). It was further Observed that the downregulation of CD62L increased slightly between the 16 and 20-h time points after the co-culture initiation and remained stable until the 32-h time point. However, the background signal was high when using CD62L as an activation marker, as unstimulated TCR-transduced and non-transduced Jurkat cells were between 20-40% CD62L negative at the various time-points analyzed after starting the co-culture (FIG. 20d). Co-expression analysis of CD69 and CD62L revealed that the effector cells that non-specifically downregulated CD62L expression did not upregulate CD69. Furthermore, a population of activated Jurkat cells with upregulated CD69 but lacking CD62L expression could be observed 16-32 h after the initiation of the co-culture (FIG. 20e).

CD25 and CD62L show promising expression profiles in this preliminary longitudinal analysis as their expression correlates with CD69 upregulation, suggesting that they could potentially be used in a two-step bead-based selection process in combination with CD69.

Lentiviral NFAT reporter system results in a high background signal in Jurkat and primary T cells.

An alternative method to using endogenous cell surface activation markers to select reactive TCRs in the TCR library screening process would be to set up an NFAT-based reporter system in Jurkat cells. Setting up this reporter system may allow for antibiotic selection of activated Jurkat cells which would circumvent flow cytometric sorting. Alternatively, the antibiotic resistance cassette can be replaced by any cell surface marker that is suitable for bead-based enrichment.

Four different lentiviral NFAT-based reporter vectors were designed. The self-inactivating (SIN) lentiviral plasmids contain a truncated inactive 5′ LTR promoter and thus upon integration into the genome, the transcription of the puromycin resistance reporter gene should solely be dependent on the binding of the NFAT transcription factors to the NFAT binding sites⁴¹. Three of the constructs contain either two, four or six identical NFAT binding sites, followed by a minimal IL2 promoter. The fourth vector is made of the full IL2 promoter which comprises at least three distinct NFAT binding sites (FIG. 21a)⁶. Different NFAT-based reporter plasmids were studied to determine the most sensitive reporter.

The NFAT-based lentiviral vectors were co-transfected with an additional pmaxGFP plasmid into HI K293T cells. The transient GFP expression of the virus-producing cells three days post-transfection was used as a measure of the transfection efficiency. The HEK293T cells were 100% GFP⁺, suggesting that the lentiviral transfections were successful (FIG. 21b). Next, the lentiviral supernatant was transduced into Jurkat TCR KO cells which were subsequently exposed to a non-physiological PMA/ionomycin stimulus for 24 h to evaluate the efficacy of the reporter system. It has been shown that NFAT activation reaches a maximum 72 h post-stimulation³⁸. Therefore, the PMA/ionomycin stimulation was followed by a 3-day puromycin selection. However, the cell viability of NFAT-transduced Jurkat cells was sharply reduced and comparable between puromycin-exposed transduced and non-transduced cells (FIG. 21c, top panel). The activated cells were >99% CD69⁺, implying that the stimulation was efficient (FIG. 21c, bottom panel).

The NFAT-transduced puromycin-selected cells were expanded for 20 days and subjected to a second round of stimulation and selection. Note that the non-transduced Jurkat cells in this experiment were different from the non-transduced cells in FIG. 21c. PMA/ionomycin stimulation followed by a 4-day selection with various concentrations of puromycin resulted in no major difference in terms of cell viability between stimulated and unstimulated NFAT-transduced cells (FIG. 21d). Surprisingly, the CD69 expression was lower for the transduced cells than for the non-transduced cells (FIG. 21d). One explanation might be that the NFAT-transduced Jurkat cells were not fully recovered from the first round of non-physiological PMA/ionomycin stimulus.

Taken together, this data shows that there is no enrichment of puromycin resistant NFAT-transduced. Jurkat cells after T cell stimulation. Even though the transfection of the pmaxGFP plasmid was efficient, the transfection and transduction of the lentiviral constructs might have been unsuccessful (FIG. 21b). Other possible explanations might be a suboptimal antibiotic selection or a leaky reporter system. In order to address some of the above-mentioned scenarios, the following experiments were performed with NFAT reporter constructs containing an EGFP reporter gene (FIG. 22a). In an alternative, the reporter gene can be any cell surface marker suitable for bead-based selection/antibiotic resistance gene.

By using EGFP NFAT plasmids, the lentiviral transfection efficiency can be directly assessed by a readout of the CMV-driven GIFP expression of the HEK293T cells. This revealed that PEI is a more efficient lentiviral transfection reagent than FuGENE (FIG. 22b). To study the efficacy of the reporter system, the NFAT-transduced Jurkat TCR KO cells (PEI transfections) were stimulated with PMA/ionomycin for 24 h and the GFP expression was read out every 24 h for three days. No noticeable difference was observed between stimulated and unstimulated Jurkat cells in terms of the percentage and MFI of GFP⁺ cells for any of the NFAT-based vectors (FIG. 22c). The upregulation of CD69 upon activation revealed that the PMA/ionomycin stimulation was effective (FIG. 22c). Altogether, this data suggests that the lcntiviral NFAT reporter system has a very high background signal in Jurkat cells.

It was next evaluated if a similar background signal was also observed in NFAT-transduced primary T cells. For that purpose, two NFAT-based lentiviral vectors were used—NFAT4× and NFAT4× new. As opposed to NFAT4×, NFAT4× new does not contain a minimal IL2 promoter but a less complex one, called minP. MinP has been described previously by Jutz et al³⁸. Primary T cells from two healthy donors were transduced with the NFAT lentiviral supernatants and subsequently stimulated with PMA/ionomycin for 24 h. The GFP expression was measured every 24 h for three days. Contrary to the data with Jurkat cells, a slightly higher percentage and MFI of GIP⁺ cells in the stimulated conditions was observed than in the unstimulated ones even though these differences were not highly significant (FIG. 23). Additionally, using an NFAT4× construct with a different minimal promoter did not result in more specific GFP expression in transduced T cells. Even though CD69 was not upregulated upon PMA/ionomycin stimulation, the variation in terms of GFP expression between stimulated and unstimulated NFAT-transduced cells suggests that the stimulation was efficient (FIG. 23).

Altogether, the data indicates that the lentiviral delivery of NFAT-based reporter vectors results in a high background signal in both Jurkat and primary cells.

Non-viral delivery of NFAT reporter constructs into Jurkat cells results in a high background signal.

It was next studied the efficacy of a non-viral NFAT reporter delivery system in Jurkat cells by designing two different constructs, NFATOx and NFAT4× (FIG. 24a). NFAT0× does not contain any NFAT binding sites and thus serves as a control to assess the background signal of the minimal promoter. Both plasmids contain an EGFP reporter gene and constitutively expressed E2-Crimson and puromycin resistance genes. By electroporating Jufkat cells with the two NFAT plasmids, the constructs will randomly become linearized and will integrate into an arbitrary location in the genome. Therefore, a low transfection efficiency was anticipated. However, the Jurkat cells with a stable integration of the plasmids in their genome will be enriched by performing a puromycin selection. The NFAT background signal will be further assessed by stimulating the selected Jurkat cells.

CDK4-17-expressing Jurkat TCR KO cells, transfected with the NFAT constructs, did not display a major reduction in cell viability over time (FIG. 24b). Surprisingly, two days post-electroporation an increase in the GFP⁺ and GIP⁺ E2-Crimson⁺ cells was observed which was more prominent for the NFAT0×-transfected Jurkat cells. The decrease of these two cell populations six days after the transfection implies that the double-positive and GFP single-positive expressions were transient (FIG. 24c). Puromycin selection was initiated six days post-transfection for seven days. During the selection the percentages of E2-Crimson and GFP single and double-positive Jurkat cells increased (FIG. 24c). Seven days after the puromycin treatment, the selection appeared to be complete as there were no surviving non-electroporated Jurkat cells (FIG. 24d). However, the percentage of GFP⁺ and GFP⁺ E2-Crimson⁺ cells was comparable to the E2-Crimson expression of the NFAT-transfected Jurkat cells. This suggests that non-viral delivery of NEAT reporter plasmids also results in a high background signal.

### Discussion Regarding Example 19

A fully personalized TCR gene therapy to treat cancer by focusing on the identification of neo-antigen specific TCRs is very useful. This approach is dependent on genetic screening and therefore the processing of a large number of TCR-expressing Jurkat cells and APCs is required. In order to be able to treat many patients, the screening platform should allow the efficient handling of effector and target cells without affecting the screening sensitivity. Here, studies were presented that were undertaken to enhance the scalability of the neo-antigen reactive TCR isolation platform.

### Blasticidin Selection of TCR-Transduced Jurkat TCR KO Cells.

Following retroviral gene transfer of a TCR library into Jurkat TCR KO cells, an efficient selection method is useful in order to enrich for TCR-expressing Jurkat cells without causing toxicity and losing TCR coverage. To this end it has been demonstrated that the antibiotic blasticidin resulted in an efficient selection of CMV-1 and CDK4-17 TCR-transduced Jurkat TCR KO cells (FIG. 18 and data not shown). Even though the differences between the different blasticidin concentrations and blasticidin selection methods tested were not major, the selection of CMV-1-transduced cells for seven days with a starting density of 0.25×10⁶ cells/ml and 4 μg/ml blasticidin which was removed on day 4 of the culture led to an enrichment of >90% mTCRβ⁺ CD8⁺ Jurkat cells and the highest fold expansion (FIG. 18).

However, this selection approach resulted in ˜70% mTCRβ⁺ CD8⁺ Jurkat cells transduced with a library of 10,000 TCRs. Additionally, the percentage of mTCRβ⁺ CD8⁺ cells further decreased upon Jurkat cell expansion prior to the co-culture assay (data not shown). Thus, for the enrichment of Jurkat cells transduced with a library of patient-derived TCRs, selection with 6 μg/ml blasticidin and adding new blasticidin on day 4 were standardly applied to achieve >70% mTCRβ⁺ CD8⁺ Jurkat cell population (data not shown). Furthermore, a starting cell density of 0.5×10⁶ cells/mil was applied in order to reduce the amount of culture flasks required.

The blasticidin selection study was performed with two known TCRs while the patient screens involve the transduction of 10,000 uncharacterized TCRs. Therefore, the antibiotic selection of Jurkat cells expressing a library of TCRs might require a more harsh treatment than the enrichment of cells expressing a single TCR. A similar observation was made in the study of Spindler et al. in which TCR library-expressing Jurkat cells were selected with puromycin for 14 days. The CD3⁺ TCRαβ⁺ cells were enriched from ˜10% to ˜50% after the selection⁴².

It is still to be addressed if adding blasticidin on day 4 results in the loss of TCR-expressing cells with a lower mTCRβ MFI or if the observed difference in MFI is merely a result of a change in the Jurkat cells' phenotype due to the longer antibiotic treatment (FIG. 26). Furthermore, it will be useful to test if low transduction efficiencies (<10% mTCRβ⁺ CD8⁺) cause a reduction in the TCR coverage. These questions can be addressed by transducing a library of different TCRs into Jurkat cells and sequencing the TCRs of the blasticidin-enriched cells.

In conclusion, the process to find the most optimal and efficient selection method to enrich for TCR-transduced Jurkat cells shows that studies with known TCRs are useful. However, the findings from the experiments are to be further validated and may involve additional adjustments when screening a library of unknown TCRs.

### Jurkat Cell-APC Co-Cultures in GMP Bags.

In order to further enhance the scalability of the screening platform, the Jurkat cell-APC co-culture format involves upscaling from the established 96W round-bottom format.

It is our understanding that this study is the first to examine the efficacy a new co-culture format in GMP bags which resulted in a similar CD69 expression as co-cultures in a 96W plate (FIGS. 19c, 19d). GMP bags can be folded in a way that mimics the round-bottom well of a 96W plate and thus enhances effector-target contact. Furthermore, the GMP bags allow for an efficient gas exchange across their entire surface area in order to prevent cell loss. However, as seen in FIG. 19d, co-cultures in GMP bags resulted in an increased cell death as compared to the 96W plate co-cultures. The decrease in cell viability might have been due to the different placement of the GMP bags that was required for the short spinning step. The cells were collected at the bottom of the GMP bags which might be the only part of the culture vessel that does not allow for an efficient gas exchange. Note that in FIG. 19c the GMP bags were not spun down and the cells were collected on the side of the bag which is possibly the reason the cell viability was not affected. Even though repeats of the GMP bag co-cultures are useful to further examine the cause of cell death in FIG. 19h, the observations underline the need to establish a consistent way of placing the flexible GMP bag in the incubator. For instance, a cast could be designed that has the appropriate shape while also maintaining CO₂-permeability.

Another benefit of using GMP bags for the Jurkat cell-APC co-cultures is that more cells can be added while maintaining a similar cell density. This could allow for the screening of greater than 10,000 TCRs. In addition, GMP bags are a closed system which would drastically decrease the possibility of cross-contamination.

Of note, the efficacy of different co-culture systems based solely on the expression of the T cell activation marker CD69 was assessed. This experimental setup does not take into account the effect a GMP bag co-culture might have on the TCR coverage. To study that, Jurkat cells expressing a library of TCRs can be co-cultured with APCs in a GMP bag and 96W plates in parallel. This will be followed by the established CD69-based flow cytometric sorting of activated Jurkat cells and next generation sequencing to compare the enrichment of specific TCRs.

Additional experiments will assess the effect of carrying out co-cultures in GMP bags on the TCR isolation process. However, the preliminary data shows a promising co-culture system which should provide an efficient processing of effector and target cells in library screens of at least 10,000 TCRs.

Replacing Flow Cytometry-Based Sorting with Bead-Based Selection.

Some TCR screening platforms currently involve flow cytometric sorting of CD69-expressing Jurkat T cells after a co-culture to identify neo-antigen specific TCRs. However, a bead-based selection of activated Jurkat cells would enhance the scalability of the TCR screening platform. Therefore, it was decided to evaluate additional or alternative activation markers for bead-based selection, which preferably give a clear separation between activated and non-activated. Jurkat populations.

Performing longitudinal co-culture analysis with two additional T cell activation markers, CD25 and CD62L, revealed that the heterogeneity of CD25 expression was greater than the heterogeneity of CD69 expression of activated Jurkat cells (FIGS. 20a, 20b). In addition, CD62L downregulation was not solely specific for activated Jurkat cells as some non-transduced and unstimulated TCR-transduced effector cells were negative for CD62L (FIG. 20d). Therefore, these two activation markers are not attractive candidates for a single marker bead-based selection approach. However, CD25 and CD62L could be used in a sequential bead-based selection process, involving depletion or enrichment of CD62L⁺ or CD25⁺ effector cells, respectively followed by the capture of activated cells by a CD69 positive selection. Of note, the CD25⁺0 and CD69⁺CD25⁺ populations were slightly elevated after the 24-h time point. This however is not surprising considering CD25 has been shown to be a late activation marker⁴³. This also suggests that the later time points would be the most ideal for the analysis of effector cells with these two activation markers. CD62L shedding has been reported to reach its peak around four hours after T cell activation and is thus an early activation marker^{44,45}. One study has shown that following the 4-h peak, CD62L undergoes a dynamic expression profile⁴⁴. However, here a very stable downregulation of CD62L was observed after 20 h of co-culture that follows a similar pattern as the CD69 upregulation (FIGS. 20a, 20d). This would allow us to perform co-expression analysis with CD69 and. CD62L 16-32 h after Jurkat cell activation (FIG. 20e). Nevertheless, this longitudinal analysis requires repetition by co-culturing TCR-transduced Jurkat TCR KO cells with EBV LCLs that are not expressing the cognate peptide. This would allow for a more accurate estimation of the background signal of the three activation markers.

Finally, the efficacy of a lentiviral NFAT-reporter system to allow antibiotic selection or bead-based enrichment of activated Jurkat cells was examined. However, the lentiviral delivery of NFAT vectors resulted in a high background signal in both Jurkat and primary cells (FIG. 22 and FIG. 24). Studies employing this reporter system mostly rely on a clonal population with a minimal background signal^{37,38}. NFAT-transduced Jurkat cells with a puromycin resistance reporter gene were exposed to two rounds of antibiotic selection (FIG. 21). However, there was no noticeable difference between stimulated and unstimulated Jurkat cells after the second selection, despite the assumption that a semi-clonal population was obtained after the initial antibiotic treatment. Another explanation might be the suboptimal non-physiological PMA/ionomycin stimulus, preceding the second puromycin selection, which yielded <30% CD69⁺ NFAT-transduced Jurkat cells (FIG. 21d). Additionally, there was no noticeable difference between stimulated and unstimulated EGFP NFAT-transduced Jurkat cells (FIG. 22). Thus, selecting a clone would not be feasible because of this lack of differential GFP expression. The results suggest that the reporter system leakiness is not caused by the minimal IL2 promoter as another minimal promoter minP revealed a similar level of background (FIG. 23). Therefore, it was concluded that the leakiness might be a characteristic of the lentiviral delivery system.

SIN NFAT retroviral vectors have been successfully used and set up to study antigen-specific T cell responses^{37,38,40}. Non-viral delivery of NFAT plasmids also resulted in a high background signal as the expression of the reporter EGFP gene was elevated in non-stimulated Jurkat cells (FIG. 24). However, T cell activation is required to further assess the leakiness of the NFAT-transfected Jurkat cells. In addition, the E2-Crimson single-positive population can be subjected to a flow cytometric sort to subsequently evaluate the efficacy and GFP leakiness of this population in a T cell stimulation assay. An area of future research could be to study the sensitivity of AP-1 and NF-κB reporter systems as these transcription factors utilize distinct signaling pathways which might have a different effect on the background signal³⁸. For instance, an NF-κB reporter system has been successfully applied in the screening of a library of chimeric antigen receptors in Jurkat cells⁴⁰.

This study aimed at enhancing the scalability of a neo-antigen specific TCR isolation platform. These experiments are useful as a more efficient processing of reporter Jurkat T cells and. APCs would ultimately allow for the treatment of more cancer patients. First, blasticidin selection of retroviral TCR-transduced Jurkat TCR KO cells was identified as the most efficient and least toxic effector cell enrichment procedure. Furthermore, upscaling the Jurkat cell-APC co-cultures from a 96W format to a more scalable GMP bag setup led to a comparable level of Jurkat cell activation. Finally, the groundwork has been laid out that should allow for the replacement of flow cytometric sorting with a more scalable bead-based selection of neo-antigen reactive TCR-expressing Jurkat cells in the TCR discovery platform.

### Materials and Methods—Example 19

### Cell Lines

The human Jurkat cell line (Clone E6-1, TIB-152) was purchased from ATCC and the human EBV-immortalized FILA-A2-positive B cell line, called JY, was purchased from ECACC. Both Jurkat and JY cell lines were cultured in RPMI-1640 medium, HEPES (Gibco) supplemented with 10% fetal bovine serum heat inactivated (FBS, Gibco), 100 U/ml penicillin (Gibco) and 100 μg/ml streptomycin (Gibco). Human embryonic kidney 293T (HEK293T, CRL-3216) and. Phoenix Ampho cell lines were purchased from ATCC and were cultured in DMEM medium (Gibco) supplemented with 10% FBS, 100 U/ml penicillin (Gibco) and 100 μg/ml streptomycin. All cell lines were maintained at 37° C. and 5% CO₂.

Isolation of T Cells and B Cells from Human Healthy Donors

Buffycoats of healthy human donors were obtained from Sanquin Blood Supply (The Netherlands) after informed consent. Peripheral blood mononucleated cells (PBMCs) were isolated from buffycoats using Ficoll Paque Plus (Sigma-ALdrich).

HLA-A2-positive B cells were isolated using a negative selection with MojoSort Human Pan B Cell Isolation Kit (BioLegend) and subsequently EBV-immortalized with human gammaherpesvirus 4 (HHV-4, ATCC VR-1492). EBV immortalized B cells (EBV LCLs) were cultured in RPMI-1640 medium, HEPES (Gibco) supplemented with 20% FBS, 100 U/ml penicillin (Gibco) and 100 μg/ml streptomycin (Gibco).

T cells were isolated and activated with CD3/CD28 Dynabeads (Life Technologies Europe) and cultured in RPMI-1640 medium, HEPES (Gibco) supplemented with 10% human serum (Sigma-Aldrich), 100 U/ml penicillin (Gibco), 100 μg/ml streptomycin (Gibco) and cytokines (5 ng/nil human IL-15 and 100 IU/ml human IL-2, Peprotech). All primary cells were maintained at 37° C. and 5% CO₂.

### Flow Cytometry and Sorting

FACS buffer was made by supplementing PBS (Gibco) with 2% FBS. To perform flow cytometric analysis, cells were stained for 20 min at 4° C. in the dark and washed once with FACS buffer. LIVE/DEAD Fixable Near-IR (Life Technologies Europe, 1:1000 dilution) or DAPI (Sigma-Aldrich, 1:1000 dilution) were used as a live/dead stain. Virus-producing cells were fixed with Cytofix (BD Biosciences) at 4° C. for 30 min and washed once with PBS before measuring. Samples were measured on a BD LSR Fortessa and analyzed using Flowio v10.6.1 software.

For sorting collection tubes were coated with FBS. 20×10⁶ cells were stained per 0.5 ml antibody solution in a 15 ml tube. The staining was performed for 20 min at 4° C. in the dark and the cells were subsequently washed with 10 ml FACS buffer. Next, the cell concentration was adjusted to 15-20×10⁶ cells/nil in FACS buffer and the samples were passed through a 35 μm cell strainer. DAPI (Sigma-Aldrich, 1:1000 dilution) was added as a live/dead stain right before the sorting. The samples were sorted on FACSAria Fusion.

Antibodies for flow cytometry and sorting were diluted in FACS buffer. The following antibodies and dilutions were used: anti-human CD8 APC (clone SK1, BD Biosciences, 1:300 dilution) and anti-human CD8 APC-R700 (clone RPA-T8, BD Biosciences, 1:600 dilution), anti-mouse TCRβ PE (clone H57-597, BD Biosciences, 1:150 dilution), anti-human CD69 PE/APC/BV510 (clone FN50, BD Biosciences, 1:100, 1:200 and 1:300 dilutions, respectively) and with CD69 PE (clone CH/4, ThermoFisher Scientific, 1:100), anti-human CD20 FITC (clone 2H7, BD Biosciences, 1:25 dilution) and with anti-human CD20 PE-Cy7 (clone L27, BD Biosciences, 1:200 dilution), anti-human CD25 BV711 (clone 2A3, BD Biosciences, 1:200 dilution), anti-human CD62L APC (clone DREG-56, BD Biosciences, 1:25 dilution), anti-human CD3 PerCPCy5.5 (clone SK7, I3D Biosciences, 1:25 dilution). Note: unless otherwise stated anti-human CD69 clone FN50 antibody was used for the CD69 readouts.

### Retroviral Transfections and Transductions

To perform retroviral transfections, ˜1.8×10⁶ Phoenix Ampho cells were seeded in a 10 cm culture dish and transfected 24 Hater with 10 μg pMP71-TCR.plasmid using FuGENE-6 (Promega) as a transfection reagent. The transduced TCRs are comprised of mouse constant regions and human variable regions to prevent dimerization of transduced TCRs with endogenous TCRs of the Jurkat cell line. Therefore, the transduction efficiency of different TCRs can be measured by staining with a single antibody against the mouse TCRβ (mTCRβ) region. The viral supernatant was collected 48 h after the transfection and passed through a 0.45 μm filter. To perform retroviral transductions, non-TC treated 24W plates were coated with 0.5 nil/well RetroNectin (RN, Takara) and incubated either overnight at 4° C. or for 2 h at RT. The plates were blocked for 30 min. with 0.5 ml/well blocking buffer (PBS supplemented with 5% FBS) at RT and washed once with PBS. 2×10⁵ Jurkat cells in 0.5 ml medium were seeded in the RN-coated 24W plates and mixed with 0.5 ml viral supernatant. The plates were spun down at 880×g for 90 min (ace 3, dec 0). The transduction efficiency was measured after 3-4 days by flow cytometry.

### Lentiviral Transfections and Transductions

To perform lentiviral transfections, ˜3×10⁶ HEK293T cells were seeded in a 10 cm culture dish and transfected 24 h later with 10 μg pSMPUW-NFAT-IL-2 based reporter construct by using third-generation packaging constructs (pRSV-Rev and pCgpV packaging vectors and pCMV-VSV-G envelope vector, Cell Biolabs) and the transfection reagent polyethylenimine (PEI, 1 mg/ml, Polysciences) or Lifpofectamine 3000 (ThermoFisher Scientific). For some lentiviral transfections pmaxGFP (from SE Cell Line 4D-Nucleofector X Kit L, Lonza) was co-transfected as a measure of transfection efficiency. The viral supernatant was collected 48 and 72 h after the transfection and passed through a 0.45 μm filter. Primary T cells were isolated and activated with CD3/CD28 Dynabeads in the presence of medium supplemented with IL-2 and IL-15 (see ‘Isolation of T cells and B cells from human healthy donors’) for 48 h prior to the transduction. Lentiviral transductions of primary T cells were performed by a spin transduction of RN-coated plates as described in the ‘Retroviral transfections and transductions’ section (2.5×10⁵ primary T cells per well in a 24W plate). To perform lentiviral transductions of Jurkat cells, 2×10⁵ cells were resuspended in 1 ml viral supernatant with polybrene (Sigma-Aldrich). The cells were plated in a 24W plate and the transduction efficiency was measured after 3-4 days by flow cytometry. Transduced primary T cells received fresh medium with cytokines every 3-4 days.

### Generation of the Jurkat TCR KO CD8⁺ Cell Line

The Jurkat E6-1 cell line was retrovirally transduced with pMP71-CD8α-P2A-CD8β to express CD8. Additionally, the endogenous TCR expression was eliminated by knocking out the TCRα and β chains as described in the study of Scheper et al⁴⁶. Single cell clones with a high expression of CD8 were sorted and expanded.

### Electroporation of Jurkat Cells

To achieve non-viral delivery of pMKRQ-NFAT plasmids into Jurkat cells (FIG. 24), the Amaxa 4D-Nucelofaector Protocol for Jurkat clone E6.1 ATCC from Lonza (Cat. No. V4XC-1024) was followed.

### Co-Culture Assays

The CDK4 mutant (ALDPHSGHFV) and the CMVpp65 (NLVPMVATV) peptides were ordered from Pepscan. The peptides were loaded on JY cells with a density of 1×10⁶ cells/ml after 1-h incubation at 37° C. and 5% CO₂. Next, the JY cells were washed twice with medium. EBV LCLs were transduced with a retroviral vector encoding a string of 25 mer polypeptides. These polypeptides contain epitopes of interests (for instance, the CI)K4 mutant and CMVpp65 peptides) and are referred to as tandem minigene (TMG) 2. L JY cells without a cognate peptide or loaded with an irrelevant peptide and non-transduced EBV LCLs were used as negative controls.

Effector and target cells were seeded at a low density (0.25/0.5×10⁶ cells/ml) 24 h before the co-culture. Unless otherwise stated, co-cultures were performed with 200,000 effector and target cells (total volume of 200 μl; E:T ration 1:1) in a 96W round-bottom plate for 20 h; the plate was spun down at 1100 rpm for 1 min before the 20-h incubation. Anti-human CD20 antibody was included in the panel in order to gate out the target cells.

MACS GMP Cell Differentiation Bags—500 used in FIGS. 19c, 19d were purchased from Miltenyi Biotec.

### Reagents

The following additional reagents were used: 50 ng/nil phorbol 12-myristate 13-acetate (PMA, Sigma-Aldrich), 1 μM ionomycin calcium salt (Sigma-Aldrich), puromycin dihydrochloride (Gibco), blasticidin S HCI (Gibco).

### Fold Expansion

The fold expansion value in FIG. 18 was used as a measure of toxicity and calculated by dividing the total cell count after the selection by the total cell count at the beginning of the selection. The obtained value was corrected for the re-plating on day 4.

### Statistical Analysis

The calculation of the EC50 values and the statistical analyses were performed with GraphPad Prism 8. In FIG. 23 a two-way ANOVA followed by Sidak!s multiple comparisons test was performed and P values ≤0.05 were considered statistically significant (*P≤0.05, **P≤0.01).

### REFERENCES

1. Shankaran V, Ikeda Bruce A T, et al. IFNγ, and lymphocytes prevent primary tumour development and shape tumour immunogenicity. Nature. 2001; 410(6832):1107-1111. doi:10.1038/35074122
2. Davis M M, Bjorkman P J. T-cell antigen receptor genes and T-cell recognition. Nature. 1988; 334(6181)395-402. doi:10.1038/334395a0
3. Neetjes J, Jongsma M L M, Paul P, Bakke O. Towards a systems understanding of MHC class i and MHC class II antigen presentation. Nat Rev Immunol. 2011; 11(12):823-836. doi:10.1038/nri3084
4. Weiss A, Littman D R. Signal transduction by lymphocyte antigen receptors. Cell. 1994; 76(2):263-274. doi:10.1016/0092-8674(94)90334-4
5. Crabtree G R, Clipstone N A. Signal Transmission Between the Plasma. Membrane and Nucleus of T Lymphocytes. Annu Rev Biochem. 1994; 63(1):1045-1083. doi:10.1146/annurev.bi.63.070194.005145
6. Rao A, Luo C, Hogan P G. Transcription factors of the NFAT family: regulation and function. Annu Rev Immunol. 1997; 15(1):707-747. doi:10.1146/annurev.immunol.15.1.707
7. Tran E, Robbins P F, Rosenberg S A. Final common pathway of human cancer immunotherapy: Targeting random somatic mutations. Nat Immnol. 2017; 18(3):255-262. doi:1.0.1038/ni.3682
8. Schumacher T N, Scheper W, Kvistborg P. Cancer Neoantigens. Annu Rev Immunol. 2019; 37(1):173-200. doi:10.1146/annurev-immunol-042617-053402
9. Leach D R, Krummel M F, Allison J P. Enhancement of antitumor immunity by CTLA-4 blockade. Science (80-). 1996; 271(5256):1734-1736. doi:10.1126/science.271.5256.1734
10. Sharma P, Allison J P. The future of immune checkpoint therapy. Science (80-). 2015; 348(6230):56-61. doi:10.1126/science.aaa8172
11. Castle J C, Uduman M, Pabla S, Stein R B, Buell J S. Mutation-derived neoantigens for cancer immunotherapy. Front Immunol. 2019; 10(AUG). doi:10.3389/fimmu.2019.01856
12. Hodi F S, O'Day S J, McDermott D F, et al. Improved survival with ipilimumab in patients with metastatic melanoma. N Engl J Med. 2010; 363(8):711-723. doi:10.1056/NEJMoa1003466
13. Van Rooij N, Van Buuren M M, Philips D, et al. Tumor exome analysis reveals neoantigen-specific T-cell reactivity in an ipilimumab-responsive melanoma. J Clin Oncol. 2013; 31(32). doi:10.1200/JCO.2012.47.7521
14. Rizvi N A, Maziéres J, Planchard D, et al. Activity and safety of nivolumab, an anti-PD-1 immune checkpoint inhibitor, for patients with advanced, refractory squamous non-small-cell lung cancer (CheckMate 063): A phase 2, single-arm trial. Lancet Oncol. 2015; 16(3):257-265. doi: 10.1016/S1470-2045(15)70054-9
15. Borghaei H, Paz-Ares L, Horn L, et al. Nivolurnab versus docetaxel in advanced nonsquamous non-small-cell lung cancer. N Engl J Med. 2015; 373(17):1627-1639. doi:10.1056/NEJMoal1507643
16. Yee C, Thompson J A, Byrd D, et al. Adoptive T cell therapy using antigen-specific CD8+ T cell clones for the treatment of patients with metastatic melanoma: In vivo persistence, migration, and antitumor effect of transferred T cells. Proc Natl Acad Sci USA. 2002; 99(25):16168-16173. doi:10.1073/pnas.242600099
17. Rosenberg S A, Yang J C, Sherry R M, et al. Durable complete responses in heavily pretreated patients with metastatic melanoma using T-cell transfer immunotherapy. Clin Cancer Res. 2011; 17(13):4550-4557. doi:10.1158/1078-0432.CCR-11-0116
18. Stevanovic S, Draper L M, Langhan M M, et al. Complete regression of metastatic cervical cancer after treatment with human papillomavirus-targeted tumor-infiltrating T cells. J Clip Oncol. 2015; 33(14):1543-1550. doi:10.1200/JCO.2014.58.9093
19. Stevanovic S, Heiman S R, Wunderlich J R, et al. A Phase II Study of Tumor-infiltrating Lymphocyte Therapy for. Human Papillomavirus—associated Epithelial Cancers. Clin Cancer Res. 2019; 25(5): 1486-1493. doi:10.1158/1078-0432.CCR-18-2722
20. Papaioannou N E, Beniata O V., Vitsos P, Tsitsilonis O, Samara P. Harnessing the immune system to improve cancer therapy. Ann Transl Med. 2016; 4(14). doi:10.21037/atm.2016.04.01
21. Zhang J, Wang L. The emerging world of TCR-T cell trials against cancer: A systematic review. Technol Cancer Res Treat. 2019; 18. doi:10.1177/1533033819831068
22. Rosenberg S A, Yang J C, Restifo N P. Cancer immunotherapy: Moving beyond current vaccines. Nat Med. 2004; 10(9):909-915. doi:10.1038/nm1100
23. Robbins P F, Kassim S H, Tran T L N, et al. A pilot trial using lymphocytes genetically engineered with an NY-ESO-1-reactive T-cell receptor: Long-term follow-up and correlates with response. Clin Cancer Res. 2015; 21(5): 1019-1027. doi:10.1158/1078-0432.CCR-14-2708
24. Parkhurst M R, Yang J C, Langan R C, et al. T cells targeting carcinoembryonic antigen can mediate regression of metastatic colorectal cancer but induce severe transient colitis. Mol Ther. 2011; 19(3):620-e6. doi:10.1038/mt.2010.272
25. Morgan R A, Chinnasamy N, Abate-Daga D, et al. Cancer regression and neurological toxicity following anti-MAGE-A3 TCR gene therapy. J. Immunother. 2013; 36(2):133-151. doi:10.1097/CJI.0b013e3182829903
26. Schumacher T N, Schreiber R D. Neoantigens in cancer immunotherapy. Science (80-). 2015; 348(6230):69-74. doi:10.1126/science.aaa4971
27. Tran E, Turcotte S, Gros A, et al. Cancer immunotherapy based on mutation-specific CD4+ T cells in a patient with epithelial cancer. Science (80-). 2014; 344(6184):641-645. doi:10.1126/science.1251102
28. Tran E, Robbins P F, Lu Y-C, et al. T-Cell Transfer Therapy Targeting Mutant KRAS in Cancer. N Engl J Med. 2016; 375(23):2255-2262. doi:10.1056/NEJMoa1609279
29. Rodenko B, Toebes M, Hadrup S R, et al. Generation of peptide-MHC class I complexes through UV-mediated ligand exchange. Nat Protoc. 2006; 1(3):1120-1132. doi:10.1038/nprot.2006.121
30. Cohen C J, Gartner J J, Horovitz-Fried M, et al. isolation of neoantigen-specific T cells from tumor and peripheral lymphocytes. J Clin Invest. 2015; 125(10):3981-3991. doi:10.1172/JC182416
31. Yadav M, Jhunjhunwala S, Phung Q T, et al. Predicting immunogenic tumour mutations by combining mass spectrometry and exome sequencing. Nature. 2014; 515(7528):572-576. doi:10.1038/nature14001
32. López-Cabrera. M, Santis A G, Fernández-Ruiz E, et al. Molecular cloning, expression, and chromosomal localization of the human earliest lymphocyte activation antigen AIM/CD69, a new member of the C-Type animal lectin superfamily of signal-transmitting receptors. J Exp Med. 1993; 178(2):537-548. doi:10.1084/jem.178.2.537
33. Krishnamani V, Stamnes M A, Piper R C. MALTA: A calculator for estimating the coverage with shRNA, CRISPR, and cDNA libraries. SoftwareX. 2019; 9:154-160. doi:10.1016/j.softx.2019.01.006
34. Wölfel T, Hauer M, Schneider J, et al. A p161NK4a-insensitive CI)K4 mutant targeted by cytolytic T lymphocytes in a human melanoma. Science (80-). 1995; 269(5228):1281-1284. doi:10.1126/science.7652577
35. Wills M R, Carmichael A J, Mynard K, et al. The human cytotoxic T-lymphocyte (CTL) response to cytomegalovirus is dominated by structural protein pp65: frequency, specificity, and T-cell receptor usage of pp65-specific CTL. J Vivol. 1996; 70(11):7569-7579. doi:10.1128/jvi.70.11.7569-7579.1996
36. Galkina E, Tanousis K, Preece G, et al. L-Selectin Shedding Does Not Regulate Constitutive T Cell Trafficking but Controls the Migration Pathways of Antigen-activated T Lymphocytes. J Exp Med. 2003; 198(9):1323-1335. doi: 10.1084/jem.20030485
37. Hooijberg E, Bakker A Q, Ruizendaal J J, Spits H. NFAT-controlled expression of GFP permits visualization and isolation of antigen-stimulated primary human T cells. Blood. 2000; 96(2):459-466. doi: 10.1182/blood. v96.2.459.014k50_459_466
38. Jutz S, Leitner J, Schmetterer K, et al. Assessment of costimulation and coinhibition in a triple parameter T cell reporter line: Simultaneous measurement of NF-κB, NFAT and AP-1. J Immunol Methods. 2016; 430:10-20. doi:10.1016/j.jim.2016.01.007
39. Morimoto S, Fujiki F, Kondo K, et al. Establishment of a novel platform cell line for efficient and precise evaluation of T cell receptor functional avidity. Oncotarget 2018; 9(75):34132-34141. doi:10.18632/oncotarget.26139
40. Rydzek J, Nerreter T, Peng H, et al. Chimeric Antigen Receptor Library Screening Using a Novel NF-κB/NFAT Reporter Cell Platform. Mol Ther. 2019; 27(2):287-299. doi:10.1.016/j.ymthe.201.8.11.015
41. Zufferey R, Dull Mandel R J, et al. Zufferey, R., Dull, T., Mandel, R. J., Bukovsky, A., Quiroz, D., Naldini, L., & Trono, D. (1998). Self-inactivating lentivirus vector for safe and efficient in vivo gene delivery. Journal of Virology, 72(12), 9873-80. Retrieved from http://www.ncbi.nlm.ni. J Virol. 1998; 72(12):9873-9880. Accessed May 21, 2020. http://www.ncbi.nlm.nih.gov/pubmed/9811723%0 Ahttp://www.pubmedcentral.nih.go v/articlerender.fcgi?artid=PMC110499
42. Spindler M J, Nelson A L, Wagner. E K, et al. Massively parallel interrogation and mining of natively paired human TCRαβ repertoires. Nat Biotechnol. Published online 2020. doi:10.1038/s41587-020-0438-y
43. Reddy M, Eirikis E, Davis C, Davis H M, Prabhakar U. Comparative analysis of lymphocyte activation marker expression and cytokine secretion profile in stimulated human peripheral blood mononuclear cell cultures: An in vitro model to monitor cellular immune function. J Immunol Methods. 2004; 293(1-2):127-142. doi:10.1016/j.jim.2004.07.006
44. Chao C C, Jensen R, Dailey M O. Mechanisms of L-selectin regulation by activated T cells. J Immunol. 1997; 159(4):1686-1694. Accessed May 15, 2020. http://www.ncbi.nlm.nih.gov/pubmed/9257829
45. Yang S, Liu F. Wang Q J, Rosenberg S A, Morgan R A. The Shedding of CD62L (L-Selectin) Regulates the Acquisition of Lytic Activity in Human Tumor Reactive T Lymphocytes. Teague R M, ed. PLaS One. 2011; 6(7):e22560. doi:10.1371/journal.pone.0022560
46. Scheper W, Kelderman S, Fanchi L F, et al. Low and variable tumor reactivity of the intratumoral TCR repertoire in human cancers. Nat Med. 2019; 25(1):89-94. doi:10.1038/s41591-018-0266-5

### Example 20

This example describes recovery of antigen-specific TCRs from a TCR library using different Effector-to-Target (E:T) ratios.

Five characterized, TCRs of known antigen reactivity, as well as 95 uncharacterized TCRs (for 100×100 libraries; FIG. 35A) were selected for TCR library generation (performed by Twist Bioscience; San Francisco/USA). In brief, the TCRα- and β-chains from these TCRs were synthesized as fragments by DNA synthesis. For library generation all selected TCRα and TCRβ fragments were combinatorially joined to continuous nucleic acid molecules encoding TCRβ-P2A-TCRα cassettes (SEQ ID NO: 1). A retroviral construct containing both beta and alpha TCR chains, as well as a puromycin selection marker can be used as a scaffold for creating the library. Retroviral transduction using this construct ultimately leads to expression of a single transcript, which results in translation of the TCR beta and alpha chains, as well as a puromycin resistance marker, due to peptide cleavage at the 2A sites. One TCRβ- and TCRα-fragment can be joined per cassette in the thrmat of TCRβ-P2A-TCRα-T2A-Puromycin resistance (SEQ ID NO: 1). The TCR-beta and TCR-alpha regions of each of the 100 TCRs were synthesized and inserted into this vector in a combinatorial cloning approach executed by Twist Bioscience.

The 100×100 libraries were transfected into Phoenix-Ampho virus producer cells (ATCC CRL-3213) using Fugate transfection reagent and protocols known to the skilled artisan. The resulting retroviral virions were used to transduce a Jurkat reporter T cell line. The Jurkat reporter T cell lacks endogenous TCR expression (generation of such a genetic knock-out being described for example in Mezzadra et al Nature 2017) and is modified to express human CD8α and CD8β after transduction with a CD8α-P2A-CD8β transgene (SEQ ID NO: 2) using methods known to the skilled artisan. Jurkat reporter T cells were transduced with the TCR library resulting in 25% TCR-modified T cells of total live T cells (based on staining 4 days after transduction—after puro selection and on the day of the assay the purity was >80%). The use of murine TCR constant domain sequences in the TCR library (SEQ ID NO: 1) allows for the detection of TCR-modified Jurkat T cells by flow cytometry using a murine TCRβ constant domain specific antibody. Jurkat T cells modified to express a TCRβ-P2A-TCRα-T2A-Puromycin resistance transgene were positively selected to high purity by addition of Puromycin to the cell culture media. Antibiotic selection of genetically modified cells is known to an ordinary person skilled in the art.

Next, TCR transduced Jurkat T cells were stimulated with APCs that were engineered to present antigens. EBV-LCL cells expressing a TMG were spiked in at 10% with EBV-LCLs that were not engineered to express specific antigens. Three days prior to the co-culture Jurkat reporter T cells were seeded at a low density (0.1×10⁶ cells per ml). APCs were mixed with Jurkat reporter T cells in 1:1, 1:2 and 1:3 ratios at a concentration of 2.5×10⁶ cells per ml. A single replicate of each E:T ratio was perfoinied (presence of TMG expression), and a single negative control replicate (absence of TMG expression) was included. 200 uL (0.5×10⁶ cells per well) was distributed over ˜360, 540 or 720 wells of a U-bottom TC-treated 96-well plate, respectively. Plates were centrifuged at 1000 rpm for 1 minute, and incubated for 20-22 hours at 37° C.

Subsequently, co-cultures were harvested and the cells were labelled with anti-CD20 microbeads from Miltenyi and were transferred to LS-columns that were placed on a magnet. Using magnetic bead selection, CD20+ cells were kept in the column while the CD20− cells passed through the column and were collected in the negative fraction. These CD20− cells were then stained with fluorochrome-labelled antibodies for CD20 and CD69. Next, the cells were fixed using a fixation buffer containing 4% formaldehyde. Using a Biosciences ARIAFusion flow cytometry sorter the following populations were sorted for each stimulation condition (FIG. 35B):
1. Lymphocyte, single cell, CD20⁻, CD69^{hi} (CD69^{hi} includes the highest 10-15% of single cell, CD20⁻ cells based on CD69 fluorescence signal)
2. Lymphocyte, single cell, CD20⁻, CD69^{lo} (CD69^{lo} includes 10-15% of the single cell, CD20⁻ cells based on a low CD69 fluorescence signal)

Genomic DNA was isolated from the sorted TCR transduced Jurkat T cells and used as template for multiple rounds of PCR with a limited number of cycles to amplify part of the TCRβ-P2A-TCRα cassette using PCR methods known to the skilled artisan. The resulting PCR product has a size of approx. 1.5 kb (FIG. 35C) and can be sequenced using an Oxford Nanopore Minion or Gridlon sequencing instrument using techniques known to a person skilled in the art. TCR identities were recovered using alignment techniques known to the skilled artisan and differentially expressed TCR combinations are identified using the DESeq2 R package. Rlog-transformed read counts were calculated using the DESeq2 R package, and the Rlog-values of bottom samples were subtracted from the Rlog-values of top samples and represented for cocultures that were performed in the presence (x-axis) and absence (y-axis) of TMG expression by B cells (FIG. 35D). The 5 characterized TCRs are depicted as larger grey dots. TCR identities, as well as key statistical metrics are represented for the five characterized TCRs (FIG. 35E).

Taken together, this example shows that increasing the ratio of target cells to effector cells improves the sensitivity of the TCR library screening platform.

### Example 21

This example describes recovery of antigen-specific TCRs from a TCR library generated by gene synthesis using a bead-based cell sorting strategy.

Five characterized TCRs of known antigen reactivity, as well as 95 uncharacterized TCRs (for 100×100 libraries; FIG. 36A) were selected for TCR library generation (performed by Twist Bioscience; San Francisco/USA). In brief, the TCRα- and β-chains from these TCRs were synthesized as fragments by DNA synthesis. For library generation all selected. TCRα and TCRβ fragments were combinatorially joined to continuous nucleic acid molecules encoding TCRβ-P2A-TCRα cassettes (SEQ ID NO: 1). A retroviral construct containing both beta and alpha TCR chains, as well as a puromycin selection marker can be used as a scaffold for creating the library. Retroviral transduction using this construct ultimately leads to expression of a single transcript, which results in translation of the TCR beta and alpha chains, as well as a puromycin resistance marker, due to peptide cleavage at the 2A sites. One TCRβ- and TCRα-fragment can be joined per cassette in the format of TCRβ-P2A-TCRα-T2A-Puromycin resistance (SEQ ID NO: 1). The TCR-beta and TCR-alpha regions of each of the 100 TCRs were synthesized and inserted into this vector in a combinatorial cloning approach executed by Twist Bioscience.

The 100×100 libraiywas transfected into Phoenix-Ampho virus producer cells (ATCC CRL-3213) using Fugene transfection reagent and protocols known to the skilled artisan. The resulting retroviral virions were used to transduce a Jurkat reporter T cell line. The Jurkat reporter T cell lacks endogenous TCR expression (generation of such a genetic knock-out being described for example in Mezzadra et al Nature 2017) and is modified to express human CD8α and CD8β after transduction with a CD8α-P2A-CD8β transgene (SEQ ID NO: 2) using methods known to the skilled artisan. Jurkat reporter T cells were transduced with the TCR library resulting in 25% TCR-modified T cells of total live T cells (based on staining 4 days after transduction—after puro selection and on the day of the assay the purity was >80%). The use of murine TCR constant domain sequences in the TCR library (SEQ ID NO: 1) allows for the detection of TCR-modified Jurkat T cells by flow cytometry using a murine TCRβ constant domain specific antibody. Jurkat T cells modified to express a TCRβ-P2A-TCRα-T2A-Puromycin resistance transgene were positively selected to high purity by addition of Puromycin to the cell culture media. Antibiotic selection of genetically modified cells is known to an ordinary person skilled in the art.

Next, TCR transduced Jurkat T cells were stimulated with APCs that were or were not engineered to present tumour-derived antigens (1 replicate each). Three days prior to the co-culture Jurkat reporter T cells were seeded at a low density (0.1×10⁶ cells per ml). APCs were mixed with Jurkat reporter T cells in a 1:1 ratio at a concentration of 2.5×10⁶ cells per ml. 200 uL (0.5×10⁶ cells per well) was distributed over ˜360 wells of a U-bottom TC-treated 96-well plate. Plates were centrifuged at 1000 rpm for 1 minute, and incubated for 20-22 hours at 37° C.

Subsequently, co-cultures were harvested and dead cells were removed using a dead cell removal kit. The live cells were separated based on a multi-step isolation strategy (FIG. 36B), labelled with anti-CD20 microbeads and transferred onto the AutoMACS (Miltenyi, Bergisch Gladbach, Germany). Using magnetic bead selection, CD20+ cells were kept in the column while the CD20− cells passed through the column and were collected in the negative fraction. These CD20− cells were then labelled with CD62L microbeads to separate the non-activated Jurkat cells from the activated Jurkat cells using autoMACS. After this separation, both the negative (CD62L− cells) and positive fraction (CD62L+ cells) were labelled with CD69-biotin antibody and CD69− and CD69+ fractions were separated using anti-biotin microbeads. The following populations were collected for further analysis (FIG. 36B):
1. CD20⁻, CD62L+, CD69− (”bottom” fraction→non-activated Jurkat T cells)
2. CD20⁻, CD62L−, CD69+ (“top” fraction→activated Jurkat T cells)

Genomic DNA was isolated from the bead-based sorted TCR transduced Jurkat T cells and used as template for multiple rounds of PCR with a limited number of cycles to amplify part of the TCRβ-P2A-TCRα cassette using PCR methods known to the skilled artisan. The resulting PCR product has a size of approx. 1.5 kb (FIG. 36C) and can be sequenced using Oxford. Nanopore Minton or Gridlon sequencing instruments using techniques known to a person skilled in the art. TCR identities were recovered using alignment techniques known to the skilled artisan and differentially expressed TCR combinations are identified using the DESeq2 R package. Riog-transformed read counts for each TCR were calculated using the DESeq2 R package, and the Rlog-values of the bottom s were subtracted from the Rlog-values of the top samples and represented for cocultures that were performed in the presence (x-axis) and absence (y-axis) of TMG expression by B cells (FIG. 36D). TCR alpha and beta chain identity, as well as key statistical metrics are represented for the top 7 most differentially represented TCRs in the top samples (FIG. 36E).

Taken together, this example shows that bead-based sorting is successful in separating activated cells from non-activated cells and that this technique can be used as a way to identify antigen-specific TCRs from a TCR library using a genetic screening approach as an alternative to FACS-based cell separation.

### Example 22

This example describes that combinatorial TMG encoding can be resolved. using pairwise TCR enrichment analysis. This example demonstrates the possibility to mix pools of TMG-expressing APCs using combinatorial TMG encoding, and resolution of the TMG that is expressing an antigen that is recognized by a TCR from a TCR library.

Combinatorial TMG encoding allows one to efficiently screen large numbers of TMGs without scaling the numbers of screens accordingly, whilst maintaining the potential to identify the TMG that is recognized by a TCR lead from TCR library screen data. Combinatorial TMG encoding is based on the principle of making pools of APCs expressing TMGs, and that each TM is represented in exactly one combination of pools. Since the combination of pools is unique for each TMG, this allows for the identification of the TMG that is recognized by a TCR in a TCR library screen. The TMG encoding the antigen recognized by a given TCR lead from a TCR library screen can be identified using pairwise TCR enrichment analysis, which serves to increase signal strength by specifically analyzing pairs of pools rather than single pools separately.

One example of combinatorial TMG design is represented in FIG. 37A). 36 TMGs are expressed in 12 pools (C1-6 and R1-6), with each pool expressing 6 TMGs. Pools of APCs expressing TMGs may be created by polyclonal APC engineering (e.g., polyclonal virus production and APC transduction or monoclonal virus production, mixing of virus into pools, and polyclonal APC transduction), or by mixing engineered APC cell lines each expressing a single TMG. As an example, if pairwise TCR enrichment analysis shows that both APC pools C1 and R1 can activate a given TCR, that TCR is recognizing an antigen expressed from TMG1. This is because TMG1 is the only TMG represented in both pools. While in FIG. 37A) the combinatorial TMG encoding is based on the presence of a TMG in exactly two pools, similar combinatorial TMG encoding designs can be made where each TMG is present in exactly 3, 4, 5, 6, 7, 8, 9, 10 or more pools. In the case where each TMG is present in exactly 3 pools, identification of the 3 pools that are recognized by a given TCR will allow deduction of the TMG that is recognized. The TMG that is recognized is the TMG that is shared by all 3 pools.

In FIG. 10G), the results for triplicate screens are shown, which include 3 control screens where TCR library-expressing reporter T cells were cocultured with APCs that have not been engineered to express a TMG, and 3 screens based on cocultures with a pool of APC cell lines each expressing a single TMG. Combinatorial TMG encoding as described above, where each TMG is represented. In exactly 2 pools, involves sufficient TCR identification sensitivity in a screening approach based on two replicates. For statistical. analyses of differential represented. TCR alpha x beta chain combinations from the data in FIG. 10G) the DESeq2 R package was used. As shown in FIG. 37B) and 37C), we tested whether either ranking or significance of TCR leads identified in the CRC patient screens (FIG. 10G) were similar based on 2 out of 3 replicates. As shown in FIG. 37B), when picking 7 TCR leads for pt2, the same TCRs would have been picked based on combined analysis of any combination of 2 out of the 3 replicates. In an analogous manner, for any of the TCR leads identified in FIG. 10G), the same (set of) TCRs would have been picked based on using 2 out of 3 replicates for statistical analysis (FIG. 37C; ‘Ranks’ column). In addition, adjusted p-values are similar for statistical analyses based on 2 or 3 replicates for any of the TCR leads identified in the 4 CRC patient TCR library screens (FIG. 37C). This shows that 2 replicates are sufficient for TCR identification with similar sensitivity to screens using 3 replicates. This allows for identification of the recognized TMG using a combinatorial TMG design where each. TMG is represented exactly twice in the TMG pools.

To test the sensitivity of pairwise TCR enrichment analysis to resolve TMG recognition from a combinatorial TMG encoding design, six pt4 samples (each consisting of a top and a bottom sample) were selected for the analysis (FIG. 37D). All 15 possible pairwise combinations of the 6 samples were analyzed using DESeq2 for differential representation of TCRs in the top sample relative to the bottom sample. An interaction model was used for the analyses, where the TCR enrichment in the top samples relative to the bottom samples of a pair of pools was contrasted against TCR enrichment in the top samples relative to the bottom samples of all other pools. All adjusted p-values were rank-ordered from lowest to highest p-value, and represented in FIG. 37E). Two adjusted p-values were standing out from the rest. These represent TCR. alpha9.beta14 in pairwise TCR enrichment analysis of samples 4 and 5, and TCR alpha43.be al6 in pairwise TCR enrichment analysis of samples 3 and 5. These results show that TMG4, which is represented in samples 4 and 5, and TMG3, which is represented in samples 3 and 5, are encoding an antigen recognized by these respective TCRs.

Taken together, this example shows that pools of APCs each expressing multiple TMGs can be used to perform TCR library screens against a large number of minigenes, and that this combinatorial TMG encoding approach allows for identification of both antigen-reactive TCRs as well as the TMG that is expressing the recognized antigen.

### Example 23

This example describes that TCR characteristics can be derived from genetic screening data. This example demonstrates that TCR library screening approaches are sufficiently sensitive to derive TCR characteristics. To this end, functional genetic screening data from the TCR identification platform are compared to independent validation experiments with respect to induced activation and background activation of TCRs.

Sixteen CRC pt2 TCRs identified in FIG. 10G) were assessed for reactivity to the recognized TMG (TMG2). A good con⁻elation is observed between the activation observed in the genetic screening approach, and the activation observed in validation experiments (FIG. 38); middle planel). In addition, a good correlation was observed between the background activation observed in the genetic screening approach and the background activation observed in the validation experiment (FIG. 38); right panel).

These data show that TCR characteristics, which can be determined in independent experiments after TCR identification using a TCR library screening approach, can be determined during the screening stage from functional genetic screening data. For instance, the relative TCR sensitivity, as well as TCR activation in the absence of antigen can be determined from TCR library screens as exemplified in FIG. 38). The relative TCR sensitivity may be important to determine the optimal TCR for engineering a T cell product for treating a cancer patient. TCR activation in the absence of antigen may constitute a negative selection criterion, as engineered T cells expressing such TCRs may recognize antigens presented on non-cancerous cells.

Additional screens may be included during the TCR library screening stage to reduce the number of TCR characterization assays that need to be performed after the screening stage. These include, but are not limited to: i) Screens with APCs expressing wild-type TMGs as a control to determine mutation-specificity of the TCR during the TCR library screening phase; ii) Screens with APCs engineered to lack essential components of class I or class II presentation (B2M or CIITA/CD74) and engineered to express TMGs as controls to determine class I/II restriction of TCRs; iii) Screens with APCs expressing TMGs from promoters with different strengths to determine TCR sensitivity.

In sum, this example shows that TCR characteristics can be determined from TCR library screens, which has the benefit of reducing the number of experiments that need to be performed for TCR characterization after the TCR library screening stage, and therefore can reduce the total amount of time required from screening up to and including TCR characterization.

### Example 24

### Recovery of Antigen-Reactive TCRs from TCRαβ Libraries Through the Isolation of a Sub-Population of Reporter T Cells (“Top-Bottom Approach”)

This example describes the recovery of antigen-reactive TCRs from TCRαβ libraries through the isolation of one or more sub-populations based on response to antigen. In short, this approach entails the following steps: i) genetic engineering of reporter T cells to allow expression of TCRs of the TCRαβ libraries; ii) performing a coculture of these cells with antigen-presenting cells expressing at least one antigen; iii) cell separation based on T cell activation markers into a) a ‘top’ population expressing one or multiple markers of T cell activation; and b) a ‘bottom’ population lacking (or having low) expression of one or multiple markers of T cell activation; iv) TCR identification from the top and bottom samples using PCR on genomic DNA and subsequent deep sequencing; and v) identification of at least one antigen-reactive TCR which is enriched in the top sample relative to the bottom sample. Expression of a marker of T cell activation can be relatively high expression of a marker demarcating activated T cells (CD69), or relatively low levels of expression of a marker demarcating non-activated T cells (CD62L).

The principle behind the top-bottom approach is that antigen-reactive TCRs will become activation-marker positive upon antigen stimulation, and therefore such TCRs will be enriched in the top population relative to the bottom population. The top-bottom approach is illustrated by various accompanying figures as described below.

In alternative embodiments, the bottom sample may be any reference population of cells or reference library of TCR plasmids. The bottom sample may be sorted from the same population of cells as the top sample, but having low activation marker expression. The bottom sample may be obtained from cocultures of reporter T cells expressing the relevant TCR library, and B cells that are not engineered to express exogenous antigens. The bottom sample may be the TCR plasmid library that was used to create the reporter T cells from which the top sample was sorted. In some embodiments, the TCR representation in top and bottom samples may be compared to TCR representation in any other additional sample during differential TCR representation analysis. In some embodiments, such additional samples may be the plasmid. TM library. In other embodiments, such additional samples may be derived from cocultures of reporter T cells expressing the relevant TRC library, and B cells that are not engineered to express exogenous antigens.

The rlog values in FIG. 39) denote a measure of TCR representation. The graph shows that five characterized TCRs which are stimulated with their cognate antigens are enriched in the top samples (lightest grey shade) relative to any of the other samples. The bottom samples derived from cocultures with B cells expressing TMG show lower rlog-values than top or bottom samples from cocultures with B cells that are not engineered to express exogenous antigens. Based on these results, a person skilled in the art will recognize that identification of antigen-reactive TCRs using a top-bottom approach will be more sensitive with i) bottom samples derived from the same population of cells as the top sample, but having low activation marker expression (Bottom+TMG); than with ii) bottom samples derived from cocultures with B cells that are not engineered to express exogenous antigens. This is because the difference between Top+TMG and Bottom+TMG samples is bigger than the difference between Top+TMG and Top/Bottom−TMG samples, allowing more sensitive detection of TCR reactivity using the former comparison. The bottom sample (i.e., reference TCR representation) may be derived from the relevant plasmid TCR library (black dots in FIG. 39), which correlates with the TCR representation in top or bottom samples from cocultures with B cells that are not engineered to express exogenous antigens.

Additional aspects of this Example are shown in 39, which shows an additional analysis of the genetic screen on the 100×100 combinatorial library from FIG. 14F, G). This example illustrates the top-bottom approach, where the cells in the bottom sample are ideally sorted from the same population as the cells in the top sample, but activation-marker negative. In FIG. 39) the data from FIG. 14 F,G) were Rlog-transformed using the DESeq2 R package and represented for every of the five characterized TCRs. Each dot represents an independent replicate and dots are colored according to the type of sample. Top samples represent the top 10% of cells with highest CD69 expression, while bottom samples represent cells that were sorted from the same population of cells but which displayed low CD69 expression. In FIG. 16E) the top-bottom approach was applied on a Jurkat hCD8+ cell pool that was created from 24 T cell lines that each express a different TCR with known identity but with unknown specificity. These cell lines were mixed in a 1:1 ratio and Jurkat hCD8+ cells expressing either the CDK4-17, CDK4-8, CMV#1, CMV#2 or GCN1L1 TCR were also mixed in various frequencies ranging from 1:10,000-1:1,000,000. After coculture with B cells expressing the cognate antigens for these 5 TCRs, cells were stained for CD69 (T cell activation marker). The top 10% of T cells expressing highest CD69 were FACS-sorted into the top sample, and a roughly equal amount of T cells expressing low levels of CD69 were FACS-sorted into the bottom sample. TCR content in both samples was analyzed using PCR on genomic DNA, followed by next-generation sequencing using the Illumina platform. The relative abundance of TCRs in the top versus the bottom samples was calculated, and enrichment of all five TCRs of known specificity was observed using this approach.

In FIGS. 13A)-E), a TCR library was created by mixing plasmids expressing six antigen-specific TCRs at 1:10,000-1:100,000 frequencies into a pool of plasmids with known identity but unknown specificity. This plasmid pool was used to produce retrovirus and infect Jurkat hCD8+ TCR KO cells. A coculture was performed between this polyclonal pool of reporter T cells and B cells expressing the cognate antigens for the antigen-specific TCRs. The top 10% of T cells expressing highest CD69 were FACS-sorted into the top sample, and a roughly equal amount of T cells expressing low levels of CD69 were FACS-sorted into the bottom sample (FIG. 13C). TCR content in both samples was analyzed using PCR on genomic DNA (FIG. 13D), followed by next-generation sequencing using the Illumina platform. The relative abundance of TCRs in the top versus the bottom samples was calculated, and enrichment of all five TCRs of known specificity was observed using this approach (FIG. 13E).

In FIGS. 10A)-J), a combinatorial TCR library was created from the most prevalent TCRα and TCRβ chains obtained using bulk TCR sequencing of a colorectal cancer (CRC) specimen. This library was used for retroviral transduction of Jurkat hCD8+ TCR KO cells (FIG. 10D), which were cocultured with EBV-B cells expressing tumor-specific neo-antigens. The top 10% of T cells expressing highest CD69 were FAGS-sorted into the top samples, and a roughly equal amount of T cells expressing low levels of CD69 were FACS-sorted into the bottom samples (FIG. 10E). TCR content in all samples was analyzed using PCR on genomic DNA (FIG. 10F), followed by next-generation sequencing using Oxford Nanopore technology. The relative abundance of TCRs in the top versus the bottom samples was calculated. Using this approach for three additional CRC specimens, we identified in total 11 candidate neoantigen-reactive TCRs (FIG. 10G). Four of these have been further validated in independent coculture experiments (FIGS. 10H-K).

In FIGS. 36A)-E), top and bottom samples are separated using bead-based sorting using autoMACS (Miltenyi, Bergisch Gladbach, Germany). Five characterized TCRs and 95 uncharacterized TCRs from ovarian cancer (OVC) or colorectal cancer (CRC) samples were used to create combinatorial TCR libraries of 100×100 design, which was expressed in reporter T cells. After coculture with B cells expressing the cognate antigens of the five characterized TCRs, B cells were depleted using CD20-microbeads. The top and bottom sample were then obtained using sequential bead-based sorting using the CD62L and CD69 markers. CD62L is a marker expressed on non-activated T cells, and CD69 is a marker expressed on activated T cells. Cells that did not bind CD62L-microbeads and that did bind CD69-microbeads were separated into the top sample, and cells that did bind CD62L-microbeads and that did not bind CD69-microbeads were separated into the bottom sample. TCR content in both samples was analyzed using PCR on genomic DNA (FIG. 36C), followed by next-generation sequencing using Oxford Nanopore technology and screen analysis (FIGS. 36D, E). The five characterized TCRs were among the 7 most significantly enriched TCRs in the top samples relative to the bottom samples. This shows that bead-based sorting can be used as an alternative strategy to separate top and bottom samples in a TCR library screening approach.

This example shows that the top-bottom approach can be generally applied to reporter T cells expressing multiple TCRs to allow functional genetic screening for antigen-reactive TCRs. The top-bottom approach can be applied independent of the manner in which polyclonal reporter T cells expressing multiple TCRs were created. This is supported by identification of antigen-reactive TCRs from TCR-expressing reporter T cells that were obtained in various ways: i) by mixing reporter T cell lines each expressing a single defined TCR; ii) by mixing plasmids encoding defined TCRs and polyclonal virus production and transduction of reporter T cells; and iii) by using a TCR library for polyclonal virus production and transduction of reporter T cells.

In addition, this example shows that the top-bottom approach can be applied to various cell separation and TCR identification techniques: i) staining with various T cell activation markers (CD69 alone or CD62L/CD69 combination); ii) separation with various cell sorting techniques (FACS sorting and bead-based sorting); and iii) analysis with various next generation sequencing techniques (Illumina and Oxford Nanopore technologies).

In some embodiments, the approach in the example above (the top-bottom approach) can be applied to any method of creating TCR libraries including, but not limited to, i) various ways of library design described in FIG. 15; ii) library design based on TCR. sequencing techniques that identify both TCRa and TCRb chain sequences that are expressed within a single cell (paired TCR sequencing); iii) library design based on in-cell linking of TCRa and TCRb sequences and subsequent cloning. The top-bottom approach may be based on cell separation by any T cell activation markers, or combinations of T cell activation markers, including but not limited to, CD69, CD62L, CD137, CD25, IFN-γ, IL-2, INF-α, GM-CSF. The top-bottom approach may be applied to any proliferation marker, including but not limited to, CellTrace staining. The top-bottom approach may be applied to cell separation based on any T cell activation reporter, including but not limited to, CD69(promoter)-EGFRt and CD69(promoter)-LNGFR reporters. The top-bottom approach may be applied to any cell separation techniques, including but not limited to, FACS-based sorting, microfiuidics-based cell sorting and bead-based cell sorting. The top-bottom approach may be analyzed using any next generation sequencing technique, including but not limited to, sequencing using IIlumina, Oxford Nanopore or Pacific Biosciences technology.

1. A method to recover a repertoire of T cell receptors (TCRs) from diverse T cell populations, the method comprising:
determining TCR-α and β nucleotide or amino acid sequences within a subject's sample;
selecting one or more subsets of TCRα- and β-chain sequences from the total repertoire;
creating a TCR repertoire by combinatorial pairing of selected TCRα- and β-chain sequences creating a library of TCRαβ pairs; and
identifying at least one TCRαβ pair with desired features from the created TCR repertoire.

2. The method of claim 1, wherein the one or more subsets of TCRα- and β-chain sequences from the total repertoire is selected based on at least one criterion:
on frequency within the T cell population,
on relative enrichment compared to a second T cell population,
on relative difference of DNA and RNA copy numbers of a given TCR chain
on biological properties of the TCR chain, wherein the properties are selected from at least one of: (predicted) antigen-specificity, (predicted) HLA-restriction, antigen-affinity, co-receptor dependency, parental T cell lineage (e.g. CD4 or CD8 T cell) or TCR sequence motifs,
on spatial patterns of gene expression, wherein spatial gene expression patterns are derived from at least one of: originating region in the tissue or co-expression patterns of other genes,
on co-occurrence or occurrence at a similar frequency in multiple samples, for example occurrence in multiple tumor lesions,
assignment to multiple groups to separately recover specific parts of the TCR repertoire,
on a combination of multiple criteria as defined in the different embodiments.

3. The method of claim 2, wherein selection based on frequency within the T cell population is based upon data of the frequency of TCR sequences, which is used to create a separate rank order for TCRα- and β-chains or a combined rank order for TCRα- and β-chains.

4. (canceled)

5. (canceled)

6. (canceled)

7. The method of claim 1, wherein creating a TCR repertoire by combinatorial pairing of selected TCRα- and β-chain sequences creating a library of TCRαβ pairs is achieved by at least one of the following:
TCR chain sequences are used to synthesize separate libraries of TCRα- and β-chain DNA or RNA fragments which are subsequently linked into one DNA or RNA fragment in which exactly one TCRα- and one β-chain are linked,
combinations of TCRα- and β-chains are generated by directly synthesizing DNA or RNA fragments in which exactly one TCRα- and one β-chain are linked,
combinations of TCRα- and β-chains are created intracellularly by modification of a pool of cells with separate collections of TCRα- and β-genes in such a way that cells will express at least one TCRα- and one β-chain, and/or
combinations of TCRα- and β-chains are linked in a single-chain TCR construct in which both TCRα and TCRβ Variable chain fragments are fused and in which the single chain TCR construct may be fused to (i) a transmembrane domain alone or (ii) additionally contain intracellular signaling domains, including but not limited to CD3ϵ or CD3ζ signaling domains alone or in combination with a CD28 signaling domain.

8. (canceled)

9. The method of claim 1, wherein the subject's sample comprises non-viable starting material.

10. (canceled)

11. The method of claim 1, wherein antigen-specific TCR sequences are recovered.

12. The method of claim 1, wherein Class I and/or Class II restricted TCR sequences are recovered.

13. (canceled)

14. The method of claim 11, further comprising the step of administering T cells expressing the neo-antigen specific TCR sequences as a cancer therapy.

15. (canceled)

16. (canceled)

17. (canceled)

18. (canceled)

19. (canceled)

20. The method of claim 1, wherein DNA and RNA isolation is from a T cell population that is a mixture of different cell types or part of a tissue sample (such as blood or tumor tissue).

21. (canceled)

22. The method of claim 20, wherein the cells are tumor-specific T cells or tumor-infiltrating lymphocytes.

23. (canceled)

24. The method of claim 1, further comprising using the TCRαβ chain sequences to treat a subject suffering from cancer, an immunological disorder, an autoimmune disease, or an infectious disease.

25. The method of claim 1, wherein identifying at least one TCRαβ pair with desired features from the created TCR repertoire is achieved by at least one of the following:
identification or selection based on at least one activation marker;
identification or selection based on proliferation in response to antigen;
identification or selection based on identification of TCR genes of higher abundance in antigen-stimulated cells as compared to unstimulated cells;
identification or selection based on reporter gene activation by TCR triggering;
identification or selection based on selective survival, including but not limited to acquired antibiotic-resistance upon TCR signaling;
identification or selection based on binding to one or more MEW complexes;
identification or selection using single-cell based droplet PCR or microfluidics;
or any combination thereof

26. (canceled)

27. (canceled)

28. (canceled)

29. A method of creating multiple T cell libraries, the method comprising:
recovering a repertoire of T cell receptors (TCRs) according to the method of claim 1; selection of TCRα- and β-chain sequences from the total repertoire into multiple groups to separately recover specific parts of the TCR repertoire, wherein multiple T cell libraries are created that are of smaller complexity or that recover specific parts of the TCR repertoire.

30. (canceled)

31. A method of identifying a nucleotide sequence from a combinatorial library of nucleic acids, comprising:
providing a combinatorial library comprising a plurality of variant nucleic acids, each of the plurality of variant nucleic acids comprising a contiguous portion of at least 600 bp, wherein the contiguous portion comprises a combination of two or more variant nucleotide subsequences, wherein a first variant nucleotide subsequence of the two or more variant nucleotide subsequences defines a first end of the contiguous portion and a second variant nucleotide subsequence of the two or more variant nucleotide subsequences defines a second end of the contiguous portion opposite the first end;
introducing the library into a population of cells configured to express one or more polypeptides encoded by a member of the plurality of variant nucleic acids;
selecting a subpopulation of the population of cells based on at least one functional property dependent on the combination of the two or more variant nucleotide subsequences, wherein the subpopulation comprises a plurality of cells;
isolating a subset of the plurality of variant nucleic acids from the subpopulation;
determining nucleotide sequences of the contiguous portion of individual members of the subset; and
identifying at least one combination of the two or more variant nucleotide subsequences based on the nucleotide sequences.

32. The method of claim 31, wherein the one or more polypeptides comprises:
T cell receptor α (TCRα)- and TCRβ-chains;
a chimeric antigen receptor (CAR);
a switch receptor; or
one or more chains of an antibody or antigen binding fragment thereof.

33. The method of claim 31, wherein the first variant nucleotide subsequence encodes a TCRα variant amino acid sequence and the second variant nucleotide subsequence encodes a TCRβ variant amino acid sequence.

34. The method of claim 31, wherein the two or more variant nucleotide subsequences encode one or more of: a TCR V region, a TCR complementarity determining region 3 (CDR3), a TCR J-segment, and a TCR constant region.

35. (canceled)

36. (canceled)

37. (canceled)

38. (canceled)

39. (canceled)

40. (canceled)

41. (canceled)

42. (canceled)

43. The method of claim 31, comprising adjusting a size of the population of cells based on a number of different combinations of the two or more variant nucleotide subsequences in the library.

44. The method of claim 31, wherein the population of cells comprises immortalized T cells or primary T cells.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. The method of claim 31, wherein selecting comprises contacting the population of cells with one or more of:
a second population of cells;
a ligand for the one or more polypeptides;
an agonist or antagonist of the one or more polypeptides; and
a small molecule,
wherein a change in the subpopulation induced by the contacting depends on the at least one functional property of the one or more polypeptides.

57. (canceled)

58. The method of claim 56, wherein the second population of cells comprises antigen-presenting cells.

59. (canceled)

60. (canceled)

61. (canceled)

62. (canceled)

63. (canceled)

64. (canceled)

65. (canceled)

66. The method of claim 31, wherein identifying the at least one combination comprises measuring an enrichment of the at least one combination in the subpopulation relative to a control population of cells.

67. The method of claim 66, wherein the population of cells comprises the control population of cells.

68. The method of claim 66, wherein the subpopulation and control population of cells are non-overlapping, wherein non-overlapping denotes that the cells in both populations have a different activation status, but can carry a same variant nucleic acid.

69. (canceled)

70. (canceled)

71. The method of claim 31, wherein the isolating does not comprise isolating single clones of the subpopulation based on the at least one functional property.

72. (canceled)

73. (canceled)

74. (canceled)

75. (canceled)

76. (canceled)

77. (canceled)

78. (canceled)

79. (canceled)

80. (canceled)

81. (canceled)

82. A method of identifying nucleotide sequences encoding T cell receptor α (TCRα)- and TCRβ-chains from a combinatorial library of nucleic acids, comprising:
providing a library comprising a plurality of variant nucleic acids, each of the plurality of variant nucleic acids comprising a contiguous portion of at least 600 bp, wherein the contiguous portion comprises:
a combination of
a first variant nucleotide subsequence encoding a TCRα variant amino acid sequence and defining a first end of the contiguous portion, and
a second variant nucleotide subsequence encoding a TCRβ variant amino acid sequence and defining a second end of the contiguous portion opposite the first end;
introducing the library into a population of immortalized T cells configured to express TCRα- and TCRβ-chains encoded by a member of the plurality of variant nucleic acids;
selecting a subpopulation of the population of immortalized T cells based on an expression of a T cell activation marker above a threshold level in response to contacting the immortalized T cells with immortalized B cells expressing an antigen, wherein the subpopulation comprises a plurality of T cells;
isolating a subset of the plurality of variant nucleic acids from the subpopulation;
determining nucleotide sequences of the contiguous portion of individual members of the subset; and
identifying at least one combination of the first and second variant nucleotide subsequences based on an enrichment of the at least one combination in the nucleotide sequences of the subset relative to a control.

83. The method of claim 82, further comprising:
selecting a second subpopulation of the population of immortalized T cells based on the expression of the T cell activation marker below a second threshold level in response to contacting the immortalized T cells with the immortalized B cells, wherein the second subpopulation comprises a second plurality of T cells, and wherein the subpopulation and second subpopulation are non-overlapping;
isolating a second subset of the plurality of variant nucleic acids from the second subpopulation; and
determining second nucleotide sequences of the contiguous portion of individual members of the second subset,
wherein the at least one combination is identified based on an enrichment of the at least one combination in the subset relative to the at least one combination in the second nucleotide sequences of the second subset.

84. A method of identifying a nucleotide sequence encoding a chimeric antigen receptor (CAR) hinge domain, transmembrane domain, and/or an intracellular signaling domain from a combinatorial library of nucleic acids, comprising:
providing a library comprising a plurality of variant nucleic acids, each of the plurality of variant nucleic acids comprising a contiguous portion of at least 600 bp, wherein the contiguous portion comprises a combination of two or more of:
a first variant nucleotide subsequence encoding a CAR hinge domain;
a second variant nucleotide subsequence encoding a CAR transmembrane domain; and
a third variant nucleotide subsequence encoding a CAR intracellular signaling domain,
wherein one of the first, second or third variant nucleotide subsequences define a first end of the contiguous portion, and wherein another one of the first, second or third variant nucleotide subsequences defines a second end of the contiguous portion opposite the first end;
introducing the library into a population of cells configured to express a CAR encoded by a member of the plurality of variant nucleic acids, wherein the population of cells comprises a population of immortalized T cells or primary human T cells;
selecting a subpopulation of the population of cells based on cell proliferation above a threshold level in response to contacting the cells with antigen-presenting cells expressing an antigen specific to an antigen-binding domain of the CAR, wherein the subpopulation comprises a plurality of cells;
isolating a subset of the plurality of variant nucleic acids from the subpopulation;
determining nucleotide sequences of the contiguous portion of individual members of the subset; and
identifying at least one combination of the first, second, and third variant nucleotide subsequences based on an enrichment of the at least one combination in the nucleotide sequences of the subset relative to a control.

85. The method of claim 84, wherein there is more than one CAR intracellular signaling domain.

86. (canceled)

87. (canceled)

88. The method of claim 84, comprising:
selecting a second subpopulation of the population of cells based on cell proliferation below a second threshold level in response to contacting the cells with the antigen-presenting cells, wherein the second subpopulation comprises a second plurality of cells, and wherein the subpopulation and second subpopulation are non-overlapping;
isolating a second subset of the plurality of variant nucleic acids from the second subpopulation;
determining second nucleotide sequences of the contiguous portion of individual members of the second subset, and
wherein the at least one combination is identified based on an enrichment of the at least one combination in the subset relative to the at least one combination in the second nucleotide sequences of the second subset.

89. (canceled)

90. (canceled)

91. A method of identifying nucleotide sequences encoding antigen-specific T cell receptor α (TCRα)- and TCRβ-chain pairs from a library of nucleic acids, comprising:
introducing a library into a population of cells able to express TCRα- and TCRβ-chains encoded by a member of a plurality of variant nucleic acids,
selecting a subpopulation of the population of cells based on an expression of a marker above a threshold level in response to antigen, wherein the subpopulation comprises a plurality of cells,
isolating a subset of the plurality of variant nucleic acids from the subpopulation,
determining nucleotide sequences of the variant nucleic acids, and
identifying at least one variant nucleotide sequence based on an enrichment of the nucleotide sequences within the subset relative to a control.

92. (canceled)

93. (canceled)

94. (canceled)

95. (canceled)

96. The method of claim 82, wherein the activation marker is CD69, and wherein two cell populations are isolated, one cell population with high expression of CD69 and the other cell population with low expression of CD69.

97. (canceled)

98. (canceled)

99. (canceled)

100. A method of identifying a nucleotide sequence encoding an antigen-specific T cell receptor α (TCRα)- and TCRβ-chain pair from a library of nucleic acids, the method comprising:
introducing the nucleic acid library into a population of cells able to express TCRα- and TCRβ-chains to make a library of cells;
selecting a first population of the library of cells based on an expression of a marker above a first threshold level in response to an antigen; and
isolating a first population of variant nucleic acids from the first population of the library.

101. The method of claim 100, further comprising:
determining at least one nucleotide sequences or nucleic acid identity of the first population of variant nucleic acids; and
identifying at least one variant nucleotide sequence based on an enrichment of the nucleotide sequences within the subset relative to a control.

102. The method of claim 100, wherein the threshold level is based on at least one of:
d) recovery of a percentage of the total pool of cells based on expression of a marker; or
e) recovery of a minimal number of cells from the total pool of cells; or
f) recovery of cells retained by a magnet based on binding of a magnetic probe to at least one marker expressed in response to an antigen

103. The method of claim 91, wherein the control is a second population of cells that is below a second threshold.

104. The method of claim 91, wherein the control is one or more of:
a reference population of cells,
the combinatorial library of nucleic acids that was introduced into the population of cells
a population of cells sorted from a same population of cells as the first population based on an expression marker below a second threshold,
at least one population of cells obtained from cocultures of reporter T cells expressing the relevant TCR library with antigen presenting cells such as B cells that are not presenting any exogenous antigens,

105. The method of claim 104, wherein the control (or bottom sample) is sorted from a same population of cells as the top sample, but having low activation marker expression or wherein the bottom sample is obtained from cocultures of reporter T cells expressing the relevant TCR library, and B cells that are not engineered to express exogenous antigens.

106. The method of claim 100, further comprising adding an antigen to the population of cells.

107. The method of claim 100, wherein the isolating a first population and/or the control is achieved by at least one of a) magnetic bead enrichment, b) flow cytometry sorting, or c) both.

108. A method of identifying a nucleotide sequence encoding a T cell receptor α (TCRα)- and TCRβ-chain from a library of nucleic acids, the method comprising:
introducing the nucleic acid library into a population of cells able to express TCRα- and TCRβ-chains to make a library of cells; and
determining at least one nucleotide sequence or nucleic acid identity of the first population of variant nucleic acids based on an enrichment of the nucleotide sequence within the subset relative to a control.

109. (canceled)

110. (canceled)

111. A method of identifying a nucleotide sequence from a library of nucleic acids, comprising:
introducing the library of nucleic acids into a population of cells to form a library of cells;
contacting the library of cells with a first population of cells;
selecting a sub-population of the library of cells based on expression of at least one marker by magnetic bead enrichment; and
identifying at least one nucleotide sequence based on a statistically significant enrichment or depletion of the nucleotide sequences within the sub-population relative to a control.

112. (canceled)

113. (canceled)

114. (canceled)

115. (canceled)

116. The method of claim 91, wherein identifying or stimulating or providing antigen comprises one or more of:
a) selecting a number of antigens;
b) creating antigen-pools in which each antigen is present in exactly two antigen pools
c) evaluating reactivity of reporter cells expressing at least one T cell receptor against each of the antigen pools; and
d) determine whether the at least one T cell receptor is reactive towards any of the selected antigens by evaluating for reactivity against exactly two antigen pools

117. The method of claim 116, wherein reactivity against exactly two antigen pools is detected by pairwise enrichment analysis.

118. (canceled)

119. (canceled)

120. (canceled)

121. The method of claim 116, wherein one employs a top-bottom comparison to evaluate reactivity.

122. (canceled)

123. The method of claim 91, wherein the antigen is presented via an antigen-presenting cell.

124. The method of claim 91, wherein the library is a combinatorial library.

125. The method of claim 1, wherein the antigen is provided by a cell.

126. (canceled)

127. The method of any of the preceding methods involving a library, wherein the library is a combinatorial library.

128. (canceled)

129. A collection of cells, the collection comprising:
a set of at least two T cells, wherein each is configured to express at least one TCR alpha and TCR beta pair, wherein the TCR alpha and the TCR beta are each from a subject, wherein the T cells do not express an endogenous TCR, and wherein the set are configured for activation of one or more T cell activation markers; and
a set of at least two B cells, wherein each of the at least two B cells is configured to express at least one exogenous neo-antigen (or antigen), such that there are at least two exogenous neo-antigens (or antigens) capable of being produced, and wherein the at least two exogenous neo-antigens (or antigens) are the same as those in the subject.

130. (canceled)

131. A library of TCR expressing cells, the library of TCR expressing cells comprising: a set of at least three T cells,
wherein at least two of the T cells are configured to express at least two TCR alpha and TCR beta pairs (at least two TCR pairs),
wherein the at least two TCR pairs are from a subject,
wherein the at least three T cells do not express an endogenous TCR,
wherein the at least three T cells are configured for activation of one or more T cell activation markers, upon binding to an antigen (or neo-antigen), presented by a B cell,
wherein an amount of genomic copies of each TCR pair as reflected in a number of TCR cells is such that one gets a read on every TCR in the sample, and
wherein at least one of the TCRs is not distributed equally throughout a composition comprising the library.

132. (canceled)

133. (canceled)

134. A method of treating a subject, the method comprising:
identifying a subject having a tumor;
providing a set of at least two T cells, each of which is configured to express at least one different TCR alpha and TCR beta pair, wherein each of the TCR alpha and the TCR beta are from the subject,
providing a set of at least two B cells, wherein the set of B cells is configured to express at least two exogenous neo-antigens, and wherein the at least two exogenous neoantigens are the same as those neo-antigens found in the subject;
combining the set of at least two T cells with the set of at least two B cells and selecting a combination of at least two TCR pairs based upon activation of the at least two T cells via the at least two exogenous neo-antigens; and
administering the combination of at least two TCR pairs to the subject, thereby treating the tumor.

135. (canceled)

136. A method of treating a subject, the method comprising:
identifying a subject having a tumor;
providing a set of at least two T cells, each of which is configured to express at least one different TCR alpha and TCR beta pair, wherein each of the TCR alpha and the TCR beta are from the subject,
providing a set of at least two antigen presenting cells, wherein the set of antigen-presenting cells originates from the subject, is configured to express at least two exogenous neo-antigens, and wherein the at least two exogenous neoantigens are the same as those neo-antigens found in the subject;
combining the set of at least two T cells with the set of at least two antigen present cells and selecting a combination of at least two TCR pairs based upon activation of the at least two T cells via the at least two exogenous neo-antigens; and
administering the combination of at least two TCR pairs to the subject, thereby treating the tumor.

137. A pharmaceutical composition comprising:
a first TCR pair, that binds to a first antigen (or neo-antigen) in a subject's tumor; and
a second TCR pair, that binds to a second antigen (or neo-antigen) in the subject's tumor.

138. The pharmaceutical composition of claim 137, wherein the first TCR pair is MHC-class I restricted and wherein the second TCR pair is MHC-class II restricted.

139. A pharmaceutical composition comprising:
a first TCR pair, that binds to a first antigen and is MHC-class I restricted; and
a second TCR pair, that binds to a second antigen and is MHC-class II restricted.

140. The pharmaceutical composition of claim 137, further comprising a third TCR pair.

141. (canceled)

142. A collection of cells, the collection comprising:
a set of at least two T cells, wherein each is configured to express at least one TCR alpha and TCR beta pair, wherein the pair is from a subject, wherein the T cells do not express an endogenous TCR, and wherein the set are configured for activation of one or more T cell activation markers; and
a set of at least two antigen present cells (APCs), wherein each of the at least two APCs is configured to express at least one exogenous neo-antigen (or antigen), such that there are at least two exogenous neo-antigens (or antigens) capable of being produced, and wherein the at least two exogenous neo-antigens (or antigens) are the same as those in the subject.

143. (canceled)

144. (canceled)

145. (canceled)


SEQUENCE LISTING (4 sequences)

SEQ ID NO: 1 - protein, 819 aa, Artificial Sequence
  Synthetic; Amino acid sequence for a TCR gene expressed as a TCRbeta-P2A-TCRalpha-T2A-Puromycin resistance expression cassette
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLYPYDGRSSYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSGSGATNFSLLKQAGDVEENPGPMTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAAYNQGGKLIFGQGTELSVKPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSSGSGEGRGSLLTCGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 2 - protein, 467 aa, Artificial Sequence
  Synthetic; Amino acid sequence for a CD8alpha-P2A-CD8beta transgene
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVGSGATNFSLLKQAGDVEENPGPMRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 3 - DNA, 2405 bp, Artificial Sequence
  Synthetic; Nucleotide sequence for K562-HLA-A*0201-IRES-FusionRed
atggctgttatggcccctagaacactggtgctgctgctgtctggtgccctggctctgacacaaacatgggccggcagccacagcatgcggtactttttcaccagcgtgtccagacctggcagaggcgagcctagattcattgccgtgggctacgtggacgacacccagttcgtcagattcgattccgatgccgccagccagcggatggaacctagagcaccttggatcgagcaagagggccccgagtattgggacggcgagacaagaaaagtgaaggcccacagccagacacacagagtggatctgggaaccctgcggggctactacaatcagtctgaggccggctctcacaccgtgcagaggatgtatggctgtgacgtgggcagcgattggcggttcctgagaggctatcaccagtacgcctacgacggcaaggactatatcgccctgaaagaggacctgcggtcttggacagccgccgatatggctgcccagaccacaaagcacaagtgggaagccgctcacgtggccgaacagctgagagcttatctggaaggcacctgtgtggaatggctgcggagatacctggaaaacggcaaagagacactgcagcggacagacgcccctaagacacacatgacacaccacgccgtgtccgaccacgaagccacacttagatgttgggccctgagcttctaccccgccgagatcacactgacatggcagagagatggcgaggatcagacccaggataccgagctggtggaaacaagacctgccggcgacggcaccttccagaaatgggctgctgtggtggtgcctagcggccaagagcagagatacacctgtcacgtgcagcacgagggcctgcctaagcctcttacactgagatgggagcccagcagccagcctacaatccccatcgtgggaatcattgccggcctggtgctgtttggcgccgtgattacaggtgcagtggtggccgctgttatgtggcggagaaagagcagcgacagaaaaggcggcagctactctcaggccgccagctctgattctgcccagggctctgatgtgtctctgaccgcctgcaaagtctgagcggccgctacgtaaattccgcccccccccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgtccgagctgatcaaagaaaacatgcccatgaagctgtacatggaagggaccgtgaacaaccaccacttcaagtgcaccagcgaaggcgagggcaagccttatgagggcacccagaccatgcggatcaaggtggtggaaggtggccctctgcctttcgcctttgatatcctggccaccagctttatgtacggcagccggaccttcatcaagcaccctccaggcatccccgatttcttcaagcagagctttcccgagggcttcacctgggagagagtgaccacatacgaggatggcggcgtgctgacagccacacaggatacaagtctgcaggacggctgcctgatctacaacgtgaaagtgcggggcgtgaacttccccgccaatggacctgtgatgcagaagaaaaccctcggctgggaagccagcaccgagacaatgtatcctgccgacggtggactggaaggggcctgtgatatggccctgaagcttgttggcggcggacacctgatctgcaacctggaaaccacctacagaagcaagaagcccgccaccaacctgaagatgcccggcgtgtacaacgtggaccaccggctggaaaggatcaaagaggccgacgacgagacatacgtggaacagcacgaagtggccgtggccagatactctacaggcggagctggcgacggcggcaaatga

SEQ ID NO: 4 - protein, 755 aa, Artificial Sequence
  Synthetic; Amino acid sequence for a TCR gene expressed as a TCRbeta-P2A-TCRalpha-T2A-Blasticidin resistance expression cassette
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASRASGREQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSGSGATNFSLLKQAGDVEENPGPMKSLRVLLVILWLQLSWVWSQDQQVKQNSPSLSVQEGRISILNCDYTNSMFDYFLWYKKYPAEGPTFLISISSIKDKNEDGRFTVFLNKSAKHLSLHIVPSQPGDSAVYFCAAFLQSNDMRFGAGTRLTVKPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSSGSGEGRGSLLTCGDVEENPGPMKTFNISQQDLELVEVATEKITMLYEDNKHHVGAAIRTKTGEIISAVHIEAYIGRVTVCAEAIAIGSAVSNGQKDFDTIVAVRHPYSDEVDRSIRVVSPCGMCRELISDYAPDCFVLIEMNGKLVKTTIEELIPLKYTRN